(19) United States
(12) Patent Application Publication
(10) Pub. No.: US 2009/0175827 A1
(43) Pub. Date: Jul. 9, 2009

(54) miR-16 REGULATED GENES AND PATHWAYS AS TARGETS FOR THERAPEUTIC INTERVENTION

(76) Inventors: Mike W. Byrom, Austin, TX (US); Lubna Patrawala, Austin, TX (US); Charles D. Johnson, Austin, TX (US); David Brown, Austin, TX (US); Andreas G. Bader, Austin, TX (US)

Correspondence Address: Fullbright & Jaworski L.L.P., 600 Congress Avenue, Suite 2400, Austin, TX, 78701, US

(21) Appl. No.: 11/967,663

(22) Filed: Dec. 31, 2007

Related U.S. Application Data
(60) Provisional application No. 60/882,758, filed on Dec. 29, 2006.

(30) Foreign Application Priority Data
Dec. 10, 2007 (US) PCT/US07/87038

(51) Int. Cl. A61K 35/76; C12N 5/06; A61K 31/7088; C12Q 1/68; A61P 31/00; A61K 31/7105; A61K 31/711

(52) U.S. Cl. 424/93.2; 435/375; 514/44; 435/6

(57) ABSTRACT

The present invention concerns methods and compositions for identifying genes or genetic pathways modulated by miR-16, using miR-16 to modulate a gene or gene pathway, using this profile in assessing the condition of a patient and/or treating the patient with an appropriate miRNA.

This application claims priority to U.S. provisional application No. 60/882,758 filed Dec. 29, 2006 and PCT application PCT/U.S.07/87038, filed Dec. 10, 2007, both of which are incorporated herein by reference in their entirety.

This application is related to U.S. patent application Ser. No. 11/141,707 filed May 31, 2005 and Ser. No. 11/273,640 filed Nov. 14, 2005, each of which is incorporated herein by reference in their entirety.

### BACKGROUND OF THE INVENTION

I. Field of the Invention

The present invention relates to the fields of molecular biology and medicine. More specifically, the invention relates to methods and compositions for the treatment of diseases or conditions that are affected by miR-16 microRNAs, microRNA expression, and genes and cellular pathways directly and indirectly modulated by such.

### II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are distinct.

miRNAs thus far observed have been approximately 21-22 nucleotides in length and they arise from longer precursors, which are transcribed from non-protein-encoding genes. See review of Carrington et al (2003). The precursors form structures that fold back on themselves in self-complementary regions; they are then processed by the nuclease Dicer in animals or DCL1 in plants. miRNA molecules interrupt translation through precise or imprecise base-pairing with their targets.

Many miRNAs are conserved among diverse organisms, and this has led to the suggestion that miRNAs are involved in essential biological processes throughout the life span of an organism (Esquela-Kerscher and Slack, 2006). In particular, miRNAs have been implicated in regulating cell growth, and cell and tissue differentiation; cellular processes that are associated with the development of cancer. For instance, lin-4 and miR-16 both regulate passage from one larval state to another during C. elegans development (Ambros, 2001). mir-14 and bantam are Drosophila miRNAs that regulate cell death, apparently by regulating the expression of genes involved in apoptosis (Brennecke et al., 2003, Xu et al., 2003).

Research on miRNAs is increasing as scientists are beginning to appreciate the broad role that these molecules play in the regulation of eukaryotic gene expression. In particular, several recent studies have shown that expression levels of numerous miRNAs are associated with various cancers (reviewed in Esquela-Kerscher and Slack, 2006). Reduced expression of two miRNAs correlates strongly with chronic lymphocytic leukemia in humans, providing a possible link between miRNAs and cancer (Calin et al, 2002). Others have evaluated the expression patterns of large numbers of miRNAs in multiple human cancers and observed differential expression of almost all miRNAs across numerous cancer types (Lu et al., 2005). Most studies link miRNAs to cancer only by indirect evidence. However, He et al. (2005) has provided more direct evidence that miRNAs may contribute directly to causing cancer by forcing the over-expression of six miRNAs in mice that resulted in a significant increase in B cell lymphomas.

Others have shown that miR-16 is down-regulated in B-cells from patients with chronic lymphocytic leukemia (Calin et al., 2002). Reduced expression of these miRNAs in B cell lymphomas results in overexpression of a miR-16 target gene, BCL2, and subsequent inhibition of apoptosis by the BCL2 gene product. Reduced expression of miR-16 results in uncontrolled cellular proliferation and B cell malignancy (reviewed in Calin and Croce, 2006). Together these data suggest that miR-16-1 appears to function as a tumor suppressor in human B cells.

The inventors previously demonstrated that hsa-miR-16 is involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005). Expression of miR-16 was reduced in lung tumors from numerous lung cancer patients when compared to its expression in normal adjacent lung tissues from the same patients. The inventors observed increased expression of miR-16 in breast and prostate tumors as compared to expression in adjacent normal cells from the same cancer patients. In human foreskin fibroblasts, hsa-miR-16 activated the hTert gene that encodes the catalytic domain of telomerase. Over 90% of human cancer samples have active telomerase (reviewed in Dong et al., 2005). Hsa-miR-16 also induces cells to enter the S phase of the cell cycle and decreases the proliferation of lung cancer cells (A549 and HTB-57 lung carcinoma cells), prostate cancer cells (22Rv1), and human basal cell carcinomas (TE354T). Anti-miR inhibitors of hsa-miR-16 increased the proliferation of non-malignant human breast epithelial cells and basal cell carcinoma cells (TE354T). In addition, the inventors previously observed that hsa-miR-16 is up-regulated in patients with prion disease and Alzheimer's disease when compared to patients without those diseases. As is the case for cancer therapy, genes and pathways that are altered by expression of hsa-miR-16 represent targets for therapeutic intervention in the treatment of certain diseases like Alzheimer's Disease and prion diseases, in which hsa-miR-16 likely plays a role. In animals, most miRNAs are thought to interact with target genes through imprecise base pairing within the 3′ untranslated regions of their gene targets. Regulation of target genes by miRNAs is thought to occur primarily by translation inhibition, but mRNA instability may also be a mechanism (Reinhart et al., 2000; Bagga et al., 2005). Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict miRNA targets with bioinformatics tools alone. Furthermore, the complicated interactive regulatory networks among miRNAs and target genes make it difficult to accurately predict which genes will actually be mis-regulated in response to a given miRNA.

Correcting gene expression errors by manipulating miRNA expression or by repairing miRNA mis-regulation represent promising methods to repair genetic disorders and cure diseases like cancer. A current, disabling limitation of this approach is that, as mentioned above, the details of the regulatory pathways and networks that are affected by any given miRNA remain largely unknown. Besides BCL2, the genes, gene pathways, and gene networks that are regulated by miR-16 in cancerous cells remain largely unknown. Currently, this represents a significant limitation for treatment of cancers in which miR-16 may play a role. A need exists to identify the genes, genetic pathways, and genetic networks that are regulated by or that may regulate hsa-miR-16 expression.

### SUMMARY OF THE INVENTION

The present invention provides additional compositions and methods to address problems in the art by identifying genes in cancer cells that are direct targets for hsa-miR-16 regulation or that are downstream targets of regulation following the hsa-miR-16-mediated modification of upstream gene expression. Furthermore, the invention describes gene, disease, and/or physiologic pathways and networks that are influenced by hsa-miR-16. Many of these genes and pathways are associated with various cancers and other diseases. The altered expression of miR-16 in cells would lead to changes in the expression of these key genes and contribute to the development of disease. Introducing miR-16 (for diseases where the miRNA is down-regulated) or a miR-16 inhibitor (for diseases where the miRNA is up-regulated) into disease cells or tissues would result in a therapeutic response. The identities of key genes that are regulated directly or indirectly by miR-16 and the disease with which they are associated are provided herein. In certain aspects a cell may be an epithelial, stromal, or mucosal cell. The cell can be, but is not limited to brain, a glial, a neuronal, a blood, an esophageal, a lung, a cardiovascular, a liver, a breast, a bone, a thyroid, a glandular, an adrenal, a pancreatic, a stomach, an intestinal, a kidney, a bladder, a prostate, a cervical, a uterus, an ovarian, a testicular, a splenic, a skin, a smooth muscle, a cardiac muscle, or a striated muscle cell. In certain aspects, the cell, tissue, or target may not be defective in miRNA expression yet may still respond therapeutically to expression or over expression of a miRNA. miR-16 could be used as a therapeutic target for any of these diseases. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in miRNA expression, or biologic and/or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioma, glioblastoma, gastric carcinoma, hepatoblastoma, hepatocellular carcinoma, Hodgkin lymphoma, laryngeal squamous cell carcinoma, lung carcinoma, melanoma, medulloblastoma, mantle cell lymphoma, myxofibrosarcoma, myeloid leukemia, multiple myeloma, neurofibroma, non-small cell lung carcinoma, ovarian carcinoma, esophageal carcinoma, pancreatic carcinoma, prostate carcinoma, pheochromocytoma, renal cell carcinoma, rhabdomyosarcoma, squamous cell carcinoma of the head and neck, testicular tumor or thyroid carcinoma wherein the modulation of one or more gene is sufficient for a therapeutic response. Typically a cancerous condition is an aberrant hyperproliferative condition associated with the uncontrolled growth or inability to undergo cell death, including apoptosis.

In certain aspect, the cancerous condition is prostate carcinoma, which can be positive or negative for PSA, and/or androgen dependent or androgen independent. Cells of the prostate require male hormones, known as androgens, to work properly. Androgens include testosterone, which is made in the testes; dehydroepiandrosterone, made in the adrenal glands; and dihydrotestosterone, which is converted from testosterone within the prostate itself. Some prostate carcinomas retain androgen dependence while others are independent of androgen. Prostate cancer screening is an attempt to find unsuspected cancers. Screening tests may lead to more specific follow-up tests such as a biopsy, where small pieces of the prostate are removed for closer study. Typical prostate cancer screening options include the digital rectal exam and the prostate specific antigen (PSA) blood test. Prostate cancer is usually a slow-growing cancer, very common among older men.

A cell, tissue, or subject may be a cancer cell, a cancerous tissue, harbor cancerous tissue, or be a subject or patient diagnosed or at risk of developing a disease or condition. In certain aspects a cancer cell is a neuronal, glial, lung, liver, brain, breast, bladder, blood, leukemic, colon, endometrial, stomach, skin, ovarian, fat, bone, cervical, esophageal, pancreatic, prostate, kidney, testicular or thyroid cell. In still a further aspect cancer includes, but is not limited to astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioma, glioblastoma, gastric carcinoma, hepatoblastoma, hepatocellular carcinoma, Hodgkin lymphoma, laryngeal squamous cell carcinoma, lung carcinoma, melanoma, medulloblastoma, mantle cell lymphoma, myxofibrosarcoma, myeloid leukemia, multiple myeloma, neurofibroma, non-small cell lung carcinoma, ovarian carcinoma, esophageal carcinoma, pancreatic carcinoma, prostate carcinoma, pheochromocytoma, renal cell carcinoma, rhabdomyosarcoma, squamous cell carcinoma of the head and neck, testicular tumor or thyroid carcinoma.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or any combination of genes identified in Table 1, 2, 4 and 5. In certain aspects the expression of a gene is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or all of genes identified in Table 1, 2, 4 and 5, in various combinations and permutations. In particular embodiments, the invention may exclude or choose not to include 1, 2, 3, 4, 5, 6, 7, 8, 9, 10, 11, 12, 13, 14, 15, 20, 25, 30, 35, 40, 45, 50, 100, 150, 200 or more genes or any combination of genes identified in Table 1, 2, 4 and 5, e.g., BCL2, RARS (arginyl-tRNA synthetase), BTG2, WT1, PPM1D, PAK7, and/or RAB9B. In one particular aspect the gene modulated or selected to modulate includes one or more genes of Table 1, 2, 4 and/or 5 provided that RARS (arginyl-tRNA synthetase), BTG2, WT1, PPM1D, PAK7, and/or RAB9B is not included.

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-16 nucleic acid, mimetic, or inhibitor sequence in an amount sufficient to modulate the expression of a gene positively or negatively modulated by a miR-16 miRNA. A “miR-16 nucleic acid sequence” or “miR-16 inhibitor” includes the full length precursor of miR-16, or complement thereof or processed (i.e., mature) sequence of miR-16 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-16 nucleic acid sequence or miR-16 inhibitor contains the full-length processed miRNA sequence or complement thereof and is referred to as the “miR-16 full-length processed nucleic acid sequence” or “miR-16 full-length processed inhibitor sequence.” In still further aspects, the miR-16 nucleic acid comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-16 that is at least 75, 80, 85, 90, 95, 98, 99 or 100% identical to SEQ ID NOs provided herein. The general term miR-16 includes all members of the miR-16 family that share at least part of a mature miR-16 sequence. In still further aspects, the miR-16 nucleic acid comprises at least one 5, 6, 7, 8, 9, 10, 11, 12, 13, 14, 15, 16, 17, 18, 19, 20, 21, 22, 232, 24, 25, 50 nucleotide (including all ranges and integers there between) segment of miR-16 that is at least 75, 80, 85, 90, 95, 98, 99 or 100% identical to SEQ ID NOs:1-3. SEQ ID NO:1 uagcagcacguaaauauuggcg (accession-MIMAT0000069), SEQ ID NO:2 (hsa-mir-16-1, accession-M10000070) gucagcagugccuuagcagcacguaaauauuggcguuaagauucuaaaauuau cuccaguauuaacugugcugcugaaguaagguugac; SEQ ID NO:3 (hsa-mir-16-2, accession MI0000115) guuccacucuagcagcacguaaauauuggcguagugaaauauauauuaaacaccaauauuacug ugcugcuuuagugugac). In certain embodiments the gene modulated or selected to modulate is from Table 1. In further embodiments the gene modulated or selected to modulate is from Table 2. In still further embodiments the gene modulated or selected to modulate is from Table 4. In yet further embodiments the gene modulated or selected to modulate is from Table 5. Embodiments of the invention may also include obtaining or assessing a gene expression profile or miRNA profile of a target cell prior to selecting the mode of treatment, e.g., administration of a miR-16 nucleic acid.

In certain aspects, a miR-16 nucleic acid, or a segment or a mimetic thereof, will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-16 nucleic acid sequence contains the full-length processed miRNA sequence and is referred to as the “miR-16 full-length processed nucleic acid sequence.” In still further aspects, a miR-16 comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-16 that is at least 75, 80, 85, 90, 95, 98, 99 or 100% identical to SEQ ID NOs provided herein.

In specific embodiments, a miR-16 or miR-16 inhibitor containing nucleic acid is a hsa-miR-16 or hsa-miR-16 inhibitor, or a variation thereof. In a further aspect, a miR-16 nucleic acid or miR-16 inhibitor can be administered with 1, 2, 3, 4, 5, 6, 7, 8, 9, 10 or more miRNAs or miRNA inhibitors. miRNAs or their complements can be administered concurrently, sequentially, or in an ordered progression. In certain aspects, a miR-16 or miR-16 inhibitor can be administered in combination with one or more of let-7, miR-15, miR-126, miR-20, miR-21, miR-26a, miR-34a, miR-143, miR-147, miR-188, miR-200, miR-215, miR-216, miR-292-3p, and/or miR-331. All or combinations of miRNAs or inhibitors thereof may be administered in a single formulation. Administration may be before, during or after a second therapy. miR-16 nucleic acids or complement thereof may also include various heterologous nucleic acid sequences, i.e., those sequences not typically found operatively coupled with miR-16 in nature, such as promoters, enhancers, and the like. The miR-16 nucleic acid is a recombinant nucleic acid, and can be a ribonucleic acid or a deoxyribonucleic acid. The recombinant nucleic acid may comprise a miR-16 or miR-16 inhibitor expression cassette, i.e., a nucleic acid segment that expresses a nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In a particular aspect, the miR-16 nucleic acid is a synthetic nucleic acid. Moreover, nucleic acids of the invention may be fully or partially synthetic. In certain aspects, viral vectors can be administered at 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-16 nucleic acid or miR-16 inhibitor is a synthetic nucleic acid. Moreover, nucleic acids of the invention may be fully or partially synthetic. In still further aspects, a nucleic acid of the invention or a DNA encoding such a nucleic acid of the invention can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

A cell, tissue, or subject may be or suffer from an abnormal or pathologic condition, or in the case of a cell or tissue, the component of a pathological condition. In certain aspects, a cell, tissue, or subject is a cancer cell, a cancerous tissue or harbor cancerous tissue, or a cancer patient. In a particular aspect the cancer is neuronal, glial, lung, liver, brain, breast, bladder, blood, leukemic, cervical, testicular, colon, endometrial, stomach, skin, ovarian, esophageal, pancreatic, prostate, kidney, or thyroid cancer. The database content related to all nucleic acids and genes designated by an accession number or a database submission are incorporated herein by reference as of the filing date of this application.

A further embodiment of the invention is directed to methods of modulating a cellular pathway comprising administering to the cell an amount of an isolated nucleic acid comprising a miR-16 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene. Modulation of a gene can include inhibiting the function of an endogenous miRNA or providing a functional miRNA to a cell, tissue, or subject. Modulation refers to the expression levels or activities of a gene or its related gene product or protein, e.g., the mRNA levels may be modulated or the translation of an mRNA may be modulated, etc. Modulation may increase or up regulate a gene or gene product or it may decrease or down regulate a gene or gene product.

Still a further embodiment includes methods of treating a patient with a pathological condition comprising one or more of step (a) administering to the patient an amount of an isolated nucleic acid comprising a miR-16 nucleic acid sequence in an amount sufficient to modulate the expression of a cellular pathway; and (b) administering a second therapy, wherein the modulation of the cellular pathway sensitizes the patient to the second therapy. A cellular pathway may include, but is not limited to one or more pathway described in Table 2 below or a pathway that is known to include one or more gene of Table 1, 3, 4, and/or 5. A second therapy can include a second miRNA or other nucleic acid therapy or one or more standard therapies, such as chemotherapy, drug therapy, radiation therapy, immunotherapy, thermal therapy, and the like.

Embodiments of the invention include methods of treating a subject with a pathological condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using selected therapy. Typically, the pathological condition will have as a component, indicator, or result the mis-regulation of one or more gene of Table 1, 3, 4, and/or 5.

Further embodiments include the identification and assessment of an expression profile indicative of miR-16 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers from Table 1, 3, 4, and/or 5); it is contemplated that the nucleic acid profile can be obtained using a set of RNAs, using for example nucleic acid amplification or hybridization techniques well known to one of ordinary skill in the art. The difference in the expression profile in the sample from the patient and a reference expression profile, such as an expression profile from a normal or non-pathologic sample, is indicative of a pathologic, disease, or cancerous condition. A nucleic acid or probe set comprising or identifying a segment of a corresponding mRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more, including any integer or range derivable there between, of a gene or genetic marker, or a nucleic acid, mRNA or a probe representative thereof that is listed in Table 1, 3, 4, and/or 5, or identified by the methods described herein.

Certain embodiments of the invention are directed to compositions and methods for assessing, prognosing, or treating a pathological condition in a patient comprising measuring or determining an expression profile of one or more marker(s) in a sample from the patient, wherein a difference in the expression profile in the sample from the patient and an expression profile of a normal sample or reference expression profile is indicative of pathological condition and particularly cancer. In certain aspects of the invention, the cellular pathway, gene, or genetic marker is or is representative of one or more pathway or marker described in Table 1, 3, 4, and/or 5, including any combination thereof and excluding 0, 1, 2, 3, 4, 5, 6, 7, 8, 9, 10 or more genes.

Aspects of the invention include treating, diagnosing, or prognosing a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

Certain embodiments of the invention include determining expression of one or more marker, gene, or nucleic acid representative thereof, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, mass spectrometry or other methods known to those of ordinary skill in the art.

A further embodiment of the invention is directed to methods of modulating a cellular pathway comprising administering to the cell an amount of an isolated nucleic acid comprising a miR-16 nucleic acid sequence or a miR-16 inhibitor. A cell, tissue, or subject may be a cancer cell, a cancerous tissue or harbor cancerous tissue, or a cancer patient. The database content related to all nucleic acids and genes designated by an accession number or a database submission are incorporated herein by reference as of the filing date of this application.

A further embodiment of the invention is directed to methods of modulating a cellular pathway comprising administering to the cell an amount of an isolated nucleic acid comprising a miR-16 nucleic acid sequence in an amount sufficient to modulate the expression, function, status, or state of a cellular pathway, in particular those pathways described or the pathways known to include one or more genes described herein. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-16 nucleic acid sequence or a miR-16 inhibitor in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described herein or a pathway that is know to include one or more genes in the tables herein. The second therapy may be administered before, during, and/or after the isolated nucleic acid or miRNA or inhibitor is administered

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifamib, bevacizumab, sirolimus, temsirolimus, everolimus, lonafamib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from the tables; (b) assessing the sensitivity of the subject to therapy based on the expression profile; (c) selecting a therapy based on the assessed sensitivity; and (d) treating the subject using a selected therapy. Typically, the disease or condition will have as a component, indicator, or resulting mis-regulation of one or more gene described herein.

In certain aspects, 2, 3, 4, 5, 6, 7, 8, 9, 10, or more miRNA may be used in sequence or in combination. For instance, any combination of miR-16 or a miR-16 inhibitor with another miRNA. Further embodiments include the identification and assessment of an expression profile indicative of miR-16 status in a cell or tissue comprising expression assessment of one or more gene from the tables, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker or miRNA in the sample (e.g., a plurality of nucleic acid probes that identify one or more markers from the tables; it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile of one or more genes or miRNAs, are indicative of which miRNAs to be administered.

In certain aspects, miR-16 or miR-16 inhibitor and let-7 or let-7 inhibitor are administered to patients with astrocytoma, breast carcinoma, bladder carcinoma, cervical carcinoma, chronic lymphoblastic leukemia, colorectal carcinoma, endometrial carcinoma, glioblastoma, gastric carcinoma, hepatoblastoma, hepatocellular carcinoma, Hodgkin lymphoma, lung carcinoma, melanoma, medulloblastoma, myxofibrosarcoma, myeloid leukemia, multiple myeloma, non-small cell lung carcinoma, ovarian carcinoma, oesophageal carcinoma, pancreatic carcinoma, prostate carcinoma, renal cell carcinoma, rhabdomyosarcoma, squamous cell carcinoma of the head and neck, thyroid carcinoma.

Further aspects include administering miR-16 or miR-16 inhibitor and miR-10 or miR-10 inhibitor to patients with astrocytoma, breast carcinoma, bladder carcinoma, cervical carcinoma, chronic lymphoblastic leukemia, colorectal carcinoma, endometrial carcinoma, glioblastoma, gastric carcinoma, hepatoblastoma, hepatocellular carcinoma, Hodgkin lymphoma, lung carcinoma, melanoma, mantle cell lymphoma, multiple myeloma, non-small cell lung carcinoma, ovarian carcinoma, oesophageal carcinoma, pancreatic carcinoma, prostate carcinoma, renal cell carcinoma, squamous cell carcinoma of the head and neck, thyroid carcinoma

In yet another aspect, miR-16 or miR-16 inhibitor and miR-15 or miR-15 inhibitor can be administered to patients with astrocytoma, breast carcinoma, B-cell lymphoma, bladder carcinoma, cervical carcinoma, colorectal carcinoma, endometrial carcinoma, glioblastoma, gastric carcinoma, hepatoblastoma, hepatocellular carcinoma, Hodgkin lymphoma, lung carcinoma, laryngeal squamous cell carcinoma, melanoma, medulloblastoma, mantle cell lymphoma, myxofibrosarcoma, myeloid leukemia, multiple myeloma, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, thyroid carcinoma.

In still further aspects, miR-16 or miR-16 inhibitor and miR-20 or miR-20 inhibitor are administered to patients with astrocytoma, breast carcinoma, bladder carcinoma, cervical carcinoma, colorectal carcinoma, endometrial carcinoma, glioblastoma, gastric carcinoma, hepatocellular carcinoma, Hodgkin lymphoma, melanoma, mantle cell lymphoma, myxofibrosarcoma, multiple myeloma, non-small cell lung carcinoma, ovarian carcinoma, oesophageal carcinoma, pancreatic carcinoma, prostate carcinoma, squamous cell carcinoma of the head and neck, thyroid carcinoma.

In certain aspects, miR-16 or miR-16 inhibitor and miR-21 or miR-21 inhibitor are administered to patients with astrocytoma, breast carcinoma, bladder carcinoma, colorectal carcinoma, endometrial carcinoma, glioblastoma, gastric carcinoma, hepatocellular carcinoma, melanoma, mantle cell lymphoma, myeloid leukemia, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck.

Aspects of the invention include methods where miR-16 or miR-16 inhibitor and miR-26 or miR-26 inhibitor are administered to patients with anaplastic large cell lymphoma, breast carcinoma, B-cell lymphoma, bladder carcinoma, cervical carcinoma, chronic lymphoblastic leukemia, colorectal carcinoma, glioblastoma, gastric carcinoma, hepatocellular carcinoma, lung carcinoma, melanoma, multiple myeloma, non-small cell lung carcinoma, ovarian carcinoma, oesophageal carcinoma, pancreatic carcinoma, prostate carcinoma, renal cell carcinoma, rhabdomyosarcoma, testicular tumor.

In still further aspects, miR-16 or miR-16 inhibitor and miR-34 or miR-34 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatoblastoma, hepatocellular carcinoma, Hodgkin lymphoma, lung carcinoma, laryngeal squamous cell carcinoma, melanoma, medulloblastoma, mantle cell lymphoma, myeloid leukemia, multiple myeloma, neurofibroma, non-small cell lung carcinoma, ovarian carcinoma, oesophageal carcinoma, pancreatic carcinoma, prostate carcinoma, pheochromocytoma, rhabdomyosarcoma, squamous cell carcinoma of the head and neck, thyroid carcinoma, testicular tumor.

In still a further aspect, miR-16 or miR-16 inhibitor and miR-124 or miR-124 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatoblastoma, hepatocellular carcinoma, Hodgkin lymphoma, lung carcinoma, laryngeal squamous cell carcinoma, melanoma, medulloblastoma, mantle cell lymphoma, myxofibrosarcoma, multiple myeloma, non-small cell lung carcinoma, ovarian carcinoma, oesophageal carcinoma, pancreatic carcinoma, prostate carcinoma, renal cell carcinoma, rhabdomyosarcoma, squamous cell carcinoma of the head and neck, thyroid carcinoma, testicular tumor.

In yet further aspects, miR-16 or miR-16 inhibitor and miR-126 or miR-126 inhibitor are administered to patients with astrocytoma, breast carcinoma, bladder carcinoma, cervical carcinoma, colorectal carcinoma, endometrial carcinoma, glioblastoma, gastric carcinoma, hepatoblastoma, hepatocellular carcinoma, Hodgkin lymphoma, lung carcinoma, melanoma, mantle cell lymphoma, myeloid leukemia, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, thyroid carcinoma.

In yet further aspects, miR-16 or miR-16 inhibitor and miR-143 or miR-143 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatocellular carcinoma, Hodgkin lymphoma, lung carcinoma, melanoma, medulloblastoma, mantle cell lymphoma, multiple myeloma, non-small cell lung carcinoma, ovarian carcinoma, oesophageal carcinoma, pancreatic carcinoma, prostate carcinoma, renal cell carcinoma, squamous cell carcinoma of the head and neck, thyroid carcinoma, testicular tumor.

In a further aspect, miR-16 or miR-16 inhibitor and miR-147 or miR-147 inhibitor are administered to patients with astrocytoma, breast carcinoma, bladder carcinoma, cervical carcinoma, colorectal carcinoma, endometrial carcinoma, glioblastoma, gastric carcinoma, hepatocellular carcinoma, Hodgkin lymphoma, melanoma, mantle cell lymphoma, myxofibrosarcoma, multiple myeloma, non-small cell lung carcinoma, ovarian carcinoma, oesophageal carcinoma, pancreatic carcinoma, prostate carcinoma, renal cell carcinoma, squamous cell carcinoma of the head and neck, thyroid carcinoma.

In still a further aspect, miR-16 or miR-16 inhibitor and miR-188 or miR-188 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatocellular carcinoma, lung carcinoma, melanoma, multiple myeloma, non-small cell lung carcinoma, ovarian carcinoma, oesophageal carcinoma, pancreatic carcinoma, prostate carcinoma, renal cell carcinoma, squamous cell carcinoma of the head and neck, thyroid carcinoma, testicular tumor.

In a further aspect, miR-16 or miR-16 inhibitor and miR-200 or miR-200 inhibitor are administered to patients with anaplastic large cell lymphoma, breast carcinoma, B-cell lymphoma, cervical carcinoma, chronic lymphoblastic leukemia, colorectal carcinoma, glioblastoma, gastric carcinoma, hepatocellular carcinoma, lung carcinoma, multiple myeloma, non-small cell lung carcinoma, ovarian carcinoma, oesophageal carcinoma, pancreatic carcinoma, prostate carcinoma, rhabdomyosarcoma, squamous cell carcinoma of the head and neck, thyroid carcinoma, testicular tumor.

In yet another aspect, miR-16 or miR-16 inhibitor and miR-215 or miR-215 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatoblastoma, hepatocellular carcinoma, Hodgkin lymphoma, lung carcinoma, melanoma, mantle cell lymphoma, myxofibrosarcoma, myeloid leukemia, multiple myeloma, neurofibroma, non-small cell lung carcinoma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, thyroid carcinoma, testicular tumor.

In yet a further aspect, miR-16 or miR-16 inhibitor and miR-216 or miR-216 inhibitor are administered to patients with astrocytoma, breast carcinoma, cervical carcinoma, colorectal carcinoma, endometrial carcinoma, glioblastoma, gastric carcinoma, hepatocellular carcinoma, Hodgkin lymphoma, lung carcinoma, myeloid leukemia, neurofibroma, non-small cell lung carcinoma, ovarian carcinoma, oesophageal carcinoma, prostate carcinoma, pheochromocytoma, squamous cell carcinoma of the head and neck, testicular tumor.

In other aspects, miR-16 or miR-16 inhibitor and miR-292-3p or miR-292-3p inhibitor are administered to patients with astrocytoma, anaplastic large cell lymphoma, breast carcinoma, B-cell lymphoma, bladder carcinoma, cervical carcinoma, colorectal carcinoma, endometrial carcinoma, glioblastoma, gastric carcinoma, hepatoblastoma, hepatocellular carcinoma, lung carcinoma, laryngeal squamous cell carcinoma, melanoma, myxofibrosarcoma, multiple myeloma, non-small cell lung carcinoma, ovarian carcinoma, oesophageal carcinoma, pancreatic carcinoma, prostate carcinoma, renal cell carcinoma, rhabdomyosarcoma, squamous cell carcinoma of the head and neck, thyroid carcinoma, testicular tumor.

In certain aspects, miR-16 or miR-16 inhibitor and miR-331 or miR-331 inhibitor are administered to patients with astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioblastoma, gastric carcinoma, hepatocellular carcinoma, lung carcinoma, laryngeal squamous cell carcinoma, melanoma, myxofibrosarcoma, myeloid leukemia, multiple myeloma, neurofibroma, ovarian carcinoma, oesophageal carcinoma, pancreatic carcinoma, prostate carcinoma, pheochromocytoma, renal cell carcinoma, rhabdomyosarcoma, squamous cell carcinoma of the head and neck, thyroid carcinoma, testicular tumor.

It is contemplated that when miR-16 or a miR-16 inhibitor is given in combination with one or more other miRNA molecules, the two different miRNAs or inhibitors may be given at the same time or sequentially. In some embodiments, therapy proceeds with one miRNA or inhibitor and that therapy is followed up with therapy with the other miRNA or inhibitor 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-16 status in a cell or tissue comprising expression assessment of one or more gene from the tables herein, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself or a mimetic thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from the tables); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) developing such a condition(s). Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in tables or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in the tables, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of the tables, including any combination thereof.

The present invention also concerns kits containing compositions of the invention or compositions to implement methods of the invention. In some embodiments, kits can be used to evaluate one or more marker molecules, and/or express one or more miRNA or miRNA inhibitor. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA or miRNA inhibitor to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in the tables.

It is contemplated that any method or composition described herein can be implemented with respect to any other method or composition described herein and that different embodiments may be combined. It is specifically contemplated that any methods and compositions discussed herein with respect to miRNA molecules, miRNA, genes and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments thereof), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the sequence or mature sequence of the specified miRNA, mRNA, gene, or representative nucleic acid.

It will be further understood that shorthand notations are employed such that a generic description of a gene or marker thereof, or of a miRNA refers to any of its gene family members (distinguished by a number) or representative fragments thereof, unless otherwise indicated. It is understood by those of skill in the art that a “gene family” refers to a group of genes having the same or similar coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Thus, “let-7,” for example, refers to let-7a, let-7b, let-7c, etc. Exceptions to this shorthand notation will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Percent (%) proliferation of hsa-miR-16 treated cells relative to cells treated with negative control miRNA (100%). Cell lines used include the prostate cancer cell lines PPC-1, Dul45 and RWPE2. Abbreviations: miR-16, hsa-miR-16; NC, negative control miRNA; siEg5, siRNA against the motor protein kinesin 11 (Eg5). Standard deviations are indicated in the graph.

FIG. 2. Equal number of cells were electroporated with 1.6 μM hsa-miR-16 or negative control miRNA (NC) and grown in standard growth media (day 0). Cells were repeatedly electroporated on days 4 and 11. At each electroporation event, fifty-thousand cells were plated separately in multiple wells of a 6-well plate, and cells were harvested and counted every other day. The population doubling was calculated using the formula PD=ln(N_{f}/N₀)/ln 2, and cell numbers were extrapolated and plotted on a linear scale. Electroporation events are indicated by arrowheads. The graph shows one representative experiment.

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications. In particular, the present invention is directed to those methods and compositions related to assessing and/or identifying pathological conditions directly or indirectly related to miR-16 expression or the aberrant expression thereof. The mature sequence of miR-16 is typically comprised of uagcagcacguaaauauuggcg SEQ ID NO:1 (MIMAT0000069).

In certain aspects, the invention is directed to methods for the assessment, analysis, and/or therapy of a cell or subject where certain genes have a reduced expression (relative to normal) as a result of an increased or decreased expression of miR-16 and/or genes with an increased expression (relative to normal) as a result of an increased or decreased expression of miR-16. The expression profile and/or response to miR-16 expression or lack of expression are indicative of an individual with a pathological condition, e.g., cancer.

Prognostic assays featuring any one or combination of the miRNAs listed or the markers listed (including nucleic acids representative thereof) could be used to assess a patient to determine what if any treatment regimen is justified. As with the diagnostic assays mentioned above, the absolute values that define low expression will depend on the platform used to measure the miRNA(s). The same methods described for the diagnostic assays could be used for a prognostic assays.

### I. THERAPEUTIC METHODS

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges range derivable there between. The nucleic acid molecules are typically synthetic. The term “synthetic” refers to a nucleic acid molecule that is isolated and not produced naturally in a cell. In certain aspects the sequence (the entire sequence) and/or chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification, a 2° F. modification, a 2′H modification, a 2′amino modification, a 4′thioribose modification or a phosphorothioate modification on the carboxy group linked to the carbon at position 6′. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor (which may be described generally herein as an miRNA, so that a description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s).

In certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that the methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

In some embodiments, there is a method for reducing or inhibiting cell proliferation comprising introducing into or providing to the cell an effective amount of (i) a miRNA inhibitor molecule or (ii) a synthetic or nonsynthetic miRNA molecule that corresponds to a miRNA sequence. In certain embodiments the methods involves introducing into the cell an effective amount of (i) an miRNA inhibitor molecule having a 5′ to 3′ sequence that is at least 90% complementary to the 5′ to 3′ sequence of one or more mature miRNA.

Certain embodiments of the invention include methods of treating a pathologic condition, in particular cancer, e.g., lung or liver cancer. In one aspect, the method comprises contacting a target cell with one or more nucleic acid, synthetic miRNA, or miRNA comprising at least one nucleic acid segment having all or a portion of a miRNA sequence. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a cancer cell.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, a synthetic miRNA or a nonsynthetic miRNA is provided a synthetic miRNA or a nonsynthetic miRNA, such as one that becomes processed into a mature and active miRNA once it has access to the cell's miRNA processing machinery. In certain embodiments, it is specifically contemplated that the miRNA molecule provided to the biological matter is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, cisplatin (CDDP), carboplatin, cetuximab, chlorambucil, cisplatin (CDDP), EGFR inhibitors (gefitinib and cetuximab), procarbazine, mechlorethamine, cyclophosphamide, camptothecin, COX-2 inhibitors (e.g., celecoxib), cyclophosphamide, cytarabine,) ifosfamide, melphalan, chlorambucil, busulfan, nitrosurea, dactinomycin, dasatinib, daunorubicin, dexamethasone, docetaxel, doxorubicin (adriamycin), EGFR inhibitors (gefitinib and cetuximab), erlotinib, estrogen receptor binding agents, bleomycin, plicomycin, mitomycin, etoposide (VP16), everolimus, tamoxifen, raloxifene, estrogen receptor binding agents, taxol, taxotere, gemcitabien, navelbine, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, transplatinum, 5-fluorouracil, vincristin, vinblastin and methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifamib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. For example, inhibitors of miRNA molecules that increase cell proliferation can be provided to cells to increase proliferation or inhibitors of such molecules can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk) ERK, activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### II. PHARMACEUTICAL FORMULATIONS AND DELIVERY

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve a miRNA, or expression construct encoding such. These miRNA compositions can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy, or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, and chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). A non-limiting list of several known anti-cancer immunotherapeutic agents and their targets includes, but is not limited to (Generic Name (Target)) Cetuximab (EGFR), Panitumumab (EGFR), Trastuzumab (erbB2 receptor), Bevacizumab (VEGF), Alemtuzumab (CD52), Gemtuzumab ozogamicin (CD33), Rituximab (CD20), Tositumomab (CD20), Matuzumab (EGFR), Ibritumomab tiuxetan (CD20), Tositumomab (CD20), HuPAM4 (MUC1), MORAb-009 (Mesothelin), G250 (carbonic anhydrase IX), mAb 8H9 (8H9 antigen), M195 (CD33), Ipilimumab (CTLA4), HuLuc63 (CS1), Alemtuzumab (CD53), Epratuzumab (CD22), BC8 (CD45), HuJ591 (Prostate specific membrane antigen), hA20 (CD20), Lexatumumab (TRAIL receptor-2), Pertuzumab (HER-2 receptor), Mik-beta-1 (IL-2R), RAV12 (RAAG12), SGN-30 (CD30), AME-133v (CD20), HeFi-1 (CD30), BMS-663513 (CD137), Volociximab (anti-α5β1 integrin), GC1008 (TGFβ), HCD122 (CD40), Siplizumab (CD2), MORAb-003 (Folate receptor alpha), CNTO 328 (IL-6), MDX-060 (CD30), Ofatumumab (CD20), or SGN-33 (CD33). It is contemplated that one or more of these therapies may be employed with the miRNA therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p161NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR-1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al, 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### III. MIRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

A. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-16 miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

C. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity, that profile is relevant to whether that patient is an appropriate patient for receiving a drug, for receiving a combination of drugs, or for receiving a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005 in the names of David Brown, Lance Ford, Angie Cheng and Rich Jarvis, which is hereby incorporated by reference in its entirety.

E. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### IV. NUCLEIC ACIDS

The present invention concerns nucleic acids, modified or mimetic nucleic acids, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein and includes the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used.

“5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (e.g., mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NOs described herein, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, miRNA nucleic acids may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not a miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in the SEQ IDs provided herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence. Any 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130 140, 150, 160, 170, 180, 190, 200, 210, 220, 230, 240, 250, 260 or any number or range of sequences there between may be selected to the exclusion of all non-selected sequences.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, carboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Komberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments is alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### V. LABELS AND LABELING TECHNIQUES

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red;, fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VI. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art. Unless otherwise designated, catalog numbers refer to products available by that number from Ambion, Inc.®, The RNA Company.

### Example 1

### Gene Expression Analysis Following Transfection with HSA-miR-16

miRNAs are believed to primarily influence gene expression at the level of translation. Translational regulation leading to an up or down change in protein expression may lead to changes in activity and expression of downstream gene products and genes that are in turn regulated by those proteins. These regulatory effects would be revealed as changes in the global mRNA expression profile. Furthermore, it has recently been reported that, in some instances, miRNAs may reduce the mRNA levels of their direct targets (Bagga et al., 2005; Lim et al., 2005), and such changes can be observed upon microarray gene expression analysis. Microarray gene expression analyses were performed to identify genes that are mis-regulated by hsa-miR-16.

Synthetic Pre-miR-16 (Ambion) was reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1.

TABLE 1
Genes with increased (positive values) or decreased (negative values) expression
following transfection of human cancer cells with pre-miR hsa-miR-16.

| Gene Symbol | RefSeq Transcript ID | Δ log₂ |
|---|---|---|
| ABCB6 /// ATG9A | NM_005689 /// NM_024085 | −0.774183 |
| ACOX2 | NM_003500 | −0.747677 |
| ACTR2 | NM_001005386 /// NM_005722 | 0.706621 |
| ADARB1 | NM_001033049 /// NM_001112 /// | 1.12042 |
| | NM_015833 /// NM_015834 | |
| ADRB2 | NM_000024 | 0.822471 |
| ANKRD12 | NM_015208 | 0.920296 |
| AOX1 | NM_001159 | 0.71218 |
| ARHGDIA | NM_004309 | −1.31009 |
| ARHGDIB | NM_001175 | 0.974886 |
| ARL2 | NM_001667 | −1.26863 |
| ARL2BP | NM_012106 | 1.35222 |
| ATP6V0E | NM_003945 | 1.25179 |
| AXL | NM_001699 /// NM_021913 | 1.17272 |
| BAMBI | NM_012342 | −0.890685 |
| C4BPB | NM_000716 /// NM_001017364 /// | 1.48739 |
| | NM_001017365 /// NM_001017366 /// NM_001017367 | |
| CA12 | NM_001218 /// NM_206925 | −1.09634 |
| CCND1 | NM_053056 | −0.747979 |
| CCNG2 | NM_004354 | 0.94188 |
| CDC37L1 | NM_017913 | −0.851037 |
| CDH1 | NM_004360 | −0.735543 |
| CDH17 | NM_004063 | −0.805907 |
| CDKN2C | NM_001262 /// NM_078626 | −0.77508 |
| CDS2 | NM_003818 | −0.948554 |
| CFH /// CFHL1 | NM_000186 /// NM_001014975 /// NM_002113 | −0.917773 |
| CGI-48 | NM_016001 | 1.48424 |
| CHAF1A | NM_005483 | −0.704031 |
| CHUK | NM_001278 | −1.05995 |
| COL11A1 | NM_001854 /// NM_080629 /// NM_080630 | 0.7736 |
| COL1A1 | NM_000088 | −0.705029 |
| CPS1 | NM_001875 | −0.713235 |
| CTGF | NM_001901 | 1.22906 |
| CYP4F11 | NM_021187 | −0.829511 |
| CYP4F3 | NM_000896 | −1.12563 |
| DDAH1 | NM_012137 | 0.822493 |
| DIO2 | NM_000793 /// NM_001007023 /// NM_013989 | 0.814143 |
| DSU | NM_018000 | 0.74556 |
| DUSP1 | NM_004417 | 0.773277 |
| E2F8 | NM_024680 | −0.773773 |
| EEF1D | NM_001960 /// NM_032378 | 0.95742 |
| EFEMP1 | NM_004105 /// NM_018894 | 0.882177 |
| ENO1 | NM_001428 | 1.00751 |
| FBXO11 | NM_012167 /// NM_018693 /// NM_025133 | 0.924295 |
| FGF2 | NM_002006 | −1.19115 |
| FGFR4 | NM_002011 /// NM_022963 /// NM_213647 | −0.872234 |
| FGG | NM_000509 /// NM_021870 | −0.813252 |
| FLJ13910 | NM_022780 | 0.846746 |
| FNBP1 | NM_015033 | 0.743257 |
| GALNT7 | NM_017423 | −1.01457 |
| GBP1 | NM_002053 | 0.807432 |
| HAS2 | NM_005328 | −0.861488 |
| HEG | XM_087386 | 0.738182 |
| IFI16 | NM_005531 | 0.829221 |
| INHBC | NM_005538 | 0.797435 |
| INSL4 | NM_002195 | −0.916801 |
| KCNJ2 | NM_000891 | 0.857436 |
| KIAA0485 | — | 0.743897 |
| KLF4 | NM_004235 | −0.992125 |
| KRT7 | NM_005556 | 1.17333 |
| LCN2 | NM_005564 | −0.811381 |
| LRP12 | NM_013437 | −0.882349 |
| MAP7 | NM_003980 | −0.940371 |
| MCL1 | NM_021960 /// NM_182763 | 1.11653 |
| MYL9 | NM_006097 /// NM_181526 | 1.15849 |
| NAB1 | NM_005966 | −0.724633 |
| NALP1 | NM_001033053 /// NM_0149221 /// NM_033004 | 0.914964 |
| | /// NM_033006 /// NM_033007 | |
| NF1 | NM_000267 | −1.03572 |
| NNMT | NM_006169 | 0.997492 |
| NPC1 | NM_000271 | 0.911858 |
| NUCKS | NM_022731 | 2.31221 |
| NUPL1 | NM_001008564 /// NM_001008565 /// NM_014089 | −0.908999 |
| PGK1 | NM_000291 | 1.70175 |
| PHACTR2 | NM_014721 | −1.1275 |
| PLA2G4A | NM_024420 | −0.878708 |
| PLSCR4 | NM_020353 | −1.92309 |
| PMCH | NM_002674 | 1.09088 |
| PODXL | NM_001018111 /// NM_005397 | 0.927375 |
| PPAP2C | NM_003712 /// NM_177526 /// NM_177543 | −0.792886 |
| PRO1843 | — | 1.14274 |
| PTENP1 | — | 0.952354 |
| PTGS2 | NM_000963 | −1.72596 |
| PTK9 | NM_002822 /// NM_198974 | 0.970336 |
| PTPN12 | NM_002835 | 0.711122 |
| QKI | NM_006775 /// NM_206853 /// | 0.795792 |
| | NM_206854 /// NM_206855 | |
| RAB2 | NM_002865 | 1.24122 |
| RAFTLIN | NM_015150 | 1.16163 |
| RBL1 | NM_002895 /// NM_183404 | −0.766312 |
| RDX | NM_002906 | 0.704751 |
| RHEB | NM_005614 | 1.07577 |
| RIP | NM_001033002 /// NM_032308 | 1.34286 |
| RPL14 | NM_001034996 /// NM_003973 | 0.934016 |
| RPL38 | NM_000999 | 1.3638 |
| RPS11 | NM_001015 | 1.22134 |
| RPS6KA3 | NM_004586 | −0.875649 |
| RPS6KA5 | NM_004755 /// NM_182398 | 0.806899 |
| S100P | NM_005980 | −0.840949 |
| SCARB2 | NM_005506 | 0.857602 |
| SEPT6 /// N-PAC | NM_015129 /// NM_032569 /// NM_145799 | 0.703914 |
| | /// NM_145800 /// NM_145802 | |
| SKP2 | NM_005983 /// NM_032637 | 0.728768 |
| SLC11A2 | NM_000617 | −1.01869 |
| SLC4A7 | NM_003615 | −0.80415 |
| SMARCA2 | NM_003070 /// NM_139045 | 0.967136 |
| SPARC | NM_003118 | 1.07583 |
| STC1 | NM_003155 | 0.787502 |
| SULT1C1 | NM_001056 /// NM_176825 | 1.12689 |
| SUMO2 | NM_001005849 /// NM_006937 | 0.792739 |
| SYNE1 | NM_015293 /// NM_033071 /// | 0.852103 |
| | NM_133650 /// NM_182961 | |
| TACC1 | NM_006283 | −1.02015 |
| TAGLN | NM_001001522 /// NM_003186 | 1.8698 |
| TFG | NM_001007565 /// NM_006070 | 0.981989 |
| THBD | NM_000361 | 0.840966 |
| THBS1 | NM_003246 | −0.872199 |
| THUMPD1 | NM_017736 | −0.721243 |
| TMEM45A | NM_018004 | −0.874868 |
| TNFSF9 | NM_003811 | −1.13877 |
| TOX | NM_014729 | 1.16189 |
| TPM1 | NM_000366 /// NM_001018004 /// NM_001018005 | 0.792231 |
| | /// NM_001018006 /// NM_001018007 // | |
| TRA1 | NM_003299 | 2.10346 |
| TRIM22 | NM_006074 | 1.24509 |
| TXN | NM_003329 | 1.37224 |
| UBE2I | NM_003345 /// NM_194259 /// | 0.882609 |
| | NM_194260 /// NM_194261 | |
| UBE2L6 | NM_004223 /// NM_198183 | 0.709343 |
| USP34 | NM_014709 | 0.818893 |
| VDAC3 | NM_005662 | 1.14436 |
| VIL2 | NM_003379 | 0.899532 |
| WISP2 | NM_003881 | 0.703121 |
| XTP2 | NM_015172 | 1.05499 |
| ZBED2 | NM_024508 | 0.770913 |

Manipulation of the expression levels of the genes listed in Table 1 represents a potentially useful therapy for cancer and other diseases in which increased or reduced expression of hsa-miR-16 has a role in the disease.

### Example 2

### Cellular Pathways Affected by hsa-miR-16

The mis-regulation of gene expression by hsa-miR-16 (Table 1) affects many cellular pathways that represent potential therapeutic targets for the control of cancer and other diseases and disorders. The inventors determined the identity and nature of the cellular genetic pathways affected by the regulatory cascade induced by hsa-miR-16 expression. Cellular pathway analyses were performed using Ingenuity Pathways Analysis (Ingenuity® Systems, Redwood City, Calif.). The most significantly affected pathways following over-expression of hsa-miR-16 in A549 cells are shown in Table 2.

TABLE 2
Significantly affected functional cellular pathways following hsa-miR-16
over-expression in human cancer cells.

| Number | |
|---|---|
| of Genes | Pathway Functions |
| 15 | Drug Metabolism, Lipid Metabolism, |
| | Small Molecule Biochemistry |
| 14 | Cancer, Cell Morphology, Cell Cycle |
| 13 | Cellular Growth and Proliferation, Cancer, |
| | Cellular Development |
| 1 | Molecular Transport, Protein Trafficking, |
| | Cell-To-Cell Signaling and Interaction |
| 1 | Cellular Assembly and Organization, Cell |
| | Morphology, Molecular Transport |

These data demonstrate that hsa-miR-16 directly or indirectly affects the expression of numerous metabolic-, cellular proliferation-, cellular development-, and cell cycle-related genes and thus primarily affects functional pathways related to cellular growth, development, and proliferation. Those cellular processes all have integral roles in the development and progression of various cancers. Manipulation of the expression levels of genes in the cellular pathways shown in Table 2 represents a potentially useful therapy for cancer and other diseases in which increased or reduced expression of hsa-miR-16 has a role in the disease.

### Example 3

### Predicted Gene Targets of hsa-miR-16

Gene targets for binding of and regulation by hsa-miR-16-1 were predicted using the proprietary algorithm miRNATarget™ (Asuragen) and are shown in Table 3.

TABLE 3
Predicted target genes of hsa-miR-16.

| | RefSeq | |
|---|---|---|
| Gene Symbol | Transcript ID | Description |
| AAA1 | NM_207285 | AAA1 protein isoform III |
| AACS | NM_023928 | acetoacetyl-CoA synthetase |
| AADAT | NM_016228 | alpha-aminoadipate aminotransferase |
| AASDHPPT | NM_015423 | aminoadipate-semialdehyde |
| AATF | NM_012138 | apoptosis antagonizing transcription factor |
| ABAT | NM_000663 | 4-aminobutyrate aminotransferase precursor |
| ABCA1 | NM_005502 | ATP-binding cassette, sub-family A member 1 |
| ABCA3 | NM_001089 | ATP-binding cassette, sub-family A member 3 |
| ABCB8 | NM_007188 | ATP-binding cassette, sub-family B, member 8 |
| ABCB9 | NM_203445 | ATP-binding cassette, sub-family B (MDR/TAP), |
| ABCC10 | NM_033450 | ATP-binding cassette, sub-family C, member 10 |
| ABCC13 | NM_138726 | ATP-binding cassette protein C13 isoform a |
| ABCC3 | NM_020038 | ATP-binding cassette, sub-family C, member 3 |
| ABCC5 | NM_005688 | ATP-binding cassette, sub-family C, member 5 |
| ABCF1 | NM_001025091 | ATP-binding cassette, sub-family F, member 1 |
| ABCF2 | NM_005692 | ATP-binding cassette, sub-family F, member 2 |
| ABCF3 | NM_018358 | ATP-binding cassette, sub-family F (GCN20), |
| ABCG4 | NM_022169 | ATP-binding cassette, subfamily G, member 4 |
| ABHD11 | NM_031295 | abhydrolase domain containing 11 isoform 4 |
| ABHD13 | NM_032859 | hypothetical protein LOC84945 |
| ABHD2 | NM_007011 | alpha/beta hydrolase domain containing protein |
| ABI3 | NM_016428 | NESH protein |
| ABL1 | NM_005157 | v-abl Abelson murine leukemia viral oncogene |
| ABLIM1 | NM_001003407 | actin-binding LIM protein 1 isoform b |
| ABTB2 | NM_145804 | ankyrin repeat and BTB (POZ) domain containing |
| ACAA1 | NM_001607 | acetyl-Coenzyme A acyltransferase 1 |
| ACACA | NM_198834 | acetyl-Coenzyme A carboxylase alpha isoform 1 |
| ACACB | NM_001093 | acetyl-Coenzyme A carboxylase beta |
| ACAD9 | NM_014049 | acyl-Coenzyme A dehydrogenase family, member 9 |
| ACCN4 | NM_018674 | amiloride-sensitive cation channel 4 isoform 1 |
| ACE | NM_152831 | angiotensin I converting enzyme isoform 3 |
| ACOT11 | NM_147161 | thioesterase, adipose associated isoform BFIT2 |
| ACOT7 | NM_007274 | acyl-CoA thioesterase 7 isoform hBACHa |
| ACOT8 | NM_183385 | peroxisomal acyl-CoA thioesterase 1 isoform b |
| ACOX1 | NM_004035 | acyl-Coenzyme A oxidase isoform a |
| ACOX3 | NM_003501 | acyl-Coenzyme A oxidase 3, pristanoyl |
| ACP2 | NM_001610 | lysosomal acid phosphatase 2 precursor |
| ACPT | NM_080789 | testicular acid phosphatase isoform b precursor |
| ACSBG1 | NM_015162 | lipidosin |
| ACSBG2 | NM_030924 | bubblegum related protein |
| ACSL1 | NM_001995 | acyl-CoA synthetase long-chain family member 1 |
| ACSL4 | NM_004458 | acyl-CoA synthetase long-chain family member 4 |
| ACSL5 | NM_016234 | acyl-CoA synthetase long-chain family member 5 |
| ACSS2 | NM_018677 | acyl-CoA synthetase short-chain family member 2 |
| ACTR1A | NM_005736 | ARP1 actin-related protein 1 homolog A, |
| ACTR2 | NM_001005386 | actin-related protein 2 isoform a |
| ACTR3B | NM_020445 | actin-related protein 3-beta isoform 1 |
| ACTR8 | NM_022899 | actin-related protein 8 |
| ACVR2A | NM_001616 | activin A receptor, type IIA precursor |
| ADAM10 | NM_001110 | ADAM metallopeptidase domain 10 |
| ADAM11 | NM_002390 | ADAM metallopeptidase domain 11 preproprotein |
| ADAM12 | NM_021641 | ADAM metallopeptidase domain 12 isoform 2 |
| ADAMTS1 | NM_006988 | ADAM metallopeptidase with thrombospondin type 1 |
| ADAMTS13 | NM_139028 | ADAM metallopeptidase with thrombospondin type 1 |
| ADAMTS18 | NM_199355 | ADAM metallopeptidase with thrombospondin type 1 |
| ADAMTS3 | NM_014243 | ADAM metallopeptidase with thrombospondin type 1 |
| ADAMTS4 | NM_005099 | ADAM metallopeptidase with thrombospondin type 1 |
| ADAMTS5 | NM_007038 | ADAM metallopeptidase with thrombospondin type 1 |
| ADAMTS6 | NM_197941 | ADAM metallopeptidase with thrombospondin type 1 |
| ADAMTSL1 | NM_139238 | ADAMTS-like 1 isoform 1 |
| ADAMTSL2 | NM_014694 | ADAMTS-like 2 |
| ADAMTSL3 | NM_207517 | ADAMTS-like 3 |
| ADAR | NM_001025107 | adenosine deaminase, RNA-specific isoform d |
| ADARB1 | NM_001033049 | RNA-specific adenosine deaminase B1 isoform 4 |
| ADARB2 | NM_018702 | adenosine deaminase, RNA-specific, B2 |
| ADCY1 | NM_021116 | brain adenylate cyclase 1 |
| ADCY7 | NM_001114 | adenylate cyclase 7 |
| ADCY9 | NM_001116 | adenylate cyclase 9 |
| ADD1 | NM_001119 | adducin 1 (alpha) isoform a |
| ADD2 | NM_017482 | adducin 2 isoform b |
| ADM2 | NM_024866 | adrenomedullin 2 precusor |
| ADORA1 | NM_000674 | adenosine A1 receptor |
| ADORA2A | NM_000675 | adenosine A2a receptor |
| ADPRH | NM_001125 | ADP-ribosylarginine hydrolase |
| ADRA1B | NM_000679 | alpha-1B-adrenergic receptor |
| ADRA2A | NM_000681 | alpha-2A-adrenergic receptor |
| ADRA2B | NM_000682 | alpha-2B-adrenergic receptor |
| ADRB2 | NM_000024 | adrenergic, beta-2-, receptor, surface |
| ADRBK1 | NM_001619 | beta adrenergic receptor kinase 1 |
| ADSS | NM_001126 | adenylosuccinate synthase |
| AEBP2 | NM_153207 | AE binding protein 2 |
| AFAP | NM_021638 | actin filament associated protein |
| AFF2 | NM_002025 | fragile X mental retardation 2 |
| AFF4 | NM_014423 | ALL1 fused gene from 5q31 |
| AFM | NM_001133 | afamin precursor |
| AGA | NM_000027 | aspartylglucosaminidase precursor |
| AGPAT2 | NM_001012727 | 1-acylglycerol-3-phosphate O-acyltransferase 2 |
| AGPAT4 | NM_001012733 | 1-acylglycerol-3-phosphate O-acyltransferase 4 |
| AGPAT5 | NM_018361 | 1-acylglycerol-3-phosphate O-acyltransferase 5 |
| AGPAT6 | NM_178819 | lysophosphatidic acid acyltransferase zeta |
| AGPAT7 | NM_153613 | PLSC domain containing protein |
| AGRN | NM_198576 | agrin |
| AGTR2 | NM_000686 | angiotensin II receptor, type 2 |
| AHCYL1 | NM_006621 | S-adenosylhomocysteine hydrolase-like 1 |
| AHNAK | NM_024060 | AHNAK nucleoprotein isoform 2 |
| AHSA1 | NM_012111 | AHA1, activator of heat shock 90 kDa protein |
| AIM1 | NM_001624 | absent in melanoma 1 |
| AK3L1 | NM_001005353 | adenylate kinase 3-like 1 |
| AKAP1 | NM_003488 | A-kinase anchor protein 1 isoform 1 precursor |
| AKAP11 | NM_016248 | A-kinase anchor protein 11 isoform 1 |
| AKAP12 | NM_005100 | A-kinase anchor protein 12 isoform 1 |
| AKAP13 | NM_006738 | A-kinase anchor protein 13 isoform 1 |
| AKNA | NM_030767 | AT-hook transcription factor |
| AKR1CL1 | NM_001007536 | aldo-keto reductase family 1, member C-like 1 |
| AKR1D1 | NM_005989 | aldo-keto reductase family 1, member D1 |
| AKT3 | NM_005465 | v-akt murine thymoma viral oncogene homolog 3 |
| ALAD | NM_000031 | delta-aminolevulinic acid dehydratase isoform b |
| ALDH1A3 | NM_000693 | aldehyde dehydrogenase 1A3 |
| ALDH3A2 | NM_000382 | aldehyde dehydrogenase 3A2 isoform 2 |
| ALDH3B1 | NM_000694 | aldehyde dehydrogenase 3B1 isoform a |
| ALDH5A1 | NM_001080 | aldehyde dehydrogenase 5A1 precursor, isoform 2 |
| ALKBH3 | NM_139178 | alkB, alkylation repair homolog 3 |
| ALKBH5 | NM_017758 | hypothetical protein LOC54890 |
| ALKBH6 | NM_032878 | hypothetical protein LOC84964 isoform 2 |
| ALOX12 | NM_000697 | arachidonate 12-lipoxygenase |
| ALPK3 | NM_020778 | alpha-kinase 3 |
| ALPPL2 | NM_031313 | placental-like alkaline phosphatase |
| ALS2 | NM_020919 | alsin |
| ALS2CL | NM_147129 | ALS2 C-terminal like isoform 1 |
| ALS2CR16 | NM_205543 | amyotrophic lateral sclerosis 2 (juvenile) |
| ALS2CR2 | NM_018571 | amyotrophic lateral sclerosis 2 (juvenile) |
| AMIGO3 | NM_198722 | amphoterin-induced gene and ORF 3 |
| AMMECR1 | NM_001025580 | AMMECR1 protein isoform 2 |
| AMOT | NM_133265 | angiomotin |
| AMOTL1 | NM_130847 | angiomotin like 1 |
| AMOTL2 | NM_016201 | angiomotin like 2 |
| AMPD2 | NM_004037 | adenosine monophosphate deaminase 2 (isoform L) |
| AMPD3 | NM_000480 | erythrocyte adenosine monophosphate deaminase |
| AMT | NM_000481 | aminomethyltransferase (glycine cleavage system |
| ANAPC11 | NM_001002244 | APC11 anaphase promoting complex subunit 11 |
| ANAPC13 | NM_015391 | anaphase promoting complex subunit 13 |
| ANGEL1 | NM_015305 | angel homolog 1 |
| ANK1 | NM_000037 | ankyrin 1 isoform 3 |
| ANK2 | NM_001148 | ankyrin 2 isoform 1 |
| ANK3 | NM_001149 | ankyrin 3 isoform 2 |
| ANKRD11 | NM_013275 | ankyrin repeat domain 11 |
| ANKRD12 | NM_015208 | ankyrin repeat domain 12 |
| ANKRD13B | NM_152345 | hypothetical protein LOC124930 |
| ANKRD13D | NM_207354 | ankyrin repeat domain 13 family, member D |
| ANKRD15 | NM_015158 | ankyrin repeat domain protein 15 isoform a |
| ANKRD17 | NM_032217 | ankyrin repeat domain protein 17 isoform a |
| ANKRD19 | NM_001010925 | ankyrin repeat domain 19 |
| ANKRD29 | NM_173505 | ankyrin repeat domain 29 |
| ANKRD39 | NM_016466 | ankyrin repeat domain 39 |
| ANKRD46 | NM_198401 | ankyrin repeat domain 46 |
| ANKRD53 | NM_024933 | hypothetical protein LOC79998 |
| ANKS1A | NM_015245 | ankyrin repeat and sterile alpha motif domain |
| ANKS4B | NM_145865 | harmonin-interacting ankyrin-repeat containing |
| ANKZF1 | NM_018089 | ankyrin repeat and zinc finger domain containing |
| ANLN | NM_018685 | anillin, actin binding protein (scraps homolog, |
| ANP32E | NM_030920 | acidic (leucine-rich) nuclear phosphoprotein 32 |
| ANXA11 | NM_001157 | annexin Al1 |
| AP1G1 | NM_001030007 | adaptor-related protein complex 1, gamma 1 |
| AP1GBP1 | NM_007247 | AP1 gamma subunit binding protein 1 isoform 1 |
| AP1S1 | NM_001283 | adaptor-related protein complex 1, sigma 1 |
| AP1S2 | NM_003916 | adaptor-related protein complex 1 sigma 2 |
| AP2A1 | NM_014203 | adaptor-related protein complex 2, alpha 1 |
| AP2A2 | NM_012305 | adaptor-related protein complex 2, alpha 2 |
| AP2B1 | NM_001030006 | adaptor-related protein complex 2, beta 1 |
| AP3B1 | NM_003664 | adaptor-related protein complex 3, beta 1 |
| AP3M1 | NM_012095 | adaptor-related protein complex 3, mu 1 subunit |
| AP3S2 | NM_005829 | adaptor-related protein complex 3, sigma 2 |
| APBA1 | NM_001163 | amyloid beta A4 precursor protein-binding, |
| APBB3 | NM_133175 | amyloid beta precursor protein-binding, family |
| APC2 | NM_005883 | adenomatosis polyposis coli 2 |
| APLN | NM_017413 | apelin preproprotein |
| APLP2 | NM_001642 | amyloid beta (A4) precursor-like protein 2 |
| APOA4 | NM_000482 | apolipoprotein A-IV precursor |
| APOA5 | NM_052968 | apolipoprotein AV |
| APOBEC2 | NM_006789 | apolipoprotein B mRNA editing enzyme, catalytic |
| APOC3 | NM_000040 | apolipoprotein C-III precursor |
| APP | NM_000484 | amyloid beta A4 protein precursor, isoform a |
| APPBP1 | NM_001018159 | amyloid beta precursor protein-binding protein 1 |
| APPBP2 | NM_006380 | amyloid beta precursor protein-binding protein |
| APTX | NM_017692 | aprataxin isoform d |
| AQP1 | NM_198098 | aquaporin 1 |
| AQP11 | NM_173039 | aquaporin 11 |
| AQP2 | NM_000486 | aquaporin 2 |
| AQP4 | NM_001650 | aquaporin 4 isoform a |
| AQP8 | NM_001169 | aquaporin 8 |
| ARC | NM_015193 | activity-regulated cytoskeleton-associated |
| ARCN1 | NM_001655 | archain |
| ARF3 | NM_001659 | ADP-ribosylation factor 3 |
| ARFGAP1 | NM_018209 | ADP-ribosylation factor GTPase activating |
| ARFRP1 | NM_003224 | ADP-ribosylation factor related protein 1 |
| ARHGAP1 | NM_004308 | Rho GTPase activating protein 1 |
| ARHGAP10 | NM_024605 | Rho GTPase activating protein 10 |
| ARHGAP12 | NM_018287 | Rho GTPase activating protein 12 |
| ARHGAP18 | NM_033515 | Rho GTPase activating protein 18 |
| ARHGAP19 | NM_032900 | Rho GTPase activating protein 19 |
| ARHGAP20 | NM_020809 | Rho GTPase activating protein 20 |
| ARHGAP22 | NM_021226 | Rho GTPase activating protein 2 |
| ARHGAP26 | NM_015071 | GTPase regulator associated with the focal |
| ARHGAP27 | NM_199282 | Rho GTPase activating protein 27 |
| ARHGAP28 | NM_001010000 | Rho GTPase activating protein 28 isoform a |
| ARHGAP4 | NM_001666 | Rho GTPase activating protein 4 |
| ARHGAP5 | NM_001030055 | Rho GTPase activating protein 5 isoform a |
| ARHGDIA | NM_004309 | Rho GDP dissociation inhibitor (GDI) alpha |
| ARHGDIG | NM_001176 | Rho GDP dissociation inhibitor (GDI) gamma |
| ARHGEF10 | NM_014629 | Rho guanine nucleotide exchange factor 10 |
| ARHGEF12 | NM_015313 | Rho guanine nucleotide exchange factor (GEF) 12 |
| ARHGEF4 | NM_015320 | Rho guanine nucleotide exchange factor 4 isoform |
| ARHGEF5 | NM_001002861 | rho guanine nucleotide exchange factor 5 isoform |
| ARHGEF7 | NM_145735 | Rho guanine nucleotide exchange factor 7 isoform |
| ARHGEF9 | NM_015185 | Cdc42 guanine exchange factor 9 |
| ARID5A | NM_006673 | AT rich interactive domain 5A isoform 2 |
| ARL1 | NM_001177 | ADP-ribosylation factor-like 1 |
| ARL10 | NM_173664 | ADP-ribosylation factor-like 10 |
| ARL11 | NM_138450 | ADP-ribosylation factor-like 11 |
| ARL2 | NM_001667 | ADP-ribosylation factor-like 2 |
| ARL3 | NM_004311 | ADP-ribosylation factor-like 3 |
| ARL5B | NM_178815 | ADP-ribosylation factor-like 8 |
| ARL6IP5 | NM_006407 | ADP-ribosylation-like factor 6 interacting |
| ARL8B | NM_018184 | ADP-ribosylation factor-like 10C |
| ARMC1 | NM_018120 | armadillo repeat-containing protein |
| ARMC5 | NM_024742 | armadillo repeat containing 5 |
| ARMC6 | NM_033415 | armadillo repeat containing 6 |
| ARMCX1 | NM_016608 | armadillo repeat containing, X-linked 1 |
| ARMCX2 | NM_014782 | ALEX2 protein |
| ARNT | NM_001668 | aryl hydrocarbon receptor nuclear translocator |
| ARNT2 | NM_014862 | aryl hydrocarbon receptor nuclear translocator |
| ARPC1B | NM_005720 | actin related protein 2/3 complex subunit 1B |
| ARPP-19 | NM_006628 | cyclic AMP phosphoprotein, 19 kD |
| ARPP-21 | NM_001025068 | cyclic AMP-regulated phosphoprotein, 21 kD |
| ARRDC4 | NM_183376 | arrestin domain containing 4 |
| ARSD | NM_001669 | arylsulfatase D isoform a precursor |
| ARTS-1 | NM_016442 | type 1 tumor necrosis factor receptor shedding |
| ARVCF | NM_001670 | armadillo repeat protein |
| AS3MT | NM_020682 | arsenic (+3 oxidation state) methyltransferase |
| ASB1 | NM_016114 | ankyrin repeat and SOCS box-containing protein |
| ASB13 | NM_024701 | ankyrin repeat and SOCS box-containing protein |
| ASB15 | NM_080928 | ankyrin repeat and SOCS box-containing 15 |
| ASB6 | NM_017873 | ankyrin repeat and SOCS box-containing 6 isoform |
| ASCC3 | NM_022091 | activating signal cointegrator 1 complex subunit |
| ASCL2 | NM_005170 | achaete-scute complex homolog-like 2 |
| ASNSD1 | NM_019048 | asparagine synthetase domain containing 1 |
| ASPH | NM_032466 | aspartate beta-hydroxylase isoform c |
| ASTN | NM_004319 | astrotactin isoform 1 |
| ASXL1 | NM_015338 | additional sex combs like 1 |
| ASXL2 | NM_018263 | additional sex combs like 2 |
| ATAD4 | NM_024320 | ATPase family, AAA domain containing 4 |
| ATF3 | NM_004024 | activating transcription factor 3 isoform 2 |
| ATF6 | NM_007348 | activating transcription factor 6 |
| ATF7IP2 | NM_024997 | activating transcription factor 7 interacting |
| ATG4B | NM_013325 | APG4 autophagy 4 homolog B isoform a |
| ATG4D | NM_032885 | APG4 autophagy 4 homolog D |
| ATG9A | NM_024085 | APG9 autophagy 9-like 1 |
| ATG9B | NM_173681 | nitric oxide synthase 3 antisense |
| ATHL1 | NM_025092 | hypothetical protein LOC80162 |
| ATN1 | NM_001007026 | atrophin-1 |
| ATOH8 | NM_032827 | atonal homolog 8 |
| ATP11A | NM_015205 | ATPase, Class VI, type 11A isoform a |
| ATP11C | NM_001010986 | ATPase, Class VI, type 11C isoform b |
| ATP13A2 | NM_022089 | ATPase type 13A2 |
| ATP1B2 | NM_001678 | Na+/K+-ATPase beta 2 subunit |
| ATP1B4 | NM_012069 | ATPase, (Na+)/K+ transporting, beta 4 |
| ATP2A1 | NM_004320 | ATPase, Ca++ transporting, fast twitch 1 isoform |
| ATP2A3 | NM_005173 | sarco/endoplasmic reticulum Ca2+-ATPase isoform |
| ATP2B2 | NM_001001331 | plasma membrane calcium ATPase 2 isoform a |
| ATP2B3 | NM_001001344 | plasma membrane calcium ATPase 3 isoform 3b |
| ATP2B4 | NM_001001396 | plasma membrane calcium ATPase 4 isoform 4a |
| ATP4B | NM_000705 | ATPase, H+/K+ exchanging, beta polypeptide |
| ATP6V0B | NM_004047 | ATPase, H+ transporting, lysosomal 21 kDa, V0 |
| ATP6V0E2L | NM_145230 | ATPase, H+ transporting, V0 subunit |
| ATP6V1B2 | NM_001693 | vacuolar H+ATPase B2 |
| ATP6V1C1 | NM_001007254 | ATPase, H+ transporting, lysosomal 42 kDa, V1 |
| ATP6V1C2 | NM_144583 | vacuolar H+ ATPase C2 isoform b |
| ATP6V1G1 | NM_004888 | vacuolar H+ ATPase G1 |
| ATP7A | NM_000052 | ATPase, Cu++ transporting, alpha polypeptide |
| ATP7B | NM_000053 | ATPase, Cu++ transporting, beta polypeptide |
| ATP8B3 | NM_138813 | ATPase, Class I, type 8B, member 3 |
| ATPBD1C | NM_016301 | ATP binding domain 1 family, member C |
| ATRNL1 | NM_207303 | attractin-like 1 |
| ATXN2 | NM_002973 | ataxin 2 |
| ATXN7L2 | NM_153340 | ataxin 7-like 2 |
| AURKAIP1 | NM_017900 | aurora-A kinase interacting protein |
| AVEN | NM_020371 | cell death regulator aven |
| AXIN2 | NM_004655 | axin 2 |
| AXUD1 | NM_033027 | AXIN1 up-regulated 1 |
| B3GALNT1 | NM_003781 | UDP-Gal:betaGlcNAc beta |
| B3GALT5 | NM_006057 | UDP-Gal:betaGlcNAc beta |
| B3GALT6 | NM_080605 | UDP-Gal:betaGal beta 1,3-galactosyltransferase |
| B3GAT1 | NM_018644 | beta-1,3-glucuronyltransferase 1 |
| B3GAT3 | NM_012200 | beta-1,3-glucuronyltransferase 3 |
| B3GNT2 | NM_006577 | UDP-GlcNAc:betaGal |
| B3GNT3 | NM_014256 | UDP-GlcNAc:betaGal |
| B3GNT4 | NM_030765 | UDP-GlcNAc:betaGal |
| B4GALT1 | NM_001497 | UDP-Gal:betaGlcNAc beta 1,4- |
| B4GALT2 | NM_001005417 | UDP-Gal:betaGlcNAc beta 1,4- |
| B4GALT4 | NM_003778 | UDP-Gal:betaGlcNAc beta 1,4- |
| B4GALT5 | NM_004776 | UDP-Gal:betaGlcNAc beta 1,4- |
| bA16L21.2.1 | NM_001015882 | hypothetical protein LOC548645 |
| BAAT | NM_001701 | bile acid Coenzyme A: amino acid |
| BACE1 | NM_012104 | beta-site APP-cleaving enzyme 1 isoform A |
| BACE2 | NM_138992 | beta-site APP-cleaving enzyme 2 isoform B |
| BACH1 | NM_001011545 | BTB and CNC homology 1 isoform b |
| BACH2 | NM_021813 | BTB and CNC homology 1, basic leucine zipper |
| BAG3 | NM_004281 | BCL2-associated athanogene 3 |
| BAG4 | NM_004874 | BCL2-associated athanogene 4 |
| BAG5 | NM_001015048 | BCL2-associated athanogene 5 isoform b |
| BAHD1 | NM_014952 | bromo adjacent homology domain containing 1 |
| BAI1 | NM_001702 | brain-specific angiogenesis inhibitor 1 |
| BAIAP2 | NM_006340 | BAI1-associated protein 2 isoform 3 |
| BAP1 | NM_004656 | BRCA1 associated protein-1 |
| BAT2D1 | NM_015172 | HBxAg transactivated protein 2 |
| BAT4 | NM_033177 | HLA-B associated transcript 4 |
| BAZ1B | NM_032408 | bromodomain adjacent to zinc finger domain, 1B |
| BAZ2A | NM_013449 | bromodomain adjacent to zinc finger domain, 2A |
| BBC3 | NM_014417 | BCL2 binding component 3 |
| BCAP29 | NM_001008406 | B-cell receptor-associated protein BAP29 isoform |
| BCAP31 | NM_005745 | B-cell receptor-associated protein 31 |
| BCAS1 | NM_003657 | breast carcinoma amplified sequence 1 |
| BCAS4 | NM_001010974 | breast carcinoma amplified sequence 4 isoform c |
| BCL11B | NM_022898 | B-cell CLL/lymphoma 11B isoform 2 |
| BCL2 | NM_000633 | B-cell lymphoma protein 2 alpha isoform |
| BCL2L1 | NM_001191 | BCL2-like 1 isoform 2 |
| BCL2L11 | NM_006538 | BCL2-like 11 isoform 6 |
| BCL2L12 | NM_052842 | BCL2-like 12 isoform 2 |
| BCL2L14 | NM_030766 | BCL2-like 14 isoform 2 |
| BCL2L2 | NM_004050 | BCL2-like 2 protein |
| BCL7A | NM_001024808 | B-cell CLL/lymphoma 7A isoform b |
| BCL7B | NM_001707 | B-cell CLL/lymphoma 7B isoform 1 |
| BCL9 | NM_004326 | B-cell CLL/lymphoma 9 |
| BCL9L | NM_182557 | B-cell CLL/lymphoma 9-like |
| BCOR | NM_020926 | BCL-6 interacting corepressor isoform 2 |
| BCORL1 | NM_021946 | BCL6 co-repressor-like 1 |
| BCR | NM_004327 | breakpoint cluster region isoform 1 |
| BDH2 | NM_020139 | 3-hydroxybutyrate dehydrogenase, type 2 |
| BDKRB2 | NM_000623 | bradykinin receptor B2 |
| BDNF | NM_001709 | brain-derived neurotrophic factor isoform a |
| BET1L | NM_016526 | blocked early in transport 1 homolog (S. |
| BHLHB2 | NM_003670 | basic helix-loop-helix domain containing, class |
| BHLHB3 | NM_030762 | basic helix-loop-helix domain containing, class |
| BHMT2 | NM_017614 | betaine-homocysteine methyltransferase 2 |
| BICD2 | NM_001003800 | bicaudal D homolog 2 isoform 1 |
| BIK | NM_001197 | BCL2-interacting killer |
| BIN1 | NM_004305 | bridging integrator 1 isoform 8 |
| BIRC5 | NM_001012270 | baculoviral IAP repeat-containing protein 5 |
| BLCAP | NM_006698 | bladder cancer associated protein |
| BLMH | NM_000386 | bleomycin hydrolase |
| BLR1 | NM_001716 | Burkitt lymphoma receptor 1 isoform 1 |
| BMF | NM_001003940 | Bcl2 modifying factor isoform bmf-1 |
| BMPER | NM_133468 | BMP-binding endothelial regulator precursor |
| BMPR1A | NM_004329 | bone morphogenetic protein receptor, type IA |
| BMPR2 | NM_001204 | bone morphogenetic protein receptor type II |
| BMS1L | NM_014753 | BMS1-like, ribosome assembly protein |
| BMX | NM_001721 | BMX non-receptor tyrosine kinase |
| BNIP1 | NM_001205 | BCL2/adenovirus E1B 19 kD interacting protein 1 |
| BOLA2 | NM_001031833 | BolA-like protein 2 isoform b |
| BOLA3 | NM_212552 | bolA-like 3 isoform 1 |
| BRCA1 | NM_007306 | breast cancer 1, early onset isoform |
| BRD1 | NM_014577 | bromodomain containing protein 1 |
| BRD8 | NM_139199 | bromodomain containing 8 isoform 2 |
| BRF2 | NM_018310 | RNA polymerase III transcription initiation |
| BRI3 | NM_015379 | brain protein I3 |
| BRMS1 | NM_015399 | breast cancer metastasis suppressor 1 isoform 1 |
| BRP44L | NM_016098 | brain protein 44-like |
| BRPF3 | NM_015695 | bromodomain and PHD finger containing, 3 |
| BRS3 | NM_001727 | bombesin-like receptor 3 |
| BRWD1 | NM_001007246 | bromodomain and WD repeat domain containing 1 |
| BSDC1 | NM_018045 | BSD domain containing 1 |
| BSN | NM_003458 | bassoon protein |
| BSND | NM_057176 | barttin |
| BSPRY | NM_017688 | B-box and SPRY domain containing |
| BTAF1 | NM_003972 | BTAF1 RNA polymerase II, B-TFIID transcription |
| BTBD14B | NM_052876 | transcriptional repressor NAC1 |
| BTBD15 | NM_014155 | BTB (POZ) domain containing 15 |
| BTBD2 | NM_017797 | BTB (POZ) domain containing 2 |
| BTBD3 | NM_014962 | BTB/POZ domain containing protein 3 isoform a |
| BTBD4 | NM_025224 | BTB (POZ) domain containing 4 |
| BTBD7 | NM_001002860 | BTB (POZ) domain containing 7 isoform 1 |
| BTF3 | NM_001207 | basic transcription factor 3 isoform B |
| BTG2 | NM_006763 | B-cell translocation gene 2 |
| BTN1A1 | NM_001732 | butyrophilin, subfamily 1, member A1 |
| BTRC | NM_003939 | beta-transducin repeat containing protein |
| BUB3 | NM_004725 | BUB3 budding uninhibited by benzimidazoles 3 |
| BVES | NM_007073 | blood vessel epicardial substance |
| BZW1 | NM_014670 | basic leucine zipper and W2 domains 1 |
| C10orf108 | NM_001012714 | hypothetical protein LOC414235 |
| C10orf26 | NM_017787 | hypothetical protein LOC54838 |
| C10orf39 | NM_194303 | hypothetical protein LOC282973 |
| C10orf4 | NM_145246 | FRA10AC1 protein isoform FRA10AC1-1 |
| C10orf42 | NM_138357 | hypothetical protein LOC90550 |
| C10orf46 | NM_153810 | hypothetical protein LOC143384 |
| C10orf53 | NM_182554 | hypothetical protein LOC282966 |
| C10orf54 | NM_022153 | hypothetical protein LOC64115 |
| C10orf56 | NM_153367 | hypothetical protein LOC219654 |
| C10orf6 | NM_018121 | hypothetical protein LOC55719 |
| C10orf63 | NM_145010 | enkurin |
| C10orf67 | NM_153714 | hypothetical protein LOC256815 |
| C10orf7 | NM_006023 | D123 gene product |
| C10orf72 | NM_144984 | hypothetical protein LOC196740 isoform 2 |
| C10orf76 | NM_024541 | hypothetical protein LOC79591 |
| C10orf77 | NM_024789 | hypothetical protein LOC79847 |
| C10orf81 | NM_024889 | hypothetical protein LOC79949 |
| C10orf83 | NM_178832 | hypothetical protein LOC118812 |
| C10orf9 | NM_145012 | cyclin fold protein 1 isoform 1 |
| C10orf95 | NM_024886 | hypothetical protein LOC79946 |
| C11orf10 | NM_014206 | hypothetical protein LOC746 |
| C11orf11 | NM_006133 | neural stem cell-derived dendrite regulator |
| C11orf17 | NM_182901 | chromosome 11 open reading frame 17 |
| C11orf24 | NM_022338 | hypothetical protein LOC53838 |
| C11orf42 | NM_173525 | hypothetical protein LOC160298 |
| C11orf45 | NM_145013 | hypothetical protein LOC219833 |
| C11orf46 | NM_152316 | hypothetical protein LOC120534 |
| C11orf49 | NM_001003676 | hypothetical protein LOC79096 isoform 1 |
| C11orf53 | NM_198498 | hypothetical protein LOC341032 |
| C11orf55 | NM_207428 | hypothetical protein LOC399879 |
| C11orf68 | NM_031450 | basophilic leukemia expressed protein BLES03 |
| C12orf22 | NM_030809 | TGF-beta induced apoptosis protein 12 |
| C12orf30 | NM_024953 | hypothetical protein LOC80018 |
| C12orf34 | NM_032829 | hypothetical protein LOC84915 |
| C12orf38 | NM_024809 | TECT2 |
| C12orf4 | NM_020374 | hypothetical protein LOC57102 |
| C12orf47 | NM_016534 | apoptosis-related protein PNAS-1 |
| C12orf53 | NM_153685 | hypothetical protein LOC196500 |
| C13orf1 | NM_020456 | hypothetical protein LOC57213 |
| C13orf18 | NM_025113 | hypothetical protein LOC80183 |
| C14orf1 | NM_007176 | hypothetical protein LOC11161 |
| C14orf111 | NM_015962 | hypothetical protein LOC51077 |
| C14orf129 | NM_016472 | hypothetical protein LOC51527 |
| C14orf132 | NM_020215 | hypothetical protein LOC56967 |
| C14orf139 | NM_024633 | hypothetical protein LOC79686 |
| C14orf143 | NM_145231 | hypothetical protein LOC90141 |
| C14orf150 | NM_001008726 | hypothetical protein LOC112840 |
| C14orf32 | NM_144578 | MAPK-interacting and spindle-stabilizing |
| C14orf37 | NM_001001872 | hypothetical protein LOC145407 |
| C14orf4 | NM_024496 | chromosome 14 open reading frame 4 |
| C14orf43 | NM_194278 | hypothetical protein LOC91748 |
| C14orf45 | NM_025057 | hypothetical protein LOC80127 |
| C14orf68 | NM_207117 | chromosome 14 open reading frame 68 |
| C14orf79 | NM_174891 | hypothetical protein LOC122616 |
| C15orf37 | NM_175898 | hypothetical protein LOC283687 |
| C15orf39 | NM_015492 | hypothetical protein LOC56905 |
| C15orf40 | NM_144597 | hypothetical protein LOC123207 |
| C15orf41 | NM_032499 | hypothetical protein LOC84529 |
| C15orf42 | NM_152259 | leucine-rich repeat kinase 1 |
| C16orf14 | NM_138418 | hypothetical protein LOC84331 |
| C16orf34 | NM_144570 | chromosome 16 open reading frame 34 |
| C16orf55 | NM_153025 | hypothetical protein LOC124045 |
| C16orf56 | NM_025082 | hypothetical protein LOC80152 |
| C16orf57 | NM_024598 | hypothetical protein LOC79650 |
| C16orf58 | NM_022744 | hypothetical protein LOC64755 |
| C16orf63 | NM_144600 | hypothetical protein LOC123811 |
| C16orf7 | NM_004913 | chromosome 16 open reading frame 7 |
| C16orf70 | NM_025187 | lin-10 |
| C17orf27 | NM_020914 | chromosome 17 open reading frame 27 |
| C17orf32 | NM_152464 | hypothetical protein LOC147007 |
| C17orf39 | NM_024052 | hypothetical protein LOC79018 |
| C17orf41 | NM_024857 | chromosome fragility associated gene 1 |
| C17orf49 | NM_174893 | hypothetical protein LOC124944 |
| C17orf54 | NM_182564 | hypothetical protein LOC283982 |
| C17orf56 | NM_144679 | hypothetical protein LOC146705 |
| C17orf59 | NM_017622 | hypothetical protein LOC54785 |
| C17orf62 | NM_001033046 | hypothetical protein LOC79415 |
| C17orf81 | NM_203413 | S-phase 2 protein isoform 2 |
| C17orf82 | NM_203425 | hypothetical protein LOC388407 |
| C18orf1 | NM_001003674 | hypothetical protein LOC753 isoform gamma 1 |
| C18orf25 | NM_001008239 | chromosome 18 open reading frame 25 isoform b |
| C18orf34 | NM_198995 | hypothetical protein LOC374864 |
| C18orf4 | NM_032160 | hypothetical protein LOC92126 |
| C18orf43 | NM_006553 | chromosome 18 open reading frame 43 |
| C18orf45 | NM_032933 | hypothetical protein LOC85019 |
| C18orf54 | NM_173529 | hypothetical protein LOC162681 |
| C18orf58 | NM_173817 | hypothetical protein LOC284222 |
| C19orf12 | NM_001031726 | hypothetical protein LOC83636 isoform 1 |
| C19orf23 | NM_152480 | hypothetical protein LOC148046 |
| C19orf25 | NM_152482 | hypothetical protein LOC148223 |
| C19orf26 | NM_152769 | hypothetical protein LOC255057 |
| C19orf36 | NM_001031735 | hypothetical protein LOC113177 isoform 1 |
| C19orf6 | NM_033420 | membralin isoform 2 |
| C1orf101 | NM_173807 | hypothetical protein LOC257044 |
| C1orf102 | NM_145047 | oxidored-nitro domain-containing protein isoform |
| C1orf103 | NM_001006945 | receptor-interacting factor 1 isoform 2 |
| C1orf107 | NM_014388 | hypothetical protein LOC27042 |
| C1orf113 | NM_024676 | hypothetical protein LOC79729 |
| C1orf114 | NM_021179 | hypothetical protein LOC57821 |
| C1orf115 | NM_024709 | hypothetical protein LOC79762 |
| C1orf116 | NM_023938 | specifically androgen-regulated protein |
| C1orf119 | NM_020141 | hypothetical protein LOC56900 |
| C1orf126 | NM_182534 | hypothetical protein LOC200197 |
| C1orf130 | NM_001010980 | hypothetical protein LOC400746 |
| C1orf142 | NM_053052 | hypothetical protein LOC116841 |
| C1orf151 | NM_001032363 | chromosome 1 open reading frame 151 protein |
| C1orf173 | NM_001002912 | hypothetical protein LOC127254 |
| C1orf187 | NM_198545 | chromosome 1 open reading frame 187 |
| C1orf188 | NM_173795 | hypothetical protein LOC148646 |
| C1orf19 | NM_052965 | hypothetical protein LOC116461 |
| C1orf190 | NM_001013615 | hypothetical protein LOC541468 |
| C1orf2 | NM_006589 | hypothetical protein LOC10712 isoform a |
| C1orf21 | NM_030806 | chromosome 1 open reading frame 21 |
| C1orf36 | NM_183059 | chromosome 1 open reading frame 36 |
| C1orf38 | NM_004848 | basement membrane-induced gene isoform 1 |
| C1orf54 | NM_024579 | hypothetical protein LOC79630 |
| C1orf62 | NM_152763 | hypothetical protein LOC254268 |
| C1orf69 | NM_001010867 | hypothetical protein LOC200205 |
| C1orf84 | NM_001012960 | RP11-506B15.1 protein isoform 1 |
| C1orf9 | NM_014283 | chromosome 1 open reading frame 9 protein |
| C1orf95 | NM_001003665 | hypothetical protein LOC375057 |
| C1QA | NM_015991 | complement component 1, q subcomponent, A chain |
| C1QB | NM_000491 | complement component 1, q subcomponent, B chain |
| C1QL3 | NM_001010908 | complement component 1, q subcomponent-like 3 |
| C1QL4 | NM_001008223 | hypothetical protein LOC338761 |
| C1QTNF3 | NM_030945 | C1q and tumor necrosis factor related protein 3 |
| C1QTNF5 | NM_015645 | C1q and tumor necrosis factor related protein 5 |
| C1QTNF6 | NM_031910 | C1q and tumor necrosis factor related protein 6 |
| C1QTNF8 | NM_207419 | hypothetical protein LOC390664 |
| C20orf11 | NM_017896 | chromosome 20 open reading frame 11 |
| C20orf117 | NM_080627 | hypothetical protein LOC140710 isoform 1 |
| C20orf121 | NM_024331 | hypothetical protein LOC79183 |
| C20orf160 | NM_080625 | hypothetical protein LOC140706 |
| C20orf161 | NM_033421 | sorting nexin 21 isoform a |
| C20orf166 | NM_178463 | hypothetical protein LOC128826 |
| C20orf186 | NM_182519 | antimicrobial peptide RY2G5 |
| C20orf23 | NM_024704 | kinesin-like motor protein C20orf23 |
| C20orf29 | NM_018347 | hypothetical protein LOC55317 |
| C20orf3 | NM_020531 | chromosome 20 open reading frame 3 |
| C20orf39 | NM_024893 | hypothetical protein LOC79953 |
| C20orf42 | NM_017671 | chromosome 20 open reading frame 42 |
| C20orf43 | NM_016407 | hypothetical protein LOC51507 |
| C20orf44 | NM_018244 | basic FGF-repressed Zic binding protein isoform |
| C20orf45 | NM_016045 | hypothetical protein LOC51012 |
| C20orf46 | NM_018354 | hypothetical protein LOC55321 |
| C20orf58 | NM_152864 | hypothetical protein LOC128414 |
| C20orf71 | NM_178466 | hypothetical protein LOC128861 isoform b |
| C20orf77 | NM_021215 | hypothetical protein LOC58490 |
| C20orf96 | NM_153269 | hypothetical protein LOC140680 |
| C21orf123 | NM_199175 | hypothetical protein LOC378832 |
| C21orf125 | NM_194309 | hypothetical protein LOC284836 |
| C21orf129 | NM_152506 | hypothetical protein LOC150135 |
| C21orf24 | NM_001001789 | hypothetical protein LOC400866 |
| C21orf25 | NM_199050 | hypothetical protein LOC25966 |
| C21orf33 | NM_004649 | es1 protein isoform Ia precursor |
| C21orf57 | NM_001006114 | hypothetical protein LOC54059 isoform 2 |
| C21orf58 | NM_199071 | hypothetical protein LOC54058 isoform 2 |
| C21orf6 | NM_016940 | hypothetical protein LOC10069 |
| C21orf62 | NM_019596 | hypothetical protein LOC56245 |
| C21orf69 | NM_058189 | chromosome 21 open reading frame 69 |
| C21orf84 | NM_153752 | hypothetical protein LOC114038 |
| C21orf93 | NM_145179 | hypothetical protein LOC246704 |
| C22orf13 | NM_031444 | chromosome 22 open reading frame 13 |
| C22orf5 | NM_012264 | chromosome 22 open reading frame 5 |
| C22orf9 | NM_001009880 | hypothetical protein LOC23313 isoform b |
| C2orf17 | NM_024293 | hypothetical protein LOC79137 |
| C2orf19 | NM_001024676 | chromosome 2 open reading frame 19 |
| C2orf26 | NM_023016 | hypothetical protein LOC65124 |
| C3orf10 | NM_018462 | chromosome 3 open reading frame 10 |
| C3orf18 | NM_016210 | hypothetical protein LOC51161 |
| C3orf19 | NM_016474 | hypothetical protein LOC51244 |
| C3orf23 | NM_001029839 | hypothetical protein LOC285343 isoform 2 |
| C3orf27 | NM_007354 | putative GR6 protein |
| C3orf37 | NM_001006109 | hypothetical protein LOC56941 |
| C3orf56 | NM_001007534 | hypothetical protein LOC285311 |
| C3orf58 | NM_173552 | hypothetical protein LOC205428 |
| C4orf15 | NM_024511 | hypothetical protein LOC79441 |
| C4orf19 | NM_018302 | hypothetical protein LOC55286 |
| C5orf21 | NM_032042 | hypothetical protein LOC83989 |
| C5orf24 | NM_152409 | hypothetical protein LOC134553 |
| C6orf106 | NM_022758 | chromosome 6 open reading frame 106 isoform b |
| C6orf128 | NM_145316 | hypothetical protein LOC221468 |
| C6orf142 | NM_138569 | hypothetical protein LOC90523 |
| C6orf145 | NM_183373 | hypothetical protein LOC221749 |
| C6orf151 | NM_152551 | U11/U12 snRNP 48K |
| C6orf152 | NM_181714 | hypothetical protein LOC167691 |
| C6orf155 | NM_024882 | hypothetical protein LOC79940 |
| C6orf168 | NM_032511 | hypothetical protein LOC84553 |
| C6orf199 | NM_145025 | hypothetical protein LOC221264 |
| C6orf35 | NM_018452 | hypothetical protein LOC55836 |
| C6orf47 | NM_021184 | G4 protein |
| C6orf49 | NM_013397 | over-expressed breast tumor protein |
| C6orf51 | NM_138408 | hypothetical protein LOC112495 |
| C6orf55 | NM_016485 | hypothetical protein LOC51534 |
| C6orf57 | NM_145267 | hypothetical protein LOC135154 |
| C6orf59 | NM_024929 | hypothetical protein LOC79992 |
| C6orf64 | NM_018322 | hypothetical protein LOC55776 |
| C6orf71 | NM_203395 | chromosome 6 open reading frame 71 |
| C6orf85 | NM_021945 | ion transporter protein |
| C7orf16 | NM_006658 | G-substrate |
| C7orf19 | NM_032831 | hypothetical protein LOC80228 |
| C7orf20 | NM_015949 | hypothetical protein LOC51608 |
| C7orf21 | NM_031434 | hypothetical protein LOC83590 |
| C7orf29 | NM_138434 | hypothetical protein LOC113763 |
| C8orf30A | NM_016458 | brain protein 16 |
| C8orf38 | NM_152416 | hypothetical protein LOC137682 |
| C8orf4 | NM_020130 | chromosome 8 open reading frame 4 |
| C8orf42 | NM_175075 | hypothetical protein LOC157695 |
| C8orf49 | NM_001031839 | hypothetical protein LOC606553 |
| C8orf58 | NM_001013842 | hypothetical protein LOC541565 |
| C8orf70 | NM_016010 | hypothetical protein LOC51101 |
| C9orf100 | NM_032818 | hypothetical protein LOC84904 |
| C9orf106 | NM_001012715 | hypothetical protein LOC414318 |
| C9orf10OS | NM_198841 | hypothetical protein LOC158293 |
| C9orf114 | NM_016390 | hypothetical protein LOC51490 |
| C9orf121 | NM_145283 | nucleoredoxin |
| C9orf123 | NM_033428 | hypothetical protein LOC90871 |
| C9orf128 | NM_001012446 | hypothetical protein LOC392307 |
| C9orf150 | NM_203403 | hypothetical protein LOC286343 |
| C9orf163 | NM_152571 | hypothetical protein LOC158055 |
| C9orf164 | NM_182635 | hypothetical protein LOC349236 |
| C9orf19 | NM_022343 | chromosome 9 open reading frame 19 |
| C9orf25 | NM_147202 | hypothetical protein LOC203259 |
| C9orf26 | NM_033439 | interleukin 33 |
| C9orf28 | NM_001011703 | hypothetical protein LOC89853 isoform 2 |
| C9orf3 | NM_032823 | aminopeptidase O |
| C9orf42 | NM_138333 | hypothetical protein LOC116224 |
| C9orf48 | NM_194313 | hypothetical protein LOC347240 |
| C9orf5 | NM_032012 | hypothetical protein LOC23731 |
| C9orf61 | NM_004816 | chromosome 9 open reading frame 61 |
| C9orf66 | NM_152569 | hypothetical protein LOC157983 |
| C9orf7 | NM_017586 | hypothetical protein LOC11094 |
| C9orf74 | NM_030914 | hypothetical protein LOC81605 |
| C9orf82 | NM_024828 | hypothetical protein LOC79886 |
| C9orf88 | NM_022833 | hypothetical protein LOC64855 |
| C9orf89 | NM_032310 | chromosome 9 open reading frame 89 |
| C9orf91 | NM_153045 | hypothetical protein LOC203197 |
| CA12 | NM_001218 | carbonic anhydrase XII isoform 1 precursor |
| CA2 | NM_000067 | carbonic anhydrase II |
| CA8 | NM_004056 | carbonic anhydrase VIII |
| CAB39 | NM_016289 | calcium binding protein 39 |
| CAB39L | NM_030925 | calcium binding protein 39-like isoform 2 |
| CABC1 | NM_020247 | chaperone, ABC1 activity of bc1 complex like |
| CABLES2 | NM_031215 | Cdk5 and Abl enzyme substrate 2 |
| CABP1 | NM_001033677 | calcium binding protein 1 isoform 3 |
| CABP7 | NM_182527 | calcium binding protein 7 |
| CACNA1E | NM_000721 | calcium channel, voltage-dependent, alpha 1E |
| CACNA1I | NM_001003406 | voltage-dependent T-type calcium channel |
| CACNA2D4 | NM_001005737 | voltage-gated calcium channel alpha(2)delta-4 |
| CACNB1 | NM_000723 | calcium channel, voltage-dependent, beta 1 |
| CACNB4 | NM_000726 | calcium channel, voltage-dependent, beta 4 |
| CAD | NM_004341 | carbamoylphosphate synthetase 2/aspartate |
| CALB2 | NM_001740 | calbindin 2 full length protein isoform |
| CALM1 | NM_006888 | calmodulin 1 |
| CALML4 | NM_033429 | calmodulin-like 4 isoform 2 |
| CALML5 | NM_017422 | calmodulin-like skin protein |
| CALML6 | NM_138705 | calmodulin-like 6 |
| CALN1 | NM_001017440 | calneuron 1 |
| CALU | NM_001219 | calumenin precursor |
| CAMK2A | NM_015981 | calcium/calmodulin-dependent protein kinase IIA |
| CAMK2G | NM_001222 | calcium/calmodulin-dependent protein kinase II |
| CAMKK2 | NM_006549 | calcium/calmodulin-dependent protein kinase |
| CAMKV | NM_024046 | CaM kinase-like vesicle-associated |
| CAMSAP1 | NM_015447 | calmodulin regulated spectrin-associated protein |
| CAMSAP1L1 | NM_203459 | calmodulin regulated spectrin-associated protein |
| CANX | NM_001024649 | calnexin precursor |
| CAP1 | NM_006367 | adenylyl cyclase-associated protein |
| CAP2 | NM_006366 | adenylyl cyclase-associated protein 2 |
| CAPN12 | NM_144691 | calpain 12 |
| CAPN3 | NM_212464 | calpain 3 isoform g |
| CAPN5 | NM_004055 | calpain 5 |
| CAPN6 | NM_014289 | calpain 6 |
| CAPS | NM_004058 | calcyphosine isoform a |
| CAPZA2 | NM_006136 | capping protein (actin filament) muscle Z-line, |
| CARD10 | NM_014550 | caspase recruitment domain protein 10 |
| CARD14 | NM_052819 | caspase recruitment domain protein 14 isoform 2 |
| CARD4 | NM_006092 | caspase recruitment domain family, member 4 |
| CARM1 | NM_199141 | coactivator-associated arginine |
| CARS | NM_001014437 | cysteinyl-tRNA synthetase isoform c |
| CASKIN1 | NM_020764 | CASK interacting protein 1 |
| CASP10 | NM_001230 | caspase 10 isoform a preproprotein |
| CASP4 | NM_033307 | caspase 4 isoform delta |
| CASQ2 | NM_001232 | cardiac calsequestrin 2 |
| CASR | NM_000388 | calcium-sensing receptor |
| CAST | NM_173060 | calpastatin isoform b |
| CAST1 | NM_015576 | cytomatrix protein p110 |
| CASZ1 | NM_017766 | castor homolog 1, zinc finger |
| CBARA1 | NM_006077 | calcium binding atopy-related autoantigen 1 |
| CBFA2T2 | NM_001032999 | core-binding factor, runt domain, alpha subunit |
| CBFA2T3 | NM_005187 | myeloid translocation gene-related protein 2 |
| CBFB | NM_001755 | core-binding factor, beta subunit isoform 2 |
| CBL | NM_005188 | Cas-Br-M (murine) ecotropic retroviral |
| CBLC | NM_012116 | Cas-Br-M (murine) ecotropic retroviral |
| CBR3 | NM_001236 | carbonyl reductase 3 |
| CBX2 | NM_005189 | chromobox homolog 2 isoform 1 |
| CBX4 | NM_003655 | chromobox homolog 4 |
| CC2D1B | NM_032449 | coiled-coil and C2 domain containing 1B |
| CCDC18 | NM_206886 | sarcoma antigen NY-SAR-41 |
| CCDC19 | NM_012337 | nasopharyngeal epithelium specific protein 1 |
| CCDC21 | NM_022778 | coiled-coil domain containing 21 |
| CCDC25 | NM_001031708 | coiled-coil domain containing 25 isoform 1 |
| CCDC28A | NM_015439 | hypothetical protein LOC25901 |
| CCDC3 | NM_031455 | coiled-coil domain containing 3 |
| CCDC32 | NM_052849 | coiled-coil domain containing 32 |
| CCDC4 | NM_207406 | hypothetical protein LOC389206 |
| CCDC44 | NM_016360 | clone HQ0477 PRO0477p |
| CCDC47 | NM_020198 | hypothetical protein LOC57003 |
| CCDC52 | NM_144718 | coiled-coil domain containing 52 |
| CCDC55 | NM_001033563 | hypothetical protein LOC84081 isoform 2 |
| CCDC6 | NM_005436 | coiled-coil domain containing 6 |
| CCDC68 | NM_025214 | CTCL tumor antigen se57-1 |
| CCDC80 | NM_199511 | steroid-sensitive protein 1 |
| CCDC81 | NM_021827 | hypothetical protein LOC60494 |
| CCDC83 | NM_173556 | hypothetical protein LOC220047 |
| CCDC88 | NM_032251 | hypothetical protein LOC283234 |
| CCDC94 | NM_018074 | hypothetical protein LOC55702 |
| CCDC95 | NM_173618 | coiled-coil domain containing 95 |
| CCDC97 | NM_052848 | hypothetical protein LOC90324 |
| CCL15 | NM_004167 | chemokine (C-C motif) ligand 15 precursor |
| CCL22 | NM_002990 | small inducible cytokine A22 precursor |
| CCND1 | NM_053056 | cyclin D1 |
| CCND2 | NM_001759 | cyclin D2 |
| CCND3 | NM_001760 | cyclin D3 |
| CCNE1 | NM_001238 | cyclin E1 isoform 1 |
| CCNE2 | NM_057735 | cyclin E2 isoform 2 |
| CCNF | NM_001761 | cyclin F |
| CCNJ | NM_019084 | cyclin J |
| CCNT2 | NM_001241 | cyclin T2 isoform a |
| CCR7 | NM_001838 | chemokine (C-C motif) receptor 7 precursor |
| CCR9 | NM_006641 | chemokine (C-C motif) receptor 9 isoform B |
| CCRK | NM_012119 | cell cycle related kinase isoform 2 |
| CCS | NM_005125 | copper chaperone for superoxide dismutase |
| CD151 | NM_004357 | CD151 antigen |
| CD163 | NM_004244 | CD163 antigen isoform a |
| CD164 | NM_006016 | CD164 antigen, sialomucin |
| CD180 | NM_005582 | CD180 antigen |
| CD200R1 | NM_138806 | CD200 receptor 1 isoform a |
| CD209 | NM_021155 | CD209 antigen |
| CD22 | NM_001771 | CD22 antigen |
| CD274 | NM_014143 | CD274 antigen |
| CD276 | NM_001024736 | CD276 antigen isoform a |
| CD28 | NM_006139 | CD28 antigen |
| CD300C | NM_006678 | CD300C antigen |
| CD300LG | NM_145273 | triggering receptor expressed on myeloid cells |
| CD302 | NM_014880 | CD302 antigen |
| CD37 | NM_001774 | CD37 antigen isoform A |
| CD3E | NM_000733 | CD3E antigen, epsilon polypeptide (TiT3 |
| CD4 | NM_000616 | CD4 antigen precursor |
| CD40 | NM_001250 | CD40 antigen isoform 1 precursor |
| CD47 | NM_001025079 | CD47 molecule isoform 3 precursor |
| CD48 | NM_001778 | CD48 antigen (B-cell membrane protein) |
| CD5 | NM_014207 | CD5 antigen (p56-62) |
| CD6 | NM_006725 | CD6 antigen |
| CD69 | NM_001781 | CD69 antigen (p60, early T-cell activation |
| CD80 | NM_005191 | CD80 antigen (CD28 antigen ligand 1, B7-1 |
| CD82 | NM_001024844 | CD82 antigen isoform 2 |
| CD83 | NM_004233 | CD83 antigen isoform a |
| CD93 | NM_012072 | CD93 antigen precursor |
| CD97 | NM_001025160 | CD97 antigen isoform 3 precursor |
| CD99L2 | NM_031462 | CD99 antigen-like 2 isoform E3′-E4′-E3-E4 |
| CDADC1 | NM_030911 | cytidine and dCMP deaminase domain containing 1 |
| CDC14A | NM_003672 | CDC14 homolog A isoform 1 |
| CDC14B | NM_003671 | CDC14 homolog B isoform 1 |
| CDC23 | NM_004661 | cell division cycle protein 23 |
| CDC25A | NM_001789 | cell division cycle 25A isoform a |
| CDC25B | NM_004358 | cell division cycle 25B isoform 2 |
| CDC25C | NM_001790 | cell division cycle 25C protein isoform a |
| CDC27 | NM_001256 | cell division cycle protein 27 |
| CDC34 | NM_004359 | cell division cycle 34 |
| CDC37L1 | NM_017913 | cell division cycle 37 homolog (S. |
| CDC42 | NM_044472 | cell division cycle 42 isoform 2 |
| CDC42BPA | NM_003607 | CDC42-binding protein kinase alpha isoform B |
| CDC42BPB | NM_006035 | CDC42-binding protein kinase beta |
| CDC42EP2 | NM_006779 | Cdc42 effector protein 2 |
| CDC42EP4 | NM_012121 | Cdc42 effector protein 4 |
| CDC7 | NM_003503 | CDC7 cell division cycle 7 |
| CDCA4 | NM_017955 | cell division cycle associated 4 |
| CDCA5 | NM_080668 | cell division cycle associated 5 |
| CDCA7L | NM_018719 | transcription factor RAM2 |
| CDCP2 | NM_201546 | hypothetical protein LOC200008 |
| CDH1 | NM_004360 | cadherin 1, type 1 preproprotein |
| CDH22 | NM_021248 | cadherin 22 precursor |
| CDK10 | NM_052988 | cyclin-dependent kinase 10 isoform 3 |
| CDK5R1 | NM_003885 | cyclin-dependent kinase 5, regulatory subunit 1 |
| CDK5RAP1 | NM_016082 | CDK5 regulatory subunit associated protein 1 |
| CDK5RAP3 | NM_025197 | CDK5 regulatory subunit associated protein 3 |
| CDK6 | NM_001259 | cyclin-dependent kinase 6 |
| CDKN1A | NM_000389 | cyclin-dependent kinase inhibitor 1A |
| CDKN2A | NM_058197 | cyclin-dependent kinase inhibitor 2A isoform 3 |
| CDKN2B | NM_078487 | cyclin-dependent kinase inhibitor 2B isoform 2 |
| CDKN2D | NM_001800 | cyclin-dependent kinase inhibitor 2D |
| CDR2 | NM_001802 | cerebellar degeneration-related protein 2 |
| CDS2 | NM_003818 | phosphatidate cytidylyltransferase 2 |
| CDT1 | NM_030928 | DNA replication factor |
| CDV3 | NM_017548 | CDV3 homolog |
| CDX1 | NM_001804 | caudal type homeo box transcription factor 1 |
| CDX2 | NM_001265 | caudal type homeo box transcription factor 2 |
| CEACAM19 | NM_020219 | carcinoembryonic antigen-like 1 |
| CEACAM6 | NM_002483 | carcinoembryonic antigen-related cell adhesion |
| CEACAM7 | NM_006890 | carcinoembryonic antigen-related cell adhesion |
| CEBPG | NM_001806 | CCAAT/enhancer binding protein gamma |
| CECR1 | NM_017424 | cat eye syndrome critical region protein 1 |
| CECR6 | NM_031890 | cat eye syndrome chromosome region, candidate 6 |
| CENTA1 | NM_006869 | centaurin, alpha 1 |
| CENTD1 | NM_015230 | centaurin delta 1 isoform a |
| CENTG1 | NM_014770 | centaurin, gamma 1 |
| CEP152 | NM_014985 | hypothetical protein LOC22995 |
| CEP170 | NM_014812 | centrosomal protein 170 kDa |
| CEP27 | NM_018097 | hypothetical protein LOC55142 |
| CEP350 | NM_014810 | centrosome-associated protein 350 |
| CEP55 | NM_018131 | centrosomal protein 55 kDa |
| CERK | NM_022766 | ceramide kinase isoform a |
| CERKL | NM_201548 | ceramide kinase-like isoform a |
| CGGBP1 | NM_001008390 | CGG triplet repeat binding protein 1 |
| CGI-38 | NM_015964 | hypothetical protein LOC51673 |
| CGI-69 | NM_016016 | hypothetical protein LOC51629 |
| CGN | NM_020770 | cingulin |
| CGNL1 | NM_032866 | cingulin-like 1 |
| CHAC1 | NM_024111 | hypothetical protein LOC79094 |
| CHD5 | NM_015557 | chromodomain helicase DNA binding protein 5 |
| CHD6 | NM_032221 | chromodomain helicase DNA binding protein 6 |
| CHD7 | NM_017780 | chromodomain helicase DNA binding protein 7 |
| CHD8 | NM_020920 | chromodomain helicase DNA binding protein 8 |
| CHD9 | NM_025134 | chromodomain helicase DNA binding protein 9 |
| CHDH | NM_018397 | choline dehydrogenase |
| CHEK1 | NM_001274 | CHK1 checkpoint homolog |
| CHERP | NM_006387 | calcium homeostasis endoplasmic reticulum |
| CHFR | NM_018223 | checkpoint with forkhead and ring finger |
| CHGA | NM_001275 | chromogranin A precursor |
| CHID1 | NM_023947 | hypothetical protein LOC66005 |
| CHKB | NM_152253 | choline/ethanolamine kinase isoform b |
| CHMP4B | NM_176812 | chromatin modifying protein 4B |
| CHMP6 | NM_024591 | chromatin modifying protein 6 |
| CHORDC1 | NM_012124 | cysteine and histidine-rich domain |
| CHP | NM_007236 | calcium binding protein P22 |
| CHPT1 | NM_020244 | choline phosphotransferase 1 |
| CHRAC1 | NM_017444 | chromatin accessibility complex 1 |
| CHRD | NM_177978 | chordin isoform b |
| CHRFAM7A | NM_139320 | CHRNA7-FAM7A fusion isoform 1 |
| CHRNA3 | NM_000743 | cholinergic receptor, nicotinic, alpha |
| CHRNA4 | NM_000744 | cholinergic receptor, nicotinic, alpha 4 subunit |
| CHRNA5 | NM_000745 | cholinergic receptor, nicotinic, alpha |
| CHRNB2 | NM_000748 | cholinergic receptor, nicotinic, beta |
| CHRNB3 | NM_000749 | cholinergic receptor, nicotinic, beta |
| CHRNB4 | NM_000750 | cholinergic receptor, nicotinic, beta |
| CHRNE | NM_000080 | nicotinic acetylcholine receptor epsilon |
| CHST10 | NM_004854 | HNK-1 sulfotransferase |
| CHST3 | NM_004273 | carbohydrate (chondroitin 6) sulfotransferase 3 |
| CHST6 | NM_021615 | carbohydrate (N-acetylglucosamine 6-O) |
| CHUK | NM_001278 | conserved helix-loop-helix ubiquitous kinase |
| CHX10 | NM_182894 | ceh-10 homeo domain containing homolog |
| CIAPIN1 | NM_020313 | cytokine induced apoptosis inhibitor 1 |
| CIB2 | NM_006383 | DNA-dependent protein kinase catalytic |
| CIDEB | NM_014430 | cell death-inducing DFFA-like effector b |
| CINP | NM_032630 | cyclin-dependent kinase 2-interacting protein |
| CKAP5 | NM_001008938 | colonic and hepatic tumor over-expressed protein |
| CKB | NM_001823 | brain creatine kinase |
| CLASP1 | NM_015282 | CLIP-associating protein 1 |
| CLASP2 | NM_015097 | CLIP-associating protein 2 |
| CLCN3 | NM_001829 | chloride channel 3 isoform b |
| CLCN4 | NM_001830 | chloride channel 4 |
| CLCN5 | NM_000084 | chloride channel 5 |
| CLCN6 | NM_001286 | chloride channel 6 isoform ClC-6a |
| CLCN7 | NM_001287 | chloride channel 7 |
| CLDN1 | NM_021101 | claudin 1 |
| CLDN12 | NM_012129 | claudin 12 |
| CLDN14 | NM_012130 | claudin 14 |
| CLDN2 | NM_020384 | claudin 2 |
| CLDN4 | NM_001305 | claudin 4 |
| CLDN5 | NM_003277 | claudin 5 |
| CLDN6 | NM_021195 | claudin 6 |
| CLEC12A | NM_201625 | myeloid inhibitory C-type lectin-like receptor |
| CLEC12B | NM_205852 | macrophage antigen h |
| CLEC2D | NM_001004419 | osteoclast inhibitory lectin isoform 2 |
| CLEC4F | NM_173535 | C-type lectin, superfamily member 13 |
| CLEC4M | NM_214677 | CD299 antigen isoform 3 |
| CLIC5 | NM_016929 | chloride intracellular channel 5 |
| CLK1 | NM_001024646 | CDC-like kinase 1 isoform 2 |
| CLK4 | NM_020666 | CDC-like kinase 4 |
| CLLU1 | NM_001025233 | hypothetical protein LOC574028 |
| CLN8 | NM_018941 | CLN8 protein |
| CLOCK | NM_004898 | clock |
| CLSTN1 | NM_001009566 | calsyntenin 1 isoform 1 |
| CLTB | NM_001834 | clathrin, light polypeptide isoform a |
| CLU | NM_001831 | clusterin isoform 1 |
| CLUAP1 | NM_024793 | clusterin associated protein 1 isoform 2 |
| CMIP | NM_030629 | c-Maf-inducing protein Tc-mip isoform |
| CMPK | NM_016308 | cytidylate kinase |
| CMTM1 | NM_052999 | chemokine-like factor superfamily 1 isoform 13 |
| CMTM3 | NM_144601 | chemokine-like factor superfamily 3 isoform a |
| CMTM4 | NM_178818 | chemokine-like factor superfamily 4 isoform 1 |
| CMTM6 | NM_017801 | CKLF-like MARVEL transmembrane domain |
| | | containing |
| CNIH2 | NM_182553 | cornichon homolog 2 |
| CNIH3 | NM_152495 | cornichon homolog 3 |
| CNN1 | NM_001299 | calponin 1, basic, smooth muscle |
| CNNM2 | NM_017649 | cyclin M2 isoform 1 |
| CNNM3 | NM_017623 | cyclin M3 isoform 1 |
| CNOT6 | NM_015455 | CCR4-NOT transcription complex, subunit 6 |
| CNTD2 | NM_024877 | hypothetical protein LOC79935 |
| CNTN3 | NM_020872 | contactin 3 |
| CNTNAP1 | NM_003632 | contactin associated protein 1 |
| COBLL1 | NM_014900 | COBL-like 1 |
| COG3 | NM_031431 | component of golgi transport complex 3 |
| COG7 | NM_153603 | component of oligomeric golgi complex 7 |
| COL11A2 | NM_080679 | collagen, type XI, alpha 2 isoform 3 |
| COL12A1 | NM_004370 | collagen, type XII, alpha 1 long isoform |
| COL23A1 | NM_173465 | collagen, type XXIII, alpha 1 |
| COL24A1 | NM_152890 | collagen, type XXIV, alpha 1 |
| COL3A1 | NM_000090 | procollagen, type III, alpha 1 |
| COL4A1 | NM_001845 | alpha 1 type IV collagen preproprotein |
| COL6A1 | NM_001848 | collagen, type VI, alpha 1 precursor |
| COL8A2 | NM_005202 | collagen, type VIII, alpha 2 |
| COL9A2 | NM_001852 | alpha 2 type IX collagen |
| COLEC12 | NM_030781 | collectin sub-family member 12 isoform II |
| COLQ | NM_005677 | acetylcholinesterase collagen-like tail subunit |
| COMMD5 | NM_014066 | hypertension-related calcium-regulated gene |
| COMMD9 | NM_014186 | COMM domain containing 9 |
| COPA | NM_004371 | coatomer protein complex, subunit alpha |
| COPG2 | NM_012133 | coatomer protein complex, subunit gamma 2 |
| COPS2 | NM_004236 | COP9 constitutive photomorphogenic homolog |
| COPS7A | NM_016319 | COP9 complex subunit 7a |
| COPS7B | NM_022730 | COP9 constitutive photomorphogenic homolog |
| COQ10B | NM_025147 | hypothetical protein LOC80219 |
| COQ5 | NM_032314 | hypothetical protein LOC84274 |
| COQ9 | NM_020312 | hypothetical protein LOC57017 |
| CORO6 | NM_032854 | coronin 6 |
| CORO7 | NM_024535 | coronin 7 |
| COX10 | NM_001303 | heme A: farnesyltransferase |
| COX15 | NM_078470 | COX15 homolog isoform 1 precursor |
| CPD | NM_001304 | carboxypeptidase D precursor |
| CPEB2 | NM_182485 | cytoplasmic polyadenylation element binding |
| CPEB3 | NM_014912 | cytoplasmic polyadenylation element binding |
| CPEB4 | NM_030627 | cytoplasmic polyadenylation element binding |
| CPLX1 | NM_006651 | complexin 1 |
| CPLX3 | NM_001030005 | complexin 3 |
| CPLX4 | NM_181654 | complexin 4 |
| CPNE1 | NM_003915 | copine I |
| CPSF3L | NM_032179 | related to CPSF subunits 68 kDa isoform 2 |
| CPT1B | NM_004377 | carnitine palmitoyltransferase 1B isoform a |
| CPXM2 | NM_198148 | carboxypeptidase X (M14 family), member 2 |
| CRAMP1L | NM_020825 | Crm, cramped-like |
| CRB2 | NM_173689 | crumbs homolog 2 |
| CREB3L1 | NM_052854 | cAMP responsive element binding protein 3-like |
| CREB5 | NM_001011666 | cAMP responsive element binding protein 5 |
| CREBL1 | NM_004381 | cAMP responsive element binding protein-like 1 |
| CREBL2 | NM_001310 | cAMP responsive element binding protein-like 2 |
| CREG1 | NM_003851 | cellular repressor of E1A-stimulated genes |
| CREG2 | NM_153836 | cellular repressor of E1A-stimulated genes 2 |
| CRELD1 | NM_001031717 | cysteine-rich with EGF-like domains 1 isoform 1 |
| CRHR1 | NM_004382 | corticotropin releasing hormone receptor 1 |
| CRIM1 | NM_016441 | cysteine-rich motor neuron 1 |
| CRISPLD2 | NM_031476 | cysteine-rich secretory protein LCCL domain |
| CRKL | NM_005207 | v-crk sarcoma virus CT10 oncogene homolog |
| CRP | NM_000567 | C-reactive protein, pentraxin-related |
| CRSP7 | NM_004831 | cofactor required for Sp1 transcriptional |
| CRSP8 | NM_004269 | cofactor required for Sp1 transcriptional |
| CRSP9 | NM_004270 | cofactor required for Sp1 transcriptional |
| CRTAC1 | NM_018058 | cartilage acidic protein 1 |
| CRY2 | NM_021117 | cryptochrome 2 (photolyase-like) |
| CRYM | NM_001014444 | crystallin, mu isoform 2 |
| CRYZL1 | NM_145858 | crystallin, zeta-like 1 |
| CSDC2 | NM_014460 | RNA-binding protein pippin |
| CSDE1 | NM_001007553 | upstream of NRAS isoform 1 |
| CSF2 | NM_000758 | colony stimulating factor 2 precursor |
| CSH1 | NM_022640 | chorionic somatomammotropin hormone 1 isoform 2 |
| CSH2 | NM_022644 | chorionic somatomammotropin hormone 2 isoform 2 |
| CSNK1A1 | NM_001025105 | casein kinase 1, alpha 1 isoform 1 |
| CSNK1G1 | NM_022048 | casein kinase 1, gamma 1 isoform S |
| CSNK1G2 | NM_001319 | casein kinase 1, gamma 2 |
| CSNK2A1 | NM_001895 | casein kinase II alpha 1 subunit isoform a |
| CSPG4 | NM_001897 | melanoma-associated chondroitin sulfate |
| CSPG5 | NM_006574 | chondroitin sulfate proteoglycan 5 (neuroglycan |
| CST6 | NM_001323 | cystatin M precursor |
| CST9 | NM_001008693 | cystatin 9 |
| CST9L | NM_080610 | cystatin 9-like precursor |
| CSTA | NM_005213 | cystatin A |
| CTAGE1 | NM_172241 | cutaneous T-cell lymphoma-associated antigen 1 |
| CTDP1 | NM_004715 | CTD (carboxy-terminal domain, RNA polymerase II, |
| CTDSP1 | NM_021198 | CTD (carboxy-terminal domain, RNA polymerase II, |
| CTDSP2 | NM_005730 | nuclear LIM interactor-interacting factor 2 |
| CTDSPL | NM_001008392 | small CTD phosphatase 3 isoform 1 |
| CTH | NM_001902 | cystathionase isoform 1 |
| CTNNA1 | NM_001903 | catenin, alpha 1 |
| CTNNBIP1 | NM_001012329 | catenin, beta interacting protein 1 |
| CTNND1 | NM_001331 | catenin (cadherin-associated protein), delta 1 |
| CTSB | NM_001908 | cathepsin B preproprotein |
| CTSC | NM_148170 | cathepsin C isoform b precursor |
| CTSF | NM_003793 | cathepsin F |
| CTSO | NM_001334 | cathepsin O preproprotein |
| CTTN | NM_005231 | cortactin isoform a |
| CUL2 | NM_003591 | cullin 2 |
| CUL3 | NM_003590 | cullin 3 |
| CX3CL1 | NM_002996 | chemokine (C—X3—C motif) ligand 1 |
| CX3CR1 | NM_001337 | chemokine (C—X3—C motif) receptor 1 |
| CXCL10 | NM_001565 | small inducible cytokine B10 precursor |
| CXCR3 | NM_001504 | chemokine (C—X—C motif) receptor 3 |
| CXCR6 | NM_006564 | G protein-coupled receptor TYMSTR |
| CXorf1 | NM_004709 | hypothetical protein LOC9142 |
| CXorf40A | NM_178124 | chromosome X open reading frame 40 |
| CXorf40B | NM_001013845 | hypothetical protein LOC541578 |
| CXorf6 | NM_005491 | hypothetical protein LOC10046 |
| CYB561 | NM_001017916 | cytochrome b-561 isoform 1 |
| CYB561D1 | NM_182580 | cytochrome b-561 domain containing 1 |
| CYB5D1 | NM_144607 | hypothetical protein LOC124637 |
| CYBASC3 | NM_153611 | cytochrome b, ascorbate dependent 3 |
| CYBRD1 | NM_024843 | cytochrome b reductase 1 |
| CYCS | NM_018947 | cytochrome c |
| CYFIP1 | NM_001033028 | cytoplasmic FMR1 interacting protein 1 isoform |
| CYGB | NM_134268 | cytoglobin |
| CYP1B1 | NM_000104 | cytochrome P450, family 1, subfamily B, |
| CYP26B1 | NM_019885 | cytochrome P450, family 26, subfamily b, |
| CYP27A1 | NM_000784 | cytochrome P450, family 27, subfamily A, |
| CYP27B1 | NM_000785 | cytochrome P450, family 27, subfamily B, |
| CYP2C8 | NM_000770 | cytochrome P450, family 2, subfamily C, |
| CYP2C9 | NM_000771 | cytochrome P450, family 2, subfamily C, |
| CYP2S1 | NM_030622 | cytochrome P450, family 2, subfamily S, |
| CYP2U1 | NM_183075 | cytochrome P450, family 2, subfamily U, |
| CYP4F3 | NM_000896 | cytochrome P450, family 4, subfamily F, |
| D2HGDH | NM_152783 | D-2-hydroxyglutarate dehydrogenase |
| D4S234E | NM_014392 | brain neuron cytoplasmic protein 1 |
| D4ST1 | NM_130468 | dermatan 4 sulfotransferase 1 |
| DAB2IP | NM_032552 | DAB2 interacting protein isoform 1 |
| DACH1 | NM_004392 | dachshund homolog 1 isoform c |
| DACT2 | NM_214462 | dapper homolog 2, antagonist of beta-catenin |
| DAPK3 | NM_001348 | death-associated protein kinase 3 |
| DBF4B | NM_025104 | DBF4 homolog B isoform 2 |
| DBH | NM_000787 | dopamine beta-hydroxylase precursor |
| DBNDD2 | NM_033542 | SCF apoptosis response protein 1 isoform 2 |
| DCAKD | NM_024819 | dephospho-CoA kinase domain containing |
| DCAMKL1 | NM_004734 | doublecortin and CaM kinase-like 1 |
| DCBLD2 | NM_080927 | discoidin, CUB and LCCL domain containing 2 |
| DCTN3 | NM_024348 | dynactin 3 isoform 2 |
| DCTN4 | NM_016221 | dynactin 4 (p62) |
| DCTN5 | NM_032486 | dynactin 4 |
| DCUN1D1 | NM_020640 | RP42 homolog |
| DCUN1D2 | NM_001014283 | hypothetical protein LOC55208 isoform b |
| DCUN1D4 | NM_015115 | DCN1, defective in cullin neddylation 1, domain |
| DCX | NM_000555 | doublecortin isoform a |
| DDEF1 | NM_018482 | development and differentiation enhancing factor |
| DDEF2 | NM_003887 | development- and differentiation-enhancing |
| DDHD2 | NM_015214 | DDHD domain containing 2 |
| DDI1 | NM_001001711 | hypothetical protein LOC414301 |
| DDX11 | NM_030655 | DEAD/H (Asp-Glu-Ala-Asp/His) box polypeptide 11 |
| DDX17 | NM_006386 | DEAD box polypeptide 17 isoform p82 |
| DDX19A | NM_018332 | DDX19-like protein |
| DDX26B | NM_182540 | hypothetical protein LOC203522 |
| DDX28 | NM_018380 | DEAD (Asp-Glu-Ala-Asp) box polypeptide 28 |
| DDX31 | NM_138620 | DEAD (Asp-Glu-Ala-Asp) box polypeptide 31 |
| DDX3X | NM_001356 | DEAD/H (Asp-Glu-Ala-Asp/His) box polypeptide 3 |
| DDX3Y | NM_004660 | DEAD (Asp-Glu-Ala-Asp) box polypeptide 3, |
| DDX52 | NM_007010 | ATP-dependent RNA helicase ROK1 isoform a |
| DDX54 | NM_024072 | DEAD (Asp-Glu-Ala-Asp) box polypeptide 54 |
| DDX59 | NM_031306 | DEAD (Asp-Glu-Ala-Asp) box polypeptide 59 |
| DEADC1 | NM_182503 | deaminase domain containing 1 |
| DEC1 | NM_017418 | deleted in esophageal cancer 1 |
| DEDD | NM_032998 | death effector domain-containing protein |
| DEFB4 | NM_004942 | defensin, beta 4 precursor |
| DENND1A | NM_020946 | hypothetical protein LOC57706 isoform 1 |
| DENND2C | NM_198459 | DENN/MADD domain containing 2C |
| DENND4A | NM_005848 | c-myc promoter binding protein |
| DENR | NM_003677 | density-regulated protein |
| DEPDC4 | NM_152317 | DEP domain containing 4 |
| DEPDC5 | NM_014662 | DEP domain containing 5 isoform 1 |
| DERL3 | NM_001002862 | derlin-3 protein isoform b |
| DFFB | NM_001004285 | DNA fragmentation factor, 40 kD, beta |
| DGAT2L4 | NM_001002254 | diacylglycerol O-acyltransferase 2-like 4 |
| DGCR13 | NM_001024733 | DiGeorge syndrome gene H |
| DGCR2 | NM_005137 | integral membrane protein DGCR2 |
| DGCR6 | NM_005675 | DiGeorge syndrome critical region protein 6 |
| DGCR6L | NM_033257 | DiGeorge syndrome critical region gene 6 like |
| DGCR8 | NM_022720 | DiGeorge syndrome critical region gene 8 |
| DGKD | NM_003648 | diacylglycerol kinase, delta 130 kDa isoform 1 |
| DHDDS | NM_024887 | dehydrodolichyl diphosphate synthase isoform a |
| DHFR | NM_000791 | dihydrofolate reductase |
| DHFRL1 | NM_176815 | dihydrofolate reductase-like 1 |
| DHTKD1 | NM_018706 | dehydrogenase E1 and transketolase domain |
| DHX30 | NM_138614 | DEAH (Asp-Glu-Ala-His) box polypeptide 30 |
| DHX33 | NM_020162 | DEAH (Asp-Glu-Ala-His) box polypeptide 33 |
| DHX35 | NM_021931 | DEAH (Asp-Glu-Ala-His) box polypeptide 35 |
| DIAPH1 | NM_005219 | diaphanous 1 |
| DICER1 | NM_030621 | dicer1 |
| DIO2 | NM_000793 | deiodinase, iodothyronine, type II isoform a |
| DIP | NM_015124 | death-inducing-protein |
| DIP2A | NM_015151 | DIP2-like protein isoform a |
| DIRAS1 | NM_145173 | small GTP-binding tumor suppressor 1 |
| DIRAS2 | NM_017594 | Di-Ras2 |
| DIRC1 | NM_052952 | hypothetical protein LOC116093 |
| DISC1 | NM_001012957 | disrupted in schizophrenia 1 isoform Lv |
| DISP2 | NM_033510 | dispatched B |
| DIXDC1 | NM_033425 | DIX domain containing 1 isoform b |
| dJ341D10.1 | NM_001007535 | hypothetical protein LOC286453 |
| DKC1 | NM_001363 | dyskerin |
| DKFZp434I1020 | NM_194295 | hypothetical protein LOC196968 |
| DKFZp434K191 | NM_001029950 | hypothetical protein LOC29797 |
| DKFZp434N035 | NM_032262 | hypothetical protein LOC84222 |
| DKFZp451A211 | NM_001003399 | hypothetical protein LOC400169 |
| DKFZP564O0823 | NM_015393 | DKFZP564O0823 protein |
| DKFZP586D0919 | NM_206914 | hypothetical protein LOC25895 isoform b |
| DKFZp666G057 | NM_001008226 | hypothetical protein LOC283726 |
| DKFZp667M2411 | NM_207323 | hypothetical protein LOC147172 |
| DKFZp686I15217 | NM_207495 | hypothetical protein LOC401232 |
| DKFZp686O24166 | NM_001009913 | hypothetical protein LOC374383 |
| DKFZp761E198 | NM_138368 | hypothetical protein LOC91056 |
| DKFZP761H1710 | NM_031297 | hypothetical protein LOC83459 |
| DKFZp761I2123 | NM_031449 | hypothetical protein LOC83637 isoform 1 |
| DKFZp779B1540 | NM_001010903 | hypothetical protein LOC389384 |
| DLEC1 | NM_007335 | deleted in lung and esophageal cancer 1 isoform |
| DLEU7 | NM_198989 | deleted in lymphocytic leukemia, 7 |
| DLGAP2 | NM_004745 | discs large-associated protein 2 |
| DLGAP4 | NM_014902 | disks large-associated protein 4 isoform a |
| DLK1 | NM_001032997 | delta-like 1 homolog isoform 2 |
| DLL1 | NM_005618 | delta-like 1 |
| DLL4 | NM_019074 | delta-like 4 protein precursor |
| DLST | NM_001933 | dihydrolipoamide S-succinyltransferase (E2 |
| DMAP1 | NM_019100 | DNA methyltransferase 1 associated protein 1 |
| DMD | NM_000109 | dystrophin Dp427c isoform |
| DMPK | NM_004409 | myotonic dystrophy protein kinase |
| DMRT2 | NM_006557 | doublesex and mab-3 related transcription factor |
| DMRTB1 | NM_033067 | DMRT-like family B with proline-rich C-terminal, |
| DMTF1 | NM_021145 | cyclin D binding myb-like transcription factor |
| DNAJA2 | NM_005880 | DnaJ subfamily A member 2 |
| DNAJA3 | NM_005147 | DnaJ (Hsp40) homolog, subfamily A, member 3 |
| DNAJA4 | NM_018602 | DnaJ (Hsp40) homolog, subfamily A, member 4 |
| DNAJB12 | NM_001002762 | DnaJ (Hsp40) homolog, subfamily B, member 12 |
| DNAJB14 | NM_024920 | DnaJ (Hsp40) homolog, subfamily B, member 14 |
| DNAJB4 | NM_007034 | DnaJ (Hsp40) homolog, subfamily B, member 4 |
| DNAJB5 | NM_012266 | DnaJ (Hsp40) homolog, subfamily B, member 5 |
| DNAJB6 | NM_058246 | DnaJ (Hsp40) homolog, subfamily B, member 6 |
| DNAJC18 | NM_152686 | DnaJ (Hsp40) homolog, subfamily C, member 18 |
| DNAJC5G | NM_173650 | DnaJ (Hsp40) homolog, subfamily C, member 5 |
| DNAJC9 | NM_015190 | DnaJ homolog, subfamily C, member 9 |
| DNAL4 | NM_005740 | dynein light chain 4, axonemal |
| DNALI1 | NM_003462 | axonemal dynein light chain |
| DNASE1L1 | NM_001009932 | deoxyribonuclease I-like 1 precursor |
| DNASE1L2 | NM_001374 | deoxyribonuclease I-like 2 |
| DNM1L | NM_012062 | dynamin 1-like protein isoform 1 |
| DOCK2 | NM_004946 | dedicator of cytokinesis 2 |
| DOCK3 | NM_004947 | dedicator of cytokinesis 3 |
| DOCK5 | NM_024940 | dedicator of cytokinesis 5 |
| DOK2 | NM_003974 | docking protein 2 |
| DOK4 | NM_018110 | downstream of tyrosine kinase 4 |
| DOLPP1 | NM_020438 | dolichyl pyrophosphate phosphatase 1 |
| DPF3 | NM_012074 | D4, zinc and double PHD fingers, family 3 |
| DPH2 | NM_001384 | diphthamide biosynthesis protein 2 isoform a |
| DPP9 | NM_139159 | dipeptidylpeptidase 9 |
| DPPA4 | NM_018189 | developmental pluripotency associated 4 |
| DPT | NM_001937 | dermatopontin precursor |
| DPY19L4 | NM_181787 | hypothetical protein LOC286148 |
| DPYSL2 | NM_001386 | dihydropyrimidinase-like 2 |
| DPYSL3 | NM_001387 | dihydropyrimidinase-like 3 |
| DRD1 | NM_000794 | dopamine receptor D1 |
| DRD2 | NM_000795 | dopamine receptor D2 isoform long |
| DRD5 | NM_000798 | dopamine receptor D5 |
| DREV1 | NM_016025 | hypothetical protein LOC51108 |
| DSC3 | NM_024423 | desmocollin 3 isoform Dsc3b preproprotein |
| DSCR10 | NM_148676 | hypothetical protein LOC259234 |
| DSCR3 | NM_006052 | Down syndrome critical region protein 3 |
| DTNA | NM_001390 | dystrobrevin alpha isoform 1 |
| DUOX2 | NM_014080 | dual oxidase 2 precursor |
| DUS1L | NM_022156 | PP3111 protein |
| DUSP10 | NM_007207 | dual specificity phosphatase 10 isoform a |
| DUSP13 | NM_001007271 | muscle-restricted dual specificity phosphatase |
| DUSP2 | NM_004418 | dual specificity phosphatase 2 |
| DUSP26 | NM_024025 | dual specificity phosphatase 26 |
| DUSP3 | NM_004090 | dual specificity phosphatase 3 |
| DUSP9 | NM_001395 | dual specificity phosphatase 9 |
| DUX1 | NM_012146 | double homeobox, 1 |
| DUXA | NM_001012729 | hypothetical protein LOC503835 |
| DVL1 | NM_004421 | dishevelled 1 isoform a |
| DVL2 | NM_004422 | dishevelled 2 |
| DVL3 | NM_004423 | dishevelled 3 |
| DXYS155E | NM_005088 | DNA segment on chromosome X and Y (unique) 155 |
| DYNC1I1 | NM_004411 | dynein, cytoplasmic, intermediate polypeptide 1 |
| DYNC1LI2 | NM_006141 | dynein, cytoplasmic, light intermediate |
| DYNLT3 | NM_006520 | t-complex-associated-testis-expressed 1-like |
| DYRK1A | NM_101395 | dual-specificity tyrosine-(Y)-phosphorylation |
| DYRK1B | NM_004714 | dual-specificity tyrosine-(Y)-phosphorylation |
| DZIP1 | NM_014934 | DAZ interacting protein 1 isoform 1 |
| DZIP3 | NM_014648 | zinc finger DAZ interacting protein 3 |
| E2F3 | NM_001949 | E2F transcription factor 3 |
| E2F7 | NM_203394 | E2F transcription factor 7 |
| EBI3 | NM_005755 | Epstein-Barr virus induced gene 3 precursor |
| ECE2 | NM_014693 | endothelin converting enzyme 2 isoform A |
| ECHDC1 | NM_018479 | enoyl Coenzyme A hydratase domain containing 1 |
| ECHS1 | NM_004092 | mitochondrial short-chain enoyl-coenzyme A |
| ECOP | NM_030796 | EGFR-coamplified and overexpressed protein |
| EDA | NM_001005609 | ectodysplasin A isoform EDA-A2 |
| EDA2R | NM_021783 | X-linked ectodysplasin receptor |
| EDAR | NM_022336 | ectodysplasin A receptor |
| EDARADD | NM_080738 | EDAR-associated death domain isoform B |
| EDG1 | NM_001400 | endothelial differentiation, sphingolipid |
| EDN2 | NM_001956 | endothelin 2 |
| EED | NM_152991 | embryonic ectoderm development isoform b |
| EEFSEC | NM_021937 | elongation factor for selenoprotein translation |
| EFCAB1 | NM_024593 | EF-hand calcium binding domain 1 |
| EFCAB4A | NM_173584 | hypothetical protein LOC283229 |
| EFCAB5 | NM_198529 | EF-hand calcium binding domain 5 isoform 1 |
| EFNA3 | NM_004952 | ephrin A3 |
| EFNB1 | NM_004429 | ephrin-B1 precursor |
| EFNB2 | NM_004093 | ephrin B2 |
| EFNB3 | NM_001406 | ephrin-B3 precursor |
| EFTUD1 | NM_024580 | elongation factor Tu GTP binding domain |
| EGFL7 | NM_016215 | EGF-like-domain, multiple 7 |
| EGLN1 | NM_022051 | egl nine homolog 1 |
| EGLN2 | NM_017555 | EGL nine (C. elegans) homolog 2 isoform 2 |
| EGR3 | NM_004430 | early growth response 3 |
| EHD1 | NM_006795 | EH-domain containing 1 |
| EHMT1 | NM_024757 | euchromatic histone methyltransferase 1 |
| EHMT2 | NM_006709 | HLA-B associated transcript 8 isoform a |
| EIF1AX | NM_001412 | X-linked eukaryotic translation initiation |
| EIF2B2 | NM_014239 | eukaryotic translation initiation factor 2B, |
| EIF2B5 | NM_003907 | eukaryotic translation initiation factor 2B, |
| EIF2C1 | NM_012199 | eukaryotic translation initiation factor 2C, 1 |
| EIF2C2 | NM_012154 | eukaryotic translation initiation factor 2C, 2 |
| EIF2C4 | NM_017629 | eukaryotic translation initiation factor 2C, 4 |
| EIF2S2 | NM_003908 | eukaryotic translation initiation factor 2 beta |
| EIF3S10 | NM_003750 | eukaryotic translation initiation factor 3, |
| EIF3S8 | NM_003752 | eukaryotic translation initiation factor 3, |
| EIF4B | NM_001417 | eukaryotic translation initiation factor 4B |
| EIF4E | NM_001968 | eukaryotic translation initiation factor 4E |
| EIF4E3 | NM_173359 | eukaryotic translation initiation factor 4E |
| EIF4EBP2 | NM_004096 | eukaryotic translation initiation factor 4E |
| EIF4G1 | NM_004953 | eukaryotic translation initiation factor 4 |
| EIF5A | NM_001970 | eukaryotic translation initiation factor 5A |
| EIF5A2 | NM_020390 | eIF-5A2 protein |
| ELAC1 | NM_018696 | elaC homolog 1 |
| ELAVL1 | NM_001419 | ELAV-like 1 |
| ELF4 | NM_001421 | E74-like factor 4 (ets domain transcription |
| ELL | NM_006532 | elongation factor RNA polymerase II |
| ELL2 | NM_012081 | elongation factor, RNA polymerase II, 2 |
| Ells1 | NM_152793 | hypothetical protein LOC222166 |
| ELMO2 | NM_133171 | engulfment and cell motility 2 |
| ELMOD1 | NM_018712 | ELMO domain containing 1 |
| ELOVL1 | NM_022821 | elongation of very long chain fatty acids |
| ELOVL2 | NM_017770 | elongation of very long chain fatty acids |
| ELOVL5 | NM_021814 | homolog of yeast long chain polyunsaturated |
| ELOVL6 | NM_024090 | ELOVL family member 6, elongation of long chain |
| ELOVL7 | NM_024930 | ELOVL family member 7, elongation of long chain |
| ELP3 | NM_018091 | elongation protein 3 homolog |
| EMCN | NM_016242 | endomucin |
| EMILIN3 | NM_052846 | elastin microfibril interfacer 3 |
| EML5 | NM_183387 | echinoderm microtubule associated protein like |
| EMR2 | NM_013447 | egf-like module containing, mucin-like, hormone |
| EMR3 | NM_152939 | egf-like module-containing mucin-like receptor 3 |
| EMX1 | NM_004097 | empty spiracles homolog 1 isoform 1 |
| EN2 | NM_001427 | engrailed homolog 2 |
| ENAH | NM_001008493 | enabled homolog isoform a |
| ENC1 | NM_003633 | ectodermal-neural cortex (with BTB-like domain) |
| ENG | NM_000118 | endoglin precursor |
| ENPP4 | NM_014936 | ectonucleotide pyrophosphatase/phosphodiesterase |
| ENSA | NM_207043 | endosulfine alpha isoform 2 |
| ENTPD6 | NM_001247 | ectonucleoside triphosphate diphosphohydrolase |
| ENTPD7 | NM_020354 | ectonucleoside triphosphate diphosphohydrolase |
| EPB41L1 | NM_012156 | erythrocyte membrane protein band 4.1-like 1 |
| EPB41L4B | NM_018424 | erythrocyte membrane protein band 4.1 like 4B |
| EPB41L5 | NM_020909 | erythrocyte membrane protein band 4.1 like 5 |
| EPB49 | NM_001978 | erythrocyte membrane protein band 4.9 (dematin) |
| EPHA1 | NM_005232 | ephrin receptor EphA1 |
| EPHA7 | NM_004440 | ephrin receptor EphA7 |
| EPHB2 | NM_004442 | ephrin receptor EphB2 isoform 2 precursor |
| EPHB4 | NM_004444 | ephrin receptor EphB4 precursor |
| EPHX2 | NM_001979 | epoxide hydrolase 2, cytoplasmic |
| EPM2AIP1 | NM_014805 | EPM2A interacting protein 1 |
| EPS8L2 | NM_022772 | epidermal growth factor receptor pathway |
| ERGIC1 | NM_001031711 | endoplasmic reticulum-golgi intermediate |
| ERN2 | NM_033266 | endoplasmic reticulum to nucleus signalling 2 |
| ESAM | NM_138961 | endothelial cell adhesion molecule |
| ESPN | NM_031475 | espin |
| ESR1 | NM_000125 | estrogen receptor 1 |
| ESRRA | NM_004451 | estrogen-related receptor alpha |
| ESRRG | NM_001438 | estrogen-related receptor gamma isoform 1 |
| ET | NM_024311 | hypothetical protein LOC79157 |
| ETS1 | NM_005238 | v-ets erythroblastosis virus E26 oncogene |
| ETS2 | NM_005239 | v-ets erythroblastosis virus E26 oncogene |
| ETV1 | NM_004956 | ets variant gene 1 |
| ETV6 | NM_001987 | ets variant gene 6 |
| EVI5 | NM_005665 | ecotropic viral integration site 5 |
| EVL | NM_016337 | Enah/Vasp-like |
| EXOC2 | NM_018303 | Sec5 protein |
| EXOC4 | NM_021807 | SEC8 protein isoform a |
| EXOC5 | NM_006544 | SEC10 protein |
| EXOC7 | NM_001013839 | exocyst complex component 7 isoform a |
| EXOD1 | NM_080663 | hypothetical protein LOC112479 |
| EXOSC1 | NM_016046 | exosomal core protein CSL4 |
| EXOSC10 | NM_001001998 | exosome component 10 isoform 1 |
| EXT2 | NM_000401 | exostosin 2 |
| EXTL3 | NM_001440 | Reg receptor |
| EYA1 | NM_000503 | eyes absent 1 isoform b |
| EZH1 | NM_001991 | enhancer of zeste homolog 1 |
| F11R | NM_016946 | F11 receptor isoform a precursor |
| F2RL1 | NM_005242 | coagulation factor II (thrombin) receptor-like 1 |
| F7 | NM_000131 | coagulation factor VII precursor, isoform a |
| FABP2 | NM_000134 | intestinal fatty acid binding protein 2 |
| FADS1 | NM_013402 | fatty acid desaturase 1 |
| FADS2 | NM_004265 | fatty acid desaturase 2 |
| FADS6 | NM_178128 | fatty acid desaturase domain family, member 6 |
| FAIM2 | NM_012306 | Fas apoptotic inhibitory molecule 2 |
| FALZ | NM_004459 | fetal Alzheimer antigen isoform 2 |
| FAM101A | NM_181709 | hypothetical protein LOC144347 |
| FAM102A | NM_203305 | early estrogen-induced gene 1 protein isoform b |
| FAM107A | NM_007177 | downregulated in renal cell carcinoma |
| FAM107B | NM_031453 | hypothetical protein LOC83641 |
| FAM111A | NM_022074 | hypothetical protein LOC63901 |
| FAM116A | NM_152678 | hypothetical protein LOC201627 |
| FAM11A | NM_032508 | family with sequence similarity 11, member A |
| FAM18B | NM_016078 | hypothetical protein LOC51030 |
| FAM20B | NM_014864 | family with sequence similarity 20, member B |
| FAM29A | NM_017645 | hypothetical protein LOC54801 |
| FAM32A | NM_014077 | hypothetical protein LOC26017 |
| FAM38A | NM_014745 | family with sequence similarity 38, member A |
| FAM3A | NM_021806 | family 3, member A protein |
| FAM43B | NM_207334 | hypothetical protein LOC163933 |
| FAM46C | NM_017709 | hypothetical protein LOC54855 |
| FAM50A | NM_004699 | XAP-5 protein |
| FAM53A | NM_001013622 | dorsal neural-tube nuclear protein |
| FAM54B | NM_019557 | hypothetical protein LOC56181 |
| FAM55C | NM_145037 | hypothetical protein LOC91775 |
| FAM57B | NM_031478 | hypothetical protein LOC83723 |
| FAM58A | NM_152274 | hypothetical protein LOC92002 |
| FAM59A | NM_022751 | hypothetical protein LOC64762 |
| FAM60A | NM_021238 | family with sequence similarity 60, member A |
| FAM62A | NM_015292 | family with sequence similarity 62 (C2 domain |
| FAM63A | NM_018379 | hypothetical protein LOC55793 isoform 1 |
| FAM63B | NM_019092 | hypothetical protein LOC54629 |
| FAM70A | NM_017938 | hypothetical protein LOC55026 |
| FAM73A | NM_198549 | hypothetical protein LOC374986 |
| FAM78A | NM_033387 | hypothetical protein LOC286336 |
| FAM78B | NM_001017961 | hypothetical protein LOC149297 |
| FAM79A | NM_182752 | hypothetical protein LOC127262 |
| FAM79B | NM_198485 | hypothetical protein LOC285386 |
| FAM81A | NM_152450 | hypothetical protein LOC145773 |
| FAM84B | NM_174911 | breast cancer membrane protein 101 |
| FAM86B1 | NM_032916 | hypothetical protein LOC85002 |
| FAM86C | NM_018172 | hypothetical protein LOC55199 isoform 1 |
| FAM89A | NM_198552 | hypothetical protein LOC375061 |
| FAM89B | NM_152832 | Mouse Mammary Turmor Virus Receptor homolog 1 |
| FAM91A1 | NM_144963 | hypothetical protein LOC157769 |
| FAM98B | NM_173611 | hypothetical protein LOC283742 |
| FAM99A | NM_001014374 | hypothetical protein LOC387742 |
| FANCA | NM_000135 | Fanconi anemia, complementation group A isoform |
| FANCE | NM_021922 | Fanconi anemia, complementation group E |
| FARSLA | NM_004461 | phenylalanine-tRNA synthetase-like protein |
| FASN | NM_004104 | fatty acid synthase |
| FAT2 | NM_001447 | FAT tumor suppressor 2 precursor |
| FBLN1 | NM_006487 | fibulin 1 isoform A precursor |
| FBXO17 | NM_024907 | F-box protein FBG4 isoform 2 |
| FBXO21 | NM_015002 | F-box only protein 21 isoform 2 |
| FBXO22 | NM_147188 | F-box only protein 22 isoform a |
| FBXO24 | NM_012172 | F-box only protein 24 isoform 2 |
| FBXO27 | NM_178820 | F-box protein 27 |
| FBXO31 | NM_024735 | F-box protein 31 |
| FBXO33 | NM_203301 | F-box protein 33 |
| FBXO44 | NM_001014765 | F-box protein 44 isoform 1 |
| FBXW11 | NM_012300 | F-box and WD-40 domain protein 1B isoform C |
| FBXW4 | NM_022039 | F-box and WD-40 domain protein 4 |
| FBXW5 | NM_018998 | F-box and WD-40 domain protein 5 |
| FBXW7 | NM_001013415 | F-box protein FBW7 isoform 3 |
| FCHO1 | NM_015122 | FCH domain only 1 |
| FCHSD1 | NM_033449 | FCH and double SH3 domains 1 |
| FCHSD2 | NM_014824 | FCH and double SH3 domains 2 |
| FCMD | NM_006731 | fukutin |
| FCRL2 | NM_030764 | Fc receptor-like 2 isoform b |
| FCRL5 | NM_031281 | Fc receptor-like 5 |
| FDFT1 | NM_004462 | farnesyl-diphosphate farnesyltransferase 1 |
| FECH | NM_000140 | ferrochelatase isoform b precursor |
| FEM1C | NM_020177 | feminization 1 homolog a |
| FES | NM_002005 | V-FES feline sarcoma viral/V-FPS fujinami avian |
| FEZ1 | NM_022549 | zygin 1 isoform 2 |
| FEZ2 | NM_005102 | zygin 2 |
| FFAR3 | NM_005304 | G protein-coupled receptor 41 |
| FGD3 | NM_033086 | FYVE, RhoGEF and PH domain containing 3 |
| FGF11 | NM_004112 | fibroblast growth factor 11 |
| FGF19 | NM_005117 | fibroblast growth factor 19 precursor |
| FGF2 | NM_002006 | fibroblast growth factor 2 |
| FGF23 | NM_020638 | fibroblast growth factor 23 precursor |
| FGF7 | NM_002009 | fibroblast growth factor 7 precursor |
| FGFR1 | NM_023107 | fibroblast growth factor receptor 1 isoform 5 |
| FGFR1OP | NM_007045 | FGFR1 oncogene partner isoform a |
| FGFR2 | NM_000141 | fibroblast growth factor receptor 2 isoform 1 |
| FGFR3 | NM_000142 | fibroblast growth factor receptor 3 isoform 1 |
| FGFR4 | NM_002011 | fibroblast growth factor receptor 4 isoform 1 |
| FGL1 | NM_004467 | fibrinogen-like 1 precursor |
| FGR | NM_005248 | Gardner-Rasheed feline sarcoma viral (v-fgr) |
| FHL1 | NM_001449 | four and a half LIM domains 1 |
| FHL2 | NM_001450 | four and a half LIM domains 2 |
| FIBCD1 | NM_032843 | fibrinogen C domain containing 1 |
| FIGF | NM_004469 | vascular endothelial growth factor D |
| FIS | NM_175616 | hypothetical protein LOC202299 |
| FKBP10 | NM_021939 | FK506 binding protein 10, 65 kDa |
| FKBP1A | NM_000801 | FK506-binding protein 1A |
| FKBP1B | NM_004116 | FK506-binding protein 1B isoform a |
| FKBP5 | NM_004117 | FK506 binding protein 5 |
| FKBP9 | NM_007270 | FK506 binding protein 9 |
| FKBP9L | NM_182827 | FK506 binding protein 9-like |
| FKRP | NM_024301 | fukutin-related protein |
| FKSG44 | NM_031904 | FKSG44 protein |
| FLCN | NM_144997 | folliculin isoform 1 |
| FLJ10159 | NM_018013 | hypothetical protein LOC55084 |
| FLJ10324 | NM_018059 | hypothetical protein LOC55698 |
| FLJ10769 | NM_018210 | hypothetical protein LOC55739 |
| FLJ10803 | NM_018224 | hypothetical protein LOC55744 |
| FLJ10916 | NM_018271 | hypothetical protein LOC55258 |
| FLJ10945 | NM_018280 | hypothetical protein LOC55267 |
| FLJ11259 | NM_018370 | hypothetical protein LOC55332 |
| FLJ11292 | NM_018382 | hypothetical protein LOC55338 |
| FLJ11506 | NM_024666 | hypothetical protein LOC79719 |
| FLJ11783 | NM_024891 | hypothetical protein LOC79951 |
| FLJ11806 | NM_024824 | nuclear protein UKp68 isoform 1 |
| FLJ12118 | NM_024537 | hypothetical protein LOC79587 |
| FLJ12529 | NM_024811 | pre-mRNA cleavage factor I, 59 kDa subunit |
| FLJ12700 | NM_024910 | hypothetical protein LOC79970 |
| FLJ12716 | NM_199053 | hypothetical protein LOC60684 isoform b |
| FLJ12788 | NM_022492 | hypothetical protein LOC64427 |
| FLJ13841 | NM_024702 | hypothetical protein LOC79755 |
| FLJ14001 | NM_024677 | hypothetical protein LOC79730 |
| FLJ14107 | NM_025026 | hypothetical protein LOC80094 |
| FLJ14154 | NM_024845 | hypothetical protein LOC79903 |
| FLJ14213 | NM_024841 | hypothetical protein LOC79899 |
| FLJ14816 | NM_032845 | hypothetical protein LOC84931 |
| FLJ16008 | NM_001001665 | hypothetical protein LOC339761 |
| FLJ16165 | NM_001004318 | hypothetical protein LOC390928 |
| FLJ20032 | NM_017628 | hypothetical protein LOC54790 |
| FLJ20186 | NM_207514 | differentially expressed in FDCP 8 isoform 1 |
| FLJ20232 | NM_019008 | hypothetical protein LOC54471 |
| FLJ20298 | NM_017752 | hypothetical protein LOC54885 isoform a |
| FLJ20487 | NM_017841 | hypothetical protein LOC54949 |
| FLJ20551 | NM_017875 | hypothetical protein LOC54977 |
| FLJ20558 | NM_017880 | hypothetical protein LOC54980 |
| FLJ20699 | NM_017931 | hypothetical protein LOC55020 |
| FLJ20701 | NM_017933 | hypothetical protein LOC55022 |
| FLJ20758 | NM_017952 | hypothetical protein LOC55037 |
| FLJ20850 | NM_017967 | hypothetical protein LOC55049 |
| FLJ21125 | NM_024627 | hypothetical protein LOC79680 |
| FLJ21687 | NM_024859 | PDZ domain containing, X chromosome |
| FLJ21736 | NM_024922 | esterase 31 |
| FLJ21742 | NM_032207 | hypothetical protein LOC84167 |
| FLJ21945 | NM_025203 | hypothetical protein LOC80304 |
| FLJ21986 | NM_024913 | hypothetical protein LOC79974 |
| FLJ22349 | NM_024821 | hypothetical protein LOC79879 |
| FLJ22374 | NM_032222 | hypothetical protein LOC84182 |
| FLJ23436 | NM_024671 | hypothetical protein LOC79724 |
| FLJ25102 | NM_182626 | hypothetical protein LOC348738 |
| FLJ25143 | NM_182500 | hypothetical protein LOC130813 |
| FLJ25169 | NM_152568 | hypothetical protein LOC157848 |
| FLJ25222 | NM_199163 | hypothetical protein LOC374666 |
| FLJ25410 | NM_144605 | hypothetical protein LOC124404 |
| FLJ25476 | NM_152493 | hypothetical protein LOC149076 |
| FLJ27255 | NM_207501 | hypothetical protein LOC401281 |
| FLJ30294 | NM_144632 | hypothetical protein LOC130827 |
| FLJ30313 | NM_152757 | hypothetical protein LOC253868 |
| FLJ31132 | NM_001004355 | hypothetical protein LOC441522 |
| FLJ32011 | NM_182516 | hypothetical protein LOC148930 |
| FLJ32028 | NM_152680 | hypothetical protein LOC201799 |
| FLJ32063 | NM_153031 | hypothetical protein LOC150538 |
| FLJ32252 | NM_182510 | hypothetical protein LOC146336 |
| FLJ33708 | NM_173675 | hypothetical protein LOC285780 |
| FLJ35220 | NM_173627 | hypothetical protein LOC284131 |
| FLJ35424 | NM_173661 | hypothetical protein LOC285492 |
| FLJ35429 | NM_001003807 | hypothetical protein LOC285830 |
| FLJ35530 | NM_207467 | hypothetical protein LOC400798 |
| FLJ35695 | NM_207444 | hypothetical protein LOC400359 |
| FLJ35740 | NM_147195 | FLJ35740 protein |
| FLJ35767 | NM_207459 | hypothetical protein LOC400629 |
| FLJ35880 | NM_153264 | hypothetical protein LOC256076 |
| FLJ36070 | NM_182574 | hypothetical protein LOC284358 |
| FLJ36208 | NM_176677 | hypothetical protein LOC283948 |
| FLJ36492 | NM_182568 | hypothetical protein LOC284047 |
| FLJ36888 | NM_178830 | hypothetical protein LOC126526 |
| FLJ37357 | NM_173645 | hypothetical protein LOC284944 |
| FLJ37478 | NM_178557 | hypothetical protein LOC339983 |
| FLJ37538 | NM_173564 | hypothetical protein FLJ37538 |
| FLJ37543 | NM_173667 | hypothetical protein LOC285668 |
| FLJ38723 | NM_173805 | hypothetical protein FLJ38723 |
| FLJ38973 | NM_153689 | hypothetical protein LOC205327 |
| FLJ39237 | NM_198571 | hypothetical protein LOC375607 |
| FLJ39827 | NM_152424 | hypothetical protein LOC139285 |
| FLJ40142 | NM_207435 | hypothetical protein LOC400073 |
| FLJ40172 | NM_173649 | hypothetical protein LOC285051 |
| FLJ40288 | NM_173682 | hypothetical protein LOC286023 |
| FLJ40432 | NM_152523 | hypothetical protein LOC151195 |
| FLJ40504 | NM_173624 | hypothetical protein LOC284085 |
| FLJ41046 | NM_207479 | hypothetical protein LOC400940 |
| FLJ41423 | NM_001001679 | hypothetical protein LOC399886 |
| FLJ41821 | NM_001001697 | hypothetical protein LOC401011 |
| FLJ41993 | NM_001001694 | hypothetical protein LOC400935 |
| FLJ42102 | NM_001001680 | hypothetical protein LOC399923 |
| FLJ42133 | NM_001001690 | hypothetical protein LOC400844 |
| FLJ42289 | NM_207383 | hypothetical protein LOC388182 |
| FLJ42291 | NM_207367 | hypothetical protein LOC346547 |
| FLJ43093 | NM_207498 | hypothetical protein LOC401258 |
| FLJ43339 | NM_207380 | hypothetical protein LOC388115 |
| FLJ43582 | NM_207412 | hypothetical protein LOC389649 |
| FLJ43980 | NM_001004299 | hypothetical protein LOC124149 |
| FLJ44385 | NM_207478 | hypothetical protein LOC400934 |
| FLJ44815 | NM_207454 | hypothetical protein LOC400591 |
| FLJ44968 | NM_198537 | hypothetical protein LOC374887 |
| FLJ45079 | NM_001001685 | hypothetical protein LOC400624 |
| FLJ45121 | NM_207451 | hypothetical protein LOC400556 |
| FLJ45248 | NM_207505 | hypothetical protein LOC401472 |
| FLJ45300 | NM_001001681 | hypothetical protein LOC399957 |
| FLJ45422 | NM_001004349 | hypothetical protein LOC441140 |
| FLJ45455 | NM_207386 | hypothetical protein LOC388336 |
| FLJ45537 | NM_001001709 | hypothetical protein LOC401535 |
| FLJ45645 | NM_198557 | hypothetical protein LOC375287 |
| FLJ45684 | NM_207462 | hypothetical protein LOC400666 |
| FLJ45831 | NM_001001684 | hypothetical protein LOC400576 |
| FLJ45964 | NM_207483 | hypothetical protein LOC401040 |
| FLJ45966 | NM_001001700 | hypothetical protein LOC401120 |
| FLJ45974 | NM_001001707 | hypothetical protein LOC401337 |
| FLJ46020 | NM_207472 | hypothetical protein LOC400863 |
| FLJ46026 | NM_207458 | hypothetical protein LOC400627 |
| FLJ46082 | NM_207417 | hypothetical protein LOC389799 |
| FLJ46154 | NM_198462 | FLJ46154 protein |
| FLJ46210 | NM_001004315 | hypothetical protein LOC389152 |
| FLJ46230 | NM_207463 | hypothetical protein LOC400679 |
| FLJ46257 | NM_001001693 | hypothetical protein LOC400932 |
| FLJ46347 | NM_001005303 | hypothetical protein LOC389064 |
| FLJ46358 | NM_207439 | hypothetical protein LOC400110 |
| FLJ46363 | NM_207434 | hypothetical protein LOC400002 |
| FLJ46365 | NM_207504 | hypothetical protein LOC401459 |
| FLJ46385 | NM_001001675 | hypothetical protein LOC390963 |
| FLJ46481 | NM_207405 | hypothetical protein LOC389197 |
| FLJ46831 | NM_207426 | forkhead box I2 |
| FLJ46838 | NM_001007546 | hypothetical protein LOC440865 |
| FLJ90166 | NM_153360 | hypothetical protein LOC164284 |
| FLJ90579 | NM_173591 | hypothetical protein LOC283310 |
| FLJ90650 | NM_173800 | laeverin |
| FLJ90709 | NM_173514 | hypothetical protein LOC153129 |
| FLNA | NM_001456 | filamin 1 (actin-binding protein-280) |
| FLNB | NM_001457 | filamin B, beta (actin binding protein 278) |
| FLOT2 | NM_004475 | flotillin 2 |
| FLRT2 | NM_013231 | fibronectin leucine rich transmembrane protein |
| FLT3 | NM_004119 | fms-related tyrosine kinase 3 |
| FLYWCH1 | NM_032296 | FLYWCH-type zinc finger 1 isoform a |
| FMNL1 | NM_005892 | formin-like 1 |
| FMNL3 | NM_175736 | formin-like 3 isoform 1 |
| FN3KRP | NM_024619 | fructosamine-3-kinase-related protein |
| FNDC3A | NM_014923 | fibronectin type III domain containing 3A |
| FNDC3B | NM_022763 | fibronectin type III domain containing 3B |
| FNDC4 | NM_022823 | fibronectin type III domain containing 4 |
| FNDC5 | NM_153756 | fibronectin type III domain containing 5 |
| FNDC7 | NM_173532 | hypothetical protein LOC163479 |
| FNDC8 | NM_017559 | hypothetical protein LOC54752 |
| FNTA | NM_001018676 | farnesyltransferase, CAAX box, alpha isoform b |
| FNTB | NM_002028 | farnesyltransferase, CAAX box, beta |
| FOLR2 | NM_000803 | folate receptor 2 precursor |
| FOSB | NM_006732 | FBJ murine osteosarcoma viral oncogene homolog |
| FOSL1 | NM_005438 | FOS-like antigen 1 |
| FOSL2 | NM_005253 | FOS-like antigen 2 |
| FOXA3 | NM_004497 | forkhead box A3 |
| FOXF1 | NM_001451 | forkhead box F1 |
| FOXL2 | NM_023067 | forkhead box L2 |
| FOXN1 | NM_003593 | forkhead box N1 |
| FOXO1A | NM_002015 | forkhead box O1A |
| FOXP4 | NM_001012426 | forkhead box P4 isoform 1 |
| FOXRED1 | NM_017547 | FAD-dependent oxidoreductase domain containing |
| FRAG1 | NM_014489 | FGF receptor activating protein 1 |
| FRAS1 | NM_032863 | Fraser syndrome 1 isoform 4 |
| FRAT1 | NM_005479 | GSK-3 binding protein FRAT1 |
| FREQ | NM_014286 | frequenin homolog |
| FRMD4A | NM_018027 | FERM domain containing 4A |
| FRMD6 | NM_152330 | FERM domain containing 6 |
| FRMPD1 | NM_014907 | FERM and PDZ domain containing 1 |
| FRMPD2 | NM_152428 | FERM and PDZ domain containing 2 isoform 1 |
| FRMPD4 | NM_014728 | PDZ domain containing 10 |
| FRY | NM_023037 | hypothetical protein CG003 |
| FSD1 | NM_024333 | fibronectin type III and SPRY domain containing |
| FSD2 | NM_001007122 | SPRY domain containing 1 |
| FSIP2 | NM_173651 | fibrous sheath interacting protein 2 |
| FSTL1 | NM_007085 | follistatin-like 1 precursor |
| FSTL3 | NM_005860 | follistatin-like 3 glycoprotein precursor |
| FSTL4 | NM_015082 | follistatin-like 4 |
| FUBP1 | NM_003902 | far upstream element-binding protein |
| FUCA1 | NM_000147 | fucosidase, alpha-L-1, tissue |
| FURIN | NM_002569 | furin preproprotein |
| FUT1 | NM_000148 | fucosyltransferase 1 |
| FUT2 | NM_000511 | fucosyltransferase 2 (secretor status included) |
| FUT3 | NM_000149 | fucosyltransferase 3 (galactoside |
| FUT4 | NM_002033 | fucosyltransferase 4 |
| FVT1 | NM_002035 | follicular lymphoma variant translocation 1 |
| FXN | NM_000144 | frataxin isoform 1 preproprotein |
| FXYD2 | NM_001680 | FXYD domain-containing ion transport regulator 2 |
| FXYD6 | NM_022003 | FXYD domain-containing ion transport regulator |
| FYCO1 | NM_024513 | FYVE and coiled-coil domain containing 1 |
| FZD10 | NM_007197 | frizzled 10 |
| FZD4 | NM_012193 | frizzled 4 |
| FZD6 | NM_003506 | frizzled 6 |
| FZD7 | NM_003507 | frizzled 7 |
| FZD9 | NM_003508 | frizzled 9 |
| G0S2 | NM_015714 | putative lymphocyte G0/G1 switch gene |
| G3BP2 | NM_012297 | Ras-GTPase activating protein SH3 domain-binding |
| G6PD | NM_000402 | glucose-6-phosphate dehydrogenase |
| GAA | NM_000152 | acid alpha-glucosidase preproprotein |
| GAB2 | NM_012296 | GRB2-associated binding protein 2 isoform b |
| GAB3 | NM_080612 | Gab3 protein |
| GABARAPL1 | NM_031412 | GABA(A) receptor-associated protein like 1 |
| GABBR1 | NM_001470 | gamma-aminobutyric acid (GABA) B receptor 1 |
| GABPA | NM_002040 | GA binding protein transcription factor, alpha |
| GABRA1 | NM_000806 | gamma-aminobutyric acid (GABA) A receptor, alpha |
| GABRE | NM_004961 | gamma-aminobutyric acid (GABA) A receptor, |
| GABRP | NM_014211 | gamma-aminobutyric acid (GABA) A receptor, pi |
| GADD45G | NM_006705 | growth arrest and DNA-damage-inducible, gamma |
| GAGE1 | NM_001468 | G antigen 1 |
| GAK | NM_005255 | cyclin G associated kinase |
| GALC | NM_000153 | galactosylceramidase isoform a precursor |
| GALM | NM_138801 | galactose mutarotase (aldose 1-epimerase) |
| GALNT1 | NM_020474 | polypeptide N-acetylgalactosaminyltransferase 1 |
| GALNT11 | NM_022087 | GALNAC-T11 |
| GALNT13 | NM_052917 | UDP-N-acetyl-alpha-D-galactosamine:polypeptide |
| GALNT2 | NM_004481 | polypeptide N-acetylgalactosaminyltransferase 2 |
| GALNT4 | NM_003774 | polypeptide N-acetylgalactosaminyltransferase 4 |
| GALNT7 | NM_017423 | polypeptide N-acetylgalactosaminyltransferase 7 |
| GALNT9 | NM_021808 | polypeptide N-acetylgalactosaminyltransferase 9 |
| GAN | NM_022041 | gigaxonin |
| GANAB | NM_198334 | alpha glucosidase II alpha subunit isoform 2 |
| GARNL1 | NM_014990 | GTPase activating Rap/RanGAP domain-like 1 |
| GARNL4 | NM_015085 | GTPase activating Rap/RanGAP domain-like 4 |
| GAS2L1 | NM_152237 | growth arrest-specific 2 like 1 isoform b |
| GAS7 | NM_003644 | growth arrest-specific 7 isoform a |
| GATA2 | NM_032638 | GATA binding protein 2 |
| GATA4 | NM_002052 | GATA binding protein 4 |
| GATA5 | NM_080473 | GATA binding protein 5 |
| GATAD2A | NM_017660 | GATA zinc finger domain containing 2A |
| GATAD2B | NM_020699 | GATA zinc finger domain containing 2B |
| GBA | NM_000157 | glucocerebrosidase precursor |
| GBF1 | NM_004193 | golgi-specific brefeldin A resistance factor 1 |
| GBL | NM_022372 | G protein beta subunit-like |
| GCC1 | NM_024523 | Golgi coiled-coil protein 1 |
| GCC2 | NM_014635 | GRIP and coiled-coil domain-containing 2 isoform |
| GCK | NM_000162 | glucokinase isoform 1 |
| GCLC | NM_001498 | glutamate-cysteine ligase, catalytic subunit |
| GCM1 | NM_003643 | glial cells missing homolog a |
| GCNT3 | NM_004751 | glucosaminyl (N-acetyl) transferase 3, mucin |
| GDI2 | NM_001494 | GDP dissociation inhibitor 2 |
| GDPD2 | NM_017711 | osteoblast differentiation promoting factor |
| Gene_symbol | hsa-miR-16 targets | Gene_name |
| GFAP | NM_002055 | glial fibrillary acidic protein |
| GFER | NM_005262 | erv1-like growth factor |
| GFI1B | NM_004188 | growth factor independent 1B (potential |
| GFM1 | NM_024996 | G elongation factor, mitochondrial 1 |
| GFPT1 | NM_002056 | glucosamine-fructose-6-phosphate |
| GFRA4 | NM_022139 | GDNF family receptor alpha 4 isoform a |
| GGA2 | NM_015044 | ADP-ribosylation factor binding protein 2 |
| GGA3 | NM_014001 | ADP-ribosylation factor binding protein 3 |
| GH1 | NM_022562 | growth hormone 1 isoform 5 |
| GH2 | NM_022557 | growth hormone 2 isoform 2 |
| GHR | NM_000163 | growth hormone receptor precursor |
| GIMAP5 | NM_018384 | GTPase, IMAP family member 5 |
| GIT1 | NM_014030 | G protein-coupled receptor kinase interactor 1 |
| GJA4 | NM_002060 | connexin 37 |
| GLCE | NM_015554 | D-glucuronyl C5-epimerase |
| GLIS3 | NM_152629 | GLIS family zinc finger 3 |
| GLRX | NM_002064 | glutaredoxin (thioltransferase) |
| GLS | NM_014905 | glutaminase C |
| GLS2 | NM_013267 | glutaminase GA isoform a |
| GLT1D1 | NM_144669 | hypothetical protein LOC144423 |
| GLT25D2 | NM_015101 | glycosyltransferase 25 domain containing 2 |
| GLTP | NM_016433 | glycolipid transfer protein |
| GLUD1 | NM_005271 | glutamate dehydrogenase 1 |
| GLUD2 | NM_012084 | glutamate dehydrogenase 2 |
| GM2A | NM_000405 | GM2 ganglioside activator precursor |
| GM632 | NM_020713 | hypothetical protein LOC57473 |
| GMEB2 | NM_012384 | glucocorticoid modulatory element binding |
| GNA12 | NM_007353 | guanine nucleotide binding protein (G protein) |
| GNA15 | NM_002068 | guanine nucleotide binding protein (G protein), |
| GNAI3 | NM_006496 | guanine nucleotide binding protein (G protein), |
| GNAL | NM_002071 | guanine nucleotide binding protein (G protein), |
| GNAO1 | NM_020988 | guanine nucleotide binding protein, alpha |
| GNAQ | NM_002072 | guanine nucleotide binding protein (G protein), |
| GNAS | NM_016592 | guanine nucleotide binding protein, alpha |
| GNB1 | NM_002074 | guanine nucleotide-binding protein, beta-1 |
| GNG12 | NM_018841 | G-protein gamma-12 subunit |
| GNG2 | NM_053064 | guanine nucleotide binding protein (G protein), |
| GNG7 | NM_052847 | guanine nucleotide binding protein (G protein), |
| GNL3L | NM_019067 | guanine nucleotide binding protein-like 3 |
| GOLGA | NM_018652 | golgin-like protein |
| GOLGA1 | NM_002077 | golgin 97 |
| GOLGA2 | NM_004486 | Golgi autoantigen, golgin subfamily a, 2 |
| GOLGA3 | NM_005895 | Golgi autoantigen, golgin subfamily a, 3 |
| GOLGA4 | NM_002078 | golgi autoantigen, golgin subfamily a, 4 |
| GOLGA7 | NM_001002296 | golgi autoantigen, golgin subfamily a, 7 |
| GOLPH4 | NM_014498 | golgi phosphoprotein 4 |
| GOLT1B | NM_016072 | golgi transport 1 homolog B |
| GORASP1 | NM_031899 | Golgi reassembly stacking protein 1 |
| GORASP2 | NM_015530 | golgi reassembly stacking protein 2 |
| GOSR1 | NM_001007024 | golgi SNAP receptor complex member 1 isoform 3 |
| GOT2 | NM_002080 | aspartate aminotransferase 2 precursor |
| GPA33 | NM_005814 | transmembrane glycoprotein A33 precursor |
| GPAM | NM_020918 | mitochondrial glycerol 3-phosphate |
| GPATC4 | NM_015590 | G patch domain containing 4 protein isoform 1 |
| GPC1 | NM_002081 | glypican 1 precursor |
| GPC3 | NM_004484 | glypican 3 |
| GPD1 | NM_005276 | glycerol-3-phosphate dehydrogenase 1 (soluble) |
| GPIAP1 | NM_005898 | membrane component chromosome 11 surface marker |
| GPR109A | NM_177551 | G protein-coupled receptor 109A |
| GPR109B | NM_006018 | G protein-coupled receptor 109B |
| GPR114 | NM_153837 | G-protein coupled receptor 114 |
| GPR124 | NM_032777 | G protein-coupled receptor 124 |
| GPR126 | NM_001032394 | G protein-coupled receptor 126 alpha 2 |
| GPR132 | NM_013345 | G protein-coupled receptor 132 |
| GPR146 | NM_138445 | G protein-coupled receptor 146 |
| GPR171 | NM_013308 | G protein-coupled receptor 171 |
| GPR180 | NM_180989 | G protein-coupled receptor 180 precursor |
| GPR23 | NM_005296 | G protein-coupled receptor 23 |
| GPR26 | NM_153442 | G protein-coupled receptor 26 |
| GPR30 | NM_001505 | G protein-coupled receptor 30 |
| GPR55 | NM_005683 | G protein-coupled receptor 55 |
| GPR6 | NM_005284 | G protein-coupled receptor 6 |
| GPR63 | NM_030784 | G protein-coupled receptor 63 |
| GPR68 | NM_003485 | G protein-coupled receptor 68 |
| GPR78 | NM_080819 | G protein-coupled receptor 78 |
| GPR83 | NM_016540 | G protein-coupled receptor 83 |
| GPR88 | NM_022049 | G-protein coupled receptor 88 |
| GPR92 | NM_020400 | putative G protein-coupled receptor 92 |
| GPS1 | NM_004127 | G protein pathway suppressor 1 isoform 2 |
| GPSM3 | NM_022107 | G-protein signalling modulator 3 (AGS3-like, C. |
| GPX1 | NM_000581 | glutathione peroxidase 1 isoform 1 |
| GRAMD2 | NM_001012642 | hypothetical protein LOC196996 |
| GRAMD3 | NM_023927 | GRAM domain containing 3 |
| GRB10 | NM_001001549 | growth factor receptor-bound protein 10 isoform |
| GRB2 | NM_002086 | growth factor receptor-bound protein 2 isoform |
| GRB7 | NM_001030002 | growth factor receptor-bound protein 7 |
| GREM2 | NM_022469 | gremlin 2 precursor |
| GRIA3 | NM_000828 | glutamate receptor 3 isoform flop precursor |
| GRIK3 | NM_000831 | glutamate receptor 7 precursor |
| GRIN1 | NM_000832 | NMDA receptor 1 isoform NR1-1 precursor |
| GRIN2B | NM_000834 | N-methyl-D-aspartate receptor subunit 2B |
| GRIN2C | NM_000835 | N-methyl-D-aspartate receptor subunit 2C |
| GRIN3A | NM_133445 | glutamate receptor, ionotropic, |
| GRK6 | NM_001004106 | G protein-coupled receptor kinase 6 isoform A |
| GRM1 | NM_000838 | glutamate receptor, metabotropic 1 |
| GRM7 | NM_000844 | glutamate receptor, metabotropic 7 isoform a |
| GRPR | NM_005314 | gastrin-releasing peptide receptor |
| GRTP1 | NM_024719 | growth hormone regulated TBC protein 1 |
| GRWD1 | NM_031485 | glutamate-rich WD repeat containing 1 |
| GSDMDC1 | NM_024736 | gasdermin domain containing 1 |
| GSG1 | NM_153823 | germ cell associated 1 isoform 2 |
| GSTT2 | NM_000854 | glutathione S-transferase theta 2 |
| GTDC1 | NM_001006636 | glycosyltransferase-like domain containing 1 |
| GTF3C5 | NM_012087 | general transcription factor IIIC, polypeptide |
| GTPBP1 | NM_004286 | GTP binding protein 1 |
| GTPBP8 | NM_001008235 | hypothetical protein LOC29083 isoform 3 |
| GUCA1B | NM_002098 | guanylate cyclase activator 1B (retina) |
| GUSBL2 | NM_206910 | hypothetical protein LOC375513 isoform 2 |
| GYLTL1B | NM_152312 | glycosyltransferase-like 1B |
| GYS1 | NM_002103 | glycogen synthase 1 (muscle) |
| H2AFJ | NM_018267 | H2A histone family, member J isoform 1 |
| H2-ALPHA | NM_080386 | alpha-tubulin isotype H2-alpha |
| H6PD | NM_004285 | hexose-6-phosphate dehydrogenase precursor |
| HADHSC | NM_005327 | L-3-hydroxyacyl-Coenzyme A dehydrogenase |
| HALPLN4 | NM_023002 | brain link protein 2 |
| HARSL | NM_012208 | histidyl-tRNA synthetase-like |
| HAS1 | NM_001523 | hyaluronan synthase 1 |
| HAS2 | NM_005328 | hyaluronan synthase 2 |
| HAS3 | NM_005329 | hyaluronan synthase 3 isoform a |
| HCCA2 | NM_053005 | HCCA2 protein |
| HCFC1 | NM_005334 | host cell factor C1 (VP16-accessory protein) |
| HD | NM_002111 | huntingtin |
| HDGF | NM_004494 | hepatoma-derived growth factor (high-mobility |
| HECTD1 | NM_015382 | HECT domain containing 1 |
| HECW1 | NM_015052 | NEDD4-like ubiquitin-protein ligase 1 |
| HELZ | NM_014877 | helicase with zinc finger domain |
| HEMK1 | NM_016173 | HemK methyltransferase family member 1 |
| HERC2 | NM_004667 | hect domain and RLD 2 |
| HERC4 | NM_001017972 | hect domain and RLD 4 isoform c |
| HERC6 | NM_001013000 | hect domain and RLD 6 isoform c |
| HERV-FRD | NM_207582 | HERV-FRD provirus ancestral Env polyprotein |
| HES2 | NM_019089 | hairy and enhancer of split homolog 2 |
| HES5 | NM_001010926 | hairy and enhancer of split 5 |
| HEXA | NM_000520 | hexosaminidase A preproprotein |
| HEY1 | NM_012258 | hairy/enhancer-of-split related with YRPW motif |
| HEY2 | NM_012259 | hairy/enhancer-of-split related with YRPW motif |
| HEYL | NM_014571 | hairy/enhancer-of-split related with YRPW |
| HIC1 | NM_006497 | hypermethylated in cancer 1 |
| HIC2 | NM_015094 | hypermethylated in cancer 2 |
| HIGD1A | NM_014056 | HIG1 domain family, member 1A |
| HIP1 | NM_005338 | huntingtin interacting protein 1 |
| HIRA | NM_003325 | HIR (histone cell cycle regulation defective, S. |
| HIST1H2AG | NM_021064 | H2A histone family, member P |
| HIST2H2BE | NM_003528 | H2B histone family, member Q |
| HK1 | NM_000188 | hexokinase 1 isoform HKI |
| HK2 | NM_000189 | hexokinase 2 |
| HKR2 | NM_181846 | GLI-Kruppel family member HKR2 |
| HLA-DQA1 | NM_002122 | major histocompatibility complex, class II, DQ |
| HMBOX1 | NM_024567 | hypothetical protein LOC79618 |
| HMBS | NM_000190 | hydroxymethylbilane synthase isoform 1 |
| HMG20A | NM_018200 | high-mobility group 20A |
| HMG2L1 | NM_001003681 | high-mobility group protein 2-like 1 isoform b |
| HMGA1 | NM_002131 | high mobility group AT-hook 1 isoform b |
| HMGA2 | NM_001015886 | high mobility group AT-hook 2 isoform c |
| HMGB3 | NM_005342 | high-mobility group box 3 |
| HMOX2 | NM_002134 | heme oxygenase (decyclizing) 2 |
| HNF4A | NM_000457 | hepatocyte nuclear factor 4 alpha isoform b |
| HNF4G | NM_004133 | hepatocyte nuclear factor 4, gamma |
| HNRPA0 | NM_006805 | heterogeneous nuclear ribonucleoprotein A0 |
| HNRPA1 | NM_002136 | heterogeneous nuclear ribonucleoprotein A1 |
| HNRPDL | NM_005463 | heterogeneous nuclear ribonucleoprotein D-like |
| HNRPU | NM_004501 | heterogeneous nuclear ribonucleoprotein U |
| HOXA10 | NM_018951 | homeobox A10 isoform a |
| HOXA3 | NM_030661 | homeobox A3 isoform a |
| HOXB13 | NM_006361 | homeobox B13 |
| HOXB4 | NM_024015 | homeobox B4 |
| HOXB7 | NM_004502 | homeobox B7 |
| HOXC11 | NM_014212 | homeobox C11 |
| HOXC13 | NM_017410 | homeobox C13 |
| HOXC8 | NM_022658 | homeobox C8 |
| HOXD1 | NM_024501 | homeobox D1 |
| HOXD9 | NM_014213 | homeobox D9 |
| HPCAL4 | NM_016257 | hippocalcin-like protein 4 |
| HPS1 | NM_182637 | Hermansky-Pudlak syndrome 1 protein isoform b |
| HPS4 | NM_022081 | light ear protein isoform a |
| HPSE2 | NM_021828 | heparanase 2 |
| HR | NM_005144 | hairless protein isoform a |
| HRH2 | NM_022304 | histamine receptor H2 |
| HRH3 | NM_007232 | histamine receptor H3 |
| HS2ST1 | NM_012262 | heparan sulfate 2-O-sulfotransferase 1 |
| HS6ST1 | NM_004807 | heparan sulfate 6-O-sulfotransferase |
| HS6ST2 | NM_147175 | heparan sulfate 6-O-sulfotransferase 2 |
| HSDL2 | NM_032303 | hydroxysteroid dehydrogenase like 2 |
| HSF2BP | NM_007031 | heat shock transcription factor 2 binding |
| HSPA1B | NM_005346 | heat shock 70 kDa protein 1B |
| HSPA4L | NM_014278 | heat shock 70 kDa protein 4-like |
| HSPA8 | NM_006597 | heat shock 70 kDa protein 8 isoform 1 |
| HSPB7 | NM_014424 | heat shock 27 kDa protein family, member 7 |
| HSPBAP1 | NM_024610 | Hspb associated protein 1 |
| HSPC049 | NM_014149 | HSPC049 protein |
| HSPC117 | NM_014306 | hypothetical protein LOG51493 |
| HSPG2 | NM_005529 | heparan sulfate proteoglycan 2 |
| HSU79303 | NM_013301 | hypothetical protein LOC29903 |
| HTF9C | NM_022727 | HpaII tiny fragments locus 9C |
| HTR2A | NM_000621 | 5-hydroxytryptamine (serotonin) receptor 2A |
| HTR2C | NM_000868 | 5-hydroxytryptamine (serotonin) receptor 2C |
| HTR4 | NM_000870 | serotonin 5-HT4 receptor isoform b |
| HTRA2 | NM_013247 | HtrA serine peptidase 2 isoform 1 preproprotein |
| HTRA3 | NM_053044 | HtrA serine peptidase 3 |
| HYOU1 | NM_006389 | oxygen regulated protein precursor |
| IARS | NM_002161 | isoleucine-tRNA synthetase |
| IBRDC1 | NM_152553 | IBR domain containing 1 |
| IBRDC2 | NM_182757 | IBR domain containing 2 |
| ICA1 | NM_022307 | islet cell autoantigen 1 |
| ICMT | NM_012405 | isoprenylcysteine carboxyl methyltransferase |
| ICOS | NM_012092 | inducible T-cell co-stimulator precursor |
| ICOSLG | NM_015259 | inducible T-cell co-stimulator ligand |
| IDH3A | NM_005530 | isocitrate dehydrogenase 3 (NAD+) alpha |
| IER2 | NM_004907 | immediate early response 2 |
| IFIT1 | NM_001548 | interferon-induced protein with |
| IFNAR1 | NM_000629 | interferon-alpha receptor 1 precursor |
| IFNGR2 | NM_005534 | interferon-gamma receptor beta chain precursor |
| IFT140 | NM_014714 | intraflagellar transport 140 |
| IFT20 | NM_174887 | intraflagellar transport protein IFT20 |
| IFT57 | NM_018010 | estrogen-related receptor beta like 1 |
| IFT74 | NM_025103 | coiled-coil domain containing 2 |
| IGF1 | NM_000618 | insulin-like growth factor 1 (somatomedin C) |
| IGF1R | NM_000875 | insulin-like growth factor 1 receptor precursor |
| IGF2BP1 | NM_006546 | insulin-like growth factor 2 mRNA binding |
| IGF2R | NM_000876 | insulin-like growth factor 2 receptor |
| IGFBP3 | NM_000598 | insulin-like growth factor binding protein 3 |
| IGSF22 | NM_173588 | hypothetical protein LOC283284 |
| IGSF3 | NM_001007237 | immunoglobulin superfamily, member 3 isoform 2 |
| IGSF4 | NM_014333 | immunoglobulin superfamily, member 4D |
| IHPK1 | NM_001006115 | inositol hexaphosphate kinase 1 isoform 2 |
| IHPK3 | NM_054111 | inositol hexaphosphate kinase 3 |
| IKBKAP | NM_003640 | inhibitor of kappa light polypeptide gene |
| IKBKB | NM_001556 | inhibitor of kappa light polypeptide gene |
| IKBKE | NM_014002 | IKK-related kinase epsilon |
| IKBKG | NM_003639 | inhibitor of kappa light polypeptide gene |
| IL10RA | NM_001558 | interleukin 10 receptor, alpha precursor |
| IL10RB | NM_000628 | interleukin 10 receptor, beta precursor |
| IL13 | NM_002188 | interleukin 13 precursor |
| IL15 | NM_000585 | interleukin 15 preproprotein |
| IL16 | NM_004513 | interleukin 16 isoform 1 precursor |
| IL17D | NM_138284 | interleukin 17D precursor |
| IL17E | NM_022789 | interleukin 17E isoform 1 precursor |
| IL17RB | NM_172234 | interleukin 17B receptor isoform 2 precursor |
| IL17RC | NM_032732 | interleukin 17 receptor C isoform 3 precursor |
| IL17RD | NM_017563 | interleukin 17 receptor D |
| IL17RE | NM_144640 | interleukin 17 receptor B isoform 3 |
| IL18BP | NM_173042 | interleukin 18 binding protein precursor |
| IL18R1 | NM_003855 | interleukin 18 receptor 1 precursor |
| IL1F5 | NM_012275 | interleukin 1 family, member 5 |
| IL1F8 | NM_173178 | interleukin 1 family, member 8 isoform 2 |
| IL1F9 | NM_019618 | interleukin 1 family, member 9 |
| IL1R1 | NM_000877 | interleukin 1 receptor, type I precursor |
| IL1RAP | NM_134470 | interleukin 1 receptor accessory protein isoform |
| IL1RAPL1 | NM_014271 | interleukin 1 receptor accessory protein-like 1 |
| IL1RL1 | NM_003856 | interleukin 1 receptor-like 1 isoform 2 |
| IL20 | NM_018724 | interleukin 20 precursor |
| IL28RA | NM_170743 | interleukin 28 receptor, alpha isoform 1 |
| IL2RA | NM_000417 | interleukin 2 receptor, alpha chain precursor |
| IL2RB | NM_000878 | interleukin 2 receptor beta precursor |
| IL3 | NM_000588 | interleukin 3 precursor |
| IL3RA | NM_002183 | interleukin 3 receptor, alpha precursor |
| IL6R | NM_000565 | interleukin 6 receptor isoform 1 precursor |
| IL9R | NM_176786 | interleukin 9 receptor isoform 2 |
| ILDR1 | NM_175924 | immunoglobulin-like domain containing receptor |
| ILF3 | NM_004516 | interleukin enhancer binding factor 3 isoform b |
| IMMP2L | NM_032549 | IMP2 inner mitochondrial membrane protease-like |
| IMPA2 | NM_014214 | inositol(myo)-1(or 4)-monophosphatase 2 |
| INCENP | NM_020238 | inner centromere protein antigens 135/155 kDa |
| ING5 | NM_032329 | inhibitor of growth family, member 5 |
| INPP5A | NM_005539 | inositol polyphosphate-5-phosphatase A |
| INSM2 | NM_032594 | insulinoma-associated protein IA-6 |
| INSR | NM_000208 | insulin receptor |
| INVS | NM_014425 | inversin isoform a |
| IPO8 | NM_006390 | importin 8 |
| IPPK | NM_022755 | inositol 1,3,4,5,6-pentakisphosphate 2-kinase |
| IQCE | NM_152558 | IQ motif containing E |
| IQGAP1 | NM_003870 | IQ motif containing GTPase activating protein 1 |
| IQGAP3 | NM_178229 | IQ motif containing GTPase activating protein 3 |
| IRAK1 | NM_001025242 | interleukin-1 receptor-associated kinase 1 |
| IRAK2 | NM_001570 | interleukin-1 receptor-associated kinase 2 |
| IRF2BP1 | NM_015649 | interferon regulatory factor 2 binding protein |
| IRF4 | NM_002460 | interferon regulatory factor 4 |
| IRF5 | NM_002200 | interferon regulatory factor 5 isoform a |
| IRS1 | NM_005544 | insulin receptor substrate 1 |
| IRS2 | NM_003749 | insulin receptor substrate 2 |
| IRX3 | NM_024336 | iroquois homeobox protein 3 |
| ISLR | NM_005545 | immunoglobulin superfamily containing |
| ISOC1 | NM_016048 | isochorismatase domain containing 1 |
| ISOC2 | NM_024710 | isochorismatase domain containing 2 |
| ITFG3 | NM_032039 | integrin alpha FG-GAP repeat containing 3 |
| ITGA10 | NM_003637 | integrin, alpha 10 precursor |
| ITGA2 | NM_002203 | integrin alpha 2 precursor |
| ITGAM | NM_000632 | integrin alpha M precursor |
| ITGAX | NM_000887 | integrin alpha X precursor |
| ITGB4BP | NM_002212 | integrin beta 4 binding protein isoform a |
| ITGB5 | NM_002213 | integrin, beta 5 |
| ITGBL1 | NM_004791 | integrin, beta-like 1 (with EGF-like repeat |
| ITIH1 | NM_002215 | inter-alpha (globulin) inhibitor H1 |
| ITIH5 | NM_001001851 | inter-alpha trypsin inhibitor heavy chain |
| ITK | NM_005546 | IL2-inducible T-cell kinase |
| ITPK1 | NM_014216 | inositol 1,3,4-triphosphate 5/6 kinase |
| ITPR1 | NM_002222 | inositol 1,4,5-triphosphate receptor, type 1 |
| ITSN1 | NM_001001132 | intersectin 1 isoform ITSN-s |
| IVNS1ABP | NM_006469 | influenza virus NS1A binding protein isoform a |
| JAGN1 | NM_032492 | jagunal homolog 1 |
| JAK2 | NM_004972 | Janus kinase 2 |
| JARID1B | NM_006618 | Jumonji, AT rich interactive domain 1B |
| JARID2 | NM_004973 | jumonji, AT rich interactive domain 2 protein |
| JMJD2D | NM_018039 | jumonji domain containing 2D |
| JMJD4 | NM_023007 | jumonji domain containing 4 |
| JMJD5 | NM_024773 | hypothetical protein LOC79831 |
| JOSD1 | NM_014876 | Josephin domain containing 1 |
| JPH1 | NM_020647 | junctophilin 1 |
| JPH2 | NM_020433 | junctophilin 2 isoform 1 |
| JUB | NM_032876 | jub, ajuba homolog isoform 1 |
| JUP | NM_002230 | junction plakoglobin |
| K6HF | NM_004693 | cytokeratin type II |
| K6IRS3 | NM_175068 | keratin 6 irs3 |
| K6IRS4 | NM_175053 | keratin 6 irs4 |
| KAL1 | NM_000216 | Kallmann syndrome 1 protein |
| KALRN | NM_001024660 | kalirin, RhoGEF kinase isoform 1 |
| KARS | NM_005548 | lysyl-tRNA synthetase |
| KATNAL1 | NM_001014380 | katanin p60 subunit A-like 1 |
| KATNB1 | NM_005886 | katanin p80 subunit B 1 |
| KBTBD2 | NM_015483 | kelch repeat and BTB (POZ) domain containing 2 |
| KBTBD4 | NM_016506 | kelch repeat and BTB (POZ) domain containing 4 |
| KBTBD5 | NM_152393 | keich repeat and BTB (POZ) domain containing 5 |
| KCNA3 | NM_002232 | potassium voltage-gated channel, shaker-related |
| KCNAB1 | NM_003471 | potassium voltage-gated channel, shaker-related |
| KCNAB2 | NM_003636 | potassium voltage-gated channel, shaker-related |
| KCNC2 | NM_139136 | Shaw-related voltage-gated potassium channel |
| KCND3 | NM_004980 | potassium voltage-gated channel, Shal-related |
| KCNE1L | NM_012282 | potassium voltage-gated channel, Isk-related |
| KCNG3 | NM_133329 | potassium voltage-gated channel, subfamily G, |
| KCNG4 | NM_133490 | potassium voltage-gated channel, subfamily G, |
| KCNH4 | NM_012285 | potassium voltage-gated channel, subfamily H, |
| KCNIP1 | NM_014592 | Kv channel interacting protein 1 isoform 2 |
| KCNIP3 | NM_013434 | Kv channel interacting protein 3 isoform 1 |
| KCNJ11 | NM_000525 | potassium inwardly-rectifying channel J11 |
| KCNJ16 | NM_018658 | potassium inwardly-rectifying channel J16 |
| KCNJ2 | NM_000891 | potassium inwardly-rectifying channel J2 |
| KCNJ9 | NM_004983 | potassium inwardly-rectifying channel subfamily |
| KCNK1 | NM_002245 | potassium channel, subfamily K, member 1 |
| KCNK2 | NM_001017424 | potassium channel, subfamily K, member 2 isoform |
| KCNK7 | NM_005714 | potassium channel, subfamily K, member 7 isoform |
| KCNMA1 | NM_001014797 | large conductance calcium-activated potassium |
| KCNN4 | NM_002250 | intermediate conductance calcium-activated |
| KCNQ1 | NM_000218 | potassium voltage-gated channel, KQT-like |
| KCNQ2 | NM_004518 | potassium voltage-gated channel KQT-like protein |
| KCNQ5 | NM_019842 | potassium voltage-gated channel, KQT-like |
| KCNRG | NM_173605 | potassium channel regulator isoform 1 |
| KCNS1 | NM_002251 | potassium voltage-gated channel |
| KCNT1 | NM_020822 | potassium channel, subfamily T, member 1 |
| KCNT2 | NM_198503 | potassium channel, subfamily T, member 2 |
| KCTD1 | NM_198991 | potassium channel tetramerisation domain |
| KCTD12 | NM_138444 | potassium channel tetramerisation domain |
| KCTD15 | NM_024076 | potassium channel tetramerisation domain |
| KCTD2 | NM_015353 | potassium channel tetramerisation domain |
| KCTD3 | NM_016121 | potassium channel tetramerisation domain |
| KCTD5 | NM_018992 | potassium channel tetramerisation domain |
| KCTD7 | NM_153033 | potassium channel tetramerisation domain |
| KCTD8 | NM_198353 | potassium channel tetramerisation domain |
| KGFLP1 | NM_174950 | hypothetical protein LOC387628 |
| KIAA0125 | NM_014792 | hypothetical protein LOC9834 |
| KIAA0143 | NM_015137 | hypothetical protein LOC23167 |
| KIAA0152 | NM_014730 | hypothetical protein LOC9761 |
| KIAA0174 | NM_014761 | putative MAPK activating protein PM28 |
| KIAA0179 | NM_015056 | hypothetical protein LOC23076 |
| KIAA0182 | NM_014615 | hypothetical protein LOC23199 |
| KIAA0232 | NM_014743 | hypothetical protein LOC9778 |
| KIAA0240 | NM_015349 | hypothetical protein LOC23506 |
| KIAA0241 | NM_015060 | hypothetical protein LOC23080 |
| KIAA0247 | NM_014734 | hypothetical protein LOC9766 |
| KIAA0251 | NM_015027 | hypothetical protein LOC23042 |
| KIAA0265 | NM_014997 | hypothetical protein LOC23008 |
| KIAA0284 | NM_015005 | hypothetical protein LOC283638 |
| KIAA0286 | NM_015257 | hypothetical protein LOC23306 |
| KIAA0319L | NM_024874 | polycystic kidney disease 1-like isoform a |
| KIAA0323 | NM_015299 | hypothetical protein LOC23351 |
| KIAA0329 | NM_014844 | hypothetical protein LOC9895 |
| KIAA0350 | NM_015226 | hypothetical protein LOC23274 |
| KIAA0355 | NM_014686 | hypothetical protein LOC9710 |
| KIAA0376 | NM_015330 | cytospin A |
| KIAA0423 | NM_015091 | hypothetical protein LOC23116 |
| KIAA0427 | NM_014772 | hypothetical protein LOC9811 |
| KIAA0446 | NM_014655 | hypothetical protein LOC9673 |
| KIAA0494 | NM_014774 | hypothetical protein LOC9813 |
| KIAA0495 | NM_207306 | KIAA0495 |
| KIAA0513 | NM_014732 | hypothetical protein LOC9764 |
| KIAA0523 | NM_015253 | hypothetical protein LOC23302 |
| KIAA0553 | NM_001002909 | hypothetical protein LOC23131 |
| KIAA0556 | NM_015202 | hypothetical protein LOC23247 |
| KIAA0562 | NM_014704 | glycine-, glutamate-, |
| KIAA0564 | NM_001009814 | hypothetical protein LOC23078 isoform b |
| KIAA0649 | NM_014811 | 1A6/DRIM (down-regulated in metastasis) |
| KIAA0652 | NM_014741 | hypothetical protein LOC9776 |
| KIAA0664 | NM_015229 | hypothetical protein LOC23277 |
| KIAA0672 | NM_014859 | hypothetical protein LOC9912 |
| KIAA0676 | NM_015043 | hypothetical protein LOC23061 isoform b |
| KIAA0683 | NM_016111 | hypothetical protein LOC9894 |
| KIAA0746 | NM_015187 | hypothetical protein LOC23231 |
| KIAA0773 | NM_014690 | hypothetical protein LOC9715 |
| KIAA0789 | NM_014653 | hypothetical protein LOC9671 |
| KIAA0804 | NM_001009921 | hypothetical protein LOC23355 isoform a |
| KIAA0828 | NM_015328 | KIAA0828 protein |
| KIAA0831 | NM_014924 | hypothetical protein LOC22863 |
| KIAA0853 | NM_015070 | KIAA0853 |
| KIAA0859 | NM_001007239 | CGI-01 protein isoform 3 |
| KIAA0863 | NM_014913 | hypothetical protein LOC22850 |
| KIAA0895 | NM_015314 | hypothetical protein LOC23366 |
| KIAA1161 | NM_020702 | hypothetical protein LOC57462 |
| KIAA1166 | NM_018684 | hepatocellular carcinoma-associated antigen 127 |
| KIAA1199 | NM_018689 | KIAA1199 |
| KIAA1267 | NM_015443 | hypothetical protein LOC284058 |
| KIAA1274 | NM_014431 | KIAA1274 |
| KIAA1303 | NM_020761 | raptor |
| KIAA1333 | NM_017769 | hypothetical protein LOC55632 |
| KIAA1411 | NM_020819 | hypothetical protein LOC57579 |
| KIAA1434 | NM_019593 | hypothetical protein LOC56261 |
| KIAA1456 | NM_020844 | hypothetical protein LOC57604 |
| KIAA1522 | NM_020888 | hypothetical protein LOC57648 |
| KIAA1530 | NM_020894 | hypothetical protein LOC57654 |
| KIAA1542 | NM_020901 | CTD-binding SR-like protein rA9 |
| KIAA1559 | NM_020917 | zinc finger protein 14-like |
| KIAA1576 | NM_020927 | hypothetical protein LOC57687 |
| KIAA1600 | NM_020940 | hypothetical protein LOC57700 |
| KIAA1609 | NM_020947 | hypothetical protein LOC57707 |
| KIAA1618 | NM_020954 | hypothetical protein LOC57714 |
| KIAA1688 | NM_025251 | KIAA1688 protein |
| KIAA1715 | NM_030650 | Lunapark |
| KIAA1727 | NM_033393 | hypothetical protein LOC85462 |
| KIAA1729 | NM_053042 | hypothetical protein LOC85460 |
| KIAA1737 | NM_033426 | KIAA1737 protein |
| KIAA1772 | NM_024935 | hypothetical protein LOC80000 |
| KIAA1804 | NM_032435 | mixed lineage kinase 4 |
| KIAA1815 | NM_024896 | hypothetical protein LOC79956 |
| KIAA1853 | NM_194286 | KIAA1853 protein |
| KIAA1862 | NM_032534 | KIAA1862 protein |
| KIAA1875 | NM_032529 | KIAA1875 protein |
| KIAA1909 | NM_052909 | hypothetical protein LOC153478 |
| KIAA1920 | NM_052919 | hypothetical protein LOC114817 |
| KIAA1924 | NM_145294 | hypothetical protein LOC197335 |
| KIAA1961 | NM_001008738 | hypothetical protein LOC96459 isoform 2 |
| KIAA2022 | NM_001008537 | hypothetical protein LOC340533 |
| KIF12 | NM_138424 | kinesin family member 12 |
| KIF13B | NM_015254 | kinesin family member 13B |
| KIF1A | NM_004321 | axonal transport of synaptic vesicles |
| KIF1B | NM_015074 | kinesin family member 1B isoform b |
| KIF1C | NM_006612 | kinesin family member 1C |
| KIF2 | NM_004520 | kinesin heavy chain member 2 |
| KIF21A | NM_017641 | kinesin family member 21A |
| KIF23 | NM_004856 | kinesin family member 23 isoform 2 |
| KIF2C | NM_006845 | kinesin family member 2C |
| KIF3B | NM_004798 | kinesin family member 3B |
| KIF5A | NM_004984 | kinesin family member 5A |
| KIF5B | NM_004521 | kinesin family member 5B |
| KIF6 | NM_145027 | kinesin family member 6 |
| KIFC3 | NM_005550 | kinesin family member C3 |
| KIR2DS4 | NM_012314 | killer cell immunoglobulin-like receptor, two |
| KITLG | NM_000899 | KIT ligand isoform b precursor |
| KL | NM_004795 | klotho isoform a |
| KLC2 | NM_022822 | likely ortholog of kinesin light chain 2 |
| KLC4 | NM_201521 | kinesin-like 8 isoform a |
| KLF12 | NM_016285 | Kruppel-like factor 12 isoform b |
| KLF13 | NM_015995 | Kruppel-like factor 13 |
| KLHDC6 | NM_207335 | hypothetical protein LOC166348 |
| KLHDC8B | NM_173546 | hypothetical protein LOC200942 |
| KLHL18 | NM_025010 | kelch-like 18 |
| KLHL2 | NM_007246 | kelch-like 2, Mayven |
| KLHL21 | NM_014851 | kelch-like 21 |
| KLHL26 | NM_018316 | hypothetical protein LOC55295 |
| KLHL3 | NM_017415 | kelch-like 3 (Drosophila) |
| KLHL4 | NM_019117 | kelch-like 4 isoform 1 |
| KLK2 | NM_001002231 | kallikrein 2, prostatic isoform 2 |
| KLKB1 | NM_000892 | plasma kallikrein B1 precursor |
| KNDC1 | NM_152643 | kinase non-catalytic C-lobe domain (KIND) |
| KNS2 | NM_005552 | kinesin 2 60/70 kDa isoform 1 |
| KPNA3 | NM_002267 | karyopherin alpha 3 |
| KPNA4 | NM_002268 | karyopherin alpha 4 |
| KRAS | NM_004985 | c-K-ras2 protein isoform b |
| KRT1B | NM_175078 | keratin 1B |
| KRT20 | NM_019010 | keratin 20 |
| KRT2B | NM_015848 | cytokeratin 2 |
| KRTAP10-1 | NM_198691 | keratin associated protein 10-1 |
| KRTAP10-12 | NM_198699 | keratin associated protein 10-12 |
| KRTAP10-8 | NM_198695 | keratin associated protein 10-8 |
| KRTAP11-1 | NM_175858 | keratin associated protein 11-1 |
| KRTAP26-1 | NM_203405 | hypothetical protein LOC388818 |
| KRTAP4-4 | NM_032524 | keratin associated protein 4.4 |
| KRTAP9-2 | NM_031961 | keratin associated protein 9.2 |
| KRTAP9-3 | NM_031962 | keratin associated protein 9.3 |
| KRTAP9-4 | NM_033191 | keratin associated protein 9-4 |
| KRTHA3B | NM_002279 | type I hair keratin 3B |
| KRTHB4 | NM_033045 | keratin, hair, basic, 4 |
| KSR1 | NM_014238 | kinase suppressor of ras |
| Kua-UEV | NM_003349 | ubiquitin-conjugating enzyme E2 Kua-UEV isoform |
| KU-MEL-3 | NM_001011540 | KU-MEL-3 protein |
| LAMC1 | NM_002293 | laminin, gamma 1 precursor |
| LAMP1 | NM_005561 | lysosomal-associated membrane protein 1 |
| LAMP2 | NM_013995 | lysosomal-associated membrane protein 2 |
| LAMP3 | NM_014398 | lysosomal-associated membrane protein 3 |
| LANCL1 | NM_006055 | lanthionine synthetase C-like protein 1 |
| LANCL2 | NM_018697 | LanC lantibiotic synthetase component C-like 2 |
| LARP2 | NM_032239 | La ribonucleoprotein domain family member 2 |
| LASP1 | NM_006148 | LIM and SH3 protein 1 |
| LASS1 | NM_021267 | longevity assurance gene 1 isoform 1 |
| LASS3 | NM_178842 | hypothetical protein LOC204219 |
| LASS6 | NM_203463 | longevity assurance homolog 6 |
| LAT | NM_001014987 | linker for activation of T cells isoform b |
| LATS1 | NM_004690 | LATS homolog 1 |
| LATS2 | NM_014572 | LATS, large tumor suppressor, homolog 2 |
| LCE1E | NM_178353 | late cornified envelope 1E |
| LCN2 | NM_005564 | lipocalin 2 (oncogene 24p3) |
| LCP1 | NM_002298 | L-plastin |
| LDB3 | NM_007078 | LIM domain binding 3 |
| LDLRAD2 | NM_001013693 | hypothetical protein LOC401944 |
| LDLRAP1 | NM_015627 | low density lipoprotein receptor adaptor protein |
| LDOC1 | NM_012317 | leucine zipper, down-regulated in cancer 1 |
| LDOC1L | NM_032287 | hypothetical protein LOC84247 |
| LEMD1 | NM_001001552 | LEM domain containing 1 |
| LENG12 | NM_033206 | hypothetical protein LOC90011 |
| LEP | NM_000230 | leptin precursor |
| LETM1 | NM_012318 | leucine zipper-EF-hand containing transmembrane |
| LGALS8 | NM_006499 | galectin 8 isoform a |
| LGI2 | NM_018176 | leucine-rich repeat LGI family, member 2 |
| LGI4 | NM_139284 | leucine-rich repeat LGI family, member 4 |
| LGR6 | NM_001017403 | leucine-rich repeat-containing G protein-coupled |
| LHFPL5 | NM_182548 | lipoma HMGIC fusion partner-like 5 |
| LHPP | NM_022126 | phospholysine phosphohistidine inorganic |
| LHX3 | NM_014564 | LIM homeobox protein 3 isoform b |
| LIF | NM_002309 | leukemia inhibitory factor (cholinergic |
| LIMD1 | NM_014240 | LIM domains containing 1 |
| LIMS3 | NM_033514 | LIM and senescent cell antigen-like domains 3 |
| LIN28 | NM_024674 | lin-28 homolog |
| LIN28B | NM_001004317 | lin-28 homolog B |
| LIPE | NM_005357 | hormone-sensitive lipase |
| LIPG | NM_006033 | endothelial lipase precursor |
| LIPH | NM_139248 | lipase, member H precursor |
| LITAF | NM_004862 | LPS-induced TNF-alpha factor |
| LKAP | NM_014647 | limkain b1 |
| LMAN2L | NM_030805 | lectin, mannose-binding 2-like |
| LMNA | NM_170707 | lamin A/C isoform 1 precursor |
| LMO7 | NM_005358 | LIM domain only 7 |
| LMOD1 | NM_012134 | leiomodin 1 (smooth muscle) |
| LNX1 | NM_032622 | multi-PDZ-domain-containing protein |
| LNX2 | NM_153371 | PDZ domain containing ring finger 1 |
| LOC112714 | NM_207312 | hypothetical protein LOC112714 |
| LOC115648 | NM_145326 | hypothetical protein LOC115648 |
| LOC116143 | NM_138458 | monad |
| LOC133308 | NM_178833 | hypothetical protein LOC133308 |
| LOC144233 | NM_181708 | hypothetical protein LOC144233 |
| LOC144363 | NM_001001660 | hypothetical protein LOC144363 |
| LOC144983 | NM_001011724 | heterogeneous nuclear ribonucleoprotein A1-like |
| LOC147650 | NM_207324 | hypothetical protein LOC147650 |
| LOC147804 | NM_001010856 | hypothetical protein LOC147804 |
| LOC150383 | NM_001008917 | hypothetical protein LOC150383 isoform 2 |
| LOC151194 | NM_145280 | hypothetical protein LOC151194 |
| LOC153222 | NM_153607 | hypothetical protein LOC153222 |
| LOC155060 | NM_001004302 | hypothetical protein LOC155060 |
| LOC158381 | NM_001029857 | hypothetical protein LOC158381 |
| LOC159090 | NM_145284 | hypothetical protein LOC159090 |
| LOC161931 | NM_139174 | hypothetical protein LOC161931 |
| LOC162427 | NM_178126 | hypothetical protein LOC162427 |
| LOC165186 | NM_199280 | hypothetical protein LOC165186 |
| LOC196463 | NM_173542 | hypothetical protein LOC196463 |
| LOC197322 | NM_174917 | hypothetical protein LOC197322 |
| LOC201164 | NM_178836 | hypothetical protein LOC201164 |
| LOC203427 | NM_145305 | mitochondrial solute carrier protein |
| LOC203547 | NM_001017980 | hypothetical protein LOC203547 |
| LOC220594 | NM_145809 | TL132 protein |
| LOC221442 | NM_001010871 | hypothetical protein LOC221442 |
| LOC255374 | NM_203397 | hypothetical protein LOC255374 |
| LOC283487 | NM_178514 | hypothetical protein LOC283487 |
| LOC283537 | NM_181785 | hypothetical protein LOC283537 |
| LOC283849 | NM_178516 | hypothetical protein LOC283849 |
| LOC284434 | NM_001007525 | hypothetical protein LOC284434 |
| LOC284757 | NM_001004305 | hypothetical protein LOC284757 |
| LOC284861 | NM_201565 | hypothetical protein LOC284861 |
| LOC285074 | NM_001012626 | hypothetical protein LOC285074 |
| LOC285382 | NM_001025266 | hypothetical protein LOC285382 |
| LOC285498 | NM_194439 | hypothetical protein LOC285498 |
| LOC285636 | NM_175921 | hypothetical protein LOC285636 |
| LOC286526 | NM_001031834 | Ras-like GTPase-like |
| LOC317671 | NM_173362 | hypothetical protein LOC317671 |
| LOC339768 | NM_194312 | hypothetical protein LOC339768 |
| LOC340156 | NM_001012418 | hypothetical protein LOC340156 |
| LOC340529 | NM_001012977 | hypothetical protein LOC340529 |
| LOC348174 | NM_182619 | secretory protein LOC348174 |
| LOC348262 | NM_207368 | hypothetical protein LOC348262 |
| LOC348840 | NM_182631 | hypothetical protein LOC348840 |
| LOC352909 | NM_001031802 | hypothetical protein LOC352909 isoform 2 |
| LOC387646 | NM_001006604 | hypothetical protein LOC387646 |
| LOC387720 | NM_001013633 | hypothetical protein LOC387720 |
| LOC387758 | NM_203371 | hypothetical protein LOC387758 |
| LOC387856 | NM_001013635 | hypothetical protein LOC387856 |
| LOC388886 | NM_207644 | hypothetical protein LOC388886 |
| LOC389541 | NM_001008395 | hypothetical protein LOC389541 |
| LOC390980 | NM_001023563 | similar to Zinc finger protein 264 |
| LOC391356 | NM_001013663 | hypothetical protein LOC391356 |
| LOC399706 | NM_001010910 | hypothetical protein LOC399706 |
| LOC399900 | NM_001013667 | hypothetical protein LOC399900 |
| LOC400120 | NM_203451 | hypothetical protein LOC400120 |
| LOC400145 | NM_001013669 | hypothetical protein LOC400145 |
| LOC400258 | NM_001008404 | hypothetical protein LOC400258 |
| LOC400451 | NM_207446 | hypothetical protein LOC400451 |
| LOC400464 | NM_001013670 | hypothetical protein LOC400464 |
| LOC400696 | NM_207646 | hypothetical protein LOC400696 |
| LOC400707 | NM_001013673 | hypothetical protein LOC400707 |
| LOC400891 | NM_001013675 | hypothetical protein LOC400891 |
| LOC400924 | NM_001013676 | hypothetical protein LOC400924 |
| LOC400965 | NM_001013677 | hypothetical protein LOC400965 |
| LOC401152 | NM_001001701 | hypothetical protein LOC401152 |
| LOC401233 | NM_001013680 | hypothetical protein LOC401233 |
| LOC401252 | NM_001013681 | hypothetical protein LOC401252 |
| LOC401286 | NM_001023565 | hypothetical protein LOC401286 |
| LOC401431 | NM_001008745 | hypothetical protein LOC401431 |
| LOC401498 | NM_212558 | hypothetical protein LOC401498 |
| LOC401589 | NM_001013687 | hypothetical protein LOC401589 |
| LOC401720 | NM_001013690 | hypothetical protein LOC401720 |
| LOC402055 | NM_001013694 | hypothetical protein LOC402055 |
| LOC405753 | NM_207581 | Numb-interacting protein |
| LOC440157 | NM_001013701 | hypothetical protein LOC440157 |
| LOC440248 | NM_199045 | hypothetical protein LOC440248 |
| LOC440742 | NM_001013710 | hypothetical protein LOC440742 |
| LOC440944 | NM_001013713 | hypothetical protein LOC440944 |
| LOC441046 | NM_001011539 | hypothetical protein LOC441046 |
| LOC441087 | NM_001013716 | hypothetical protein LOC441087 |
| LOC441120 | NM_001013718 | hypothetical protein LOC441120 |
| LOC441177 | NM_001013720 | hypothetical protein LOC441177 |
| LOC441193 | NM_001013722 | hypothetical protein LOC441193 |
| LOC441208 | NM_001013723 | hypothetical protein LOC441208 |
| LOC441257 | NM_001023562 | hypothetical protein LOC441257 |
| LOC441426 | NM_001013727 | hypothetical protein LOC441426 |
| LOC442582 | NM_001025202 | STAG3-like |
| LOC493856 | NM_001008388 | hypothetical protein LOC493856 |
| LOC497190 | NM_001011880 | hypothetical protein LOC497190 |
| LOC51057 | NM_015910 | hypothetical protein LOC51057 |
| LOC541469 | NM_001013617 | hypothetical protein LOC541469 |
| LOC55565 | NM_017530 | hypothetical protein LOC55565 |
| LOC56964 | NM_020212 | hypothetical protein LOC56964 |
| LOC619208 | NM_001033564 | hypothetical protein LOC619208 |
| LOC89944 | NM_138342 | hypothetical protein LOC89944 |
| LOC90321 | NM_001010851 | hypothetical protein LOC90321 |
| LOC90639 | NM_001031617 | hypothetical protein LOC90639 |
| LOC90693 | NM_138771 | hypothetical protein LOC90693 |
| LOC91461 | NM_138370 | hypothetical protein LOC91461 |
| LOC91689 | NM_033318 | hypothetical protein LOC91689 |
| LOC93349 | NM_138402 | hypothetical protein LOC93349 |
| LOC93622 | NM_138699 | hypothetical protein LOC93622 |
| LOXL2 | NM_002318 | lysyl oxidase-like 2 precursor |
| LPHN1 | NM_001008701 | latrophilin 1 isoform 1 precursor |
| LPHN2 | NM_012302 | latrophilin 2 precursor |
| LPIN2 | NM_014646 | lipin 2 |
| LPIN3 | NM_022896 | lipin 3 |
| LPP | NM_005578 | LIM domain containing preferred translocation |
| LPPR2 | NM_022737 | lipid phosphate phosphatase-related protein type |
| LRCH1 | NM_015116 | leucine-rich repeats and calponin homology (CH) |
| LRCH4 | NM_002319 | leucine-rich repeats and calponin homology (CH) |
| LRIG1 | NM_015541 | leucine-rich repeats and immunoglobulin-like |
| LRIG2 | NM_014813 | leucine-rich repeats and immunoglobulin-like |
| LRP10 | NM_014045 | low density lipoprotein receptor-related protein |
| LRP12 | NM_013437 | suppression of tumorigenicity |
| LRP1B | NM_018557 | low density lipoprotein-related protein 1B |
| LRP6 | NM_002336 | low density lipoprotein receptor-related protein |
| LRP8 | NM_001018054 | low density lipoprotein receptor-related protein |
| LRPPRC | NM_133259 | leucine-rich PPR motif-containing protein |
| LRRC1 | NM_018214 | leucine rich repeat containing 1 |
| LRRC14 | NM_014665 | leucine rich repeat containing 14 |
| LRRC15 | NM_130830 | leucine rich repeat containing 15 |
| LRRC21 | NM_015613 | retina specific protein PAL |
| LRRC22 | NM_001017924 | leucine rich repeat containing 22 |
| LRRC25 | NM_145256 | leucine rich repeat containing 25 |
| LRRC27 | NM_030626 | leucine rich repeat containing 27 |
| LRRC3 | NM_030891 | leucine-rich repeat-containing 3 precursor |
| LRRC32 | NM_005512 | leucine rich repeat containing 32 precursor |
| LRRC47 | NM_020710 | leucine rich repeat containing 47 |
| LRRC55 | NM_001005210 | hypothetical protein LOC219527 |
| LRRC57 | NM_153260 | hypothetical protein LOC255252 |
| LRRC61 | NM_023942 | hypothetical protein LOC65999 |
| LRRC8A | NM_019594 | leucine-rich repeat-containing 8 |
| LRRFIP2 | NM_017724 | leucine rich repeat (in FLII) interacting |
| LRRK1 | NM_024652 | leucine-rich repeat kinase 1 |
| LRRN3 | NM_018334 | leucine rich repeat neuronal 3 |
| LRRN6A | NM_032808 | leucine-rich repeat neuronal 6A |
| LRRTM2 | NM_015564 | leucine rich repeat transmembrane neuronal 2 |
| LRSAM1 | NM_001005373 | leucine rich repeat and sterile alpha motif |
| LSM11 | NM_173491 | LSM11, U7 small nuclear RNA associated |
| LSM16 | NM_025083 | LSM16 homolog (EDC3, S. cerevisiae) |
| LSM4 | NM_012321 | U6 snRNA-associated Sm-like protein 4 |
| LSM7 | NM_016199 | U6 snRNA-associated Sm-like protein LSm7 |
| LSP1 | NM_001013253 | lymphocyte-specific protein 1 isoform 2 |
| LSS | NM_002340 | lanosterol synthase |
| LTB | NM_009588 | lymphotoxin-beta isoform b |
| LTBP1 | NM_000627 | latent transforming growth factor beta binding |
| LTC4S | NM_000897 | leukotriene C4 synthase isoform 2 |
| LUZP1 | NM_033631 | leucine zipper protein 1 |
| LY6E | NM_002346 | lymphocyte antigen 6 complex, locus E |
| LY6G5C | NM_001002848 | lymphocyte antigen 6 complex G5C isoform C |
| LY6K | NM_017527 | lymphocyte antigen 6 complex, locus K |
| LY86 | NM_004271 | MD-1, RP105-associated |
| LY9 | NM_001033667 | lymphocyte antigen 9 isoform b |
| LYCAT | NM_001002257 | lysocardiolipin acyltransferase isoform 2 |
| LYK5 | NM_001003786 | protein kinase LYK5 isoform 2 |
| LYPD5 | NM_001031749 | LY6/PLAUR domain containing 5 |
| LYPLA2 | NM_007260 | lysophospholipase II |
| LYPLA3 | NM_012320 | lysophospholipase 3 (lysosomal phospholipase |
| LYSMD4 | NM_152449 | hypothetical protein LOC145748 |
| LYST | NM_000081 | lysosomal trafficking regulator isoform 1 |
| LYZL4 | NM_144634 | lysozyme-like 4 |
| LZTFL1 | NM_020347 | leucine zipper transcription factor-like 1 |
| LZTR1 | NM_006767 | leucine-zipper-like transcription regulator, 1 |
| LZTS1 | NM_021020 | leucine zipper, putative tumor suppressor 1 |
| LZTS2 | NM_032429 | leucine zipper, putative tumor suppressor 2 |
| M6PR | NM_002355 | cation-dependent mannose-6-phosphate receptor |
| MACF1 | NM_012090 | microfilament and actin filament cross-linker |
| MADD | NM_003682 | MAP-kinase activating death domain-containing |
| MAF | NM_001031804 | v-maf musculoaponeurotic fibrosarcoma oncogene |
| MAFB | NM_005461 | transcription factor MAFB |
| MAFG | NM_002359 | v-maf musculoaponeurotic fibrosarcoma oncogene |
| MAG | NM_080600 | myelin associated glycoprotein isoform b |
| MAGEB4 | NM_002367 | melanoma antigen family B, 4 |
| MAK | NM_005906 | male germ cell-associated kinase |
| MAMDC2 | NM_153267 | MAM domain containing 2 |
| MAN2A2 | NM_006122 | mannosidase, alpha, class 2A, member 2 |
| MANBAL | NM_001003897 | mannosidase, beta A, lysosomal-like |
| MAP1A | NM_002373 | microtubule-associated protein 1A |
| MAP2K1 | NM_002755 | mitogen-activated protein kinase kinase 1 |
| MAP2K1IP1 | NM_021970 | mitogen-activated protein kinase kinase 1 |
| MAP2K2 | NM_030662 | mitogen-activated protein kinase kinase 2 |
| MAP2K3 | NM_002756 | mitogen-activated protein kinase kinase 3 |
| MAP2K4 | NM_003010 | mitogen-activated protein kinase kinase 4 |
| MAP2K7 | NM_145185 | mitogen-activated protein kinase kinase 7 |
| MAP3K14 | NM_003954 | mitogen-activated protein kinase kinase kinase |
| MAP3K3 | NM_002401 | mitogen-activated protein kinase kinase kinase 3 |
| MAP3K4 | NM_005922 | mitogen-activated protein kinase kinase kinase 4 |
| MAP3K7 | NM_003188 | mitogen-activated protein kinase kinase kinase 7 |
| MAP3K9 | NM_033141 | mitogen-activated protein kinase kinase kinase |
| MAP4 | NM_002375 | microtubule-associated protein 4 isoform 1 |
| MAP6 | NM_207577 | microtubule-associated protein 6 isoform 2 |
| MAP7 | NM_003980 | microtubule-associated protein 7 |
| MAPK1 | NM_002745 | mitogen-activated protein kinase 1 |
| MAPK14 | NM_001315 | mitogen-activated protein kinase 14 isoform 1 |
| MAPK3 | NM_002746 | mitogen-activated protein kinase 3 isoform 1 |
| MAPK8 | NM_002750 | mitogen-activated protein kinase 8 isoform 2 |
| MAPK8IP1 | NM_005456 | mitogen-activated protein kinase 8 interacting |
| MAPK8IP2 | NM_012324 | mitogen-activated protein kinase 8 interacting |
| MAPK8IP3 | NM_015133 | mitogen-activated protein kinase 8 interacting |
| MAPK9 | NM_002752 | mitogen-activated protein kinase 9 isoform 1 |
| MAPKAP1 | NM_001006617 | mitogen-activated protein kinase associated |
| MAPKAPK2 | NM_004759 | mitogen-activated protein kinase-activated |
| MAPKBP1 | NM_014994 | mitogen-activated protein kinase binding protein |
| MAPRE1 | NM_012325 | microtubule-associated protein, RP/EB family, |
| MAPRE3 | NM_012326 | microtubule-associated protein, RP/EB family, |
| MARCH4 | NM_020814 | membrane-associated ring finger (C3HC4) 4 |
| MARCH5 | NM_017824 | ring finger protein 153 |
| MARCH9 | NM_138396 | membrane-associated RING-CH protein IX |
| MARK4 | NM_031417 | MAP/microtubule affinity-regulating kinase 4 |
| MASP1 | NM_001031849 | mannan-binding lectin serine protease 1 isoform |
| MAT1A | NM_000429 | methionine adenosyltransferase I, alpha |
| MBD1 | NM_002384 | methyl-CpG binding domain protein 1 isoform 4 |
| MBD3 | NM_003926 | methyl-CpG binding domain protein 3 |
| MBD6 | NM_052897 | methyl-CpG binding domain protein 6 |
| MBNL2 | NM_144778 | muscleblind-like 2 isoform 1 |
| MBP | NM_001025100 | Golli-mbp isoform 2 |
| MCART1 | NM_033412 | mitochondrial carrier triple repeat 1 |
| MCART6 | NM_001012755 | hypothetical protein LOC401612 |
| MCFD2 | NM_139279 | multiple coagulation factor deficiency 2 |
| MCM2 | NM_004526 | minichromosome maintenance protein 2 |
| MDGA1 | NM_153487 | MAM domain containing |
| MECP2 | NM_004992 | methyl CpG binding protein 2 |
| MECR | NM_001024732 | nuclear receptor-binding factor 1 isoform b |
| MED11 | NM_001001683 | hypothetical protein LOC400569 |
| MED9 | NM_018019 | mediator of RNA polymerase II transcription, |
| MEFV | NM_000243 | Mediterranean fever protein |
| MEOX1 | NM_004527 | mesenchyme homeobox 1 isoform 1 |
| MEOX2 | NM_005924 | mesenchyme homeobox 2 |
| MESDC2 | NM_015154 | mesoderm development candidate 2 |
| METTL4 | NM_022840 | methyltransferase like 4 |
| MFAP5 | NM_003480 | microfibrillar associated protein 5 |
| MFN2 | NM_014874 | mitofusin 2 |
| MFSD2 | NM_032793 | major facilitator superfamily domain containing |
| MGAT5 | NM_002410 | alpha-1,3(6)-mannosylglycoprotein |
| MGC10911 | NM_032302 | hypothetical protein LOC84262 |
| MGC11102 | NM_032325 | hypothetical protein LOC84285 |
| MGC14289 | NM_080660 | hypothetical protein LOC92092 |
| MGC16385 | NM_145039 | hypothetical protein LOC92806 |
| MGC17330 | NM_052880 | HGFL protein |
| MGC20470 | NM_145053 | hypothetical protein LOC143630 |
| MGC21675 | NM_052861 | hypothetical protein LOC92070 |
| MGC21830 | NM_182563 | hypothetical protein LOC283870 |
| MGC24381 | NM_001001410 | hypothetical protein LOC115939 |
| MGC26694 | NM_178526 | hypothetical protein LOC284439 |
| MGC26718 | NM_001029999 | hypothetical protein LOC440482 |
| MGC26885 | NM_152339 | hypothetical protein LOC124044 |
| MGC29671 | NM_182538 | hypothetical protein LOC201305 |
| MGC3123 | NM_024107 | hypothetical protein LOC79089 isoform 1 |
| MGC3265 | NM_024028 | hypothetical protein LOC78991 |
| MGC33214 | NM_153354 | hypothetical protein LOC153396 |
| MGC33556 | NM_001004307 | hypothetical protein LOC339541 |
| MGC34761 | NM_173619 | hypothetical protein LOC283971 |
| MGC35308 | NM_175922 | hypothetical protein MGC35308 |
| MGC35361 | NM_147194 | hypothetical protein LOC222234 |
| MGC3731 | NM_024313 | hypothetical protein LOC79159 |
| MGC40405 | NM_152789 | hypothetical protein LOC257415 isoform 1 |
| MGC4093 | NM_030578 | hypothetical protein LOC80776 |
| MGC42105 | NM_153361 | hypothetical protein LOC167359 |
| MGC4268 | NM_031445 | hypothetical protein LOC83607 |
| MGC52000 | NM_198943 | CXYorfl-related protein |
| MGC5242 | NM_024033 | hypothetical protein LOC78996 |
| MGC57359 | NM_001004351 | hypothetical protein LOC441272 |
| MGC87631 | NM_001004306 | hypothetical protein LOC339184 |
| MGC9712 | NM_152689 | hypothetical protein LOC202915 |
| MGC9850 | NM_152705 | hypothetical protein MGC9850 |
| MGC99813 | NM_001005209 | hypothetical protein LOC130612 |
| MGRN1 | NM_015246 | mahogunin, ring finger 1 |
| MIB1 | NM_020774 | mindbomb homolog 1 |
| MICB | NM_005931 | MHC class I polypeptide-related sequence B |
| MID1 | NM_000381 | midline 1 isoform alpha |
| MIER2 | NM_017550 | hypothetical protein LOC54531 |
| MINK1 | NM_001024937 | misshapen/NIK-related kinase isoform 4 |
| MIOX | NM_017584 | myo-inositol oxygenase |
| MKL2 | NM_014048 | megakaryoblastic leukemia 2 protein |
| MKNK1 | NM_003684 | MAP kinase interacting serine/threonine kinase 1 |
| MKX | NM_173576 | hypothetical protein LOC283078 |
| MLC1 | NM_015166 | megalencephalic leukoencephalopathy with |
| MLCK | NM_182493 | MLCK protein |
| MLR1 | NM_153686 | transcription factor MLR1 |
| MLXIPL | NM_032951 | Williams Beuren syndrome chromosome region 14 |
| MLYCD | NM_012213 | malonyl-CoA decarboxylase |
| MMAB | NM_052845 | cob(I)alamin adenosyltransferase |
| MMACHC | NM_015506 | hypothetical protein LOC25974 |
| MMD | NM_012329 | monocyte to macrophage |
| MMD2 | NM_198403 | monocyte-to-macrophage differentiation factor 2 |
| MME | NM_000902 | membrane metallo-endopeptidase |
| MMP14 | NM_004995 | matrix metalloproteinase 14 preproprotein |
| MMP15 | NM_002428 | matrix metalloproteinase 15 preproprotein |
| MMP19 | NM_001032360 | matrix metalloproteinase 19 isoform 2 precursor |
| MMP24 | NM_006690 | matrix metalloproteinase 24 preproprotein |
| MMP3 | NM_002422 | matrix metalloproteinase 3 preproprotein |
| MMS19L | NM_022362 | MMS19-like (MET18 homolog, S. cerevisiae) |
| MN1 | NM_002430 | meningioma 1 |
| MNT | NM_020310 | MAX binding protein |
| MOBKL2A | NM_130807 | MOB-LAK |
| MOBKL2B | NM_024761 | MOB1, Mps One Binder kinase activator-like 2B |
| MOCS1 | NM_005942 | molybdenum cofactor synthesis-step 1 protein |
| MON1B | NM_014940 | MON1 homolog B |
| MORF4L1 | NM_006791 | MORF-related gene 15 isoform 1 |
| MOSC1 | NM_022746 | MOCO sulphurase C-terminal domain containing 1 |
| MOV10 | NM_020963 | Mov10, Moloney leukemia virus 10, homolog |
| MOV10L1 | NM_018995 | MOV10-like 1 |
| MPDU1 | NM_004870 | mannose-P-dolichol utilization defect 1 |
| MPL | NM_005373 | myeloproliferative leukemia virus oncogene |
| MPP2 | NM_005374 | palmitoylated membrane protein 2 |
| MPPED1 | NM_001585 | hypothetical protein LOC758 |
| MPZL1 | NM_003953 | myelin protein zero-like 1 isoform a |
| MRAS | NM_012219 | muscle RAS oncogene homolog |
| MRPL11 | NM_170739 | mitochondrial ribosomal protein L11 isoform c |
| MRPL12 | NM_002949 | mitochondrial ribosomal protein L12 |
| MRPL14 | NM_032111 | mitochondrial ribosomal protein L14 |
| MRPL35 | NM_016622 | mitochondrial ribosomal protein L35 isoform a |
| MRPL37 | NM_016491 | mitochondrial ribosomal protein L37 |
| MRPL4 | NM_146388 | mitochondrial ribosomal protein L4 isoform b |
| MRPL40 | NM_003776 | mitochondrial ribosomal protein L40 |
| MRPL45 | NM_032351 | mitochondrial ribosomal protein L45 |
| MRPS18A | NM_018135 | mitochondrial ribosomal protein S18A |
| MRPS2 | NM_016034 | mitochondrial ribosomal protein S2 |
| MRPS25 | NM_022497 | mitochondrial ribosomal protein S25 |
| MRRF | NM_138777 | mitochondrial ribosome recycling factor isoform |
| MS4A10 | NM_206893 | membrane-spanning 4-domains, subfamily A, member |
| MS4A2 | NM_000139 | membrane-spanning 4-domains, subfamily A, member |
| MS4A7 | NM_021201 | membrane-spanning 4-domains, subfamily A, member |
| MSH5 | NM_002441 | mutS homolog 5 isoform c |
| MSRB2 | NM_012228 | methionine sulfoxide reductase B2 |
| MST150 | NM_032947 | putative small membrane protein NID67 |
| MTAP | NM_002451 | 5′-methylthioadenosine phosphorylase |
| MTCP1 | NM_001018024 | mature T-cell proliferation 1 isoform p8 |
| MTG1 | NM_138384 | GTP_binding protein |
| MTHFR | NM_005957 | 5,10-methylenetetrahydrofolate reductase |
| MTM1 | NM_000252 | myotubularin |
| MTMR11 | NM_181873 | myotubularin related protein 11 |
| MTMR3 | NM_021090 | myotubularin-related protein 3 isoform c |
| MTMR4 | NM_004687 | myotubularin related protein 4 |
| MTMR8 | NM_017677 | myotubularin related protein 8 |
| MTMR9 | NM_015458 | myotubularin-related protein 9 |
| MTNR1B | NM_005959 | melatonin receptor 1B |
| MTPN | NM_145808 | myotrophin |
| MTRR | NM_002454 | methionine synthase reductase isoform 1 |
| MTSS1 | NM_014751 | metastasis suppressor 1 |
| MUC1 | NM_001018021 | MUC1 mucin isoform 4 precursor |
| MUCDHL | NM_031265 | mu-protocadherin isoform 4 |
| MULK | NM_018238 | multiple substrate lipid kinase |
| MUM1 | NM_032853 | melanoma ubiquitous mutated protein |
| MXD3 | NM_031300 | MAX dimerization protein 3 |
| MXD4 | NM_006454 | MAD4 |
| MYADM | NM_001020818 | myeloid-associated differentiation marker |
| MYB | NM_005375 | v-myb myeloblastosis viral oncogene homolog |
| MYBPC1 | NM_002465 | myosin binding protein C, slow type isoform 1 |
| MYCL1 | NM_001033081 | 1-myc-1 proto-oncogene isoform 1 |
| MYD88 | NM_002468 | myeloid differentiation primary response gene |
| MYEF2 | NM_016132 | myelin gene expression factor 2 |
| MYH14 | NM_024729 | myosin, heavy polypeptide 14 |
| MYL1 | NM_079420 | fast skeletal myosin alkali light chain 1 |
| MYLK | NM_005965 | myosin light chain kinase isoform 6 |
| MYO18A | NM_078471 | myosin 18A isoform a |
| MYO1D | NM_015194 | myosin ID |
| MYO1E | NM_004998 | myosin IE |
| MYO5C | NM_018728 | myosin VC |
| MYO9B | NM_004145 | myosin IXB |
| MYOHD1 | NM_001033579 | myosin head domain containing 1 isoform 2 |
| MYOM3 | NM_152372 | myomesin family, member 3 |
| MYOZ3 | NM_133371 | myozenin 3 |
| MYRIP | NM_015460 | myosin VIIA and Rab interacting protein |
| MYT1L | NM_015025 | myelin transcription factor 1-like |
| N4BP1 | NM_153029 | Nedd4 binding protein 1 |
| N4BP3 | NM_015111 | Nedd4 binding protein 3 |
| NAALADL2 | NM_207015 | N-acetylated alpha-linked acidic dipeptidase 2 |
| NAG8 | NM_014411 | nasopharyngeal carcinoma associated gene |
| NANOG | NM_024865 | Nanog homeobox |
| NANOS1 | NM_001009553 | nanos homolog 1 isoform 2 |
| NAP1L4 | NM_005969 | nucleosome assembly protein 1-like 4 |
| NAPA | NM_003827 | N-ethylmaleimide-sensitive factor attachment |
| NAPE-PLD | NM_198990 | N-acyl-phosphatidylethanolamine-hydrolyzing |
| NARF | NM_012336 | nuclear prelamin A recognition factor isoform a |
| NARFL | NM_022493 | nuclear prelamin A recognition factor-like |
| NARG1 | NM_057175 | NMDA receptor regulated 1 |
| NARS | NM_004539 | asparaginyl-tRNA synthetase |
| NAT10 | NM_024662 | N-acetyltransferase-like protein |
| NAT11 | NM_024771 | hypothetical protein LOC79829 |
| NAV1 | NM_020443 | neuron navigator 1 |
| NBEA | NM_015678 | neurobeachin |
| NBR1 | NM_005899 | neighbor of BRCA1 gene 1 |
| NCAM1 | NM_181351 | neural cell adhesion molecule 1 isoform 2 |
| NCF4 | NM_013416 | neutrophil cytosolic factor 4 (40 kD) isoform 2 |
| NCKIPSD | NM_016453 | NCK interacting protein with SH3 domain isoform |
| NCOA4 | NM_005437 | nuclear receptor coactivator 4 |
| NCOR2 | NM_006312 | nuclear receptor co-repressor 2 |
| NDNL2 | NM_138704 | necdin-like 2 |
| NDOR1 | NM_014434 | NADPH dependent diflavin oxidoreductase 1 |
| NDP | NM_000266 | norrin |
| NDRG2 | NM_016250 | N-myc downstream-regulated gene 2 isoform b |
| NDRG4 | NM_020465 | NDRG family member 4 |
| NDST1 | NM_001543 | N-deacetylase/N-sulfotransferase (heparan |
| NDUFA4L2 | NM_020142 | NADH:ubiquinone oxidoreductase MLRQ subunit |
| NEBL | NM_006393 | nebulette sarcomeric isoform |
| NECAP1 | NM_015509 | adaptin-ear-binding coat-associated protein 1 |
| NEDD9 | NM_182966 | neural precursor cell expressed, developmentally |
| NEK10 | NM_001031741 | NIMA (never in mitosis gene a)-related kinase |
| NEK6 | NM_014397 | putative serine-threonine protein kinase |
| NEK8 | NM_178170 | NIMA-related kinase 8 |
| NELF | NM_015537 | nasal embryonic LHRH factor |
| NEU4 | NM_080741 | sialidase 4 |
| NEURL | NM_004210 | neuralized-like |
| NEUROG3 | NM_020999 | neurogenin 3 |
| NF2 | NM_000268 | neurofibromin 2 isoform 1 |
| NFASC | NM_015090 | neurofascin precursor |
| NFAT5 | NM_006599 | nuclear factor of activated T-cells 5 isoform c |
| NFATC3 | NM_004555 | cytoplasmic nuclear factor of activated T-cells |
| NFATC4 | NM_004554 | cytoplasmic nuclear factor of activated T-cells |
| NFE2L1 | NM_003204 | nuclear factor (erythroid-derived 2)-like 1 |
| NFIC | NM_005597 | nuclear factor I/C isoform 1 |
| NFKB1 | NM_003998 | nuclear factor kappa-B, subunit 1 |
| NFKBIB | NM_001001716 | nuclear factor of kappa light polypeptide gene |
| NFKBIL1 | NM_005007 | nuclear factor of kappa light polypeptide gene |
| NFKBIL2 | NM_013432 | I-kappa-B-related protein |
| NFS1 | NM_021100 | NFS1 nitrogen fixation 1 isoform a precursor |
| NFYC | NM_014223 | nuclear transcription factor Y, gamma |
| NGFR | NM_002507 | nerve growth factor receptor precursor |
| NHEJ1 | NM_024782 | XRCC4-like factor |
| NHLH1 | NM_005598 | nescient helix loop helix 1 |
| NHS | NM_198270 | Nance-Horan syndrome protein |
| NIBP | NM_031466 | NIK and IKK(beta) binding protein |
| NID1 | NM_002508 | nidogen (enactin) |
| NIN | NM_020921 | ninein isoform 2 |
| NISCH | NM_007184 | nischarin |
| NKD1 | NM_033119 | naked cuticle homolog 1 |
| NKIRAS2 | NM_001001349 | NFKB inhibitor interacting Ras-like 2 |
| NKX2-8 | NM_014360 | NK2 transcription factor related, locus 8 |
| NKX3-1 | NM_006167 | NK3 transcription factor related, locus 1 |
| NLGN1 | NM_014932 | neuroligin 1 |
| NMD3 | NM_015938 | NMD3 homolog |
| NME3 | NM_002513 | nucleoside-diphosphate kinase 3 |
| NMNAT2 | NM_015039 | nicotinamide mononucleotide adenylyltransferase |
| NMT1 | NM_021079 | N-myristoyltransferase 1 |
| NMT2 | NM_004808 | glycylpeptide N-tetradecanoyltransferase 2 |
| NOB1 | NM_014062 | nin one binding protein |
| NOC2L | NM_015658 | nucleolar complex associated 2 homolog |
| NOD9 | NM_024618 | NOD9 protein isoform 1 |
| NODAL | NM_018055 | mouse nodal homolog precursor |
| NOL3 | NM_003946 | nucleolar protein 3 |
| NOMO1 | NM_014287 | nodal modulator 1 |
| NOMO2 | NM_173614 | nodal modulator 2 isoform 2 |
| NOMO3 | NM_001004067 | nodal modulator 3 |
| NOS1 | NM_000620 | nitric oxide synthase 1 (neuronal) |
| NOS1AP | NM_014697 | nitric oxide synthase 1 (neuronal) adaptor |
| NOS2A | NM_000625 | nitric oxide synthase 2A isoform 1 |
| NOTCH2 | NM_024408 | notch 2 preproprotein |
| NP | NM_000270 | purine nucleoside phosphorylase |
| NPAL3 | NM_020448 | NIPA-like domain containing 3 |
| NPC2 | NM_006432 | Niemann-Pick disease, type C2 precursor |
| NPEPPS | NM_006310 | aminopeptidase puromycin sensitive |
| NPHP4 | NM_015102 | nephroretinin |
| NPLOC4 | NM_017921 | nuclear protein localization 4 |
| NPNT | NM_001033047 | nephronectin |
| NPR2 | NM_003995 | natriuretic peptide receptor B precursor |
| NPTXR | NM_014293 | neuronal pentraxin receptor isoform 1 |
| NR2F6 | NM_005234 | nuclear receptor subfamily 2, group F, member 6 |
| NR4A1 | NM_002135 | nuclear receptor subfamily 4, group A, member 1 |
| NR4A3 | NM_173199 | nuclear receptor subfamily 4, group A, member 3 |
| NR5A1 | NM_004959 | nuclear receptor subfamily 5, group A, member 1 |
| NRBP1 | NM_013392 | nuclear receptor binding protein |
| NRG1 | NM_013958 | neuregulin 1 isoform HRG-beta3 |
| NRIP2 | NM_031474 | nuclear receptor interacting protein 2 |
| NRN1 | NM_016588 | neuritin precursor |
| NRP2 | NM_003872 | neuropilin 2 isoform 2 precursor |
| NSF | NM_006178 | N-ethylmaleimide-sensitive factor |
| NSUN4 | NM_199044 | NOL1/NOP2/Sun domain family 4 protein |
| NT5DC3 | NM_016575 | hypothetical protein LOC51559 isoform 2 |
| NTE | NM_006702 | neuropathy target esterase |
| NTN2L | NM_006181 | netrin 2-like |
| NTNG2 | NM_032536 | netrin G2 |
| NTRK2 | NM_001007097 | neurotrophic tyrosine kinase, receptor, type 2 |
| NTSR1 | NM_002531 | neurotensin receptor 1 |
| NUAK1 | NM_014840 | AMPK-related protein kinase 5 |
| NUAK2 | NM_030952 | NUAK family, SNF1-like kinase, 2 |
| NUBP2 | NM_012225 | nucleotide binding protein 2 (MinD homolog, E. |
| NUCB1 | NM_006184 | nucleobindin 1 |
| NUDCD3 | NM_015332 | NudC domain containing 3 |
| NUDT1 | NM_002452 | nudix-type motif 1 isoform p18 |
| NUDT11 | NM_018159 | nudix-type motif 11 |
| NUDT8 | NM_181843 | nudix-type motif 8 |
| NUP188 | NM_015354 | nucleoporin 188 kDa |
| NUP210 | NM_024923 | nucleoporin 210 |
| NUP35 | NM_001008544 | nucleoporin 35 kDa isoform b |
| NUP50 | NM_007172 | nucleoporin 50 kDa isoform b |
| NUP98 | NM_005387 | nucleoporin 98 kD isoform 3 |
| NUTF2 | NM_005796 | nuclear transport factor 2 |
| NXF5 | NM_033153 | nuclear RNA export factor 5 isoform c |
| NXPH1 | NM_152745 | neurexophilin 1 precursor |
| NXPH4 | NM_007224 | neurexophilin 4 |
| OAF | NM_178507 | hypothetical protein LOC220323 |
| OAS2 | NM_001032731 | 2′-5′-oligoadenylate synthetase 2 isoform 3 |
| OAS3 | NM_006187 | 2′-5′oligoadenylate synthetase 3 |
| OATL1 | NM_001006113 | ornithine aminotransferase-like 1 isoform 1 |
| OBSCN | NM_052843 | obscurin, cytoskeletal calmodulin and |
| OCRL | NM_000276 | phosphatidylinositol polyphosphate 5-phosphatase |
| ODF2 | NM_153437 | outer dense fiber of sperm tails 2 isoform 2 |
| OGDH | NM_002541 | oxoglutarate (alpha-ketoglutarate) dehydrogenase |
| OGDHL | NM_018245 | oxoglutarate dehydrogenase-like |
| OGFR | NM_007346 | opioid growth factor receptor |
| OGT | NM_003605 | O-linked GlcNAc transferase isoform 3 |
| OIP5 | NM_007280 | Opa interacting protein 5 |
| OLFM2 | NM_058164 | olfactomedin 2 |
| OMG | NM_002544 | oligodendrocyte myelin glycoprotein |
| OPHN1 | NM_002547 | oligophrenin 1 |
| OPRL1 | NM_000913 | opiate receptor-like 1 |
| ORMDL1 | NM_016467 | ORM1-like 1 |
| ORMDL3 | NM_139280 | ORM1-like 3 |
| OS9 | NM_001017956 | amplified in osteosarcoma isoform 2 precursor |
| OSBPL3 | NM_015550 | oxysterol-binding protein-like protein 3 isoform |
| OSCAR | NM_130771 | osteoclast-associated receptor isoform 3 |
| OSM | NM_020530 | oncostatin M precursor |
| OSR1 | NM_145260 | odd-skipped related 1 |
| OSTM1 | NM_014028 | osteopetrosis associated transmembrane protein |
| OTOF | NM_004802 | otoferlin isoform b |
| OTUB1 | NM_017670 | OTU domain, ubiquitin aldehyde binding 1 |
| OTUB2 | NM_023112 | OTU domain, ubiquitin aldehyde binding 2 |
| OTUD4 | NM_199324 | OTU domain containing 4 protein isoform 1 |
| OTUD6A | NM_207320 | HIN-6 protease |
| OTX1 | NM_014562 | orthodenticle 1 |
| OVOL1 | NM_004561 | OVO-like 1 binding protein |
| P15RS | NM_018170 | hypothetical protein FLJ10656 |
| P18SRP | NM_173829 | P18SRP protein |
| P2RX2 | NM_012226 | purinergic receptor P2X2 isoform I |
| P2RX7 | NM_177427 | purinergic receptor P2X7 isoform b |
| P2RXL1 | NM_005446 | purinergic receptor P2X-like 1, orphan receptor |
| P2RY8 | NM_178129 | G-protein coupled purinergic receptor P2Y8 |
| PA2G4 | NM_006191 | proliferation-associated 2G4, 38 kDa |
| PABPN1 | NM_004643 | poly(A) binding protein, nuclear 1 |
| PACRG | NM_152410 | PARK2 co-regulated |
| PACSIN1 | NM_020804 | protein kinase C and casein kinase substrate in |
| PAEP | NM_001018049 | glycodelin precursor |
| PAFAH1B1 | NM_000430 | platelet-activating factor acetylhydrolase, |
| PAFAH2 | NM_000437 | platelet-activating factor acetylhydrolase 2 |
| PAG1 | NM_018440 | phosphoprotein associated with glycosphingolipid |
| PAGE1 | NM_003785 | P antigen family, member 1 |
| PAICS | NM_006452 | phosphoribosylaminoimidazole carboxylase |
| PAK2 | NM_002577 | p21-activated kinase 2 |
| PAK6 | NM_020168 | p21-activated kinase 6 |
| PAK7 | NM_020341 | p21-activated kinase 7 |
| PALM2-AKAP2 | NM_007203 | PALM2-AKAP2 protein isoform 1 |
| PAM | NM_000919 | peptidylglycine alpha-amidating monooxygenasexxxxxxxx |
| PANK1 | NM_138316 | pantothenate kinase 1 isoform gamma |
| PANX1 | NM_015368 | pannexin 1 |
| PAPD1 | NM_018109 | PAP associated domain containing 1 |
| PAPOLG | NM_022894 | poly(A) polymerase gamma |
| PAPPA | NM_002581 | pregnancy-associated plasma protein A |
| PARD6B | NM_032521 | PAR-6 beta |
| PARD6G | NM_032510 | PAR-6 gamma protein |
| PARP11 | NM_020367 | poly (ADP-ribose) polymerase family, member 11 |
| PARP12 | NM_022750 | zinc finger CCCH-type domain containing 1 |
| PARP14 | NM_017554 | poly (ADP-ribose) polymerase family, member 14 |
| PATE | NM_138294 | expressed in prostate and testis |
| PAX2 | NM_000278 | paired box protein 2 isoform b |
| PAX8 | NM_003466 | paired box gene 8 isoform PAX8A |
| PAXIP1 | NM_007349 | PAX interacting protein 1 |
| PBX3 | NM_006195 | pre-B-cell leukemia transcription factor 3 |
| PCBP4 | NM_020418 | poly(rC) binding protein 4 isoform a |
| PCDH1 | NM_032420 | protocadherin 1 isoform 2 precursor |
| PCDH17 | NM_014459 | protocadherin 17 |
| PCDH19 | NM_020766 | protocadherin 19 |
| PCDH21 | NM_033100 | protocadherin 21 precursor |
| PCDH9 | NM_020403 | protocadherin 9 isoform 2 precursor |
| PCDHA1 | NM_018900 | protocadherin alpha 1 isoform 1 precursor |
| PCDHA10 | NM_018901 | protocadherin alpha 10 isoform 1 precursor |
| PCDHA11 | NM_018902 | protocadherin alpha 11 isoform 1 precursor |
| PCDHA12 | NM_018903 | protocadherin alpha 12 isoform 1 precursor |
| PCDHA13 | NM_018904 | protocadherin alpha 13 isoform 1 precursor |
| PCDHA2 | NM_018905 | protocadherin alpha 2 isoform 1 precursor |
| PCDHA3 | NM_018906 | protocadherin alpha 3 isoform 1 precursor |
| PCDHA4 | NM_018907 | protocadherin alpha 4 isoform 1 precursor |
| PCDHA5 | NM_018908 | protocadherin alpha 5 isoform 1 precursor |
| PCDHA6 | NM_018909 | protocadherin alpha 6 isoform 1 precursor |
| PCDHA7 | NM_018910 | protocadherin alpha 7 isoform 1 precursor |
| PCDHA8 | NM_018911 | protocadherin alpha 8 isoform 1 precursor |
| PCDHA9 | NM_031857 | protocadherin alpha 9 isoform 1 precursor |
| PCDHAC1 | NM_018898 | protocadherin alpha subfamily C, 1 isoform 1 |
| PCDHAC2 | NM_018899 | protocadherin alpha subfamily C, 2 isoform 1 |
| PCGF5 | NM_032373 | polycomb group ring finger 5 |
| PCID2 | NM_018386 | PCI domain containing 2 |
| PCMT1 | NM_005389 | protein-L-isoaspartate (D-aspartate) |
| PCNXL2 | NM_014801 | pecanex-like 2 |
| PCOLN3 | NM_002768 | procollagen (type III) N-endopeptidase |
| PCQAP | NM_001003891 | positive cofactor 2, glutamine/Q-rich-associated |
| PCSK2 | NM_002594 | proprotein convertase subtilisin/kexin type 2 |
| PCSK6 | NM_002570 | paired basic amino acid cleaving system 4 |
| PCSK9 | NM_174936 | proprotein convertase subtilisin/kexin type 9 |
| PCTK2 | NM_002595 | PCTAIRE protein kinase 2 |
| PCTP | NM_021213 | phosphatidylcholine transfer protein |
| PCYOX1 | NM_016297 | prenylcysteine oxidase 1 |
| PDAP1 | NM_014891 | PDGFA associated protein 1 |
| PDCD1 | NM_005018 | programmed cell death 1 precursor |
| PDCD11 | NM_014976 | programmed cell death 11 |
| PDCD4 | NM_014456 | programmed cell death 4 isoform 1 |
| PDCD6IP | NM_013374 | programmed cell death 6 interacting protein |
| PDCD7 | NM_005707 | programmed cell death 7 |
| PDCL | NM_005388 | phosducin-like |
| PDDC1 | NM_182612 | hypothetical protein LOC347862 |
| PDE3B | NM_000922 | phosphodiesterase 3B, cGMP-inhibited |
| PDE4D | NM_006203 | cAMP-specific phosphodiesterase 4D |
| PDE7B | NM_018945 | phosphodiesterase 7B |
| PDGFRA | NM_006206 | platelet-derived growth factor receptor alpha |
| PDGFRB | NM_002609 | platelet-derived growth factor receptor beta |
| PDIA6 | NM_005742 | protein disulfide isomerase-associated 6 |
| PDIK1L | NM_152835 | PDLIM1 interacting kinase 1 like |
| PDK2 | NM_002611 | pyruvate dehydrogenase kinase, isoenzyme 2 |
| PDK4 | NM_002612 | pyruvate dehydrogenase kinase 4 |
| PDLIM2 | NM_176871 | PDZ and LIM domain 2 isoform 1 |
| PDLIM5 | NM_001011513 | PDZ and LIM domain 5 isoform b |
| PDPK1 | NM_002613 | 3-phosphoinositide dependent protein kinase-1 |
| PDPN | NM_001006624 | lung type-I cell membrane-associated |
| PDPR | NM_017990 | pyruvate dehydrogenase phosphatase regulatory |
| PDRG1 | NM_030815 | p53 and DNA damage-regulated protein |
| PDXK | NM_003681 | pyridoxal kinase |
| PDYN | NM_024411 | beta-neoendorphin-dynorphin preproprotein |
| PDZD2 | NM_178140 | PDZ domain containing 2 |
| PELI2 | NM_021255 | pellino 2 |
| PELI3 | NM_145065 | pellino 3 alpha |
| PEMT | NM_007169 | phosphatidylethanolamine N-methyltransferase |
| PER3 | NM_016831 | period 3 |
| PERLD1 | NM_033419 | CAB2 protein |
| PERP | NM_022121 | PERP, TP53 apoptosis effector |
| PEX10 | NM_002617 | peroxisome biogenesis factor 10 isoform 2 |
| PEX12 | NM_000286 | peroxisomal biogenesis factor 12 |
| PEX13 | NM_002618 | peroxisome biogenesis factor 13 |
| PEX16 | NM_057174 | peroxisomal biogenesis factor 16 isoform 2 |
| PEX19 | NM_002857 | peroxisomal biogenesis factor 19 |
| PEX5 | NM_000319 | peroxisomal biogenesis factor 5 |
| PFKFB2 | NM_006212 | 6-phosphofructo-2-kinase/fructose-2, |
| PFKFB4 | NM_004567 | 6-phosphofructo-2-kinase/fructose-2, |
| PFKL | NM_001002021 | liver phosphofructokinase isoform a |
| PGAM5 | NM_138575 | Bcl-XL-binding protein v68 |
| PGD | NM_002631 | phosphogluconate dehydrogenase |
| PGEA1 | NM_001002880 | PKD2 interactor, golgi and endoplasmic reticulum |
| PGLS | NM_012088 | 6-phosphogluconolactonase |
| PGM1 | NM_002633 | phosphoglucomutase 1 |
| PGM2L1 | NM_173582 | phosphoglucomutase 2-like 1 |
| PHACTR1 | NM_030948 | phosphatase and actin regulator 1 |
| PHACTR2 | NM_014721 | phosphatase and actin regulator 2 |
| PHACTR4 | NM_023923 | phosphatase and actin regulator 4 |
| PHB | NM_002634 | prohibitin |
| PHF13 | NM_153812 | PHD finger protein 13 |
| PHF15 | NM_015288 | PHD finger protein 15 |
| PHF17 | NM_024900 | Jade1 protein short isoform |
| PHF19 | NM_015651 | PHD finger protein 19 isoform a |
| PHF20 | NM_016436 | PHD finger protein 20 |
| PHF20L1 | NM_016018 | PHD finger protein 20-like 1 isoform 1 |
| PHIP | NM_017934 | pleckstrin homology domain interacting protein |
| PHLDA3 | NM_012396 | pleckstrin homology-like domain, family A, |
| PHLDB3 | NM_198850 | pleckstrin homology-like domain, family B, |
| PHLPPL | NM_015020 | PH domain and leucine rich repeat protein |
| PHOX2B | NM_003924 | paired-like homeobox 2b |
| PHYHIP | NM_014759 | phytanoyl-CoA hydroxylase interacting protein |
| PI4K2B | NM_018323 | phosphatidylinositol 4-kinase type-II beta |
| PI4KII | NM_018425 | phosphatidylinositol 4-kinase type II |
| PIAS1 | NM_016166 | protein inhibitor of activated STAT, 1 |
| PIB5PA | NM_001002837 | phosphatidylinositol (4, 5) bisphosphate |
| PIGA | NM_002641 | phosphatidylinositol |
| PIGB | NM_004855 | phosphatidylinositol glycan, class B |
| PIGQ | NM_004204 | phosphatidylinositol glycan, class Q isoform 2 |
| PIGR | NM_002644 | polymeric immunoglobulin receptor |
| PIGT | NM_015937 | phosphatidylinositol glycan, class T precursor |
| PIK3C2B | NM_002646 | phosphoinositide-3-kinase, class 2, beta |
| PIK3R1 | NM_181504 | phosphoinositide-3-kinase, regulatory subunit, |
| PIK3R2 | NM_005027 | phosphoinositide-3-kinase, regulatory subunit 2 |
| PIK3R3 | NM_003629 | phosphoinositide-3-kinase, regulatory subunit 3 |
| PIK4CB | NM_002651 | phosphatidylinositol 4-kinase, catalytic, beta |
| PILRB | NM_013440 | paired immunoglobulin-like type 2 receptor beta |
| PIM1 | NM_002648 | pim-1 oncogene |
| PIM3 | NM_001001852 | pim-3 oncogene |
| PIP3-E | NM_015553 | phosphoinositide-binding protein PIP3-E |
| PIP5K1B | NM_001031687 | phosphatidylinositol-4-phosphate 5-kinase, type |
| PIP5K1C | NM_012398 | phosphatidylinositol-4-phosphate 5-kinase, type |
| PIP5K2C | NM_024779 | phosphatidylinositol-4-phosphate 5-kinase, type |
| PIP5K3 | NM_001002881 | phosphatidylinositol-3- |
| PISD | NM_014338 | phosphatidylserine decarboxylase |
| PITPNA | NM_006224 | phosphatidylinositol transfer protein, alpha |
| PKD1 | NM_000296 | polycystin 1 isoform 2 precursor |
| PKD1L2 | NM_182740 | polycystin 1-like 2 isoform b |
| PKHD1 | NM_138694 | polyductin isoform 1 |
| PKLR | NM_000298 | pyruvate kinase, liver and RBC isoform 1 |
| PKNOX1 | NM_004571 | PBX/knotted 1 homeobox 1 isoform 1 |
| PKP1 | NM_000299 | plakophilin 1 isoform 1b |
| PLA2G2F | NM_022819 | phospholipase A2, group IIF |
| PLA2G4D | NM_178034 | phospholipase A2, group IVD |
| PLAC2 | NM_153375 | placenta-specific 2 |
| PLAG1 | NM_002655 | pleiomorphic adenoma gene 1 |
| PLAGL1 | NM_002656 | pleiomorphic adenoma gene-like 1 isoform 1 |
| PLCD1 | NM_006225 | phospholipase C, delta 1 |
| PLCXD1 | NM_018390 | phosphatidylinositol-specific phospholipase C, X |
| PLCXD3 | NM_001005473 | phosphatidylinositol-specific phospholipase C, X |
| PLD1 | NM_002662 | phospholipase D1, phophatidylcholine-specific |
| PLD2 | NM_002663 | phospholipase D2 |
| PLDN | NM_012388 | pallidin |
| PLEKHA1 | NM_001001974 | pleckstrin homology domain containing, family A |
| PLEKHA5 | NM_019012 | pleckstrin homology domain containing, family A |
| PLEKHA6 | NM_014935 | phosphoinositol 3-phosphate-binding protein-3 |
| PLEKHA7 | NM_175058 | pleckstrin homology domain containing, family A |
| PLEKHB2 | NM_017958 | pleckstrin homology domain containing, family B |
| PLEKHC1 | NM_006832 | pleckstrin homology domain containing, family C |
| PLEKHG1 | NM_001029884 | pleckstrin homology domain containing, family G |
| PLEKHG3 | NM_015549 | pleckstrin homology domain containing, family G, |
| PLEKHG5 | NM_198681 | putative NFkB activating protein isoform b |
| PLEKHH1 | NM_020715 | pleckstrin homology domain containing, family H |
| PLEKHH2 | NM_172069 | pleckstrin homology domain containing, family H |
| PLEKHJ1 | NM_018049 | pleckstrin homology domain containing, family J |
| PLEKHK1 | NM_145307 | pleckstrin homology domain containing, family K |
| PLEKHM1 | NM_014798 | pleckstrin homology domain containing, family M |
| PLEKHQ1 | NM_025201 | PH domain-containing protein |
| PLRG1 | NM_002669 | pleiotropic regulator 1 (PRL1 homolog, |
| PLS1 | NM_002670 | plastin 1 |
| PLSCR4 | NM_020353 | phospholipid scramblase 4 |
| PLUNC | NM_130852 | palate, lung and nasal epithelium carcinoma |
| PLXDC1 | NM_020405 | plexin domain containing 1 precursor |
| PLXNA1 | NM_032242 | plexin A1 |
| PLXNA2 | NM_025179 | plexin A2 |
| PLXNB1 | NM_002673 | plexin B1 |
| PLXND1 | NM_015103 | plexin D1 |
| PML | NM_033239 | promyelocytic leukemia protein isoform 9 |
| PMM1 | NM_002676 | phosphomannomutase 1 |
| PMM2 | NM_000303 | phosphomannomutase 2 |
| PMP2 | NM_002677 | peripheral myelin protein 2 |
| PMP22 | NM_000304 | peripheral myelin protein 22 |
| PNKD | NM_015488 | myofibrillogenesis regulator 1 isoform 1 |
| PNLIPRP1 | NM_006229 | pancreatic lipase-related protein 1 |
| PNMA3 | NM_013364 | paraneoplastic cancer-testis-brain antigen |
| PNMA5 | NM_052926 | hypothetical protein LOC114824 |
| PNMA6A | NM_032882 | hypothetical protein LOC84968 |
| PNPO | NM_018129 | pyridoxine 5′-phosphate oxidase |
| PNRC2 | NM_017761 | proline-rich nuclear receptor coactivator 2 |
| PODN | NM_153703 | podocan |
| PODXL | NM_001018111 | podocalyxin-like precursor isoform 1 |
| POF1B | NM_024921 | premature ovarian failure, 1B |
| POFUT1 | NM_015352 | protein O-fucosyltransferase 1 isoform 1 |
| POFUT2 | NM_015227 | protein O-fucosyltransferase 2 isoform A |
| POLD3 | NM_006591 | polymerase (DNA directed), delta 3 |
| POLDIP3 | NM_032311 | DNA polymerase delta interacting protein 3 |
| POLE | NM_006231 | DNA polymerase epsilon catalytic subunit |
| POLE4 | NM_019896 | DNA polymerase epsilon subunit 4 |
| POLL | NM_013274 | polymerase (DNA directed), lambda |
| POLR2D | NM_004805 | DNA directed RNA polymerase II polypeptide D |
| POLR2E | NM_002695 | DNA directed RNA polymerase II polypeptide E |
| POLR2G | NM_002696 | DNA directed RNA polymerase II polypeptide G |
| POLR2J | NM_006234 | DNA directed RNA polymerase II polypeptide J |
| POLR3B | NM_018082 | polymerase (RNA) III (DNA directed) polypeptide |
| POLR3D | NM_001722 | RNA polymerase III 53 kDa subunit RPC4 |
| POLR3F | NM_006466 | DNA-directed RNA polymerase III 39 kDa |
| POM121 | NM_172020 | nuclear pore membrane protein 121 |
| POMT2 | NM_013382 | putative protein O-mannosyltransferase |
| POMZP3 | NM_012230 | POMZP3 fusion protein isoform 1 |
| POU2AF1 | NM_006235 | POU domain, class 2, associating factor 1 |
| POU3F2 | NM_005604 | POU domain, class 3, transcription factor 2 |
| POU4F1 | NM_006237 | POU domain, class 4, transcription factor 1 |
| POU4F2 | NM_004575 | POU domain, class 4, transcription factor 2 |
| POU6F1 | NM_002702 | POU domain, class 6, transcription factor 1 |
| PPAP2A | NM_003711 | phosphatidic acid phosphatase type 2A isoform 1 |
| PPAP2B | NM_003713 | phosphatidic acid phosphatase type 2B |
| PPAP2C | NM_003712 | phosphatidic acid phosphatase type 2C isoform 1 |
| PPAPDC2 | NM_203453 | phosphatidic acid phosphatase type 2 domain |
| PPAPDC3 | NM_032728 | phosphatidic acid phosphatase type 2 domain |
| PPARA | NM_001001928 | peroxisome proliferative activated receptor, |
| PPARD | NM_006238 | peroxisome proliferative activated receptor, |
| PPARGC1A | NM_013261 | peroxisome proliferative activated receptor |
| PPFIA3 | NM_003660 | PTPRF interacting protein alpha 3 |
| PPFIA4 | NM_015053 | protein tyrosine phosphatase, receptor type, f |
| PPIE | NM_006112 | peptidylprolyl isomerase E isoform 1 |
| PPIF | NM_005729 | peptidylprolyl isomerase F precursor |
| PPIH | NM_006347 | peptidylprolyl isomerase H |
| PPIL1 | NM_016059 | peptidylprolyl isomerase-like 1 |
| PPIL2 | NM_014337 | peptidylprolyl isomerase-like 2 isoform a |
| PPIL4 | NM_139126 | peptidylprolyl isomerase-like 4 |
| PPL | NM_002705 | periplakin |
| PPM1A | NM_021003 | protein phosphatase 1A isoform 1 |
| PPM1D | NM_003620 | protein phosphatase 1D |
| PPM1E | NM_014906 | protein phosphatase 1E |
| PPM1F | NM_014634 | protein phosphatase 1F |
| PPM1L | NM_139245 | protein phosphatase 1 (formerly 2C)-like |
| PPM1M | NM_144641 | protein phosphatase 1M (PP2C domain containing) |
| PPM2C | NM_018444 | pyruvate dehydrogenase phosphatase precursor |
| PPME1 | NM_016147 | protein phosphatase methylesterase-1 |
| PPP1CA | NM_001008709 | protein phosphatase 1, catalytic subunit, alpha |
| PPP1R11 | NM_021959 | protein phosphatase 1, regulatory (inhibitor) |
| PPP1R12A | NM_002480 | protein phosphatase 1, regulatory (inhibitor) |
| PPP1R12B | NM_002481 | protein phosphatase 1, regulatory (inhibitor) |
| PPP1R12C | NM_017607 | protein phosphatase 1, regulatory subunit 12C |
| PPP1R13B | NM_015316 | protein phosphatase 1, regulatory (inhibitor) |
| PPP1R14C | NM_030949 | protein phosphatase 1, regulatory (inhibitor) |
| PPP1R16B | NM_015568 | protein phosphatase 1 regulatory inhibitor |
| PPP1R1A | NM_006741 | protein phosphatase 1, regulatory (inhibitor) |
| PPP1R2 | NM_006241 | protein phosphatase 1, regulatory (inhibitor) |
| PPP1R3B | NM_024607 | protein phosphatase 1, regulatory (inhibitor) |
| PPP2CA | NM_002715 | protein phosphatase 2, catalytic subunit, alpha |
| PPP2R1A | NM_014225 | alpha isoform of regulatory subunit A, protein |
| PPP2R1B | NM_002716 | beta isoform of regulatory subunit A, protein |
| PPP2R2C | NM_020416 | gamma isoform of regulatory subunit B55, protein |
| PPP2R2D | NM_001003656 | protein phosphatase 2, regulatory subunit B, |
| PPP2R4 | NM_021131 | protein phosphatase 2A, regulatory subunit B′ |
| PPP2R5C | NM_002719 | gamma isoform of regulatory subunit B56, protein |
| PPP3CB | NM_021132 | protein phosphatase 3 (formerly 2B), catalytic |
| PPP4R1L | NM_018498 | hypothetical protein LOC55370 |
| PPP6C | NM_002721 | protein phosphatase 6, catalytic subunit |
| PPRC1 | NM_015062 | PGC-1 related co-activator |
| PPT1 | NM_000310 | palmitoyl-protein thioesterase 1 |
| PPT2 | NM_005155 | palmitoyl-protein thioesterase 2 isoform a |
| PPTC7 | NM_139283 | T-cell activation protein phosphatase 2C |
| PQLC1 | NM_025078 | PQ loop repeat containing 1 |
| PRDM12 | NM_021619 | PR domain containing 12 |
| PRDM16 | NM_022114 | PR domain containing 16 isoform 1 |
| PRDM2 | NM_001007257 | retinoblastoma protein-binding zinc finger |
| PRDM4 | NM_012406 | PR domain containing 4 |
| PREI3 | NM_015387 | preimplantation protein 3 isoform 1 |
| PRELP | NM_002725 | proline arginine-rich end leucine-rich repeat |
| PRF1 | NM_005041 | perforin 1 precursor |
| PRH2 | NM_005042 | proline-rich protein HaeIII subfamily 2 |
| PRIC285 | NM_033405 | PPAR-alpha interacting complex protein 285 |
| PRICKLE2 | NM_198859 | prickle-like 2 |
| PRKAA1 | NM_006251 | protein kinase, AMP-activated, alpha 1 catalytic |
| PRKAB2 | NM_005399 | AMP-activated protein kinase beta 2 |
| PRKACA | NM_002730 | cAMP-dependent protein kinase catalytic subunit |
| PRKAR1A | NM_002734 | cAMP-dependent protein kinase, regulatory |
| PRKAR2A | NM_004157 | cAMP-dependent protein kinase, regulatory |
| PRKCA | NM_002737 | protein kinase C, alpha |
| PRKCBP1 | NM_012408 | protein kinase C binding protein 1 isoform b |
| PRKCD | NM_006254 | protein kinase C, delta |
| PRKCG | NM_002739 | protein kinase C, gamma |
| PRKCI | NM_002740 | protein kinase C, iota |
| PRKCZ | NM_001033581 | protein kinase C, zeta isoform 2 |
| PRKD2 | NM_016457 | protein kinase D2 |
| PRKD3 | NM_005813 | protein kinase D3 |
| PRKG1 | NM_006258 | protein kinase, cGMP-dependent, type I |
| PRNT | NM_177549 | prion protein (testis specific) |
| PRO0149 | NM_014117 | hypothetical protein LOC29035 |
| PROK2 | NM_021935 | prokineticin 2 |
| ProSAPiP1 | NM_014731 | ProSAPiP1 protein |
| PROSC | NM_007198 | proline synthetase co-transcribed homolog |
| PRPF38A | NM_032864 | PRP38 pre-mRNA processing factor 38 (yeast) |
| PRPS2 | NM_002765 | phosphoribosyl pyrophosphate synthetase 2 |
| PRR13 | NM_001005354 | hypothetical protein LOC54458 isoform 2 |
| PRR3 | NM_025263 | proline-rich protein 3 |
| PRRG1 | NM_000950 | proline rich Gla (G-carboxyglutamic acid) 1 |
| PRRX1 | NM_006902 | paired mesoderm homeobox 1 isoform pmx-1a |
| PRSS12 | NM_003619 | neurotrypsin precursor |
| PRSS22 | NM_022119 | protease, serine, 22 |
| PRSS23 | NM_007173 | protease, serine, 23 precursor |
| PRSS27 | NM_031948 | marapsin |
| PRSS33 | NM_152891 | protease, serine, 33 |
| PRSS7 | NM_002772 | enterokinase precursor |
| PRX | NM_020956 | periaxin isoform 1 |
| PSAP | NM_002778 | prosaposin |
| PSAT1 | NM_021154 | phosphoserine aminotransferase isoform 2 |
| PSCA | NM_005672 | prostate stem cell antigen preproprotein |
| PSCD3 | NM_004227 | pleckstrin homology, Sec7 and coiled/coil |
| PSD3 | NM_015310 | ADP-ribosylation factor guanine nucleotide |
| PSD4 | NM_012455 | pleckstrin and Sec7 domain containing 4 |
| PSKH1 | NM_006742 | protein serine kinase H1 |
| PSMB5 | NM_002797 | proteasome beta 5 subunit |
| PSMD13 | NM_002817 | proteasome 26S non-ATPase subunit 13 isoform 1 |
| PSMD7 | NM_002811 | proteasome 26S non-ATPase subunit 7 |
| PSMD9 | NM_002813 | proteasome 26S non-ATPase subunit 9 |
| PSME3 | NM_005789 | proteasome activator subunit 3 isoform 1 |
| PSME4 | NM_014614 | proteasome (prosome, macropain) activator |
| PSORS1C2 | NM_014069 | SPR1 protein |
| PSRC2 | NM_144982 | hypothetical protein LOC196441 |
| PTBP1 | NM_002819 | polypyrimidine tract-binding protein 1 isoform |
| PTCH | NM_000264 | patched |
| PTD008 | NM_016145 | hypothetical protein LOC51398 |
| PTDSS1 | NM_014754 | phosphatidylserine synthase 1 |
| PTER | NM_001001484 | phosphotriesterase related |
| PTGER3 | NM_198718 | prostaglandin E receptor 3, subtype EP3 isoform |
| PTGES2 | NM_198939 | prostaglandin E synthase 2 isoform 3 |
| PTGFRN | NM_020440 | prostaglandin F2 receptor negative regulator |
| PTGIR | NM_000960 | prostaglandin I2 (prostacyclin) receptor (IP) |
| PTGS1 | NM_000962 | prostaglandin-endoperoxide synthase 1 isoform 1 |
| PTH | NM_000315 | parathyroid hormone preproprotein |
| PTHLH | NM_198965 | parathyroid hormone-like hormone isoform 1 |
| PTK2B | NM_004103 | PTK2B protein tyrosine kinase 2 beta isoform a |
| PTK6 | NM_005975 | PTK6 protein tyrosine kinase 6 |
| PTK7 | NM_152883 | PTK7 protein tyrosine kinase 7 isoform e |
| PTPDC1 | NM_152422 | protein tyrosine phosphatase domain containing 1 |
| PTPLAD2 | NM_001010915 | hypothetical protein LOC401494 |
| PTPN18 | NM_014369 | protein tyrosine phosphatase, non-receptor type |
| PTPN20B | NM_015605 | protein tyrosine phosphatase, non-receptor type |
| PTPN3 | NM_002829 | protein tyrosine phosphatase, non-receptor type |
| PTPN4 | NM_002830 | protein tyrosine phosphatase, non-receptor type |
| PTPN7 | NM_002832 | protein tyrosine phosphatase, non-receptor type |
| PTPRF | NM_002840 | protein tyrosine phosphatase, receptor type, F |
| PTPRM | NM_002845 | protein tyrosine phosphatase, receptor type, M |
| PTPRR | NM_002849 | protein tyrosine phosphatase, receptor type, R |
| PTPRT | NM_007050 | protein tyrosine phosphatase, receptor type, T |
| PURA | NM_005859 | purine-rich element binding protein A |
| PURB | NM_033224 | purine-rich element binding protein B |
| PURG | NM_013357 | purine-rich element binding protein G isoform A |
| PUSL1 | NM_153339 | pseudouridylate synthase-like 1 |
| PWWP2 | NM_138499 | PWWP domain containing 2 |
| PXMP4 | NM_007238 | peroxisomal membrane protein 4 isoform a |
| PXN | NM_002859 | paxillin |
| PYCR1 | NM_006907 | pyrroline-5-carboxylate reductase 1 isoform 1 |
| PYCR2 | NM_013328 | pyrroline-5-carboxylate reductase family, member |
| PYCRL | NM_023078 | pyrroline-5-carboxylate reductase-like |
| PYY2 | NM_021093 | peptide YY, 2 (seminalplasmin) |
| QKI | NM_206853 | quaking homolog, KH domain RNA binding isoform |
| QPRT | NM_014298 | quinolinate phosphoribosyltransferase |
| QSCN6L1 | NM_181701 | quiescin Q6-like 1 |
| QTRTD1 | NM_024638 | queuine tRNA-ribosyltransferase domain |
| RAB10 | NM_016131 | ras-related GTP-binding protein RAB10 |
| RAB11FIP1 | NM_001002814 | Rab coupling protein isoform 3 |
| RAB11FIP2 | NM_014904 | RAB11 family interacting protein 2 (class I) |
| RAB11FIP3 | NM_014700 | rab11-family interacting protein 3 |
| RAB11FIP4 | NM_032932 | RAB11 family interacting protein 4 (class II) |
| RAB11FIP5 | NM_015470 | RAB11 family interacting protein 5 (class I) |
| RAB15 | NM_198686 | Ras-related protein Rab-15 |
| RAB1A | NM_004161 | RAB1A, member RAS oncogene family |
| RAB22A | NM_020673 | RAS-related protein RAB-22A |
| RAB23 | NM_016277 | Ras-related protein Rab-23 |
| RAB2B | NM_032846 | RAB2B protein |
| RAB39B | NM_171998 | RAB39B, member RAS oncogene family |
| RAB3B | NM_002867 | RAB3B, member RAS oncogene family |
| RAB3D | NM_004283 | RAB3D, member RAS oncogene family |
| RAB40A | NM_080879 | RAB40A, member RAS oncogene family |
| RAB40B | NM_006822 | RAB40B, member RAS oncogene family |
| RAB43 | NM_198490 | RAB43 protein |
| RAB4B | NM_016154 | ras-related GTP-binding protein 4b |
| RAB6B | NM_016577 | RAB6B, member RAS oncogene family |
| RAB6IP2 | NM_015064 | RAB6-interacting protein 2 isoform alpha |
| RAB8B | NM_016530 | RAB8B, member RAS oncogene family |
| RAB9A | NM_004251 | RAB9A, member RAS oncogene family |
| RABAC1 | NM_006423 | Rab acceptor 1 |
| RABEP2 | NM_024816 | rabaptin, RAB GTPase binding effector protein 2 |
| RABL3 | NM_173825 | RAB, member of RAS oncogene family-like 3 |
| RACGAP1 | NM_013277 | Rac GTPase activating protein 1 |
| RAD23A | NM_005053 | UV excision repair protein RAD23 homolog A |
| RAD23B | NM_002874 | UV excision repair protein RAD23 homolog B |
| RAD50 | NM_005732 | RAD50 homolog isoform 1 |
| RAD51L1 | NM_133509 | RAD51-like 1 isoform 3 |
| RAD51L3 | NM_002878 | RAD51-like 3 isoform 1 |
| RAD9A | NM_004584 | RAD9 homolog |
| RAET1G | NM_001001788 | retinoic acid early transcript 1G |
| RAF1 | NM_002880 | v-raf-1 murine leukemia viral oncogene homolog |
| RAGE | NM_014226 | MAPK/MAK/MRK overlapping kinase |
| RAI14 | NM_015577 | retinoic acid induced 14 |
| RAI17 | NM_020338 | retinoic acid induced 17 |
| RALB | NM_002881 | v-ral simian leukemia viral oncogene homolog B |
| RALBP1 | NM_006788 | ralA binding protein 1 |
| RALGPS1 | NM_014636 | Ral GEF with PH domain and SH3 binding motif 1 |
| RANBP10 | NM_020850 | RAN binding protein 10 |
| RANBP3 | NM_003624 | RAN binding protein 3 isoform RANBP3-a |
| RANGAP1 | NM_002883 | Ran GTPase activating protein 1 |
| RAP1GAP | NM_002885 | RAP1, GTPase activating protein 1 |
| RAP1GDS1 | NM_021159 | RAP1, GTP-GDP dissociation stimulator 1 |
| RAP2C | NM_021183 | RAP2C, member of RAS oncogene family |
| RAPGEF1 | NM_005312 | guanine nucleotide-releasing factor 2 isoform a |
| RAPGEFL1 | NM_016339 | Rap guanine nucleotide exchange factor |
| RAPH1 | NM_213589 | Ras association and pleckstrin homology domains |
| RARB | NM_000965 | retinoic acid receptor, beta isoform 1 |
| RARG | NM_000966 | retinoic acid receptor, gamma |
| RARRES2 | NM_002889 | retinoic acid receptor responder (tazarotene |
| RASA3 | NM_007368 | RAS p21 protein activator 3 |
| RASA4 | NM_006989 | RAS p21 protein activator 4 |
| RASAL1 | NM_004658 | RAS protein activator like 1 |
| RASGEF1B | NM_152545 | RasGEF domain family, member 1B |
| RASGEF1C | NM_001031799 | RasGEF domain family, member 1C isoform 2 |
| RASL12 | NM_016563 | RAS-like, family 12 protein |
| RASSF1 | NM_007182 | Ras association domain family 1 isoform A |
| RASSF2 | NM_014737 | Ras association domain family 2 |
| RASSF3 | NM_178169 | Ras association (RalGDS/AF-6) domain family 3 |
| RASSF4 | NM_032023 | Ras association domain family 4 isoform a |
| RASSF5 | NM_031437 | Ras association (RalGDS/AF-6) domain family 5 |
| RBBP6 | NM_006910 | retinoblastoma-binding protein 6 isoform 1 |
| RBED1 | NM_032213 | RNA binding motif and ELMO domain 1 |
| RBJ | NM_016544 | Ras-associated protein Rap1 |
| RBL2 | NM_005611 | retinoblastoma-like 2 (p130) |
| RBM12 | NM_006047 | RNA binding motif protein 12 |
| RBM12B | NM_203390 | hypothetical protein LOC389677 |
| RBM16 | NM_014892 | RNA-binding motif protein 16 |
| RBM19 | NM_016196 | RNA binding motif protein 19 |
| RBM21 | NM_022830 | RNA binding motif protein 21 |
| RBM23 | NM_018107 | hypothetical protein LOC55147 |
| RBM24 | NM_153020 | hypothetical protein LOC221662 |
| RBM33 | NM_001008408 | hypothetical protein LOC155435 |
| RBM35B | NM_024939 | hypothetical protein LOC80004 |
| RBM6 | NM_005777 | RNA binding motif protein 6 |
| RBM7 | NM_016090 | RNA binding motif protein 7 |
| RBPMS2 | NM_194272 | RNA binding protein with multiple splicing 2 |
| RCE1 | NM_001032279 | prenyl protein peptidase RCE1 isoform 2 |
| RCL1 | NM_005772 | RNA cyclase homolog |
| RCOR3 | NM_018254 | REST corepressor 3 |
| RDH13 | NM_138412 | retinol dehydrogenase 13 (all-trans and 9-cis) |
| RDM1 | NM_145654 | RAD52 motif 1 isoform 1 |
| RDS | NM_000322 | retinal degeneration slow protein |
| RECK | NM_021111 | RECK protein precursor |
| RECQL5 | NM_004259 | RecQ protein-like 5 isoform 1 |
| REEP1 | NM_022912 | receptor expression enhancing protein 1 |
| REEP3 | NM_001001330 | receptor expression enhancing protein 3 |
| RELN | NM_005045 | reelin isoform a |
| RET | NM_020975 | ret proto-oncogene isoform a |
| REXO1 | NM_020695 | transcription elongation factor B polypeptide 3 |
| REXO4 | NM_020385 | XPMC2 prevents mitotic catastrophe 2 homolog |
| RFFL | NM_001017368 | rififylin |
| RFK | NM_018339 | riboflavin kinase |
| RFT1 | NM_052859 | hypothetical protein LOC91869 |
| RFWD2 | NM_001001740 | ring finger and WD repeat domain 2 isoform d24 |
| RFWD3 | NM_018124 | ring finger and WD repeat domain 3 |
| RFX4 | NM_002920 | regulatory factor X4 isoform b |
| RGAG4 | NM_001024455 | retrotransposon gag domain containing 4 |
| RGL1 | NM_015149 | ral guanine nucleotide dissociation |
| RGMA | NM_020211 | RGM domain family, member A |
| RGMB | NM_001012761 | RGM domain family, member B isoform 1 precursor |
| RGPD5 | NM_005054 | RANBP2-like and GRIP domain containing 5 isoform |
| RGS11 | NM_003834 | regulator of G-protein signalling 11 isoform 2 |
| RGS12 | NM_002926 | regulator of G-protein signalling 12 isoform 2 |
| RGS22 | NM_015668 | regulator of G-protein signalling 22 |
| RGS3 | NM_017790 | regulator of G-protein signalling 3 isoform 3 |
| RGS5 | NM_003617 | regulator of G-protein signalling 5 |
| RGS9BP | NM_207391 | RGS9 anchor protein |
| RHBDL3 | NM_138328 | rhomboid, veinlet-like 3 |
| RHBG | NM_020407 | Rhesus blood group, B glycoprotein |
| RHOB | NM_004040 | ras homolog gene family, member B |
| RHOBTB2 | NM_015178 | Rho-related BTB domain containing 2 |
| RHOD | NM_014578 | ras homolog D |
| RHOJ | NM_020663 | TC10-like Rho GTPase |
| RHOU | NM_021205 | ras homolog gene family, member U |
| RHPN2 | NM_033103 | rhophilin-like protein |
| RIC8A | NM_021932 | resistance to inhibitors of cholinesterase 8 |
| RICTOR | NM_152756 | rapamycin-insensitive companion of mTOR |
| RIF1 | NM_018151 | RAP1 interacting factor 1 |
| RIMBP2 | NM_015347 | RIM-binding protein 2 |
| RIMS3 | NM_014747 | regulating synaptic membrane exocytosis 3 |
| RIPK4 | NM_020639 | ankyrin repeat domain 3 |
| RIPK5 | NM_015375 | receptor interacting protein kinase 5 isoform 1 |
| RKHD2 | NM_016626 | ring finger and KH domain containing 2 |
| RKHD3 | NM_032246 | ring finger and KH domain containing 3 |
| RNASEH1 | NM_002936 | ribonuclease H1 |
| RNF10 | NM_014868 | ring finger protein 10 |
| RNF111 | NM_017610 | ring finger protein 111 |
| RNF125 | NM_017831 | ring finger protein 125 |
| RNF138 | NM_016271 | ring finger protein 138 isoform 1 |
| RNF144 | NM_014746 | ring finger protein 144 |
| RNF149 | NM_173647 | ring finger protein 149 |
| RNF165 | NM_152470 | ring finger protein 165 |
| RNF166 | NM_178841 | ring finger protein 166 |
| RNF183 | NM_145051 | ring finger protein 183 |
| RNF190 | NM_152598 | hypothetical protein LOC162333 |
| RNF24 | NM_007219 | ring finger protein 24 |
| RNF31 | NM_017999 | ring finger protein 31 |
| RNF38 | NM_022781 | ring finger protein 38 isoform 1 |
| RNF39 | NM_025236 | HZFw1 protein isoform 1 |
| RNF41 | NM_005785 | ring finger protein 41 isoform 1 |
| RNF43 | NM_017763 | ring finger protein 43 |
| RNF44 | NM_014901 | ring finger protein 44 |
| RNF8 | NM_003958 | ring finger protein 8 isoform 1 |
| RNGTT | NM_003800 | RNA guanylyltransferase and 5′-phosphatase |
| RNH1 | NM_002939 | ribonuclease/angiogenin inhibitor |
| RNMT | NM_003799 | RNA (guanine-7-) methyltransferase |
| RNPC1 | NM_017495 | RNA-binding region containing protein 1 isoform |
| RNPS1 | NM_006711 | RNA-binding protein S1, serine-rich domain |
| ROBO4 | NM_019055 | roundabout homolog 4, magic roundabout |
| ROGDI | NM_024589 | leucine zipper domain protein |
| RP13-15M17.2 | NM_001010866 | hypothetical protein LOC199953 |
| RP1-32F7.2 | NM_173698 | hypothetical protein LOC286499 |
| RP3-473B4.1 | NM_138819 | hypothetical protein LOC159091 |
| RPH3AL | NM_006987 | rabphilin 3A-like (without C2 domains) |
| RPL10 | NM_006013 | ribosomal protein L10 |
| RPL28 | NM_000991 | ribosomal protein L28 |
| RPL32 | NM_000994 | ribosomal protein L32 |
| RPP14 | NM_007042 | ribonuclease P 14 kDa subunit |
| RPP25 | NM_017793 | ribonuclease P 25 kDa subunit |
| RPRM | NM_019845 | reprimo, TP53 dependant G2 arrest mediator |
| RPRML | NM_203400 | reprimo-like |
| RPS23 | NM_001025 | ribosomal protein S23 |
| RPS6KA3 | NM_004586 | ribosomal protein S6 kinase, 90 kDa, polypeptide |
| RPS6KA5 | NM_004755 | ribosomal protein S6 kinase, 90 kDa, polypeptide |
| RPS6KB1 | NM_003161 | ribosomal protein S6 kinase, 70 kDa, polypeptide |
| RPS6KB2 | NM_001007071 | ribosomal protein S6 kinase, 70 kDa, polypeptide |
| RPUSD1 | NM_058192 | RNA pseudouridylate synthase domain containing |
| RPUSD4 | NM_032795 | RNA pseudouridylate synthase domain containing |
| RRAGA | NM_006570 | Ras-related GTP binding A |
| RRAGC | NM_022157 | Ras-related GTP binding C |
| RREB1 | NM_001003698 | ras responsive element binding protein 1 isoform |
| RRH | NM_006583 | peropsin |
| RRP22 | NM_001007279 | RAS-related on chromosome 22 isoform b |
| RS1 | NM_000330 | X-linked juvenile retinoschisis protein |
| RSBN1 | NM_018364 | round spermatid basic protein 1 |
| RSNL2 | NM_024692 | restin-like 2 |
| RSPO2 | NM_178565 | R-spondin family, member 2 |
| RSPO3 | NM_032784 | thrombospondin, type I, domain containing 2 |
| RSU1 | NM_012425 | ras suppressor protein 1 isoform 1 |
| RTEL1 | NM_032957 | regulator of telomere elongation helicase 1 |
| RTF1 | NM_015138 | Paf1/RNA polymerase II complex component |
| RTN2 | NM_206902 | reticulon 2 isoform D |
| RTN3 | NM_006054 | reticulon 3 isoform a |
| RTN4 | NM_007008 | reticulon 4 isoform C |
| RTN4RL1 | NM_178568 | reticulon 4 receptor-like 1 |
| RUNX1 | NM_001001890 | runt-related transcription factor 1 isoform b |
| RUNX1T1 | NM_004349 | acute myelogenous leukemia 1 translocation 1 |
| RUTBC1 | NM_014853 | RUN and TBC1 domain containing 1 |
| RXRA | NM_002957 | retinoid X receptor, alpha |
| RYBP | NM_012234 | RING1 and YY1 binding protein |
| S100A5 | NM_002962 | S100 calcium binding protein A5 |
| S100A7L1 | NM_176823 | S100 calcium binding protein A7-like 1 |
| SACM1L | NM_014016 | suppressor of actin 1 |
| SAE1 | NM_005500 | SUMO-1 activating enzyme subunit 1 |
| SALL2 | NM_005407 | sal-like 2 |
| SALL3 | NM_171999 | sal-like 3 |
| SALL4 | NM_020436 | sal-like 4 |
| SAMD10 | NM_080621 | sterile alpha motif domain containing 10 |
| SAPS2 | NM_014678 | hypothetical protein LOC9701 |
| SAPS3 | NM_018312 | SAPS domain family, member 3 |
| SARM1 | NM_015077 | sterile alpha and TIR motif containing 1 |
| SAT | NM_002970 | spermidine/spermine N1-acetyltransferase |
| SATB2 | NM_015265 | SATB family member 2 |
| SAV1 | NM_021818 | WW45 protein |
| SBF1 | NM_002972 | SET binding factor 1 isoform a |
| SCAMP1 | NM_004866 | secretory carrier membrane protein 1 isoform 1 |
| SCAMP4 | NM_079834 | secretory carrier membrane protein 4 |
| SCAMP5 | NM_138967 | secretory carrier membrane protein 5 |
| SCAND2 | NM_022050 | SCAN domain-containing protein 2 isoform 1 |
| SCARB1 | NM_005505 | scavenger receptor class B, member 1 |
| SCARF1 | NM_145349 | scavenger receptor class F, member 1 isoform 2 |
| SCCPDH | NM_016002 | saccharopine dehydrogenase (putative) |
| SCG3 | NM_013243 | secretogranin III |
| SCMH1 | NM_001031694 | sex comb on midleg homolog 1 isoform 1 |
| SCML4 | NM_198081 | sex comb on midleg-like 4 |
| SCN2B | NM_004588 | sodium channel, voltage-gated, type II, beta |
| SCN3A | NM_006922 | sodium channel, voltage-gated, type III, alpha |
| SCN4A | NM_000334 | voltage-gated sodium channel type 4 alpha |
| SCN4B | NM_174934 | sodium channel, voltage-gated, type IV, beta |
| SCN5A | NM_000335 | voltage-gated sodium channel type V alpha |
| SCOC | NM_032547 | short coiled-coil protein |
| SCOTIN | NM_016479 | scotin |
| SCRN1 | NM_014766 | secernin 1 |
| SDC1 | NM_001006946 | syndecan 1 precursor |
| SDCBP2 | NM_015685 | syndecan binding protein 2 isoform b |
| SDHC | NM_003001 | succinate dehydrogenase complex, subunit C |
| SEC14L1 | NM_003003 | SEC14 (S. cerevisiae)-like 1 isoform a |
| SEC14L4 | NM_174977 | SEC14p-like protein TAP3 |
| SEC22C | NM_004206 | SEC22 vesicle trafficking protein homolog C |
| SEC61A1 | NM_013336 | Sec61 alpha 1 subunit |
| SECISBP2 | NM_024077 | SECIS binding protein 2 |
| SEH1L | NM_001013437 | sec13-like protein isoform 1 |
| SEL1L | NM_005065 | sel-1 suppressor of lin-12-like |
| SELE | NM_000450 | selectin E precursor |
| SELENBP1 | NM_003944 | selenium binding protein 1 |
| SELI | NM_033505 | selenoprotein I |
| SELO | NM_031454 | selenoprotein O |
| SELPLG | NM_003006 | selectin P ligand |
| SELS | NM_018445 | selenoprotein S |
| SEMA3B | NM_001005914 | semaphorin 3B isoform 2 precursor |
| SEMA3D | NM_152754 | semaphorin 3D |
| SEMA3E | NM_012431 | semaphorin 3E |
| SEMA3G | NM_020163 | semaphorin sem2 |
| SEMA4B | NM_020210 | semaphorin 4B precursor |
| SEMA4F | NM_004263 | semaphorin W |
| SEMA5A | NM_003966 | semaphorin 5A |
| SEMA5B | NM_001031702 | semaphorin 5B isoform 1 |
| SEMA6A | NM_020796 | sema domain, transmembrane domain (TM), and |
| SEMA6B | NM_032108 | semaphorin 6B isoform 3 precursor |
| SEMA6D | NM_020858 | semaphorin 6D isoform 1 precursor |
| SEMA7A | NM_003612 | semaphorin 7A |
| SENP1 | NM_014554 | sentrin/SUMO-specific protease 1 |
| SENP2 | NM_021627 | SUMO1/sentrin/SMT3 specific protease 2 |
| SEPN1 | NM_020451 | selenoprotein N, 1 isoform 1 precursor |
| SEPT11 | NM_018243 | septin 11 |
| SEPT2 | NM_001008491 | septin 2 |
| SEPT3 | NM_019106 | septin 3 isoform B |
| SEPT9 | NM_006640 | septin 9 |
| SEPW1 | NM_003009 | selenoprotein W, 1 |
| SERAC1 | NM_032861 | serine active site containing 1 |
| SERBP1 | NM_001018067 | SERPINE1 mRNA binding protein 1 isoform 1 |
| SERHL | NM_170694 | serine hydrolase-like |
| SERINC2 | NM_178865 | tumor differentially expressed 2-like |
| SERPINA10 | NM_016186 | serine (or cysteine) proteinase inhibitor, clade |
| SERPINB13 | NM_012397 | serine (or cysteine) proteinase inhibitor, clade |
| SERPINB2 | NM_002575 | serine (or cysteine) proteinase inhibitor, clade |
| SERPINB7 | NM_003784 | serine (or cysteine) proteinase inhibitor, clade |
| SERPINB9 | NM_004155 | serine (or cysteine) proteinase inhibitor, clade |
| SERPINE1 | NM_000602 | plasminogen activator inhibitor-1 |
| SERPINF2 | NM_000934 | alpha-2-plasmin inhibitor |
| SERPING1 | NM_000062 | complement component 1 inhibitor precursor |
| SESN1 | NM_014454 | sestrin 1 |
| SESN2 | NM_031459 | sestrin 2 |
| SETD3 | NM_032233 | hypothetical protein LOC84193 isoform a |
| SETD4 | NM_001007258 | hypothetical protein LOC54093 isoform b |
| SF1 | NM_201997 | splicing factor 1 isoform 4 |
| SF3A1 | NM_001005409 | splicing factor 3a, subunit 1, 120 kDa isoform 2 |
| SF3A3 | NM_006802 | splicing factor 3a, subunit 3 |
| SF4 | NM_021164 | splicing factor 4 isoform b |
| SFRS11 | NM_004768 | splicing factor p54 |
| SFRS12 | NM_139168 | splicing factor, arginine/serine-rich 12 |
| SFRS16 | NM_007056 | splicing factor, arginine/serine-rich 16 |
| SFRS2 | NM_003016 | splicing factor, arginine/serine-rich 2 |
| SFRS2IP | NM_004719 | splicing factor, arginine/serine-rich 2, |
| SFRS5 | NM_006925 | splicing factor, arginine/serine-rich 5 |
| SFRS8 | NM_152235 | splicing factor, arginine/serine-rich 8 isoform |
| SFT2D3 | NM_032740 | SFT2 domain containing 3 |
| SFTPB | NM_000542 | surfactant, pulmonary-associated protein B |
| SFXN1 | NM_022754 | sideroflexin 1 |
| SFXN2 | NM_178858 | sideroflexin 2 |
| SFXN3 | NM_030971 | sideroflexin 3 |
| SFXN5 | NM_144579 | sideroflexin 5 |
| SGCA | NM_000023 | sarcoglycan, alpha (50 kDa dystrophin-associated |
| SGCD | NM_000337 | delta-sarcoglycan isoform 1 |
| SGK | NM_005627 | serum/glucocorticoid regulated kinase |
| SGK2 | NM_016276 | serum/glucocorticoid regulated kinase 2 isoform |
| SGK3 | NM_001033578 | serum/glucocorticoid regulated kinase 3 isoform |
| SH2D2A | NM_003975 | SH2 domain protein 2A |
| SH2D3C | NM_170600 | SH2 domain containing 3C isoform 2 |
| SH3BGRL2 | NM_031469 | SH3 domain binding glutamic acid-rich protein |
| SH3BP2 | NM_003023 | SH3-domain binding protein 2 |
| SH3BP4 | NM_014521 | SH3-domain binding protein 4 |
| SH3BP5L | NM_030645 | SH3-binding domain protein 5-like |
| SH3GL2 | NM_003026 | SH3-domain GRB2-like 2 |
| SH3PX3 | NM_153271 | SH3 and PX domain containing 3 |
| SH3PXD2B | NM_001017995 | SH3 and PX domains 2B |
| SHANK2 | NM_012309 | SH3 and multiple ankyrin repeat domains 2 |
| SHC3 | NM_016848 | src homology 2 domain containing transforming |
| SHF | NM_138356 | hypothetical protein LOC90525 |
| SHOC2 | NM_007373 | soc-2 suppressor of clear homolog |
| SHOX | NM_006883 | short stature homeobox isoform b |
| SHOX2 | NM_003030 | short stature homeobox 2 isoform b |
| SHRM | NM_020859 | shroom |
| SIAH1 | NM_001006610 | seven in absentia homolog 1 isoform b |
| SIAHBP1 | NM_014281 | fuse-binding protein-interacting repressor |
| SIDT1 | NM_017699 | SID1 transmembrane family, member 1 |
| SIM2 | NM_005069 | single-minded homolog 2 long isoform |
| SIPA1L2 | NM_020808 | signal-induced proliferation-associated 1 like |
| SIRPA | NM_080792 | signal-regulatory protein alpha precursor |
| SIRPB1 | NM_006065 | signal-regulatory protein beta 1 precursor |
| SIRT4 | NM_012240 | sirtuin 4 |
| SIRT5 | NM_031244 | sirtuin 5 isoform 2 |
| SIX4 | NM_017420 | sine oculis homeobox homolog 4 |
| SKI | NM_003036 | v-ski sarcoma viral oncogene homolog |
| SKIP | NM_030623 | sphingosine kinase type 1-interacting protein |
| SLC11A2 | NM_000617 | solute carrier family 11 (proton-coupled |
| SLC12A2 | NM_001046 | solute carrier family 12 |
| SLC12A5 | NM_020708 | solute carrier family 12 member 5 |
| SLC12A7 | NM_006598 | solute carrier family 12 (potassium/chloride |
| SLC12A8 | NM_024628 | solute carrier family 12, member 8 |
| SLC13A1 | NM_022444 | solute carrier family 13 (sodium/sulfate |
| SLC13A3 | NM_001011554 | solute carrier family 13 member 3 isoform b |
| SLC13A5 | NM_177550 | solute carrier family 13 (sodium-dependent |
| SLC15A4 | NM_145648 | solute carrier family 15, member 4 |
| SLC16A14 | NM_152527 | solute carrier family 16 (monocarboxylic acid |
| SLC16A3 | NM_004207 | solute carrier family 16, member 3 |
| SLC16A8 | NM_013356 | solute carrier family 16, member 8 |
| SLC18A1 | NM_003053 | solute carrier family 18 (vesicular monoamine), |
| SLC18A3 | NM_003055 | solute carrier family 18 (vesicular |
| SLC19A2 | NM_006996 | solute carrier family 19, member 2 |
| SLC1A2 | NM_004171 | solute carrier family 1, member 2 |
| SLC20A2 | NM_006749 | solute carrier family 20, member 2 |
| SLC22A13 | NM_004256 | organic cation transporter like 3 |
| SLC22A15 | NM_018420 | solute carrier family 22 (organic cation |
| SLC22A17 | NM_016609 | solute carrier family 22 (organic cation |
| SLC22A2 | NM_003058 | solute carrier family 22 member 2 isoform a |
| SLC22A7 | NM_153320 | solute carrier family 22 member 7 isoform b |
| SLC24A1 | NM_004727 | solute carrier family 24 |
| SLC24A3 | NM_020689 | solute carrier family 24 |
| SLC24A4 | NM_153646 | solute carrier family 24 member 4 isoform 1 |
| SLC24A6 | NM_024959 | solute carrier family 24 member 6 |
| SLC25A12 | NM_003705 | solute carrier family 25 (mitochondrial carrier, |
| SLC25A15 | NM_014252 | solute carrier family 25 (mitochondrial carrier; |
| SLC25A19 | NM_021734 | solute carrier family 25 (mitochondrial |
| SLC25A2 | NM_031947 | solute carrier family 25 member 2 |
| SLC25A22 | NM_024698 | mitochondrial glutamate carrier 1 |
| SLC25A29 | NM_152333 | solute carrier family 25, member 29 isoform a |
| SLC25A3 | NM_213612 | solute carrier family 25 member 3 isoform c |
| SLC25A34 | NM_207348 | solute carrier family 25, member 34 |
| SLC25A35 | NM_201520 | solute carrier family 25, member 35 |
| SLC26A1 | NM_022042 | solute carrier family 26, member 1 isoform a |
| SLC26A10 | NM_001018084 | solute carrier family 26, member 10 isoform 1 |
| SLC26A2 | NM_000112 | solute carrier family 26 member 2 |
| SLC26A4 | NM_000441 | pendrin |
| SLC28A1 | NM_201651 | solute carrier family 28 (sodium-coupled |
| SLC29A2 | NM_001532 | solute carrier family 29 (nucleoside |
| SLC2A14 | NM_153449 | glucose transporter 14 |
| SLC2A3 | NM_006931 | solute carrier family 2 (facilitated glucose |
| SLC2A4 | NM_001042 | glucose transporter 4 |
| SLC2A8 | NM_014580 | solute carrier family 2, (facilitated glucose |
| SLC30A10 | NM_001004433 | solute carrier family 30 (zinc transporter), |
| SLC30A4 | NM_013309 | solute carrier family 30 (zinc transporter), |
| SLC30A8 | NM_173851 | solute carrier family 30 member 8 |
| SLC31A1 | NM_001859 | solute carrier family 31 (copper transporters), |
| SLC35A4 | NM_080670 | solute carrier family 35, member A4 |
| SLC35B2 | NM_178148 | solute carrier family 35, member B2 |
| SLC35C1 | NM_018389 | solute carrier family 35, member C1 |
| SLC35E1 | NM_024881 | solute carrier family 35, member E1 |
| SLC36A1 | NM_078483 | solute carrier family 36 member 1 |
| SLC36A2 | NM_181776 | solute carrier family 36 (proton/amino acid |
| SLC37A2 | NM_198277 | solute carrier family 37 (glycerol-3-phosphate |
| SLC38A3 | NM_006841 | solute carrier family 38, member 3 |
| SLC38A4 | NM_018018 | solute carrier family 38, member 4 |
| SLC39A1 | NM_014437 | solute carrier family 39 (zinc transporter), |
| SLC39A10 | NM_020342 | solute carrier family 39 (zinc transporter), |
| SLC39A7 | NM_006979 | solute carrier family 39 (zinc transporter), |
| SLC39A9 | NM_018375 | solute carrier family 39 (zinc transporter), |
| SLC3A1 | NM_000341 | solute carrier family 3, member 1 |
| SLC41A2 | NM_032148 | solute carrier family 41, member 2 |
| SLC41A3 | NM_001008487 | solute carrier family 41, member 3 isoform 4 |
| SLC43A1 | NM_003627 | solute carrier family 43, member 1 |
| SLC44A1 | NM_080546 | CDW92 antigen isoform 2 |
| SLC44A2 | NM_020428 | CTL2 protein |
| SLC45A2 | NM_001012509 | membrane-associated transporter protein isoform |
| SLC45A3 | NM_033102 | prostein |
| SLC4A4 | NM_003759 | solute carrier family 4, sodium bicarbonate |
| SLC4A7 | NM_003615 | solute carrier family 4, sodium bicarbonate |
| SLC6A1 | NM_003042 | solute carrier family 6 (neurotransmitter |
| SLC6A14 | NM_007231 | solute carrier family 6 (amino acid |
| SLC6A17 | NM_001010898 | solute carrier family 6, member 17 |
| SLC6A2 | NM_001043 | solute carrier family 6 member 2 |
| SLC6A4 | NM_001045 | solute carrier family 6 member 4 |
| SLC6A6 | NM_003043 | solute carrier family 6 (neurotransmitter |
| SLC6A8 | NM_005629 | solute carrier family 6 (neurotransmitter |
| SLC6A9 | NM_001024845 | solute carrier family 6 member 9 isoform 3 |
| SLC7A1 | NM_003045 | solute carrier family 7 (cationic amino acid |
| SLC7A2 | NM_001008539 | solute carrier family 7, member 2 isoform 1 |
| SLC7A5 | NM_003486 | solute carrier family 7 (cationic amino acid |
| SLC7A6 | NM_003983 | solute carrier family 7 (cationic amino acid |
| SLC8A3 | NM_182933 | solute carrier family 8 member 3 isoform E |
| SLC9A1 | NM_003047 | solute carrier family 9, isoform A1 |
| SLC9A3R2 | NM_004785 | solute carrier family 9 isoform 3 regulator 2 |
| SLC9A5 | NM_004594 | solute carrier family 9 (sodium/hydrogen |
| SLC9A6 | NM_006359 | solute carrier family 9 (sodium/hydrogen |
| SLC9A8 | NM_015266 | Na+/H+ exchanger isoform 8 |
| SLCO2A1 | NM_005630 | solute carrier organic anion transporter family, |
| SLCO4C1 | NM_180991 | solute carrier organic anion transporter family, |
| SLFN11 | NM_152270 | schlafen family member 11 |
| SLFN13 | NM_144682 | schlafen family member 13 |
| SLFNL1 | NM_144990 | hypothetical protein LOC200172 |
| SLITRK1 | NM_052910 | slit and trk like 1 protein |
| SLITRK2 | NM_032539 | SLIT and NTRK-like family, member 2 |
| SLITRK6 | NM_032229 | slit and trk like 6 |
| SLN | NM_003063 | sarcolipin |
| SLURP1 | NM_020427 | ARS component B precursor |
| SMAD2 | NM_001003652 | Sma- and Mad-related protein 2 |
| SMAD3 | NM_005902 | MAD, mothers against decapentaplegic homolog 3 |
| SMAD5 | NM_001001419 | SMAD, mothers against DPP homolog 5 |
| SMAD7 | NM_005904 | MAD, mothers against decapentaplegic homolog 7 |
| SMAF1 | NM_001018082 | small adipocyte factor 1 |
| SMAP1 | NM_021940 | stromal membrane-associated protein |
| SMAP1L | NM_022733 | stromal membrane-associated protein 1-like |
| SMARCA1 | NM_003069 | SWI/SNF-related matrix-associated |
| SMARCD2 | NM_003077 | SWI/SNF-related matrix-associated |
| SMC1L1 | NM_006306 | SMC1 structural maintenance of chromosomes |
| SMC6L1 | NM_024624 | SMC6 protein |
| SMCR8 | NM_144775 | Smith-Magenis syndrome chromosome region, |
| SMG5 | NM_015327 | Est1p-like protein B |
| SMG6 | NM_017575 | Smg-6 homolog, nonsense mediated mRNA decay |
| SMPD1 | NM_000543 | sphingomyelin phosphodiesterase 1, acid |
| SMPD3 | NM_018667 | sphingomyelin phosphodiesterase 3, neutral |
| SMURF1 | NM_020429 | Smad ubiquitination regulatory factor 1 isoform |
| SMURF2 | NM_022739 | SMAD specific E3 ubiquitin protein ligase 2 |
| SMYD1 | NM_198274 | SET and MYND domain containing 1 |
| SMYD4 | NM_052928 | SET and MYND domain containing 4 |
| SMYD5 | NM_006062 | SMYD family member 5 |
| SNAP23 | NM_003825 | synaptosomal-associated protein 23 isoform |
| SNAP25 | NM_003081 | synaptosomal-associated protein 25 isoform |
| SNCG | NM_003087 | synuclein, gamma (breast cancer-specific protein |
| SNF1LK | NM_173354 | SNF1-like kinase |
| SNF1LK2 | NM_015191 | SNF1-like kinase 2 |
| SNIP1 | NM_024700 | Smad nuclear interacting protein |
| SNN | NM_003498 | Stannin |
| SNPH | NM_014723 | syntaphilin |
| SNRK | NM_017719 | SNF related kinase |
| SNRPA1 | NM_003090 | small nuclear ribonucleoprotein polypeptide A′ |
| SNRPC | NM_003093 | small nuclear ribonucleoprotein polypeptide C |
| SNRPD1 | NM_006938 | small nuclear ribonucleoprotein D1 polypeptide |
| SNTB2 | NM_130845 | basic beta 2 syntrophin isoform b |
| SNURF | NM_005678 | SNRPN upstream reading frame protein |
| SNX1 | NM_003099 | sorting nexin 1 isoform a |
| SNX11 | NM_013323 | sorting nexin 11 |
| SNX16 | NM_022133 | sorting nexin 16 isoform a |
| SNX19 | NM_014758 | sorting nexin 19 |
| SNX6 | NM_021249 | sorting nexin 6 isoform a |
| SNX9 | NM_016224 | sorting nexin 9 |
| SOCS5 | NM_014011 | suppressor of cytokine signaling 5 |
| SOCS6 | NM_004232 | suppressor of cytokine signaling 6 |
| SOD3 | NM_003102 | superoxide dismutase 3, extracellular |
| SON | NM_032195 | SON DNA-binding protein isoform B |
| SORBS1 | NM_015385 | sorbin and SH3 domain containing 1 isoform 2 |
| SORBS2 | NM_003603 | sorbin and SH3 domain containing 2 isoform 1 |
| SORCS1 | NM_001013031 | SORCS receptor 1 isoform b |
| SORCS2 | NM_020777 | VPS10 domain receptor protein SORCS 2 |
| SORT1 | NM_002959 | sortilin 1 preproprotein |
| SOST | NM_025237 | sclerostin precursor |
| SOX1 | NM_005986 | SRY (sex determining region Y)-box 1 |
| SOX11 | NM_003108 | SRY-box 11 |
| SOX13 | NM_005686 | SRY-box 13 |
| SOX3 | NM_005634 | SRY (sex determining region Y)-box 3 |
| SOX4 | NM_003107 | SRY (sex determining region Y)-box 4 |
| SOX5 | NM_006940 | SRY (sex determining region Y)-box 5 isoform a |
| SOX9 | NM_000346 | transcription factor SOX9 |
| SP5 | NM_001003845 | Sp5 transcription factor |
| SP8 | NM_182700 | Sp8 transcription factor isoform 1 |
| SPATA18 | NM_145263 | spermatogenesis associated 18 homolog |
| SPATA21 | NM_198546 | spermatogenesis associated 21 |
| SPATA3 | NM_139073 | testis and spermatogenesis cell apoptosis |
| SPDEF | NM_012391 | SAM pointed domain containing ets transcription |
| SPEN | NM_015001 | spen homolog, transcriptional regulator |
| SPFH2 | NM_007175 | SPFH domain family, member 2 isoform 1 |
| SPG20 | NM_015087 | spartin |
| SPG7 | NM_199367 | paraplegin isoform 2 |
| SPHK2 | NM_020126 | sphingosine kinase type 2 isoform |
| SPINT2 | NM_021102 | serine protease inhibitor, Kunitz type, 2 |
| SPIRE2 | NM_032451 | spire homolog 2 |
| SPN | NM_001030288 | sialophorin |
| SPOCK2 | NM_014767 | sparc/osteonectin, cwcv and kazal-like domains |
| SPON2 | NM_012445 | spondin 2, extracellular matrix protein |
| SPP2 | NM_006944 | secreted phosphoprotein 2, 24 kDa |
| SPPL2B | NM_152988 | signal peptide peptidase-like 2B isoform 2 |
| SPPL3 | NM_139015 | SPPL3 protein |
| SPRED1 | NM_152594 | sprouty-related protein 1 with EVH-1 domain |
| SPRN | NM_001012508 | shadow of prion protein |
| SPRR1B | NM_003125 | small proline-rich protein 1B |
| SPRY3 | NM_005840 | sprouty homolog 3 |
| SPRY4 | NM_030964 | sprouty homolog 4 |
| SPRYD3 | NM_032840 | hypothetical protein LOC84926 |
| SPSB2 | NM_032641 | SPRY domain-containing SOCS box protein SSB-2 |
| SPSB3 | NM_080861 | SPRY domain-containing SOCS box protein SSB-3 |
| SPSB4 | NM_080862 | SPRY domain-containing SOCS box protein SSB-4 |
| SPTAN1 | NM_003127 | spectrin, alpha, non-erythrocytic 1 |
| SPTB | NM_001024858 | spectrin beta isoform a |
| SPTBN2 | NM_006946 | spectrin, beta, non-erythrocytic 2 |
| SPTLC1 | NM_006415 | serine palmitoyltransferase subunit 1 isoform a |
| SPTY2D1 | NM_194285 | hypothetical protein LOC144108 |
| SRC | NM_005417 | proto-oncogene tyrosine-protein kinase SRC |
| SRD5A2 | NM_000348 | 3-oxo-5 alpha-steroid 4-dehydrogenase 2 |
| SREBF1 | NM_001005291 | sterol regulatory element binding transcription |
| SRP72 | NM_006947 | signal recognition particle 72 kDa |
| SRPK1 | NM_003137 | SFRS protein kinase 1 |
| SRPR | NM_003139 | signal recognition particle receptor (‘docking |
| SRPRB | NM_021203 | signal recognition particle receptor, beta |
| SRPX | NM_006307 | sushi-repeat-containing protein, X-linked |
| SRXN1 | NM_080725 | sulfiredoxin 1 homolog |
| SSH3 | NM_017857 | slingshot homolog 3 isoform 1 |
| SSR1 | NM_003144 | signal sequence receptor, alpha |
| SSRP1 | NM_003146 | structure specific recognition protein 1 |
| SSU72 | NM_014188 | Ssu72 RNA polymerase II CTD phosphatase homolog |
| ST3GAL4 | NM_006278 | ST3 beta-galactoside alpha-2,3-sialyltransferase |
| ST3GAL5 | NM_003896 | sialyltransferase 9 |
| ST5 | NM_005418 | suppression of tumorigenicity 5 isoform 1 |
| ST6GAL1 | NM_003032 | sialyltransferase 1 isoform a |
| ST7L | NM_017744 | suppression of tumorigenicity 7-like isoform 1 |
| ST8SIA3 | NM_015879 | ST8 alpha-N-acetyl-neuraminide |
| ST8SIA5 | NM_013305 | ST8 alpha-N-acetyl-neuraminide |
| STAC2 | NM_198993 | SH3 and cysteine rich domain 2 |
| STARD13 | NM_052851 | START domain containing 13 isoform gamma |
| STARD3 | NM_006804 | steroidogenic acute regulatory protein related |
| STAT3 | NM_003150 | signal transducer and activator of transcription |
| STAT5B | NM_012448 | signal transducer and activator of transcription |
| STC1 | NM_003155 | stanniocalcin 1 precursor |
| STEAP2 | NM_152999 | six transmembrane epithelial antigen of the |
| STEAP3 | NM_001008410 | dudulin 2 isoform b |
| STIM1 | NM_003156 | stromal interaction molecule 1 precursor |
| STIM2 | NM_020860 | stromal interaction molecule 2 |
| STIP1 | NM_006819 | stress-induced-phosphoprotein 1 |
| STK10 | NM_005990 | serine/threonine kinase 10 |
| STK11 | NM_000455 | serine/threonine protein kinase 11 |
| STK17A | NM_004760 | serine/threonine kinase 17a |
| STK19 | NM_004197 | serine/threonine kinase 19 isoform 1 |
| STK32B | NM_018401 | serine/threonine kinase 32B |
| STK32C | NM_173575 | serine/threonine kinase 32C |
| STK33 | NM_030906 | serine/threonine kinase 33 |
| STK35 | NM_080836 | serine/threonine kinase 35 |
| STK38 | NM_007271 | serine/threonine kinase 38 |
| STK38L | NM_015000 | serine/threonine kinase 38 like |
| STOML1 | NM_004809 | stomatin (EPB72)-like 1 |
| STON1 | NM_006873 | stonin 1 |
| STOX2 | NM_020225 | storkhead box 2 |
| STX16 | NM_001001433 | syntaxin 16 isoform a |
| STX17 | NM_017919 | syntaxin 17 |
| STX1A | NM_004603 | syntaxin 1A (brain) |
| STX3 | NM_004177 | syntaxin 3A |
| STX5 | NM_003164 | syntaxin 5 |
| STX6 | NM_005819 | syntaxin 6 |
| STXBP1 | NM_001032221 | syntaxin binding protein 1 isoform b |
| STXBP3 | NM_007269 | syntaxin binding protein 3 |
| STXBP4 | NM_178509 | syntaxin binding protein 4 |
| STXBP5 | NM_139244 | tomosyn |
| SUFU | NM_016169 | suppressor of fused |
| SUHW3 | NM_017666 | suppressor of hairy wing homolog 3 |
| SUHW4 | NM_001002843 | suppressor of hairy wing homolog 4 isoform 2 |
| SULT4A1 | NM_014351 | sulfotransferase family 4A, member 1 |
| SUMO3 | NM_006936 | small ubiquitin-like modifier protein 3 |
| SUPT16H | NM_007192 | chromatin-specific transcription elongation |
| SUPT6H | NM_003170 | suppressor of Ty 6 homolog |
| SUPT7L | NM_014860 | SPTF-associated factor 65 gamma |
| SURF4 | NM_033161 | surfeit 4 |
| SURF5 | NM_133640 | surfeit 5 isoform b |
| SUSD1 | NM_022486 | sushi domain containing 1 |
| SUV420H1 | NM_016028 | suppressor of variegation 4-20 homolog 1 isoform |
| SUV420H2 | NM_032701 | suppressor of variegation 4-20 homolog 2 |
| SUZ12 | NM_015355 | joined to JAZF1 |
| SVH | NM_031905 | SVH protein |
| SVIL | NM_003174 | supervillin isoform 1 |
| SWAP70 | NM_015055 | SWAP-70 protein |
| SYBL1 | NM_005638 | synaptobrevin-like 1 |
| SYDE1 | NM_033025 | synapse defective 1, Rho GTPase, homolog 1 |
| SYN2 | NM_003178 | synapsin II isoform IIb |
| SYNE1 | NM_015293 | nesprin 1 isoform beta |
| SYNGR1 | NM_004711 | synaptogyrin 1 isoform 1a |
| SYNGR3 | NM_004209 | synaptogyrin 3 |
| SYNJ1 | NM_003895 | synaptojanin 1 isoform a |
| SYPL1 | NM_006754 | synaptophysin-like 1 isoform a |
| SYT10 | NM_198992 | synaptotagmin 10 |
| SYT12 | NM_177963 | synaptotagmin XII |
| SYT15 | NM_031912 | synaptotagmin XV isoform a |
| SYT3 | NM_032298 | synaptotagmin 3 |
| SYT4 | NM_020783 | synaptotagmin IV |
| SYT6 | NM_205848 | synaptotagmin VI |
| SYT8 | NM_138567 | synaptotagmin VIII |
| SYTL2 | NM_032379 | synaptotagmin-like 2 isoform b |
| SYTL4 | NM_080737 | synaptotagmin-like 4 (granuphilin-a) |
| TAB3 | NM_152787 | TAK1-binding protein 3 isoform 1 |
| TACC1 | NM_006283 | transforming, acidic coiled-coil containing |
| TAF15 | NM_003487 | TBP-associated factor 15 isoform 2 |
| TAF1C | NM_005679 | TBP-associated factor 1C isoform 1 |
| TAF5 | NM_006951 | TBP-associated factor 5 |
| TAF7 | NM_005642 | TATA box-binding protein-associated factor 2F |
| TAF7L | NM_024885 | TATA box binding protein-associated factor, RNA |
| TAF9B | NM_015975 | transcription associated factor 9B |
| TAGLN2 | NM_003564 | transgelin 2 |
| TAL1 | NM_003189 | T-cell acute lymphocytic leukemia 1 |
| TAOK1 | NM_020791 | TAO kinase 1 |
| TAP2 | NM_000544 | transporter 2, ATP-binding cassette, sub-family |
| TAPBP | NM_003190 | tapasin isoform 1 precursor |
| TARBP1 | NM_005646 | TAR RNA binding protein 1 |
| TARBP2 | NM_004178 | TAR RNA binding protein 2 isoform b |
| TASP1 | NM_017714 | taspase 1 |
| TAT | NM_000353 | tyrosine aminotransferase |
| TAX1BP3 | NM_014604 | Tax1 (human T-cell leukemia virus type I) |
| TAZ | NM_000116 | tafazzin isoform 1 |
| TBC1D1 | NM_015173 | TBC1 (tre-2/USP6, BUB2, cdc16) domain family, |
| TBC1D10B | NM_015527 | TBC1 domain family, member 10B |
| TBC1D13 | NM_018201 | TBC1 domain family, member 13 |
| TBC1D14 | NM_020773 | TBC1 domain family, member 14 |
| TBC1D19 | NM_018317 | TBC1 domain family, member 19 |
| TBC1D22A | NM_014346 | TBC1 domain family, member 22A |
| TBC1D22B | NM_017772 | TBC1 domain family, member 22B |
| TBC1D2B | NM_015079 | TBC1 domain family, member 2B |
| TBC1D3C | NM_001001418 | TBC1 domain family member 3C |
| TBC1D8 | NM_007063 | TBC1 domain family, member 8 |
| TBC1D9 | NM_015130 | hypothetical protein LOC23158 |
| TBCC | NM_003192 | beta-tubulin cofactor C |
| TBCCD1 | NM_018138 | TBCC domain containing 1 |
| TBK1 | NM_013254 | TANK-binding kinase 1 |
| TBL1X | NM_005647 | transducin beta-like 1X |
| TBL1XR1 | NM_024665 | nuclear receptor co-repressor/HDAC3 complex |
| TBL2 | NM_012453 | transducin (beta)-like 2 |
| TBP | NM_003194 | TATA box binding protein |
| TBPL1 | NM_004865 | TBP-like 1 |
| TBRG1 | NM_032811 | transforming growth factor beta regulator 1 |
| TBX1 | NM_005992 | T-box 1 isoform B |
| TBX2 | NM_005994 | T-box 2 |
| TBX6 | NM_004608 | T-box 6 isoform 1 |
| TCAP | NM_003673 | telethonin |
| TCEB2 | NM_007108 | elongin B isoform a |
| TCF1 | NM_000545 | transcription factor 1, hepatic |
| TCF21 | NM_198392 | transcription factor 21 |
| TCF3 | NM_003200 | transcription factor 3 |
| TCF7 | NM_003202 | transcription factor 7 (T-cell specific, |
| TCFL5 | NM_006602 | transcription factor-like 5 protein |
| TCHP | NM_032300 | trichoplein |
| TCL6 | NM_014418 | T-cell leukemia/lymphoma 6 isoform TCL6a2 |
| TDGF1 | NM_003212 | teratocarcinoma-derived growth factor 1 |
| TEAD1 | NM_021961 | TEA domain family member 1 |
| TEDDM1 | NM_172000 | putative membrane protein HE9 |
| TES | NM_015641 | testin isoform 1 |
| TEX261 | NM_144582 | testis expressed sequence 261 |
| TFAP2A | NM_001032280 | transcription factor AP-2 alpha isoform b |
| TFAP2C | NM_003222 | transcription factor AP-2 gamma |
| TFAP2D | NM_172238 | transcription factor AP-2 beta-like 1 |
| TFAP2E | NM_178548 | transcription factor AP-2 epsilon (activating |
| TFAP4 | NM_003223 | transcription factor AP-4 (activating enhancer |
| TFCP2L1 | NM_014553 | LBP-9 |
| TFEC | NM_001018058 | transcription factor EC isoform b |
| TFG | NM_001007565 | TRK-fused gene |
| TFPI2 | NM_006528 | tissue factor pathway inhibitor 2 |
| TGFBR1 | NM_004612 | transforming growth factor, beta receptor I |
| TGFBR3 | NM_003243 | transforming growth factor, beta receptor III |
| TGIF2 | NM_021809 | TGFB-induced factor 2 |
| TGIF2LY | NM_139214 | TGFB-induced factor 2-like, Y-linked |
| TGOLN2 | NM_006464 | trans-golgi network protein 2 |
| THAP2 | NM_031435 | THAP domain containing, apoptosis associated |
| THAP6 | NM_144721 | THAP domain containing 6 |
| THBS2 | NM_003247 | thrombospondin 2 precursor |
| THEM4 | NM_053055 | thioesterase superfamily member 4 isoform a |
| THSD3 | NM_182509 | thrombospondin, type I domain containing 3 |
| THSD4 | NM_024817 | hypothetical protein LOC79875 |
| THUMPD1 | NM_017736 | THUMP domain containing 1 |
| THYN1 | NM_014174 | thymocyte nuclear protein 1 isoform 1 |
| TIAF1 | NM_004740 | TGFB1-induced anti-apoptotic factor 1 |
| TIGA1 | NM_053000 | hypothetical protein LOC114915 |
| TIGD6 | NM_030953 | hypothetical protein LOC81789 |
| TIMM13 | NM_012458 | translocase of inner mitochondrial membrane 13 |
| TIMM22 | NM_013337 | translocase of inner mitochondrial membrane 22 |
| TIMM50 | NM_001001563 | translocase of inner mitochondrial membrane 50 |
| TIMP2 | NM_003255 | tissue inhibitor of metalloproteinase 2 |
| TK2 | NM_004614 | thymidine kinase 2, mitochondrial |
| TKTL1 | NM_012253 | transketolase-like 1 |
| TLE4 | NM_007005 | transducin-like enhancer protein 4 |
| TLK1 | NM_012290 | tousled-like kinase 1 |
| TLK2 | NM_006852 | tousled-like kinase 2 |
| TLL1 | NM_012464 | tolloid-like 1 |
| TLL2 | NM_012465 | tolloid-like 2 |
| TLN1 | NM_006289 | talin 1 |
| TLOC1 | NM_003262 | translocation protein 1 |
| TLR1 | NM_003263 | toll-like receptor 1 |
| TLR4 | NM_138554 | toll-like receptor 4 precursor |
| TLR7 | NM_016562 | toll-like receptor 7 |
| TLX2 | NM_016170 | T-cell leukemia, homeobox 2 |
| TM2D2 | NM_001024380 | TM2 domain containing 2 isoform b |
| TM4SF1 | NM_014220 | transmembrane 4 superfamily member 1 |
| TM9SF4 | NM_014742 | transmembrane 9 superfamily protein member 4 |
| TMCC1 | NM_001017395 | transmembrane and coiled-coil domains 1 isoform |
| TMCC3 | NM_020698 | transmembrane and coiled-coil domains 3 |
| TMED3 | NM_007364 | transmembrane emp24 domain containing 3 |
| TMED9 | NM_017510 | transmembrane emp24 protein transport domain |
| TMEM10 | NM_033207 | transmembrane protein 10 isoform a |
| TMEM100 | NM_018286 | hypothetical protein LOC55273 |
| TMEM101 | NM_032376 | hypothetical protein LOC84336 |
| TMEM104 | NM_017728 | hypothetical protein LOC54868 |
| TMEM105 | NM_178520 | hypothetical protein LOC284186 |
| TMEM106A | NM_145041 | hypothetical protein LOC113277 |
| TMEM109 | NM_024092 | transmembrane protein 109 |
| TMEM113 | NM_025222 | hypothetical protein PRO2730 |
| TMEM119 | NM_181724 | hypothetical protein LOC338773 |
| TMEM123 | NM_052932 | pro-oncosis receptor inducing membrane injury |
| TMEM127 | NM_017849 | hypothetical protein LOC55654 |
| TMEM134 | NM_025124 | hypothetical protein LOC80194 |
| TMEM135 | NM_022918 | hypothetical protein LOC65084 |
| TMEM138 | NM_016464 | hypothetical protein LOC51524 |
| TMEM139 | NM_153345 | hypothetical protein LOC135932 |
| TMEM143 | NM_018273 | hypothetical protein LOC55260 |
| TMEM16C | NM_031418 | transmembrane protein 16C |
| TMEM16F | NM_001025356 | transmembrane protein 16F |
| TMEM16G | NM_001001891 | transmembrane protein 16G isoform NGEP long |
| TMEM16K | NM_018075 | hypothetical protein LOC55129 |
| TMEM18 | NM_152834 | transmembrane protein 18 |
| TMEM20 | NM_153226 | transmembrane protein 20 |
| TMEM26 | NM_178505 | transmembrane protein 26 |
| TMEM30B | NM_001017970 | transmembrane protein 30B |
| TMEM33 | NM_018126 | transmembrane protein 33 |
| TMEM35 | NM_021637 | transmembrane protein 35 |
| TMEM43 | NM_024334 | transmembrane protein 43 |
| TMEM45B | NM_138788 | transmembrane protein 45B |
| TMEM47 | NM_031442 | transmembrane 4 superfamily member 10 |
| TMEM49 | NM_030938 | transmembrane protein 49 |
| TMEM50B | NM_006134 | transmembrane protein 50B |
| TMEM52 | NM_178545 | transmembrane protein 52 |
| TMEM55A | NM_018710 | transmembrane protein 55A |
| TMEM55B | NM_144568 | transmembrane protein 55B |
| TMEM63C | NM_020431 | transmembrane protein 63C |
| TMEM79 | NM_032323 | hypothetical protein LOC84283 |
| TMEM8 | NM_021259 | transmembrane protein 8 (five membrane-spanning |
| TMEM85 | NM_016454 | hypothetical protein LOC51234 |
| TMEM86A | NM_153347 | hypothetical protein LOC144110 |
| TMEM86B | NM_173804 | hypothetical protein LOC255043 |
| TMEM87A | NM_015497 | hypothetical protein LOC25963 |
| TMEM87B | NM_032824 | hypothetical protein LOC84910 |
| TMEPAI | NM_020182 | transmembrane prostate androgen-induced protein |
| TMIE | NM_147196 | transmembrane inner ear protein |
| TMOD1 | NM_003275 | tropomodulin 1 |
| TMPRSS13 | NM_032046 | transmembrane protease, serine 13 |
| TMPRSS3 | NM_024022 | transmembrane protease, serine 3 isoform 1 |
| TMPRSS4 | NM_019894 | transmembrane protease, serine 4 isoform 1 |
| TMTC2 | NM_152588 | hypothetical protein LOC160335 |
| TNFAIP1 | NM_021137 | tumor necrosis factor, alpha-induced protein 1 |
| TNFAIP8L1 | NM_152362 | tumor necrosis factor, alpha-induced protein |
| TNFAIP8L3 | NM_207381 | tumor necrosis factor, alpha-induced protein |
| TNFRSF10B | NM_003842 | tumor necrosis factor receptor superfamily, |
| TNFRSF10D | NM_003840 | tumor necrosis factor receptor superfamily, |
| TNFRSF13B | NM_012452 | tumor necrosis factor receptor 13B |
| TNFRSF14 | NM_003820 | tumor necrosis factor receptor superfamily, |
| TNFRSF19 | NM_148957 | tumor necrosis factor receptor superfamily, |
| TNFRSF19L | NM_032871 | tumor necrosis factor receptor superfamily, |
| TNFSF7 | NM_001252 | tumor necrosis factor ligand superfamily, member |
| TNFSF9 | NM_003811 | tumor necrosis factor (ligand) superfamily, |
| TNIP1 | NM_006058 | Nef-associated factor 1 |
| TNIP2 | NM_024309 | A20-binding inhibitor of NF-kappaB activation 2 |
| TNK2 | NM_001010938 | tyrosine kinase, non-receptor, 2 isoform 2 |
| TNNI1 | NM_003281 | troponin I, skeletal, slow |
| TNRC6B | NM_001024843 | trinucleotide repeat containing 6B isoform 2 |
| TNS1 | NM_022648 | tensin |
| TNS3 | NM_022748 | tensin-like SH2 domain containing 1 |
| TNT | NM_182831 | hypothetical protein LOC162083 |
| TOB2 | NM_016272 | transducer of ERBB2, 2 |
| TOLLIP | NM_019009 | toll interacting protein |
| TOM1 | NM_005488 | target of myb1 |
| TOM1L2 | NM_001033551 | target of myb1-like 2 isoform 1 |
| TOMM20 | NM_014765 | translocase of outer mitochondrial membrane 20 |
| TOMM34 | NM_006809 | translocase of outer mitochondrial membrane 34 |
| TOR1B | NM_014506 | torsin family 1, member B (torsin B) |
| TOR3A | NM_022371 | torsin family 3, member A |
| TP53I11 | NM_006034 | p53-induced protein |
| TP53INP2 | NM_021202 | tumor protein p53 inducible nuclear protein 2 |
| TP53TG3 | NM_016212 | hypothetical protein LOC24150 |
| TP73L | NM_003722 | tumor protein p73-like |
| TPCN2 | NM_139075 | two pore segment channel 2 |
| TPD52L3 | NM_033516 | protein kinase NYD-SP25 isoform 1 |
| TPM1 | NM_001018004 | tropomyosin 1 alpha chain isoform 3 |
| TPM2 | NM_003289 | tropomyosin 2 (beta) isoform 1 |
| TPM3 | NM_153649 | tropomyosin 3 isoform 2 |
| TPPP | NM_007030 | brain-specific protein p25 alpha |
| TPRX1 | NM_198479 | tetra-peptide repeat homeobox |
| TRAF1 | NM_005658 | TNF receptor-associated factor 1 |
| TRAF4 | NM_004295 | TNF receptor-associated factor 4 isoform 1 |
| TRAF5 | NM_001033910 | TNF receptor-associated factor 5 |
| TRAF7 | NM_032271 | ring finger and WD repeat domain 1 isoform 1 |
| TRAFD1 | NM_006700 | FLN29 gene product |
| TRAK1 | NM_014965 | OGT(O-Glc-NAc transferase)-interacting protein |
| TRAM1 | NM_014294 | translocating chain-associating membrane |
| TRAM2 | NM_012288 | translocation-associated membrane protein 2 |
| TREML2 | NM_024807 | triggering receptor expressed on myeloid |
| TRIAD3 | NM_207111 | TRIAD3 protein isoform a |
| TRIM10 | NM_006778 | tripartite motif-containing 10 isoform 1 |
| TRIM11 | NM_145214 | tripartite motif-containing 11 |
| TRIM14 | NM_014788 | tripartite motif protein TRIM14 isoform alpha |
| TRIM2 | NM_015271 | tripartite motif-containing 2 |
| TRIM29 | NM_012101 | tripartite motif protein TRIM29 isoform alpha |
| TRIM35 | NM_015066 | tripartite motif-containing 35 isoform 1 |
| TRIM36 | NM_018700 | tripartite motif-containing 36 isoform 1 |
| TRIM37 | NM_015294 | tripartite motif-containing 37 protein |
| TRIM56 | NM_030961 | tripartite motif-containing 56 |
| TRIM6 | NM_001003818 | tripartite motif-containing 6 isoform 1 |
| TRIM62 | NM_018207 | tripartite motif-containing 62 |
| TRIM68 | NM_018073 | ring finger protein 137 |
| TRIM7 | NM_203293 | tripartite motif-containing 7 isoform 1 |
| TRIM9 | NM_015163 | tripartite motif protein 9 isoform 1 |
| TRIP10 | NM_004240 | thyroid hormone receptor interactor 10 |
| TRIT1 | NM_017646 | tRNA isopentenyltransferase 1 |
| TRMT5 | NM_020810 | tRNA-(N1G37) methyltransferase |
| TRMU | NM_001008568 | tRNA 5-methylaminomethyl-2-thiouridylate |
| TRPC1 | NM_003304 | transient receptor potential cation channel, |
| TRPC4AP | NM_015638 | TRPC4-associated protein isoform a |
| TRPM2 | NM_001001188 | transient receptor potential cation channel, |
| TRPV1 | NM_018727 | transient receptor potential cation channel, |
| TSC1 | NM_000368 | tuberous sclerosis 1 protein isoform 1 |
| TSC22D1 | NM_006022 | TSC22 domain family 1 isoform 2 |
| TSC22D2 | NM_014779 | TSC22 domain family 2 |
| TSC22D3 | NM_001015881 | TSC22 domain family, member 3 isoform 3 |
| TSGA13 | NM_052933 | testis specific, 13 |
| TSHR | NM_001018036 | thyroid stimulating hormone receptor isoform 2 |
| TSN | NM_004622 | translin |
| TSPAN14 | NM_030927 | tetraspanin 14 |
| TSPAN15 | NM_012339 | transmembrane 4 superfamily member 15 |
| TSPAN17 | NM_001006616 | transmembrane 4 superfamily member 17 isoform c |
| TSPAN18 | NM_130783 | tetraspanin 18 isoform 2 |
| TSPAN3 | NM_005724 | transmembrane 4 superfamily member 8 isoform 1 |
| TSPAN33 | NM_178562 | penumbra |
| TSPAN5 | NM_005723 | transmembrane 4 superfamily member 9 |
| TSPAN9 | NM_006675 | tetraspanin 9 |
| TSPYL2 | NM_022117 | TSPY-like 2 |
| TSPYL4 | NM_021648 | TSPY-like 4 |
| TSPYL5 | NM_033512 | TSPY-like 5 |
| TSPYL6 | NM_001003937 | TSPY-like 6 |
| TSSK6 | NM_032037 | serine/threonine protein kinase SSTK |
| TTBK1 | NM_032538 | tau tubulin kinase 1 |
| TTC1 | NM_003314 | tetratricopeptide repeat domain 1 |
| TTC13 | NM_024525 | tetratricopeptide repeat domain 13 |
| TTC21B | NM_024753 | tetratricopeptide repeat domain 21B |
| TTC23 | NM_001018029 | tetratricopeptide repeat domain 23 isoform 1 |
| TTC25 | NM_031421 | hypothetical protein LOC83538 |
| TTLL12 | NM_015140 | hypothetical protein LOC23170 |
| TTLL5 | NM_015072 | tubulin tyrosine ligase-like family, member 5 |
| TTLL9 | NM_001008409 | tubulin tyrosine ligase-like family, member 9 |
| TTYH3 | NM_025250 | tweety 3 |
| TUB | NM_003320 | tubby isoform a |
| TUBA2 | NM_006001 | tubulin, alpha 2 isoform 1 |
| TUBA3 | NM_006009 | tubulin, alpha 3 |
| TUBB | NM_178014 | tubulin, beta polypeptide |
| TUBB3 | NM_006086 | tubulin, beta, 4 |
| TUFT1 | NM_020127 | tuftelin 1 |
| TULP3 | NM_003324 | tubby like protein 3 |
| TUSC5 | NM_172367 | LOST1 |
| TXLNA | NM_175852 | taxilin |
| TXN2 | NM_012473 | thioredoxin 2 precursor |
| TXNDC5 | NM_022085 | thioredoxin domain containing 5 isoform 2 |
| TXNIP | NM_006472 | thioredoxin interacting protein |
| TXNL4A | NM_006701 | thioredoxin-like 4A |
| TYRO3 | NM_006293 | TYRO3 protein tyrosine kinase |
| TYSND1 | NM_173555 | trypsin domain containing 1 isoform a |
| UAP1L1 | NM_207309 | UDP-N-acteylglucosamine pyrophosphorylase 1-like |
| UBADC1 | NM_016172 | ubiquitin associated domain containing 1 |
| UBAP1 | NM_016525 | ubiquitin associated protein 1 |
| UBASH3A | NM_001001895 | ubiquitin associated and SH3 domain containing, |
| UBE2A | NM_003336 | ubiquitin-conjugating enzyme E2A isoform 1 |
| UBE2B | NM_003337 | ubiquitin-conjugating enzyme E2B |
| UBE2H | NM_003344 | ubiquitin-conjugating enzyme E2H isoform 1 |
| UBE2I | NM_003345 | ubiquitin-conjugating enzyme E2I |
| UBE2J1 | NM_016021 | ubiquitin-conjugating enzyme E2, J1 |
| UBE2J2 | NM_058167 | ubiquitin conjugating enzyme E2, J2 isoform 2 |
| UBE2O | NM_022066 | ubiquitin-conjugating enzyme E2O |
| UBE2Q1 | NM_017582 | ubiquitin-conjugating enzyme E2Q |
| UBE2Q2 | NM_173469 | ubiquitin-conjugating enzyme E2Q (putative) 2 |
| UBE2R2 | NM_017811 | ubiquitin-conjugating enzyme UBC3B |
| UBE2V1 | NM_001032288 | ubiquitin-conjugating enzyme E2 variant 1 |
| UBE2Z | NM_023079 | ubiquitin-conjugating enzyme E2Z (putative) |
| UBE3C | NM_014671 | ubiquitin protein ligase E3C |
| UBE4A | NM_004788 | ubiquitination factor E4A |
| UBE4B | NM_006048 | ubiquitination factor E4B |
| UBL3 | NM_007106 | ubiquitin-like 3 |
| UBL4A | NM_014235 | ubiquitin-like 4 |
| UBL4B | NM_203412 | hypothetical protein LOC164153 |
| UBN1 | NM_016936 | ubinuclein 1 |
| UBOX5 | NM_014948 | U-box domain containing 5 isoform a |
| UBP1 | NM_014517 | upstream binding protein 1 (LBP-1a) |
| UBTD1 | NM_024954 | ubiquitin domain containing 1 |
| UBXD2 | NM_014607 | UBX domain containing 2 |
| UBXD3 | NM_152376 | UBX domain containing 3 |
| UBXD8 | NM_014613 | UBX domain containing 8 |
| UCP2 | NM_003355 | uncoupling protein 2 |
| UHMK1 | NM_175866 | kinase interacting stathmin |
| ULK1 | NM_003565 | unc-51-like kinase 1 |
| UMOD | NM_001008389 | uromodulin precursor |
| UNC13D | NM_199242 | unc-13 homolog D |
| UNC5D | NM_080872 | netrin receptor Unc5h4 |
| UNC84A | NM_025154 | unc-84 homolog A |
| UNC84B | NM_015374 | unc-84 homolog B |
| UNG | NM_003362 | uracil-DNA glycosylase isoform UNG1 precursor |
| UNG2 | NM_001024592 | uracil-DNA glycosylase 2 isoform b |
| UNQ9370 | NM_207447 | hypothetical protein LOC400454 |
| UPF1 | NM_002911 | regulator of nonsense transcripts 1 |
| UQCR | NM_006830 | ubiquinol-cytochrome c reductase, 6.4 kDa |
| URG4 | NM_017920 | hypothetical protein LOC55665 |
| UROS | NM_000375 | uroporphyrinogen III synthase |
| USH2A | NM_206933 | usherin isoform B |
| USP14 | NM_005151 | ubiquitin specific protease 14 isoform a |
| USP15 | NM_006313 | ubiquitin specific protease 15 |
| USP18 | NM_017414 | ubiquitin specific protease 18 |
| USP19 | NM_006677 | ubiquitin specific protease 19 |
| USP2 | NM_004205 | ubiquitin specific protease 2 isoform a |
| USP20 | NM_001008563 | ubiquitin specific protease 20 |
| USP25 | NM_013396 | ubiquitin specific protease 25 |
| USP3 | NM_006537 | ubiquitin specific protease 3 |
| USP32 | NM_032582 | ubiquitin specific protease 32 |
| USP36 | NM_025090 | ubiquitin specific protease 36 |
| UTX | NM_021140 | ubiquitously transcribed tetratricopeptide |
| VAC14 | NM_018052 | Vac14 homolog |
| VAMP1 | NM_014231 | vesicle-associated membrane protein 1 isoform 1 |
| VAMP2 | NM_014232 | vesicle-associated membrane protein 2 |
| VAMP8 | NM_003761 | vesicle-associated membrane protein 8 |
| VAPB | NM_004738 | VAMP-associated protein B/C |
| VASH1 | NM_014909 | vasohibin 1 |
| VAT1 | NM_006373 | vesicle amine transport protein 1 |
| VAV2 | NM_003371 | vav 2 oncogene |
| VAX1 | NM_199131 | ventral anterior homeobox 1 |
| VCL | NM_003373 | vinculin isoform VCL |
| VDR | NM_000376 | vitamin D (1,25-dihydroxyvitamin D3) receptor |
| VEGF | NM_001025366 | vascular endothelial growth factor isoform a |
| VEZT | NM_017599 | transmembrane protein vezatin |
| VGLL2 | NM_153453 | vestigial-like 2 isoform 2 |
| VGLL3 | NM_016206 | colon carcinoma related protein |
| VIL2 | NM_003379 | villin 2 |
| VIPR2 | NM_003382 | vasoactive intestinal peptide receptor 2 |
| VISA | NM_020746 | virus-induced signaling adapter |
| VIT | NM_053276 | vitrin |
| VMD2L2 | NM_153274 | vitelliform macular dystrophy 2-like 2 |
| VMD2L3 | NM_152439 | vitelliform macular dystrophy 2-like 3 |
| VPS13B | NM_017890 | vacuolar protein sorting 13B isoform 5 |
| VPS13D | NM_015378 | vacuolar protein sorting 13D isoform 1 |
| VPS24 | NM_001005753 | vacuolar protein sorting 24 isoform 2 |
| VPS33B | NM_018668 | vacuolar protein sorting 33B (yeast homolog)) |
| VPS36 | NM_016075 | vacuolar protein sorting 36 |
| VPS37B | NM_024667 | vacuolar protein sorting 37B |
| VPS37C | NM_017966 | vacuolar protein sorting 37C |
| VPS41 | NM_014396 | vacuolar protein sorting 41 (yeast homolog) |
| VPS4A | NM_013245 | vacuolar protein sorting factor 4A |
| VSIG4 | NM_007268 | V-set and immunoglobulin domain containing 4 |
| VTI1B | NM_006370 | vesicle transport through interaction with |
| VWF | NM_000552 | von Willebrand factor preproprotein |
| WAPAL | NM_015045 | wings apart-like homolog |
| WARS2 | NM_015836 | mitochondrial tryptophanyl tRNA synthetase 2 |
| WASF2 | NM_006990 | WAS protein family, member 2 |
| WASL | NM_003941 | Wiskott-Aldrich syndrome gene-like protein |
| WASPIP | NM_003387 | WASP-interacting protein |
| WBP11 | NM_016312 | WW domain binding protein 11 |
| WBP2 | NM_012478 | WW domain binding protein 2 |
| WBSCR17 | NM_022479 | UDP-GalNAc:polypeptide |
| WBSCR18 | NM_032317 | Williams Beuren syndrome chromosome region 18 |
| WBSCR19 | NM_175064 | Williams Beuren syndrome chromosome region 19 |
| WDFY3 | NM_178583 | WD repeat and FYVE domain containing 3 isoform |
| WDHD1 | NM_001008396 | WD repeat and HMG-box DNA binding protein 1 |
| WDR13 | NM_017883 | WD repeat domain 13 protein |
| WDR20 | NM_181291 | WD repeat domain 20 isoform 1 |
| WDR21A | NM_015604 | WD repeat domain 21A isoform 1 |
| WDR21C | NM_152418 | hypothetical protein LOC138009 |
| WDR22 | NM_003861 | Breakpoint cluster region protein, uterine |
| WDR31 | NM_001006615 | WD repeat domain 31 isoform 2 |
| WDR33 | NM_001006623 | WD repeat domain 33 isoform 3 |
| WDR37 | NM_014023 | WD repeat domain 37 |
| WDR4 | NM_018669 | WD repeat domain 4 protein |
| WDR41 | NM_018268 | WD repeat domain 41 |
| WDR42A | NM_015726 | H326 |
| WDR47 | NM_014969 | WD repeat domain 47 |
| WDR59 | NM_030581 | WD repeat domain 59 |
| WDR62 | NM_173636 | WD repeat domain 62 |
| WDR68 | NM_005828 | WD-repeat protein |
| WDR7 | NM_015285 | rabconnectin-3 beta isoform 1 |
| WDR73 | NM_032856 | WD repeat domain 73 |
| WDTC1 | NM_015023 | WD and tetratricopeptide repeats 1 |
| WEE1 | NM_003390 | wee1 tyrosine kinase |
| WFDC5 | NM_145652 | WAP four-disulfide core domain 5 precursor |
| WFIKKN2 | NM_175575 | WFIKKN2 protein |
| WHSC1 | NM_007331 | Wolf-Hirschhorn syndrome candidate 1 protein |
| WHSC2 | NM_005663 | Wolf-Hirschhorn syndrome candidate 2 protein |
| WIBG | NM_032345 | within bgcn homolog |
| WIF1 | NM_007191 | Wnt inhibitory factor-1 precursor |
| WIPI2 | NM_001033518 | hypothetical protein LOC26100 isoform c |
| WIRE | NM_133264 | WIRE protein |
| WISP1 | NM_003882 | WNT1 inducible signaling pathway protein 1 |
| WNK3 | NM_001002838 | WNK lysine deficient protein kinase 3 isoform 2 |
| WNT2B | NM_004185 | wingless-type MMTV integration site family, |
| WNT3A | NM_033131 | wingless-type MMTV integration site family, |
| WNT5A | NM_003392 | wingless-type MMTV integration site family, |
| WNT5B | NM_030775 | wingless-type MMTV integration site family, |
| WNT7A | NM_004625 | wingless-type MMTV integration site family, |
| WNT8A | NM_058244 | wingless-type MMTV integration site family, |
| WNT9A | NM_003395 | wingless-type MMTV integration site family, |
| WSB1 | NM_015626 | WD repeat and SOCS box-containing 1 isoform 1 |
| WT1 | NM_000378 | Wilms tumor 1 isoform A |
| WWC1 | NM_015238 | KIBRA protein |
| WWP1 | NM_007013 | WW domain containing E3 ubiquitin protein ligase |
| WWP2 | NM_007014 | WW domain containing E3 ubiquitin protein ligase |
| XAB1 | NM_007266 | XPA binding protein 1 |
| XKR5 | NM_207411 | XK-related protein 5a |
| XKR8 | NM_018053 | X Kell blood group precursor-related family, |
| XPC | NM_004628 | xeroderma pigmentosum, complementation group C |
| XPO4 | NM_022459 | exportin 4 |
| XPO5 | NM_020750 | exportin 5 |
| XPO6 | NM_015171 | exportin 6 |
| XPR1 | NM_004736 | xenotropic and polytropic retrovirus receptor |
| XRN1 | NM_019001 | 5′-3′ exoribonuclease 1 |
| XTP7 | NM_138568 | protein 7 transactivated by hepatitis B virus X |
| YAF2 | NM_001012424 | YY1 associated factor 2 isoform b |
| YAP1 | NM_006106 | Yes-associated protein 1, 65 kD |
| YARS2 | NM_015936 | tyrosyl-tRNA synthetase 2 (mitochondrial) |
| YEATS2 | NM_018023 | YEATS domain containing 2 |
| YIF1B | NM_033557 | Yip1 interacting factor homolog B isoform 2 |
| YIPF7 | NM_182592 | Yip1 domain family, member 7 |
| YKT6 | NM_006555 | YKT6 v-SNARE protein |
| YOD1 | NM_018566 | hypothetical protein LOC55432 |
| YPEL1 | NM_013313 | yippee-like 1 |
| YPEL4 | NM_145008 | yippee-like 4 |
| YRDC | NM_024640 | ischemia/reperfusion inducible protein |
| YTHDC1 | NM_001031732 | splicing factor YT521-B isoform 1 |
| YTHDF1 | NM_017798 | YTH domain family, member 1 |
| YWHAG | NM_012479 | tyrosine 3-monooxygenase/tryptophan |
| YWHAH | NM_003405 | tyrosine 3/tryptophan 5-monooxygenase |
| YWHAQ | NM_006826 | tyrosine 3/tryptophan 5-monooxygenase |
| ZA20D2 | NM_006007 | zinc finger protein 216 |
| ZA20D3 | NM_019006 | zinc finger, A20 domain containing 3 |
| ZADH2 | NM_175907 | zinc binding alcohol dehydrogenase, domain |
| ZAK | NM_133646 | MLK-related kinase isoform 2 |
| ZBED1 | NM_004729 | Ac-like transposable element |
| ZBP1 | NM_030776 | tumor stroma and activated macrophage protein |
| ZBTB10 | NM_023929 | zinc finger and BTB domain containing 10 |
| ZBTB11 | NM_014415 | zinc finger protein ZNF-U69274 |
| ZBTB2 | NM_020861 | zinc finger and BTB domain containing 2 |
| ZBTB24 | NM_014797 | zinc finger and BTB domain containing 24 |
| ZBTB3 | NM_024784 | zinc finger and BTB domain containing 3 |
| ZBTB32 | NM_014383 | testis zinc finger protein |
| ZBTB33 | NM_006777 | kaiso |
| ZBTB39 | NM_014830 | zinc finger and BTB domain containing 39 |
| ZBTB40 | NM_014870 | zinc finger and BTB domain containing 40 |
| ZBTB41 | NM_194314 | zinc finger and BTB domain containing 41 |
| ZBTB43 | NM_014007 | zinc finger protein 297B |
| ZBTB5 | NM_014872 | zinc finger and BTB domain containing 5 |
| ZBTB8 | NM_144621 | zinc finger and BTB domain containing 8 |
| ZBTB9 | NM_152735 | zinc finger and BTB domain containing 9 |
| ZC3H11A | NM_014827 | hypothetical protein LOC9877 |
| ZC3H12B | NM_001010888 | hypothetical protein LOC340554 |
| ZC3H6 | NM_198581 | zinc finger CCCH-type domain containing 6 |
| ZCCHC2 | NM_017742 | zinc finger, CCHC domain containing 2 |
| ZCCHC3 | NM_033089 | zinc finger, CCHC domain containing 3 |
| ZCCHC5 | NM_152694 | zinc finger, CCHC domain containing 5 |
| ZCSL3 | NM_181706 | zinc finger, CSL domain containing 3 |
| ZDHHC11 | NM_024786 | zinc finger, DHHC domain containing 11 |
| ZDHHC12 | NM_032799 | zinc finger, DHHC domain containing 12 |
| ZDHHC14 | NM_024630 | NEW1 domain containing protein isoform 1 |
| ZDHHC15 | NM_144969 | zinc finger, DHHC domain containing 15 |
| ZDHHC16 | NM_032327 | Abl-philin 2 isoform 1 |
| ZDHHC17 | NM_015336 | huntingtin interacting protein 14 |
| ZDHHC18 | NM_032283 | zinc finger, DHHC domain containing 18 |
| ZDHHC22 | NM_174976 | zinc finger, DHHC domain containing 22 |
| ZDHHC23 | NM_173570 | zinc finger, DHHC domain containing 23 |
| ZDHHC9 | NM_001008222 | zinc finger, DHHC domain containing 9 |
| ZFAND3 | NM_021943 | testis expressed sequence 27 |
| ZFP106 | NM_022473 | zinc finger protein 106 homolog |
| ZFP28 | NM_020828 | zinc finger protein 28 |
| ZFP41 | NM_173832 | zinc finger protein 41 homolog |
| ZFP95 | NM_014569 | zinc finger protein 95 homolog |
| ZFYVE1 | NM_021260 | zinc finger, FYVE domain containing 1 isoform 1 |
| ZFYVE20 | NM_022340 | FYVE-finger-containing Rab5 effector protein |
| ZFYVE28 | NM_020972 | zinc finger, FYVE domain containing 28 |
| ZHX1 | NM_001017926 | zinc fingers and homeoboxes 1 |
| ZHX3 | NM_015035 | zinc fingers and homeoboxes 3 |
| ZIC1 | NM_003412 | zinc finger protein of the cerebellum 1 |
| ZKSCAN1 | NM_003439 | zinc finger protein 36 |
| ZMYM6 | NM_007167 | zinc finger protein 258 |
| ZMYND10 | NM_015896 | zinc finger, MYND domain-containing 10 |
| ZNF10 | NM_015394 | zinc finger protein 10 |
| ZNF134 | NM_003435 | zinc finger protein 134 |
| ZNF135 | NM_003436 | zinc finger protein 135 (clone pHZ-17) |
| ZNF187 | NM_001023560 | zinc finger protein 187 |
| ZNF192 | NM_006298 | zinc finger protein 192 |
| ZNF193 | NM_006299 | zinc finger protein 193 |
| ZNF198 | NM_003453 | zinc finger protein 198 |
| ZNF212 | NM_012256 | zinc finger protein 212 |
| ZNF213 | NM_004220 | zinc finger protein 213 |
| ZNF215 | NM_013250 | zinc finger protein 215 |
| ZNF236 | NM_007345 | zinc finger protein 236 |
| ZNF259 | NM_003904 | zinc finger protein 259 |
| ZNF264 | NM_003417 | zinc finger protein 264 |
| ZNF267 | NM_003414 | zinc finger protein 267 |
| ZNF282 | NM_003575 | zinc finger protein 282 |
| ZNF285 | NM_152354 | zinc finger protein 285 |
| ZNF289 | NM_032389 | zinc finger protein 289, ID1 regulated |
| ZNF295 | NM_020727 | zinc finger protein 295 |
| ZNF304 | NM_020657 | zinc finger protein 304 |
| ZNF306 | NM_024493 | zinc finger protein 306 |
| ZNF307 | NM_019110 | zinc finger protein 307 |
| ZNF313 | NM_018683 | zinc finger protein 313 |
| ZNF317 | NM_020933 | zinc finger protein 317 |
| ZNF319 | NM_020807 | zinc finger protein 319 |
| ZNF323 | NM_030899 | zinc finger protein 323 isoform 1 |
| ZNF326 | NM_181781 | zinc finger protein 326 isoform 2 |
| ZNF329 | NM_024620 | zinc finger protein 329 |
| ZNF343 | NM_024325 | zinc finger protein 343 |
| ZNF346 | NM_012279 | zinc finger protein 346 |
| ZNF365 | NM_014951 | zinc finger protein 365 isoform A |
| ZNF367 | NM_153695 | zinc finger protein 367 |
| ZNF395 | NM_018660 | zinc finger protein 395 |
| ZNF406 | NM_001029939 | zinc finger protein 406 isoform TR-ZFAT |
| ZNF417 | NM_152475 | zinc finger protein 417 |
| ZNF423 | NM_015069 | zinc finger protein 423 |
| ZNF436 | NM_030634 | zinc finger protein 436 |
| ZNF445 | NM_181489 | zinc finger protein 445 |
| ZNF449 | NM_152695 | zinc finger protein 449 |
| ZNF454 | NM_182594 | zinc finger protein 454 |
| ZNF488 | NM_153034 | zinc finger protein 488 |
| ZNF497 | NM_198458 | zinc finger protein 497 |
| ZNE498 | NM_145115 | zinc finger protein 498 |
| ZNF500 | NM_021646 | zinc finger protein 500 |
| ZNF501 | NM_145044 | zinc finger protein 501 |
| ZNF503 | NM_032772 | zinc finger protein 503 |
| ZNF512 | NM_032434 | zinc finger protein 512 |
| ZNF532 | NM_018181 | zinc finger protein 532 |
| ZNF536 | NM_014717 | zinc finger protein 536 |
| ZNF548 | NM_152909 | zinc finger protein 548 |
| ZNF569 | NM_152484 | zinc finger protein 569 |
| ZNF572 | NM_152412 | zinc finger protein 572 |
| ZNF592 | NM_014630 | zinc finger protein 592 |
| ZNF600 | NM_198457 | zinc finger protein 600 |
| ZNF609 | NM_015042 | zinc finger protein 609 |
| ZNF621 | NM_198484 | zinc finger protein 621 |
| ZNF622 | NM_033414 | zinc finger protein 622 |
| ZNF623 | NM_014789 | zinc finger protein 623 |
| ZNF626 | NM_145297 | zinc finger protein 626 |
| ZNF627 | NM_145295 | zinc finger protein 627 |
| ZNF650 | NM_172070 | zinc finger protein 650 |
| ZNF651 | NM_145166 | zinc finger protein 651 |
| ZNF660 | NM_173658 | zinc finger protein 660 |
| ZNF691 | NM_015911 | zinc finger protein 691 |
| ZNF694 | NM_001012981 | zinc finger protein 694 |
| ZNF695 | NM_020394 | zinc finger protein SBZF3 |
| ZNF696 | NM_030895 | zinc finger protein 696 |
| ZNF701 | NM_018260 | zinc finger protein 701 |
| ZNF704 | NM_001033723 | zinc finger protein 704 |
| ZNF705A | NM_001004328 | hypothetical protein LOC440077 |
| ZNF71 | NM_021216 | zinc finger protein 71 |
| ZNF74 | NM_003426 | zinc finger protein 74 (Cos52) |
| ZNF747 | NM_023931 | hypothetical protein LOC65988 |
| ZNF76 | NM_003427 | zinc finger protein 76 (expressed in testis) |
| ZNF81 | NM_007137 | zinc finger protein 81 (HFZ20) |
| ZNFN1A1 | NM_006060 | zinc finger protein, subfamily 1A, 1 (Ikaros) |
| ZNFN1A4 | NM_022465 | zinc finger protein, subfamily 1A, 4 |
| ZNHIT3 | NM_004773 | thyroid hormone receptor interactor 3 isoform 2 |
| ZNRF1 | NM_032268 | zinc and ring finger protein 1 |
| ZNRF2 | NM_147128 | zinc finger/RING finger 2 |
| ZPLD1 | NM_175056 | hypothetical protein LOC131368 |
| ZSWIM3 | NM_080752 | zinc finger, SWIM domain containing 3 |
| ZSWIM4 | NM_023072 | zinc finger, SWIM domain containing 4 |
| ZW10 | NM_004724 | centromere/kinetochore protein zw10 |
| ZYG11A | NM_001004339 | hypothetical protein LOC440590 |
| ZYG11BL | NM_006336 | zyg-11 homolog B (C. elegans)-like |
| ZYX | NM_001010972 | zyxin |
| ZZEF1 | NM_015113 | zinc finger, ZZ type with EF hand domain 1 |
| ZZZ3 | NM_015534 | zinc finger, ZZ domain containing 3 |

The predicted gene targets that exhibited altered mRNA expression levels in human cancer cells, following transfection with pre-miR hsa-miR-16, are shown in Table 4 below.

TABLE 4
Predicted hsa-miR-16 targets that exhibited altered mRNA expression levels in human
cancer cells after transfection with pre-miR hsa-miR-16.

| Gene Symbol | RefSeq Transcript ID | Description |
|---|---|---|
| ACTR2 | NM_001005386 | actin-related protein 2 isoform a |
| ADARB1 | NM_001033049 | RNA-specific adenosine deaminase B1 isoform 4 |
| ADRB2 | NM_000024 | adrenergic, beta-2-, receptor, surface |
| ANKRD12 | NM_015208 | ankyrin repeat domain 12 |
| ARHGDIA | NM_004309 | Rho GDP dissociation inhibitor (GDI) alpha |
| ARL2 | NM_001667 | ADP-ribosylation factor-like 2 |
| CA12 | NM_001218 | carbonic anhydrase XII isoform 1 precursor |
| CCND1 | NM_053056 | cyclin D1 |
| CDC37L1 | NM_017913 | cell division cycle 37 homolog (S. |
| CDH1 | NM_004360 | cadherin 1, type 1 preproprotein |
| CDS2 | NM_003818 | phosphatidate cytidylyltransferase 2 |
| CHUK | NM_001278 | conserved helix-loop-helix ubiquitous kinase |
| CYP4F3 | NM_000896 | cytochrome P450, family 4, subfamily F, |
| DIO2 | NM_000793 | deiodinase, iodothyronine, type II isoform a |
| FGF2 | NM_002006 | fibroblast growth factor 2 |
| FGFR4 | NM_002011 | fibroblast growth factor receptor 4 isoform 1 |
| GALNT7 | NM_017423 | polypeptide N-acetylgalactosaminyltransferase 7 |
| HAS2 | NM_005328 | hyaluronan synthase 2 |
| KCNJ2 | NM_000891 | potassium inwardly-rectifying channel J2 |
| LCN2 | NM_005564 | lipocalin 2 (oncogene 24p3) |
| LRP12 | NM_013437 | suppression of tumorigenicity |
| MAP7 | NM_003980 | microtubule-associated protein 7 |
| PHACTR2 | NM_014721 | phosphatase and actin regulator 2 |
| PLSCR4 | NM_020353 | phospholipid scramblase 4 |
| PODXL | NM_001018111 | podocalyxin-like precursor isoform 1 |
| PPAP2C | NM_003712 | phosphatidic acid phosphatase type 2C isoform 1 |
| QKI | NM_206853 | quaking homolog, KH domain RNA binding isoform |
| RPS6KA3 | NM_004586 | ribosomal protein S6 kinase, 90 kDa, polypeptide |
| RPS6KA5 | NM_004755 | ribosomal protein S6 kinase, 90 kDa, polypeptide |
| SLC11A2 | NM_000617 | solute carrier family 11 (proton-coupled |
| SLC4A7 | NM_003615 | solute carrier family 4, sodium bicarbonate |
| STC1 | NM_003155 | stanniocalcin 1 precursor |
| SYNE1 | NM_015293 | nesprin 1 isoform beta |
| TACC1 | NM_006283 | transforming, acidic coiled-coil containing |
| TFG | NM_001007565 | TRK-fused gene |
| THUMPD1 | NM_017736 | THUMP domain containing 1 |
| TNFSF9 | NM_003811 | tumor necrosis factor (ligand) superfamily, |
| TPM1 | NM_001018004 | tropomyosin 1 alpha chain isoform 3 |
| UBE2I | NM_003345 | ubiquitin-conjugating enzyme E2I |
| VIL2 | NM_003379 | villin 2 |

The predicted gene targets of hsa-miR-16 whose mRNA expression levels are affected by hsa-miR-16 represent particularly useful candidate targets for cancer therapy and therapy of other diseases through manipulation of their expression levels.

### Example 4

### Cancer Related Gene Expression Altered by hsa-miR-16

Cell proliferation and survival pathways are commonly altered in tumors (Hanahan and Weinberg, 2000). The inventors have shown that hsa-miR-16 directly or indirectly regulates the transcripts of proteins that are critical in the regulation of these pathways. Many of these targets have inherent oncogenic or tumor suppressor activity. Hsa-miR-16 targets that are associated with various cancer types are shown in Table 5.

Among these targets are regulators of the cell cycle, including cyclin D1 (CCND1), cyclin G2 (CCNG2) and the transforming acidic coiled coil 1 protein (TACC1). While cyclin D1 forms a functional complex with the cyclin-dependent kinases 4 and 6 (CDK4/6) and is necessary to promote cells from the G1 phase into S phase, cyclin G2-unlike conventional cyclins—negatively regulates the cell cycle (Donnellan and Chetty, 1998; Home et al., 1997). The growth-promoting activity of cyclin D1 correlates with the observation that a broad roster of cancers show elevated levels of cyclin D1 (Donnellan and Chetty, 1998). In contrast, cyclin G2 is down-regulated in multiple cancers, such as oral cancer and papillary carcinomas (Alevizos et al., 2001; Ito et al., 2003). Since hsa-miR-16 over-expression leads to suppression of the cyclin D1 transcript and up regulation of cyclin G2, hsa-miR-16 may function as a tumor suppressor. This view is supported by the fact that hsa-miR-16 negatively regulates the TACC1 message which encodes a putative oncogene located within a breast cancer amplicon on chromosome 8p11 (Cully et al., 2005). Over-expression of TACC1 induces oncogenic transformation of fibroblasts in culture and cooperates with Ras to form tumors in mice with a PTEN+/−background (Cully et al., 2005).

Other hsa-miR-16 targets include the fibroblast growth factor 2 (FGF2), fibroblast growth factor receptor 4 (FGFR4) and IkappaB kinase alpha (IKKalpha, CHUK), all of which are components of the intracellular signaling network. FGF2 is a secretory protein with potent mitogenic and angiogenic activity that transmits the signal into cells via transmembrane receptors (FGFRs) composed of 2-3 extracellular immunoglobulin-like domains and an intracellular tyrosine kinase domain (Chandler et al., 1999). While FGF2 mRNAs levels are increased in renal, oral, and non-small lung cancer cells, FGFR4 is up-regulated in numerous types of cancer (Chandler et al., 1999). Similarly, IKKalpha is a positive regulator of the intracellular signaling cascade and functions to activate the transcription factor nuclear factor kappa B (NFkappaB) (Karin et al 2002). NFkappaB is constitutively activated in several cancer types and promotes anti-apoptotic and survival pathways.

Based on our data, hsa-miR-16 negatively regulates these proteins and therefore is likely to function as a tumor-suppressor. In contrast, hsa-miR-16 may also have oncogenic activity. This view is supported by the observation that hsa-miR-16 negatively regulates the tumor-suppressor RBL-1 (p107) and induces an up-regulation of MCL1, thioredoxin (TXN) and the oncogenic E3 ubiquitin ligase Skp2 (Gstaiger et al., 2001; Huang et al., 2005; Jiang et al., 2005). Skp2 is a component of the multi-subunit E3 ubiquitin ligase complex that ear-marks proteins for proteasomal degradation. A well characterized target is the CDK inhibitor p27 which offers an explanation for the cell cycle promoting activity of Skp2 (Carrano et al., 1999). Skp2 is inherently oncogenic and shows elevated levels in various cancer types (Gstaiger et al., 2001; Kamata et al., 2005; Saigusa et al., 2005; Einama et al., 2006). MCL1 is a member of the BCL-2 (B cell lymphoma 2) gene family. MCL1 gives rise to two alternatively spliced gene products with opposing functions (Bae et al., 2000). The predominant species is MCL1-L that has anti-apoptotic activity. High levels of MCL1 are correlated with poor prognosis of patients with ovarian carcinoma and is indicative for leukemic relapse (Kaufmann et al., 1998; Shigemasa et al., 2002). RNA interference against MCL1 induces a therapeutic response in gastric and hepatocellular carcinoma cells (Schulze-Bergkamen et al., 2006; Zangemeister-Wittke and Huwiler, 2006).

Thioredoxin (TXN) is a 12-kDa thiol reductase targeting various proteins and multiple pathways. Thioredoxin modulates the activity of transcription factors, induces the expression of angiogenic Hif-1alpha (hypoxia induced factor 1 alpha) as well as VEGF (vascular endothelial growth factor) and can act as a proliferative and anti-apoptotic agent (Marks, 2006). In accord, carcinomas of the lung, pancreas, cervix and liver show increased levels of thioredoxin. Thioredoxin expression is also correlated with aggressive tumor growth, poor prognosis and chemoresistance (Marks, 2006). In addition, hsa-miR-16 regulates genes that may have either oncogenic or growth-inhibitory activity, depending on the cellular context: among these are connective tissue growth factor (CTGF) and neutrophil gelatinase-associated lipocalin (NGAL), also known as lipocalin-2 (LCN2) (Croci et al., 2004; Hishikawa et al., 1999; Lin et al., 2005; Yang et al., 2005; Fernandez et al., 2005; Lee et al., 2006).

In summary, hsa-miR-16 governs the activity of proteins that are critical regulators of cell proliferation and survival. These targets are frequently deregulated in human cancer. Based on this review of the genes and related pathways that are regulated by miR-16, introduction of hsa-miR-16 or an anti-hsa-miR-16 into a variety of cancer cell types would likely result in a therapeutic response.

TABLE 5
Tumor associated mRNAs altered by hsa-miR-16 having prognostic or therapeutic
value for the treatment of various malignancies.

| Gene | | | | |
|---|---|---|---|---|
| Symbol | Gene Title | Cellular Process | Cancer Type | Reference [PMID] |
| CCND1 | cyclin D1 | cell cycle | MCL, BC, SCCHN, | Donnellan and Chetty, 1998 |
| | | | OepC, HCC, CRC, BldC, | |
| | | | EC, OC, M, AC, GB, | |
| | | | GC, PaC | |
| CCNG2 | cyclin G2 | cell cycle | TC, SCCHN | Ito et al., 2003b; Alevizos et al., |
| | | | | 2001 |
| CDKN2C | CDK | cell cycle | HB, MB, HCC, HL, MM | Iolascon et al., 1998; Kulkarni et |
| | inhibitor 2C | | | al., 2002; Morishita et al., 2004; |
| | | | | Sanchez-Aguilera et al., 2004 |
| CHUK | IKK alpha | signal | LSCC, BC | Cao et al., 2001; Nakayama et |
| | | transduction | | al., 2001; Romieu-Mourez et al., |
| | | | | 2001 |
| CTGF | CTGF/IGFB | cell adhesion, | BC, GB, OepC, RMS, | Hishikawa et al., 1999; Shimo et |
| | P-8 | migration | CRC, PC | al., 2001; Koliopanos et al., |
| | | | | 2002; Pan et al., 2002; Croci et |
| | | | | al., 2004; Lin et al., 2005; Yang |
| | | | | et al., 2005 |
| FGF2 | FGF-2 | signal | BC, RCC, OC, M, | Chandler et al., 1999 |
| | | transduction | NSCLC | |
| FGFR4 | FGF-R4 | signal | TC, BC, OC, PaC | Jaakkola et al., 1993; Shah et al., |
| | | transduction | | 2002; Ezzat et al., 2005 |
| LCN2 | lipocalin 2/ | cell adhesion | PaC, CRC, HCC, BC, | Bartsch and Tschesche, 1995; |
| | NGAL | | OC | Furutani et al., 1998; Fernandez |
| | | | | et al., 2005; Lee et al., 2006 |
| MCL1 | Mcl-1 | apoptosis | HCC, MM, TT, CLL, | Krajewska et al., 1996; Kitada et |
| | | | ALCL, BCL, PC | al., 1998; Cho-Vega et al., 2004; |
| | | | | Rust et al., 2005; Sano et al., |
| | | | | 2005; Wuilleme-Toumi et al., |
| | | | | 2005; Fleischer et al., 2006; |
| | | | | Sieghart et al., 2006 |
| NF1 | NF-1 | signal | G, AC, NF, PCC, ML | Rubin and Gutmann, 2005 |
| | | transduction | | |
| RBL1 | p107 | cell cycle | BCL, PC, CRC, TC | Takimoto et al., 1998; Claudio et |
| | | | | al., 2002; Wu et al., 2002; Ito et |
| | | | | al., 2003a; Rubin and Gutmann, |
| | | | | 2005 |
| SKP2 | SKP-2 | proteasomal | PaC, OC, BC, MFS, GB, | Kamata et al., 2005; Saigusa et |
| | | degradation | EC, NSCLC, PC | al., 2005; Shibahara et al., 2005; |
| | | | | Takanami, 2005; Einama et al., |
| | | | | 2006; Huang et al., 2006; Sui et |
| | | | | al., 2006; Traub et al., 2006 |
| TACC1 | TACC1 | cell cycle | BC, OC | Cully et al., 2005; Lauffart et al., |
| | | | | 2005 |
| TXN | thioredoxin | thioredoxin redox | LC, PaC, CeC, HCC | Marks, 2006 |
| | (trx) | system | | |
| WISP2 | WISP-2 | signal | CRC, BC | Pennica et al., 1998; Saxena et |
| | | transduction | | al., 2001 |

Abbreviations:
AC, astrocytoma;
ALCL, anaplastic large cell lymphoma;
BC, breast carcinoma;
BCL, B-cell lymphoma;
BldC, bladder carcinoma;
CeC, cervical carcinoma;
CLL, chronic lymphoblastic leukemia;
CRC, colorectal carcinoma;
EC, endometrial carcinoma;
G, glioma;
GB, glioblastoma;
GC, gastric carcinoma;
HB, hepatoblastoma;
HCC, hepatocellular carcinoma;
HL, Hodgkin lymphoma;
LC, lung carcinoma;
LSCC, laryngeal squamous cell carcinoma;
M, melanoma;
MB, medulloblastoma;
MCL, mantle cell lymphoma;
MFS, myxofibrosarcoma;
ML, myeloid leukemia;
MM, multiple myeloma;
NF, neurofibroma;
NSCLC, non-small cell lung carcinoma;
OC, ovarian carcinoma;
OepC, oesophageal carcinoma;
PaC, pancreatic carcinoma;
PC, prostate carcinoma;
PCC, pheochromocytoma;
RCC, renal cell carcinoma;
RMS, rhabdomyosarcoma;
SCCHN, squamous cell carcinoma of the head and neck;
TC, thyroid carcinoma;
TT, testicular tumor.

### Example 5

### Delivery of Synthetic hsa-miR-16 Reduces Cellular Proliferation of Prostate Cancer Cells

The inventors have previously demonstrated that hsa-miR-16 is involved in the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005). For example, overexpression of hsa-miR-16 decreases the proliferation and/or viability of certain normal or cancerous cell lines.

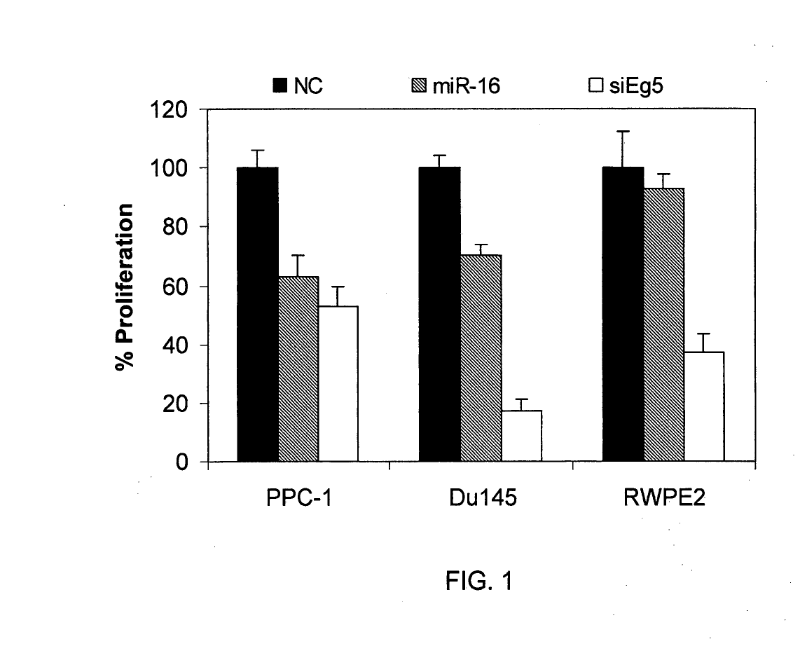

The inventors assessed the therapeutic effect of hsa-miR-16 for prostate cancer by using the prostate cancer cell lines PPC-1, Du145, and RWPE2. Synthetic hsa-miR-16 (Pre-miR™-hsa-miR-16, Ambion cat. no. AM17100) or negative control (NC) miRNA (Pre-miR™ microRNA Precursor Molecule-Negative Control #2; Ambion cat. no. AM17111) was delivered via lipid-based reverse transfections in triplicate according to a published protocol and the following parameters: 6000-7000 cells per 96 well, 0.2 μl Lipofectamine™ 2000 (cat. no. 11668-019, Invitrogen Corp., Carlsbad, Calif., USA) in 20 μl OptiMEM (Invitrogen), 30 nM final concentration of miRNA in 100 μl (Ovcharenko et al., 2005). Proliferation of PPC-1 cells was assessed 4 days post transfection and profilferation of Dul45 and RPWE2 cells was evaluated 6 days post transfection using Alamar Blue (Invitrogen) following the manufacturer's instructions. As a control for inhibition of cellular proliferation, siRNA against the motor protein kinesin 11, also known as Eg5, was used. Eg5 is essential for cellular survival of most eukaryotic cells and a lack thereof leads to reduced cell proliferation and cell death (Weil et al., 2002). siEg5 was used in lipid-based transfection following the same experimental parameters that apply to miRNA. Percent (%) proliferation values from the Alamar Blue assay were normalized to values from cells treated with negative control miRNA. Percent proliferation of hsa-miR-16 treated cells relative to cells treated with negative control miRNA (100%) is shown in Table 6 and in FIG. 1.

TABLE 6
Proliferation of prostate cancer cells following transfection with
hsa-miR-16, negative control miRNA (NC), or siRNA against
Eg5 (siEg5). % SD, % standard deviation. % proliferation values are
normalized to values obtained from cells transfected with
negative control miRNA.

| | hsa-miR-16 | | | | | |
|---|---|---|---|---|---|---|
| | (30 nM) | | siEg5 (30 nM) | | NC (30 nM) | |
| | % | | % | | % | |
| Cells | proliferation | % SD | proliferation | % SD | proliferation | % SD |
| PPC-1 | 63.09 | 7.00 | 52.90 | 6.97 | 100.00 | 5.82 |
| Du145 | 70.00 | 3.70 | 17.26 | 4.23 | 100.00 | 4.12 |
| RWPE2 | 93.03 | 4.72 | 36.96 | 6.56 | 100.00 | 12.28 |

Delivery of hsa-miR-16 inhibits cellular proliferation of prostate cancer cells PPC-1, Du145 and RWPE2 (Table 6 and FIG. 1). On average, hsa-miR-16 inhibits cellular proliferation by 25% (Table 6 and FIG. 1). Hsa-miR-16 has maximal inhibitory activity in PPC-1 cells, reducing proliferation by 37%. Since hsa-miR-16 induces a therapeutic response in all prostate cancer cells tested, hsa-miR-16 may provide therapeutic benefit to patients with prostate cancer and other malignancies.

To evaluate the inhibitory phenotype of hsa-miR-16 over an extended period of time, the inventors conducted growth curve experiments in the presence of hsa-miR-16 for up to 22 days in PPC-1 cells. Since in vitro transfections of naked interfering RNAs, such as synthetic miRNA, are transient by nature and compromised by the dilution of the oligonucleotide during ongoing cell divisions, hsa-miR-16 was administered at multiple time points via electroporation (Bartlett et al., 2006, Bartlett et al., 2007). Equal numbers of PPC-1 cells were electroporated with 1.6 μM synthetic hsa-miR-16 (Pre-miR™-hsa-miR-16, Ambion cat. no. AM17100) or negative control miRNA (Pre-miR™ microRNA Precursor Molecule-Negative Control #2; Ambion cat. no. AM17111) in 200 μl OptiMEM (Invitrogen) on days 0, 4, and 11 using the BioRad GenePulserXcell™ instrument (BioRad Laboratories, Inc.; Hercules, Calif., USA). One million cells were electroporated on day 0; however, to ensure similar treatment of both conditions as well as to accommodate exponential growth, the cell numbers used for the second and third electroporation were titrated down to the lowest count i.e. that of hsa-miR-16 treated cells. At each electroporation event, fifty-thousand cells were plated separately in multiple wells of a 6-well plate, and cells were harvested and counted every other day. The population doubling was calculated using the formula PD=ln(N_{f}/N₀)/ln 2, and cell numbers were extrapolated and plotted on a linear scale.

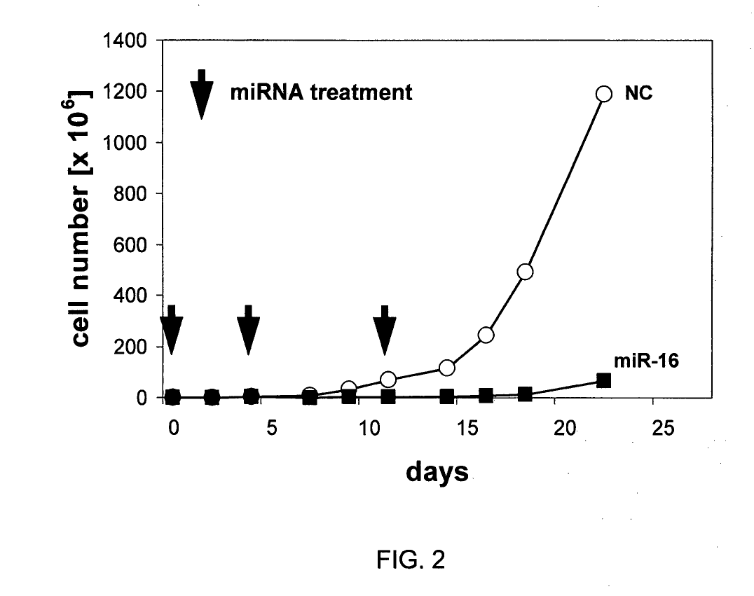

As shown in FIG. 2, three equal doses of synthetic hsa-miR-16 miRNA over 22 days in 4-7 day intervals resulted in an approximate 94.3% inhibition of PPC-1 cell growth relative to cells that received negative control miRNA. The data suggest that multiple administrations of enhance the therapeutic effect of miR-16 miRNA and reinforce previous data, indicating the therapeutic potential of hsa-miR-16 miRNA.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent Ser. No. 09/545,207
U.S. patent Ser. No. 10/667,126
U.S. patent Ser. No. 11/141,707
U.S. Prov. Appln. 60/575,743
U.S. Prov. Appln. 60/649,584
U.S. Prov. Appln. Methods and Compositions Involving miRNA and miRNA Inhibitor Molecules,
EP 266,032
EP 373 203
EP 785 280
EP 799 897
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
PCT Appln. WO0138580
PCT Appln. WO03100012
UK Patent 1,529,202
UK Patent 8 803 000
Alevizos et al, Oncogene, 20(43):6196-6204, 2001.
Ambros, Cell, 107(7):823-826, 2001.
Bae et al., J. Biol. Chem., 275(33):25255-25261, 2000.
Bagga et al., Cell, 122(4):553-563, 2005.
Bartlett et al, Biotechnol. Bioeng., 97(4): 909-921, 2007.
Bartlett et al., Nucleic Acids Res., 43(1):322-333, 2006.
Bartsch and Tschesche, FEBS Lett., 357(3):255-259, 1995.
Brennecke et al., Cell, 113(1):25-36, 2003.
Calin and Croce, Nat. Rev. Cancer, 6(11):857-866, 2006.
Calin et al., Proc. Natl. Acad. Sci. USA, 99(24):15524-15529, 2002.
Cao et al., Cell, 107(6):763-775, 2001.
Carrano et al., Nat. Cell Biol., 1(4):193-199, 1999.
Carrington and Ambros, Science, 301(5631):336-338, 2003.
Chandler et al., Int. J. Cancer, 81(3):451-458, 1999.
Cho-Vega et al., Hum. Pathol., 35(9):1095-1100, 2004.
Claudio et al., Clin. Cancer Res, 8(6):1808-1815, 2002.
Croci et al., Cancer Res., 64(5):1730-1736, 2004.
Cully et al., Cancer Res., 65(22):10363-10370, 2005.
Cummins et al., In: IRT: Nucleosides and nucleosides, La Jolla Calif., 72, 1996.
Denli et al., Trends Biochem. Sci., 28:196, 2003.
Didenko, Biotechniques, 31(5):1106-1116, 1118, 1120-1121, 2001.
Dong et al., Crit. Rev. Oncol. Hematol., 54(2):85-93, 2005.
Donnellan and Chetty, Mol. Pathol., 51(1):1-7, 1998.
Einama et al., Pancreas, 32(4):376-381, 2006.
Emptage et al., Neuron., 29(1):197-208, 2001.
Esquela-Kerscher and Slack, Nat. Rev. Cancer, 6(4):259-269, 2006.
Ezzat et al., Clin. Cancer Res., 11(3):1336-1341, 2005.
Fernandez et al., Clin. Cancer Res., 11(15):5390-5395, 2005.
Fleischer et al., Int. J. Oncol., 28(1):25-32, 2006.
Fodor et al., Science, 251:767-777, 1991.
Froehler et al., Nucleic Acids Res., 14(13):5399-5407, 1986.
Furutani et al., Cancer Lett., 122(1-2):209-214, 1998.
Griffey et al., J Mass Spectrom, 32(3):305-13, 1997.
Gstaiger et al., Proc. Natl. Acad. Sci. USA, 98(9):5043-5048, 2001.
Hanahan and Weinberg, Cell, 100(1):57-70, 2000.
He et al., Nature, 435(7043):828-833, 2005.
He et al., Proc. Natl. Acad. Sci. USA, 102(52):19075-19080, 2005.
Hishikawa et al., J. Biol. Chem., 274(52):37461-37466, 1999.
Home et al., J. Biol. Chem., 272(19):12650-12661, 1997.
Huang et al., Proc. Natl. Acad. Sci. USA, 102(5):1649-1654, 2005.
Huang et al., Clin. Cancer Res., 12(2):487-498, 2006.
Iolascon et al., Hepatology, 27(4):989-995, 1998.
Itakura and Riggs, Science, 209:1401-1405, 1980.
Ito et al., Anticancer Res., 23(3B):2335-2338, 2003b.
Ito et al., Anticancer Res., 23(5A):3819-3824, 2003a.
Jaakkola et al., Int. J. Cancer, 54(3):378-382, 1993.
Jiang et al., Oncogene, 24(21):3409-3418, 2005.
Kamata et al., J. Cancer Res. Clin. Oncol., 131(9):591-596, 2005.
Karin et al., Nat. Rev. Cancer, 2(4):301-310, 2002.
Kaufmann et al., Blood, 91(3):991-1000, 1998.
Kitada et al., Blood, 91(9):3379-3389, 1998.
Klostermeier and Millar, Biopolymers, 61(3):159-79, 2001-2002.
Koliopanos et al., World J. Surg., 26(4):420-427, 2002.
Komberg and Baker, In: DNA Replication, 2^{nd} Ed., Freeman, San Francisco, 1992.
Krajewska et al., Am. J. Pathol., 148(5):1567-1576, 1996.
Kulkami et al., Leukemia, 16(1):127-134, 2002.
Lagos-Quintana et al., Science, 294(5543):853-858, 2001.
Lau et al., Science, 294(5543):858-862, 2001.
Lauffart et al., BMC Womens Health, 5:8, 2005.
Lee and Ambros, Science, 294(5543):862-864, 2001.
Lee et al., Int. J. Cancer, 118(10):2490-2497, 2006.
Lim et al., Nature, 433(7027):769-773, 2005.
Lin et al., Gastroenterology, 128(1):9-23, 2005.
Lu et al., Nature, 435(7043):834-838, 2005.
Marks, Semin. Cancer Biol., 16(6):436-443, 2006.
Morishita et al., Hepatology, 40(3):677-686, 2004.
Mrozek et al., Blood, 06-001149v1, 2006.
Nakayama et al., Cancer, 92(12):3037-3044, 2001.
Olsen et al., Dev. Biol., 216:671, 1999.
Ovcharenko et al., RNA, 11(6):985-93, 2005.
Pan et al., Neurol Res., 24(7):677-683, 2002.
Pennica et al., Proc. Natl. Acad. Sci. USA, 95(25):14717-14722, 1998.
Reinhart et al., Nature, 403(6772):901-906, 2000.
Romieu-Mourez et al., Cancer Res., 61 (9):3810-3818, 2001.
Rubin and Gutmann, Nat. Rev. Cancer, 5(7):557-564, 2005.
Rust et al., J. Clin. Pathol., 58(5):520-524, 2005.
Saigusa et al., Cancer Sci, 96(10):676-683, 2005.
Sambrook et al., In: DNA microaarays: a molecular cloning manual, Cold Spring Harbor Laboratory Press, Cold Spring Harbor, N.Y., 1989, 2001, 2003.
Sanchez-Aguilera et al., Blood, 103(6):2351-2357, 2004.
Sano et al., Histopathology, 46(5):532-539, 2005.
Saxena et al., Mol. Cell. Biochem., 228(1-2):99-104, 2001.
Scheit, In: Synthesis and Biological Function, Wiley-Interscience, New York, 171-172, 1980.
Schulze-Bergkamen et al., BMC Cancer, 6:232, 2006.
Seggerson et al., Dev. Biol, 243:215, 2002.
Shah et al., Oncogene, 21(54):8251-8261, 2002.
Shibahara et al., Anticancer Res., 25(3B): 1881-1888, 2005.
Shigemasa et al., Jpn. J. Cancer Res., 93(5):542-550, 2002.
Shimo et al., Cancer Lett., 174(1):57-64, 2001.
Sieghart et al., J. Hepatol., 44(1):151-157, 2006.
Sui et al., Oncol Rep., 15(4):765-771, 2006.
Takanami, Oncol. Rep., 13(4):727-731, 2005.
Takimoto et al., Biochem. Biophys. Res. Commun., 251(1):264-268, 1998.
Traub et al., Breast Cancer Res. Treat., 99(2):185-191, 2006.
Weil et al., Biotechniques., 33(6):1244-8, 2002.
Wu et al., Eur J Cancer, 38(14):1838-1848, 2002.
Wuilleme-Toumi et al., Leukemia, 19(7):1248-1252, 2005.
Xu et al., Curr. Biol., 13(9):790-795, 2003.
Yang et al., Cancer Res., 65(19):8887-8895, 2005.
Zangemeister-Wittke and Huwiler, Cancer Biol. Ther., 5(10):1355-1356, 2006.

1. A method of modulating gene expression in a cell comprising administering to the cell an amount of an isolated nucleic acid comprising a miR-16 nucleic acid sequence in an amount sufficient to modulate the expression of one or more gene identified in Table 1, 3, 4, or 5.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

3. The method of claim 2, wherein the infectious disease or condition is a parasitic, bacterial, viral, or fungal infection.

4. The method of claim 2, wherein the cancerous condition is astrocytoma, anaplastic large cell lymphoma, breast carcinoma, B-cell lymphoma, bladder carcinoma, cervical carcinoma, chronic lymphoblastic leukemia, colorectal carcinoma, endometrial carcinoma, glioma, glioblastoma, gastric carcinoma, hepatoblastoma, hepatocellular carcinoma, Hodgkin lymphoma, laryngeal squamous cell carcinoma, lung carcinoma, melanoma, medulloblastoma, mantle cell lymphoma, myxofibrosarcoma, myeloid leukemia, multiple myeloma, neurofibroma, non-small cell lung carcinoma, ovarian carcinoma, esophageal carcinoma, pancreatic carcinoma, prostate carcinoma, pheochromocytoma, renal cell carcinoma, rhabdomyosarcoma, squamous cell carcinoma of the head and neck, testicular tumor or thyroid carcinoma, wherein the modulation of one or more gene is sufficient for a therapeutic response.

5. The method of claim 4, wherein the cancerous condition is androgen dependent prostate carcinoma.

6. The method of claim 5, wherein the prostate carcinoma is associated with detectable prostate specific antigen (PSA, PSMA).

7. (canceled)

8. The method of claim 1, wherein the expression of a gene is down-regulated.

9. The method of claim 1, wherein the expression of a gene is up-regulated.

10. The method of claim 1, wherein the miR-16 nucleic acid is one or more of hsa-miR-16-1, hsa-miR-16-2, or a segment thereof.

11. The method of claim 1, wherein the miR-16 nucleic acid is an inhibitor of miR-16 function.

12. (canceled)

13. The method of claim 1, wherein the cell is a cancer cell.

14. The method of claim 13, wherein the cancer cell is a neuronal, glial, lung, liver, brain, breast, bladder, blood, cervical, leukemic, lymphoid, colon, endometrial, stomach, skin, ovarian, esophageal, pancreatic, prostate, kidney, testicular or thyroid cell.

15. The method of claim 1, wherein the isolated miR-16 nucleic acid is a recombinant nucleic acid.

16. The method of claim 15, wherein the recombinant nucleic acid is an RNA.

17. The method of claim 15, wherein the recombinant nucleic acid is DNA.

18. The method of claim 17, wherein the recombinant nucleic acid comprises a miR-16 expression cassette comprised in a viral vector or plasmid DNA vector.

19. (canceled)

20. The method of claim 18, wherein the viral vector is administered at a dose of 1×10⁵ to 1×10¹⁴ viral particles per dose or the plasmid DNA vector is administered at a dose of 100 mg per patient to 4000 mg per patient.

21. The method of claim 1, wherein the miR-16 nucleic acid is a synthetic nucleic acid.

22. The method of claim 21, wherein the nucleic acid is administered at a dose of 0.01 mg/kg of body weight to 10 mg/kg of body weight.

23.-25. (canceled)

26. The method of claim 1, wherein the nucleic acid is comprised in a pharmaceutical formulation.

27. The method of claim 26, wherein the pharmaceutical formulation is a lipid or nanoparticle composition.

28. (canceled)

29. The method of claim 26, wherein the pharmaceutical formulation consists of biocompatible and/or biodegradable molecules.

30. (canceled)

31. The method of claim 1, further comprising administering 2, 3, 4, 5, 6, or more miRNAs.

32.-45. (canceled)

46. A method of treating a patient diagnosed with or suspected of having or suspected of developing a pathological condition or disease related to a gene modulated by a miRNA comprising the steps of:
(a) administering to the patient an amount of an isolated nucleic acid comprising a miR-16 nucleic acid sequence in an amount sufficient to modulate a cellular pathway or a physiologic pathway; and
(b) administering a second therapy, wherein the modulation of the cellular pathway or physiologic pathway sensitizes the patient to the second therapy.

47. (canceled)

48. A method of selecting a miRNA to be administered to a subject with, suspected of having, or having a propensity for developing a pathological condition or disease comprising:
(a) determining an expression profile of one or more genes selected from Table 1, 3, 4, and 5;
(b) assessing the sensitivity of the subject to miRNA therapy based on the expression profile; and
(c) selecting one or more miRNA based on the assessed sensitivity.

49.-53. (canceled)


SEQUENCE LISTING (3 sequences)

SEQ ID NO: 1 - RNA, 22 nt, Homo sapiens
uagcagcacguaaauauuggcg

SEQ ID NO: 2 - RNA, 89 nt, Homo sapiens
gucagcagugccuuagcagcacguaaauauuggcguuaagauucuaaaauuaucuccaguauuaacugugcugcugaaguaagguugac

SEQ ID NO: 3 - RNA, 81 nt, Homo sapiens
guuccacucuagcagcacguaaauauuggcguagugaaauauauauuaaacaccaauauuacugugcugcuuuagugugac